(19) United States
(12) Patent Application Publication
(10) Pub. No.: US 2024/0376155 A1
(43) Pub. Date: Nov. 14, 2024

(54) NOVEL COMPOUNDS AND COMPOSITIONS FOR TARGETED THERAPY OF RENAL CANCERS

(71) Applicant: SHANGHAI MICURX PHARMACEUTICAL CO., LTD., New District, Shanghai (CN)

(72) Inventors: Mikhail Fedorovich Gordeev, Foster City, CA (US); Jinqian Liu, Foster City, CA (US); Shouning Xu, Shanghai (CN); Xinghai Wang, Shanghai (CN)

(21) Appl. No.: 18/580,921

(22) PCT Filed: Jul. 21, 2022
(86) PCT No.: PCT/CN2022/106976

§ 371 Date: Jan. 19, 2024

Related U.S. Application Data
(60) Provisional application No. 63/224,406, filed on Jul. 21, 2021.

(51) Int. Cl. C07K 7/62; A61K 38/12; A61K 47/64; A61P 35/00

(52) U.S. Cl. CPC C07K 7/62; A61K 38/12; A61K 47/64; A61P 35/00

(57) ABSTRACT

Provided are therapeutic compounds, for example compounds of Formula I, or pharmaceutically acceptable salts, hydrates, or solvates thereof that are therapeutic or anticancer agents, pharmaceutical compositions thereof, methods for their use, and methods for preparing these compounds.

### CROSS-REFERENCE TO RELATED APPLICATIONS

This application claims the benefit of U.S. Provisional Application No. 63/224,406, filed Jul. 21, 2021, the entirety of which is hereby incorporated by reference for all purposes.

### FIELD

Provided herein are novel active compounds, pharmaceutical compositions thereof, methods for their use, and methods for preparing the same. These novel agents and compositions possess therapeutic activities useful in the therapy of kidney-associated cancers.

### BACKGROUND

Cancers comprise a multi-faceted class of deadly diseases with a profound impact on human lives worldwide. Among these, renal cell carcinoma (RCC) is the most common form of kidney cancer. Globally, RCC is diagnosed in over 200,000 patients annually (as reviewed, for example, by Escudier and Gore in Drugs R&D. 2011, vol. 11, p. 113). This serious disease accounts for about 100,000 deaths annually. Alarmingly, the incidence of RCC is increasing. A 126% increase in incidence and a 36.5% increase in mortality since 1950 was reported for US alone. In particular, metastatic RCC (mRCC) is known to be highly resistant to conventional therapy, with a low 5-year survival rate for a commonly diagnosed stage IV disease of only 0-10% (Motzer et al. N. Engl. J. Med. 1996, vol. 335, pp. 865-75).

A small number of pharmaceutical agents have been developed for the treatment of several forms of renal cancers, including renal cell carcinoma (RCC) and metastatic RCC (mRCC), including standard-of-care drugs used to treat renal cancers, such as axitinib and sunitinib. However, anticancer drugs typically exhibit high levels of undesired adverse effects, severely limiting therapeutic utility thereof. These adverse effects could be generally ascribed to cytotoxicity of the anticancer drugs. A cytotoxic mode of activity of chemotherapeutic agents is required for anticancer therapeutic effect thereof. As a result, virtually all anticancer pharmaceuticals are inherently cytotoxic. This toxicity may manifest as adverse effects, including serious adverse effects, with mortality often attributable to chemotherapy. Thus, one of the current standard-of-care drugs for treatment of renal cancer, sunitinib, is known to exhibit high incidence of hematotoxicity (as reported, for example, by Kato et al. in BMC Cancer. 2017, vol. 17, p. 214). This undesired toxicity (also referred to as myelosuppression or a bone marrow toxicity) severely restricts the use of sunitinib in some patient populations, potentially limiting the prescribed dosing regimen required for an optimal anticancer effect (Kato et al. in BMC Cancer. 2017, vol. 17, p. 214). A fatality due to toxicity of such drugs was reported. For example, Prescribing Information for the renal cancer drug axitinib (Inlyta^{R}) includes Warnings on severe hypertension (including hypertensive crisis) and that cardiac failure has been observed for the drug and can be fatal (as described in Prescribing Information. INLYTA-Axitinib Tablet. June 2020, Pfizer). These adverse effects could be generally ascribed to an off-target action of cytotoxic chemotherapeutic compounds, wherein the inherent mode of action toxicity impacts unintended biological compartments, such as bone marrow or heart. Related to such toxicity, therapy-induced “bystander killing” of healthy human cells in proximity of cancer cells was also reported (see, for example, by Staudacher and Brown in British Journal of Cancer. 2017, vol. 117, p. 1736).

Therefore, safer anticancer agents are urgently needed. More specifically, novel anti-cancer therapies must offer an improved selectivity of cytotoxic effects that target only cancerous cells of the affected biological compartments (organs), while leaving healthy tissues and organs minimally affected.

One emerging approach to achieve improved selectivity of anticancer drugs is a targeted delivery of active, but toxic agents, solely to an organ affected by the disease or, even more specifically, to cancerous cells therein (as reviewed, for example by Tekewe et al. Int. J. Pharm Sci. Res. 2013; Vol. 4, p. 1). In recent years, this urgent need has prompted an emergence of monoclonal antibody drug conjugates (ADCs) that harness innate affinity of antibody(ies) towards the cancer cells, with subsequent release of an anticancer drug “payload” directly at the target site (as reviewed, for example, by Cazzamalli et al. in J. Am. Chem. Soc. 2018, vol. 140, p. 1617). However, development of ADCs as viable therapeutics presents several serious challenges, including high cost-of-goods to manufacture, variability in active payload/antibody ratios that require specialized bioanalytical characterization, relatively low chemical stability, excessively long circulation time in vivo, toxic payload release in unintended biological compartments, and limited ability of ADCs to penetrate into solid tumors, such as kidney-associated cancers.

Other approaches include efforts to achieve target drug delivery using non-antibody constructs, such as organic molecule ligands (target-complexing structures), also referred to as small molecule—drug conjugates (SMDCs), that typically utilize molecules capable of recognition of certain targets present in cancer cells, such as folate receptor, prostate-specific membrane antigen, somatostatin receptors and carbonic anhydrase IX (as cited by Cazzamalli et al. in J. Am. Chem. Soc. 2018, vol. 140, p. 1617). However, this approach is limited by serious difficulty in identification of unique small molecules capable of selective recognition of cancer-affected organ(s). Furthermore, the majority of such ligands comprise linear peptides that are generally unstable in vivo due to their rapid metabolism by ubiquitous peptidase enzymes present throughout the body (as reviewed, for example, by Page and Cera in Cell. Mol. Life Sci. 2008, vol. 65, p. 1220).

Provided herein are unique derivatives of acyclic and cyclic peptides (cyclopeptides) particularly suitable for a targeted therapy of various cancers, including kidney cancers.

Various cyclopeptides have been described, for example, in publications WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687. Certain acyclic peptide structures with potential for targeted delivery of active agents have been described, for example in the publications WO 2019136298, US 20180015173, WO 2021/150792, and references cited therein. None of these references specifically describe or generally contemplate the compositions provided herein.

### SUMMARY

Provided herein are novel compounds and composition useful for targeted therapy of cancers, in particular, kidney-associated cancers.

These novel compounds are surprisingly able to target kidney tissues and, in particular, cancerous cells therein. The unique affinity of the compositions described herein to tissues affected by kidney cancers permits a selective delivery to and accumulation of such molecules at the site of the cancer, with minimal or no accumulation of these therapeutic agents in other healthy tissues.

As a result, a selective and generally safer anticancer therapy is achieved, with significantly minimized adverse effect(s) on other normal organs of a mammal under therapy, for example as compared to current standard-of-care drugs used to treat kidney cancers, such as axitinib, brivanib, pazopanib, and sunitinib.

In one embodiment, the therapeutic action of the compounds described herein is achieved by release of one or more of an anticancer element(s) (for example, bioactive payloads and/or drugs) incorporated into the compound. The active payload (drug) may comprise a cytotoxic structure, antibody structure, and/or immunomodulating structure, selected from bioactive structures with the ability to kill cancer cells or inhibit growth of cancer cells, or activate immunomodulating response resulting in similar anticancer action.

Generally, the compounds provided herein are comprised of a peptide, cyclopeptide, or another “target seeker” (ligand) structure with a high affinity (ability to bind) towards kidney cancer and/or kidney cells, along with the active drug(s) substructure, within a single molecule. The active drug(s) (payloads) is(are) connected to a kidney-affinity structure via a framework of uniquely designed linker(s) and spacer(s). This unique design allows for an efficient release of an active drug (payload) directly into kidney cancer cells, or in close proximity thereof, resulting in a targeted anticancer effect.

In one embodiment, said composition possesses cytotoxic property(ies) against cancer cells, without a release of an active drug payload (comprised within the administered structure) at the site of a kidney cancer. Upon accumulation at the site of a kidney cancer, such compound(s) kill(s) or inhibit(s) growth of cancer cells directly, and may subsequently break-down into generally non-toxic metabolites.

In yet another embodiment, the compositions exhibit modest or no innate anticancer cytotoxicity as intact molecules, but instead accumulate in kidneys and then are metabolized in the organ affected by renal cancers, thereby releasing an anticancer drug (or a cytotoxic agent), at the site of a cancer, to result in anticancer therapeutic effect.

In yet another embodiment, the anticancer effect is achieved (upon accumulation at the cancer site) through a combined effect of (i) direct cytotoxic effect of said compound(s), and (ii) a release of an active payload drug comprised within the structure.

In yet another aspect is provided a cyclic peptide conjugate of a tyrosine kinase inhibitor. In some or any embodiments, the cyclic peptide is a polymyxin cyclic peptide as provided herein.

Surprisingly, certain compounds and compositions provided herein are devoid of significant antibiotic and/or other biological activity (such as antibacterial activity), and only exert the desired cytotoxic effect on kidneys affected by a cancer disease.

Furthermore, while certain compositions provided herein incorporate cyclopeptide moieties (structures) of chemical classes generally known to cause renal toxicity (such as polymyxins), the therapeutic compounds described herein exhibit little or no renal toxicity at the therapeutic dosing levels required for treatment of kidney cancers.

One skilled in art would readily appreciate that not every molecular construct incorporating cytotoxic element(s) (payload) with “heat-seeker” affinity structure (ligand targeting kidney and/or renal cancer cells) with appropriate linkers and deliberately positioned spacers (strategically placed between a ligand and cytotoxic payload) is suitable for use as a therapeutic agent. Surprisingly, compounds and compositions provided herein possess a good pharmacological profile, with appropriate stability in blood plasma that precludes premature cytotoxic action, coupled with preferential accumulation thereof in renal cancer cells and/or in kidney affected by a renal cancer(s).

Even more surprisingly, certain compounds provided herein exert their cytotoxic anticancer effect by self-targeted delivery either directly into kidney cancer cells, or only in close proximity of cancer-affected tissues. In part, this composition comprises a class of molecules capable of specifically releasing cytotoxic payloads (incorporated within their structures) as a result of metabolic cleavage by classes of enzymes either specific to or overexpressed (enriched) within the cancer cells (such as cathepsin, glutaminase, and peptide deformylase enzyme (PDF), peptidases, reductases, and similar known enzymes).

In addition to a metabolic degradation by classes of enzymes overexpressed in cancer cells (such as cathepsin, glutaminases, PDF, or similar enzymes) certain compounds provided herein are degraded in vivo through a chemical cleavage, such as pH-dependent self-cleavage known for molecules bearing both a cleavable group (such as an ester, an amide, or a carbamate group) and a free nucleophilic group (such as amine, alcohol, or thiol group). When these two types of cleavable and nucleophilic groups are in certain spacial proximity to each other, and the nucleophile group is essentially free (for example, amine group under neutral, basic, or physiological pH conditions), the nucleophilic group may be acylated by the ester group, resulting in the acyl group transfer onto the nucleophilic atom (such as nitrogen atom in amine group). In another scenario, the free amine may activate an adjacent to the carbamate group amide functionality, leading to the carbamate reaction with the latter, to result in a conversion of the native amide into a bis-acylated imide group. In some compositions herein, the cleavage of a chemical designer linker takes place after initial enzymatic metabolism of an auxiliary enzyme-cleavable linker (e.g., peptide substructure, or similar linkers), to overall effect the release of a cytotoxic payload at the cancer target.

In one aspect, provided herein is a compound of Formula I-P-1:

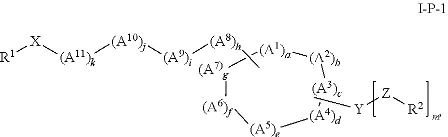

or a pharmaceutically acceptable salt, solvate, or hydrate thereof wherein:
R¹ and R² are optional groups, with at least one of the groups R¹ and R² being present in the Formula I-P-1; and
R¹ and R² are independently selected from alkyl, aryl, biaryl, heteroaryl, heteroarylaryl, and arylheteroaryl; or
R¹ and R² are residues independently attached to X and Z, respectively, by subtracting a single or multiple H atom(s) from respective parent (precursor) structure(s) (H)ₙR¹ and (H)ₒR² at any one of the following optional H-containing group(s) independently selected from NH, OH, SH, C(═O)OH, CONH, SO₂NH, and S(═O)NH when present in (H)ₙR¹ and (H)ₒR²; and wherein
a) (H)ₙR¹ and (H)ₒR² are independently compound(s) possessing biological or therapeutic activity; or
b) (H)ₙR¹ and (H)ₒR² are independently a cytotoxic compound(s), an antibody(ies), or an immunomodulating compound(s) possessing an activity or capable of inducing an activity against one or more cancer cells, including compounds with activity against one or more renal cancer cells; or
c) (H)ₙR¹ and (H)ₒR² are independently mono- or multi-valent antibody(ies) with activity against one or more cancer cells; or
d) (H)ₙR¹ and (H)ₒR² are independently afatinib ((E)-N-[4-(3-chloro-4-fluoroanilino)-7-[(3S)-oxolan-3-yl]oxyquinazolin-6-yl]-4-(dimethylamino)but-2-enamide), ARS-1630 ((R)-1-(4-(6-chloro-8-fluoro-7-(2-fluoro-6-hydroxyphenyl)quinazolin-4-yl)piperazin-1-yl)prop-2-en-1-one), axitinib (N-methyl-2-[[3-[(E)-2-pyridin-2-ylethenyl]-1H-indazol-6-yl]sulfanyl]benzamide), BGB-324 (1-(6,7-dihydro-5H-benzo[2,3]cyclohepta[2,4-d]pyridazin-3-yl)-3-N-[(7 S)-7-pyrrolidin-1-yl-6,7,8,9-tetrahydro-5H-benzo[7]annulen-3-yl]-1,2,4-triazole-3,5-diamine), BLU-554 (N-[(3 S,4S)-3-[[6-(2,6-dichloro-3,5-dimethoxyphenyl)quinazolin-2-yl]amino]oxan-4-yl]prop-2-enamide), brivanib ((S)—(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate), (R)-1-((4-((4-fluoro-2-methyl-1 H-indol-5-yl)oxy)-methylpyrrolo[2,1-f][1,2,4]triazin-6-yl)oxy)propan-2-ol, cabozantinib, cediranib, ceritinib, ciforadenant, derazantinib, dovitinib (same as 4-amino-5-fluoro-3-(6-(4-methylpiperazin-1-yl)-1H-benzo[d]imidazol-2-yl)quinolin-2(1H)-one), E-7046, (same as 4-[(1S)-1-[[3-(difluoromethyl)-1-methyl-5-[3-(trifluoromethyl)phenoxy]pyrazole-4-carbonyl]amino]ethyl]benzoic acid), emtansine, englerin ((1R,3aR,4S,5R,7R,8S,8aR)-5-(glycoloyloxy)-7-isopropyl-1,4-dimethyldecahydro-4,7-epoxyazulen-8-yl (2E)-3-phenylacrylate), foretinib, lenvatinib (4-[3-chloro-4-(cyclopropylcarbamoylamino)phenoxy]-7-methoxyquinoline-6-carboxamide), monomethyl auristatin E ((S)—N-((3R,4S,5S)-1-((S)-2-((1R,2R)-3-(((1 S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N, 3-dimethyl-2-((S)-3-methyl-2-(methylamino)butanamido)butanamide), irinotecan, maytansinoid, neratinib, nilotinib, nintedanib, ozogamicin, paclitaxel, pazopanib (5-[[4-[(2,3-dimethylindazol-6-yl)-methylamino]pyrimidin-2-yl]amino]-2-methylbenzenesulfonamide), regorafenib, sacituzumab, selpercatinib, semaxanib ((Z)-3-((3,5-dimethyl-1 H-pyrrol-2-yl)methylene)indolin-2-one), sorafenib (4-[4-[[4-chloro-3-(trifluoromethyl)phenyl]carbamoylamino]phenoxy]-N-methylpyridine-2-carboxamide), sunitinib ((Z)—N-(2-(diethylamino)ethyl)-5-((5-fluoro-2-oxoindolin-3-ylidene)methyl)-2,4-dimethyl-1H-pyrrole-3-carboxamide), SN-38 (7-ethyl-10-hydroxy-camptothecin), sorafenib (4-[4-[[4-chloro-3-(trifluoromethyl)phenyl]carbamoylamino], phenoxy]-N-methyl-pyridine-2-carboxamide), trastuzumab, tesirine (same as [4-[[(2S)-2-[[(2S)-2-[3-[2-[2-[2-[2-[2-[2-[2 [2-[3-(2,5-dioxopyrrol-1-yl)propanoylamino]ethoxy]ethoxy]ethoxy]ethoxy]ethoxy]ethoxy]ethoxy]ethoxy]propanoyla mino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy]pentoxy]-6-hydroxy-2-methoxy-8-methyl-11-oxo-6a,7-dihydro-6H-pyrrolo[2,1-c][1,4]benzodiazepine-5-carboxylate), temsirolimus ([(1R,2R,4S)-4-[(2R)-2-[(1R,9S,12S,15R,16E,18R,19R,21R,23 S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl] 3-hydroxy-2-(hydroxymethyl)-2-methylpropanoate), tivantinib, tivozanib (1-{2-chloro-4-[(6,7-dimethoxyquinolin-4-yl)oxy]phenyl}-3-(5-methylisoxazol-3-yl)urea), vatalanib, veliparib, or vinblastine; or a varianttherof thereof, or
e) (H)ₙR¹ and (H)ₒR² are independently compound(s) active against a kidney cancer disease; or
f) (H)ₙR¹ is a heterocyclic structure(s) connected to X at one of heterocyclic nitrogen atom(s) present within the structure (H)ₙR¹; wherein said nitrogen atom becomes a nitrogen atom with a single positive charge, such as imidazolium, pyrazolium, pyridinium, or indazolium group; and
when an optional group R¹ is absent, then the fragment R¹X is replaced with R^{11a} wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is replaced with R^{12a} wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
integers n and o are independently selected from 0, 1, 2, 3, 4, 5, 6, and 7, such that [n+o]≥1; and
A¹ through A¹¹ are independently optional and when present are independently selected from amino acid residues independently selected from unsubstituted or substituted at any N atom and where each of the amino acid residues, when present, is independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[30.2.1]octane-2-carboxylic acid, 4-amino-8-azabicyclo[3.2.1]octane-2-carboxylic acid, 3-amino-8-azabicyclo[30.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers a through k, m, and zz are independently selected from 0, 1, and 2, and wherein
[m+zz]≥1; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, and heterocyclyl; or the group A⁸ is directly connected to the group Y; and
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene, (C₁₋₁₂alkylene)OC(═O), OC(═O)(C₁₋₁₂alkylene), (C₁₋₁₂alkylene)OC(═O), C(═O)O(C₁₋₁₂alkylene), (C₁₋₁₂alkylene)C(═O), N(R⁵)C(═O)(C₁₋₁₂alkylene), (C₁₋₁₂alkylene)N(R⁵)C(═O), C(═O)N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R₈)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)CR⁵═CR⁷—S—S—(CR⁹R₁₀)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH₂CH₂CH₂C(═O), C(═O)N{CH₂CH₂NHC(═O)—[(R)—CH(NH₂)]CH₂CH₂COOH₂CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH₂CH₂CH₂C(═O), C(═O)N{CH₂CH₂NHC(═O)—[(R)—CH(NH₂)]CH₂COOH₂CH₂CH₂C(═O), C(═O)N{CH₂CH₂NHC(═O)CH[NHC(═O)H]CH₂CH₂COOH}CH₂H₂C(═O), C(═O)N{CH₂CH₂NHC(═O)CH[NHC(═O)Me]CH₂CH₂COOH}CH₂H₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene-C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene-C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), and C(═O)N[CH₂CH₂NHC(═O)CH(NH₂)CH₂CH₂COOC₁₋₆alkyl]CH₂CH₂C(═O);
or any variant of the above groups X formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein
R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁶ and R⁷, or ii) R⁹ and R¹⁰, together with the atom to which they are attached form a saturated or unsaturated C₃₋₆cycloalkylene; or wherein
any two of R⁵ through R¹⁰ together with the atom(s) to which they are attached form a to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³ with the following structures: (C₁₋₁₂alkylene)OC(═O), OC(═O)(C₁₋₁₂alkylene), (C₁₋₁₂alkylene)OC(═O), C(═O)O(C₁₋₁₂alkylene), (C₁₋₁₂alkylene)C(═O), N(R⁵)C(═O)(C₁₋₁₂alkylene), (C₁₋₁₂alkylene)N(R⁵)C(═O), C(═O)N(R⁵)(C₁₋₁₂alkylene), C(═O)(CR⁵R₆)ₚ(CR⁷R⁸)ᵣ(CR⁹R¹⁰)ₛC(═O), C(═O)O(CR⁵R₆)O(CR⁷R⁸)ᵣ(CR⁹R¹⁰)ₛC(═O), P(═O)(OCR⁵R₆)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R₆)ₘ, P(═O)(NHCR⁵R₆)ₚ(CR⁷R⁸)ᵣ(CR⁹R¹⁰)ₛC(═O), P(═O)(OCR⁵R₆)CF₂, P(═O)(OCR⁵R₆)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R₆)ₚ, C(═O)O(CR⁵R₆)ₚ(CR⁷R⁸)ᵣ(CR⁹R¹⁰)ₛC(═O), C(═O)N(R⁵)(CR⁵R₆)ₚ(CR⁷R⁸)ᵣ(CR⁹R¹⁰)ₛC(═O), C(═O)(CR⁵R₆)ₚ(CR⁷R⁸)ᵣ(CR⁹R¹⁰)ₛOC(═O), C(═O)(CR⁵R₆)ₚ(CR⁷R⁸)ᵣ(CR⁹R¹⁰)ₛN(R⁵)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)r(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R′)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R′)C(═O), C(═O)CR^{S}═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), or C(═O)N[CH₂CH₂NHC(═O)CH(NH₂)CH₂CH₂COOC₁₋₆alkyl]CH₂CH₂C(═O);
or any variant of the above groups X formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates additional divalent groups selected from C₁₋₁₂alkylene, C₂₋₂alkenylene, C₂₋₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₄alkyl)C(═O), N(C₁₋₄alkyl)(CH₂)ₚO(CH₂)ᵣO(CH₂)ₛC(═O), N(C₁₋₄alky)(CH₂)ₚO(CH₂)ᵣO(CH₂)ₛOC(═O), N(C₁₋₄alkyl)(CH₂)ₚO(CH₂)ᵣO(CH₂)ₛNHC(═O), N(C₁₋₄alkyl)(CH₂)ₚO(CH₂)ᵣO(CH₂)ₛN(C₁₋₄alkyl)C(═O), and similar linear groups;
optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene, (C₁₋₁₂alkylene)OC(═O), OC(═O)(C₁₋₁₂alkylene), (C₁₋₁₂alkylene)OC(═O), C(═O)O(C₁₋₁₂alkylene), (C₁₋₁₂alkylene)C(═O), N(R⁵)C(═O)(C₁₋₁₂alkylene), (C₁₋₁₂alkylene)N(R⁵)C(═O), C(═O)N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O) C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁵)C(═O) C(═O)CR⁵═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR—(CR⁹R¹⁰)ₛOC(═O), C(═O)CR⁵═CR⁷—(CR⁹R¹⁰)ₛN(R⁵)C(═O); C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), and C(═O)N[CH₂CH₂NHC(═O)CH(NH₂)CH₂CH₂COOC₁₋₆alkyl]CH₂CH₂C(═O);
or any variant of above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein R⁵—R¹⁰ are as defined above; or alternatively, the optional group Z is comprised of the following structures, optionally connected to one to two amino acid residue(s) A¹² or A¹³, with the following structures: (C₁₋₁₂alkylene)OC(═O), OC(═O)(C₁₋₁₂alkylene), (C₁₋₁₂alkylene)OC(═O), C(═O)O(C₁₋₁₂alkylene), (C₁₋₁₂alkylene)C(═O), N(R⁵)C(═O)(C₁₋₁₂alkylene), (C₁₋₁₂alkylene)N(R⁵)C(═O), C(═O) N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O) C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R′)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R′)C(═O), C(═O)CR^{S}═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR—(CR⁹R¹⁰)ₛOC(═O), C(═O)CR⁵═CR⁷—(CR⁹R¹⁰)ₛN(R⁵)C(═O); C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), or C(═O)N[CH₂CH₂NHC(═O)CH(NH₂)CH₂CH₂COOC₁₋₆alkyl]CH₂CH₂C(═O);
or any variant of above groups Z formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸.

In an alternative embodiment of Formula I-P-1, R⁵ and R⁶ together with the atom to which they are attached form a saturated or unsaturated C₃₋₆cycloalkylene.

In an alternative embodiment of Formula I-P-1, fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present and [p+r+s]≥1.

In an alternative embodiment of Formula I-P-1, fragments (CR⁵R⁶)ₚ(CR⁷R⁸)ᵣ or (OCR⁵R⁶)ₚ(CR⁷R⁸)ᵣ are present and [p+r]≥1.

In an alternative embodiment of Formula I-P-1, each optional divalent group X is selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), and C(═O)CR^{S}═CR—(CR⁹R¹⁰)ₛOC(═O), C(═O)CR^{S}═CR⁷—(CR⁹R¹⁰)ₛN(R⁴)C(═O), or any variant of the above X groups formed by the repositioning, addition, or deletion of the fragment N(R⁴)C(═O) or C(═O)N(R⁴)SO₂; or
X is independently selected from C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)(CR⁵R⁶)ₚ(CR⁷R⁸)ᵣ(CR⁹R¹⁰)ₛOC(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O), and C(═O)(CR⁵R⁶)ₚ(CR⁷R⁸)ᵣ(CR⁹R¹⁰)ₛN(R⁵)C(═O);
wherein R⁸ is independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl; or R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle.

In an alternative embodiment of Formula I-P-1, each optional divalent group X is selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), and C(═O)CR^{S}═CR⁷—(CR⁹R¹⁰)ₛN(R⁴)C(═O), or any variant of the above X groups formed by the repositioning, addition, or deletion of the fragment N(R⁴)C(═O) or C(═O)N(R⁴)SO₂;
wherein the group X is optionally connected to one to two amino acid residue(s) A¹² or A¹³; or
X is independently selected from C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O),
wherein the group X is optionally connected to one to two amino acid residue(s) A² or A¹³.

In an alternative embodiment of Formula I-P-1, each optional divalent group X is S(CR⁵R⁶)ₚ(CR⁷R⁸)ᵣ(CR⁹R¹⁰)ₛOC(═O) or a group selected from

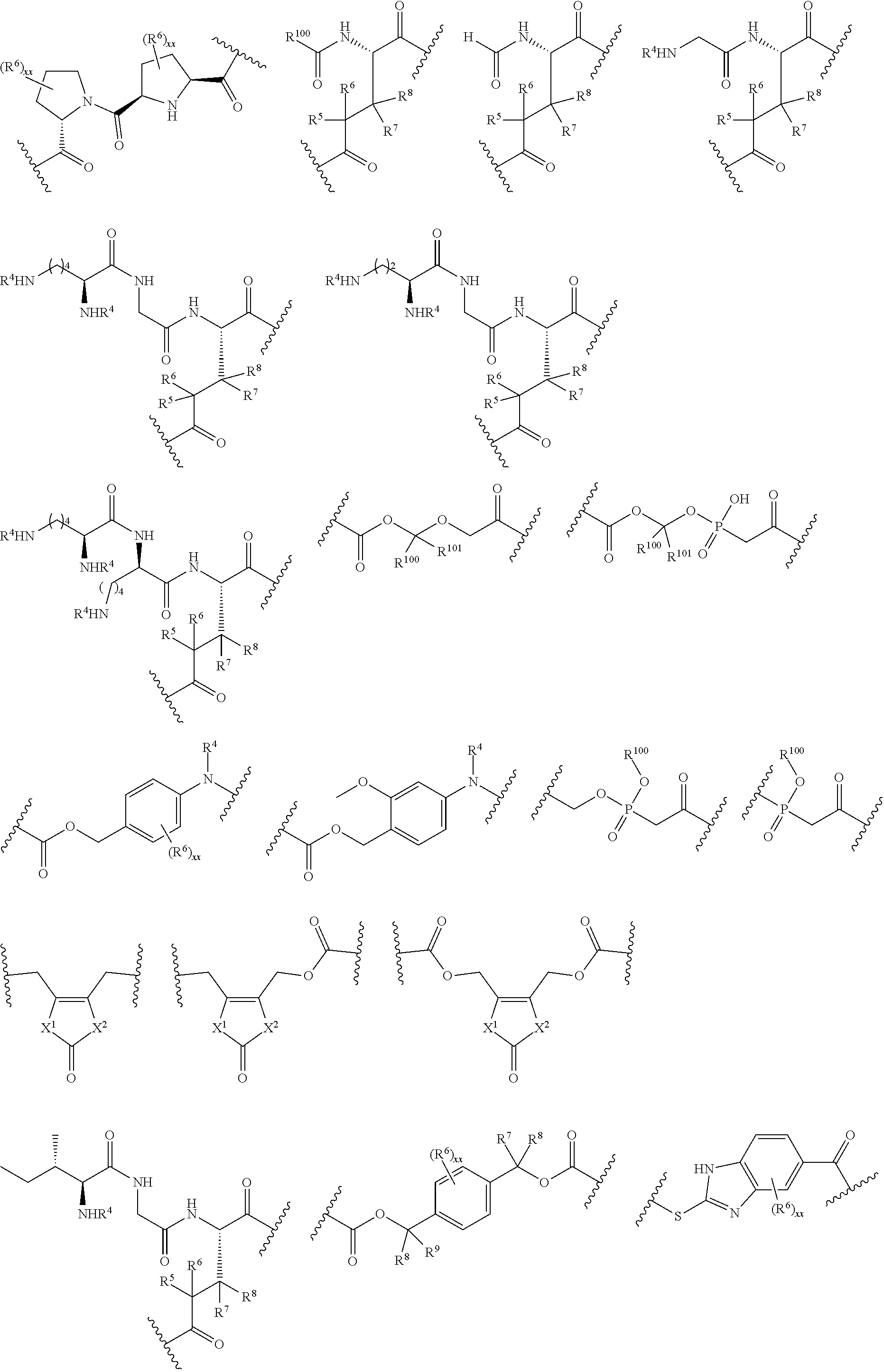

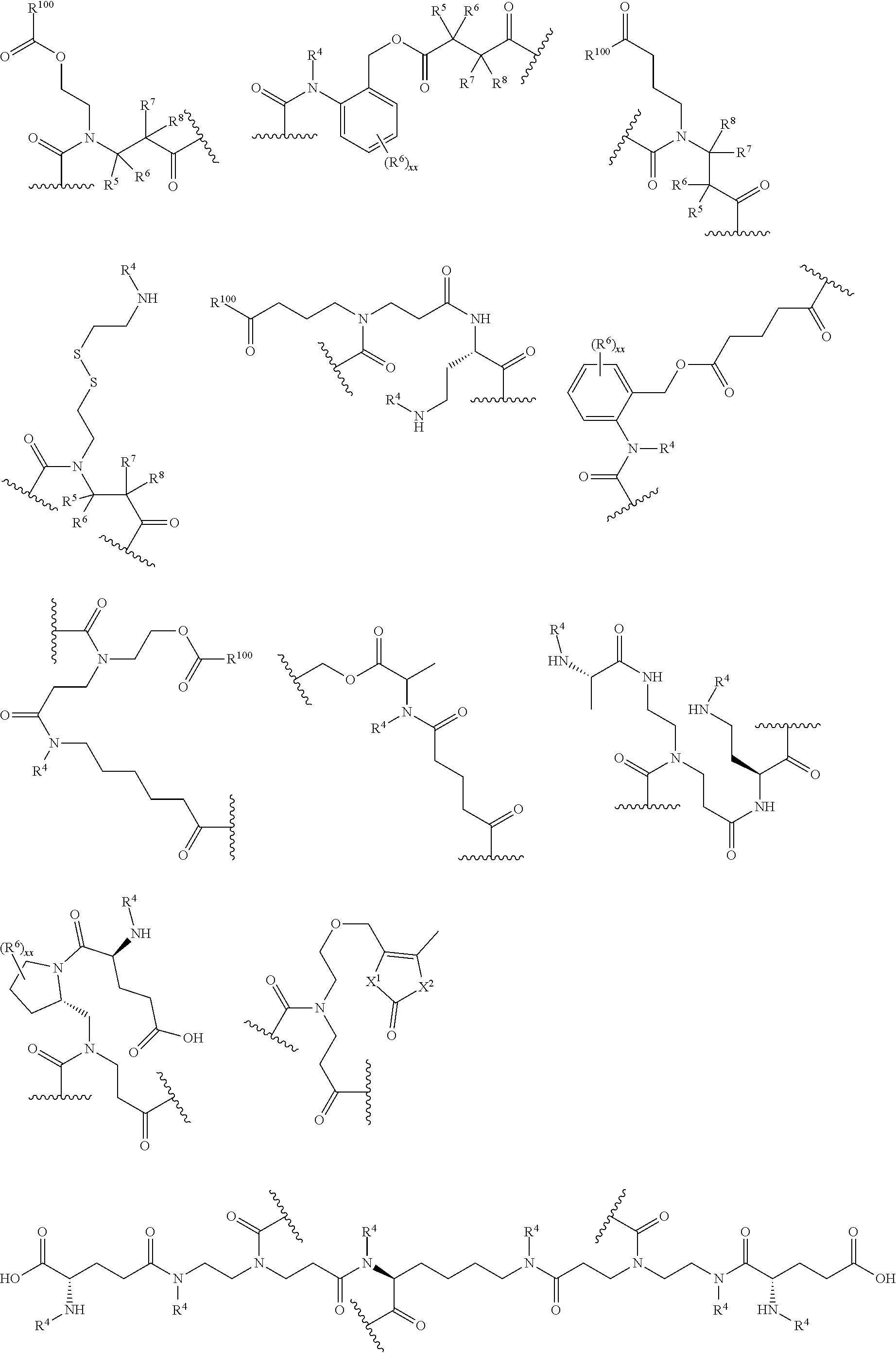

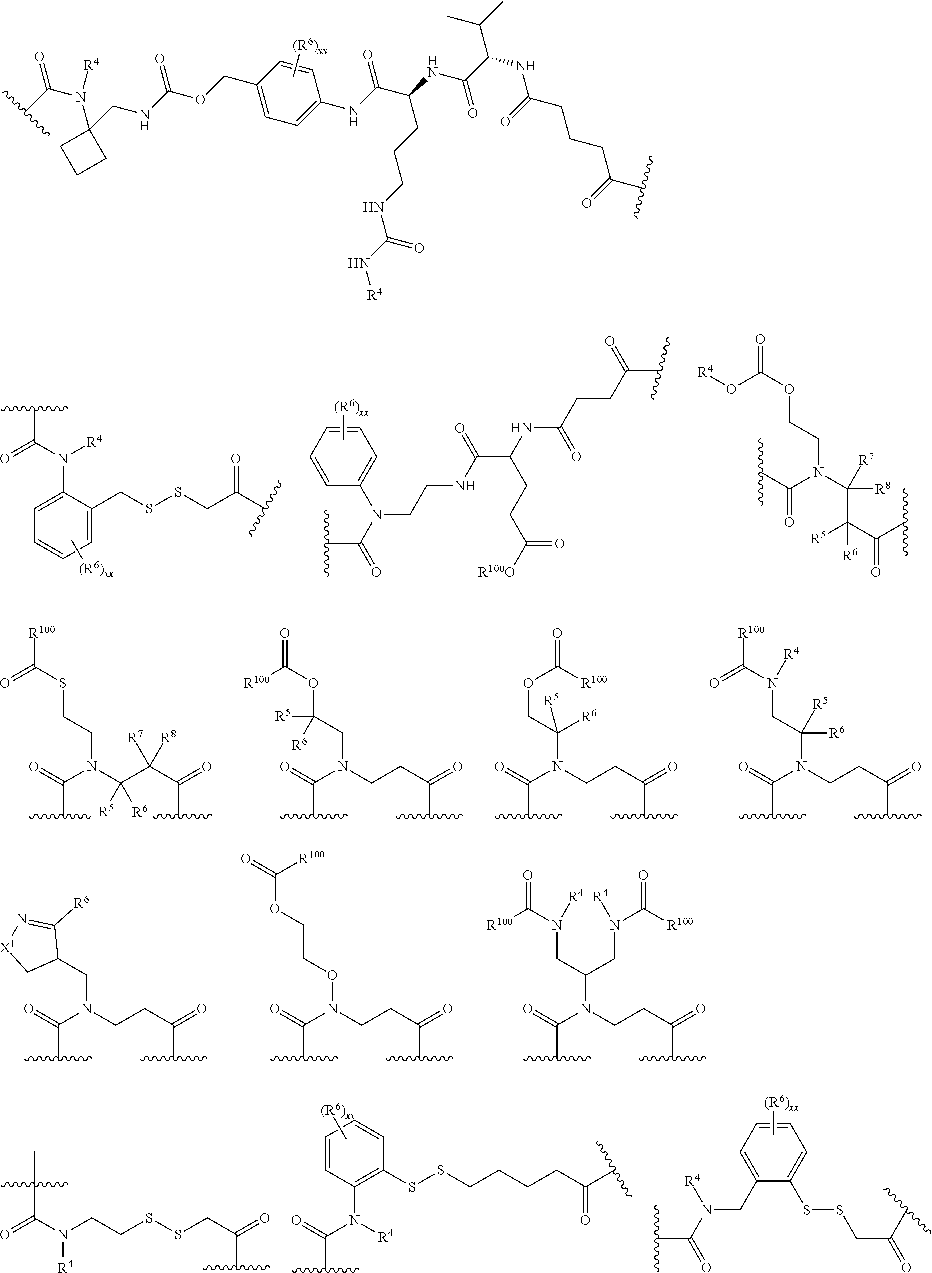

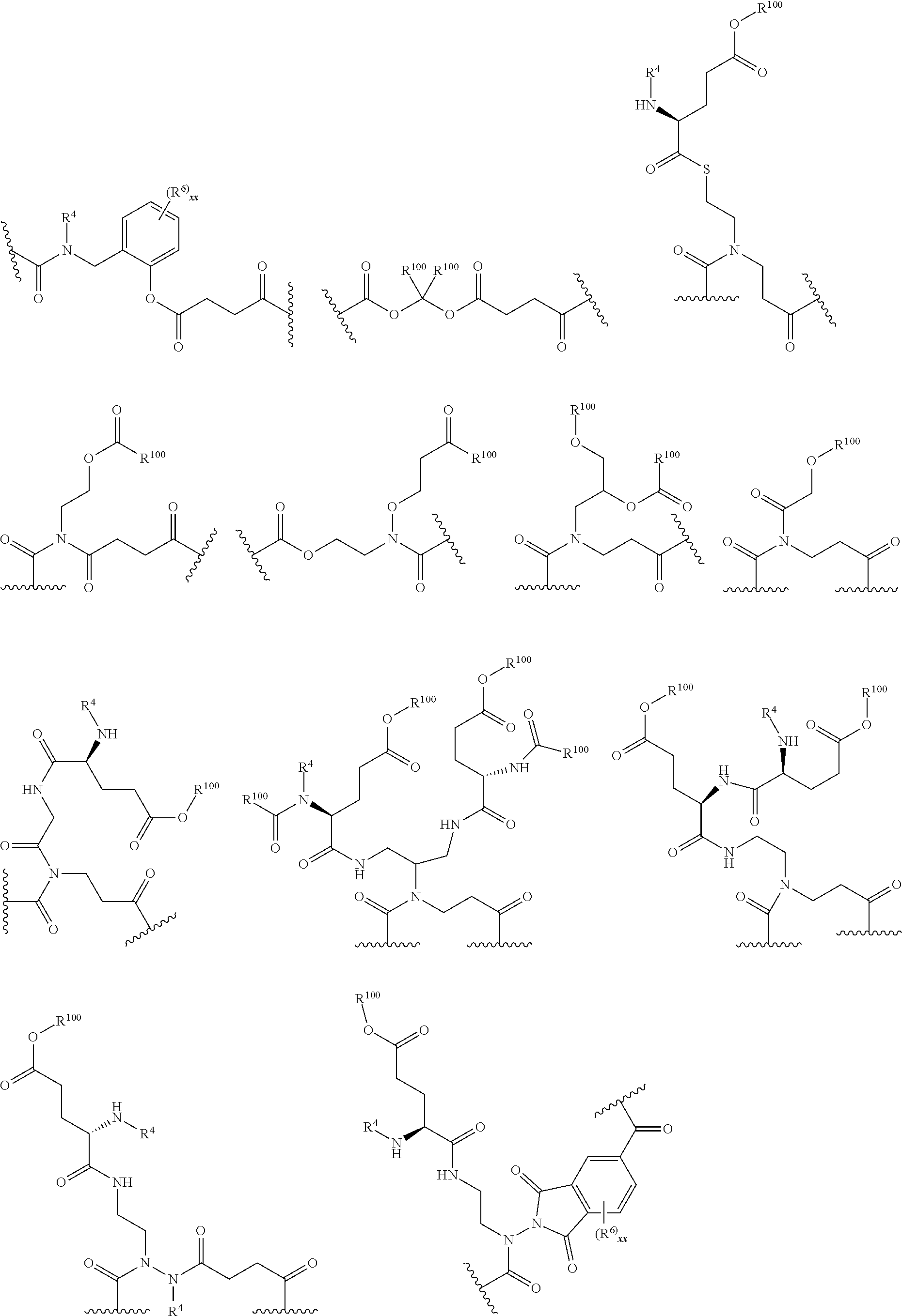

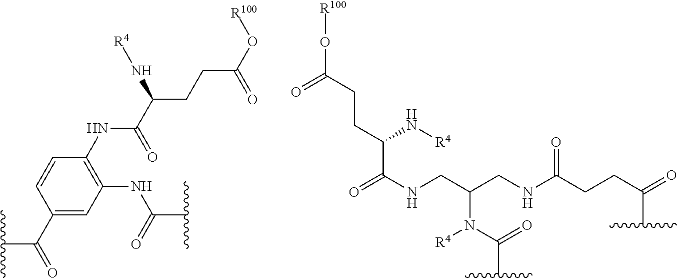

and any variant of the above X groups formed by the repositioning, addition, or deletion of the fragment N(R⁴)C(═O); or
wherein the group X is optionally connected to one to two amino acid residue(s) A¹² or A¹³;
wherein xx is 1, 2, or 3;
R¹⁰⁰ and R¹⁰¹ are independently selected from hydrogen and alkyl;
X¹ and X² are independently selected from O, NH, and CR⁵R⁶; and
all other variables are as defined herein in Formula I-P-1.

In an alternative embodiment of Formula I-P-1, each optional divalent group Y and Z are independently selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), and C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), or any variant of the above groups formed by the repositioning, addition, or deletion of the fragment N(R⁴)C(═O) or C(═O)N(R⁴)SO₂; or
each optional divalent group Y and Z are independently selected from C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O); and C(═O)(CR⁵R⁶)ₚS—S(CR⁷R⁸)ᵣS—S(CR⁹R¹⁰)ₛOC(═O).

In an alternative embodiment of Formula I-P-1, each optional divalent group Z is independently selected from N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), and C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), or any variant of the above groups formed by the repositioning, addition, or deletion of the fragment N(R⁴)C(═O) or C(═O)N(R⁴)SO₂;
wherein the group Z is optionally connected to one to two amino acid residue(s) A¹² or A¹³; or
each optional divalent group Z is independently selected from C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O), and C(═O)(CR⁵R⁶)ₚ(CR⁷R⁸)ᵣ(CR⁹R¹⁰)ₛN(R)C(═O);
wherein the group Z is optionally connected to one to two amino acid residue(s) A¹² or A¹³.

In one embodiment, the compound of Formula I-P-1 is a compound of Formula I:

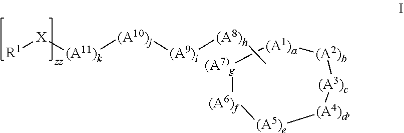

or a pharmaceutically acceptable salt, solvate, or hydrate thereof wherein:
R is a residue formed by subtracting a single H atom from respective parent (precursor) structure(s) (H)ₙR¹ at one of the following H-containing groups independently selected from NH, OH, SH, C(═O)OH, CONH, SO₂NH, and S(═O)NH when present in (H)ₙR¹; and wherein each (H)ₙR¹ is independently selected from:
a) a cytotoxic compound and an immunomodulating compound possessing an activity or capable of inducing an activity against one or more cancer cells;
b) afatinib, ARS-1630, axitinib, BGB-324, BLU-554, brivanib ((R)-1-((4-((4-fluoro-2-methyl-1H-indol-5-yl)oxy)-5-methylpyrrolo[2,1-f][1,2,4]triazin-6-yl)oxy)propan-2-ol), cabozantinib, cediranib, ceritinib, ciforadenant, derazantinib, dovitinib, emtansine, englerin, monomethyl auristatin E, irinotecan, maytansinoid, neratinib, nilotinib, nintedanib, ozogamicin, paclitaxel, pazopanib, regorafenib, sacituzumab, selpercatinib, semaxanib, sorafenib, sunitinib, SN-38, temsirolimus, tivantinib, tivozanib, vatalanib, veliparib, and vinblastine; and variants thereof, and
c) a nitrogen-containing heterocyclic structure connected to X at one of the heterocyclic nitrogen atom(s) present within (H)ₙR¹; wherein the nitrogen atom becomes a nitrogen atom with a single positive charge, such as imidazolium, pyrazolium, pyridinium, or indazolium group;
integer n is independently selected from 1, 2, and 3;
integers a through k are independently selected from 0, 1, and 2; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, and heterocyclyl; or the group A⁸ is directly connected to the group Y;
integer zz is independently selected from 1, 2, and 3;
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene, (C₁₋₁₂alkylene)C(═O)O, OC(═O)(C₁₋₁₂alkylene), (C₁₋₁₂alkylene)OC(═O), C(═O)O(C₁₋₁₂alkylene), (C₁₋₁₂alkylene)C(═O)N(R⁴), N(R⁴)C(═O)(C₁₋₁₂alkylene), (C₁₋₁₂alkylene)N(R⁴)C(═O), C(═O)N(R⁴)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹)ₛC(═O), C(═O)(CR⁵R⁶)ₚ(CR⁷R⁸)ᵣ(CR⁹R¹⁰)ₛN(R⁴)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁴)C(═O) C(═O)CR⁵═CR⁷—S—S—(CR⁹R¹⁰)ₛC(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁴)C(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁰R⁶)ₚ(CR⁷R⁸)ᵣN(C₁₋₆alkyl)C(═O)A¹⁴](CR⁹R¹⁰)ₛC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵(CR⁹R¹⁰)ₛN(C═O), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵(CR⁹R¹⁰)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O) C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), C(═O)N[CH₂CH₂NHC(═O)CH₂CH₂CH(NH₂)COOH](CR⁵R⁶)ₚOC(═O)(CR⁷R⁸)ᵣ (CR⁹R¹⁰)ₛC(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂c(═O), C(═O)N[CH₂CH₂NHC(═O)CH₂CH₂CH(NH₂)COOH]CH₂CH₂OC(═O)CH(Me)—CH₂C(═O), C(═O)N[CH₂cH₂N(C₆alkyl(═O)(HCHCH₂CH(NH₂)COOH]CH₂CH₂C(═O), (S)—C(═O)N[CH₂CH₂NHC(═O)CH₂CH₂CH(NH₂CO)COOH]HCH₂CH((═O), (R)—C(═O)N[CH₂CH₂NHC(═O)CH₂CH₂CH(NH₂) COOH]CH₂CH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), and (R)—C(═O)N[CH₂CH₂NHC(═O)CHNH(CH₂CH₂NH₂)]CH₂CH₂C(═O); or each optional divalent group X is independently selected from:

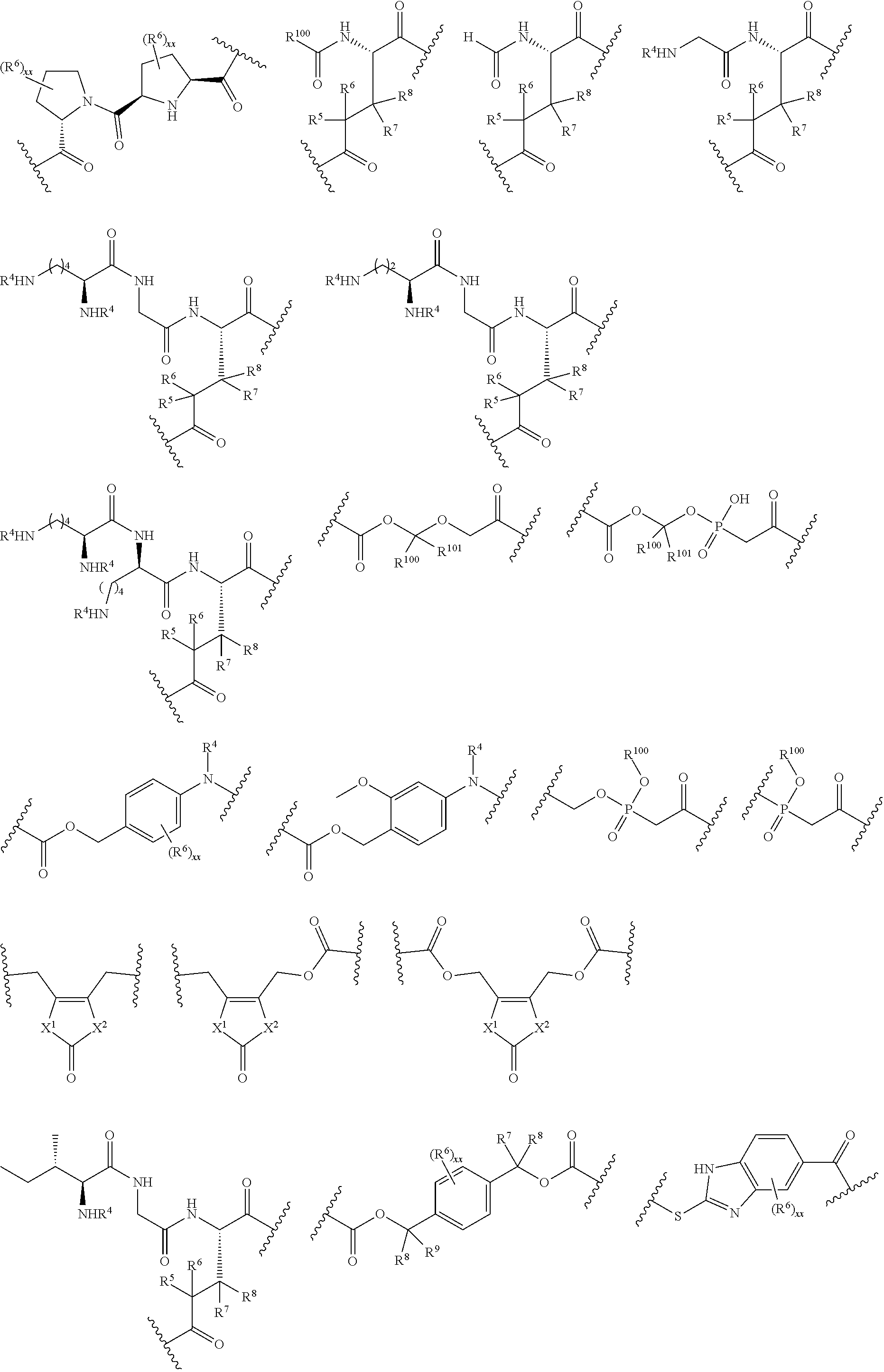

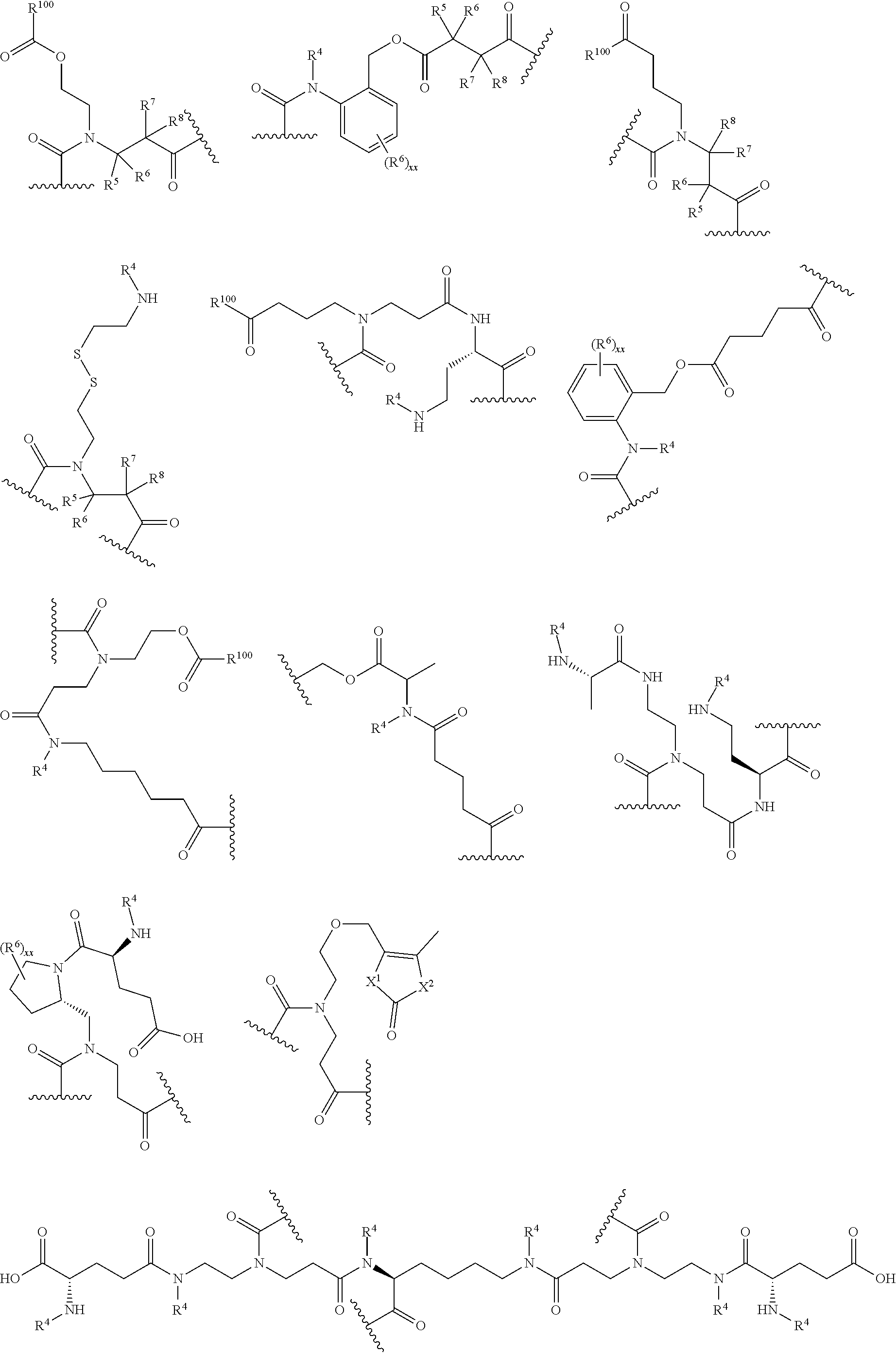

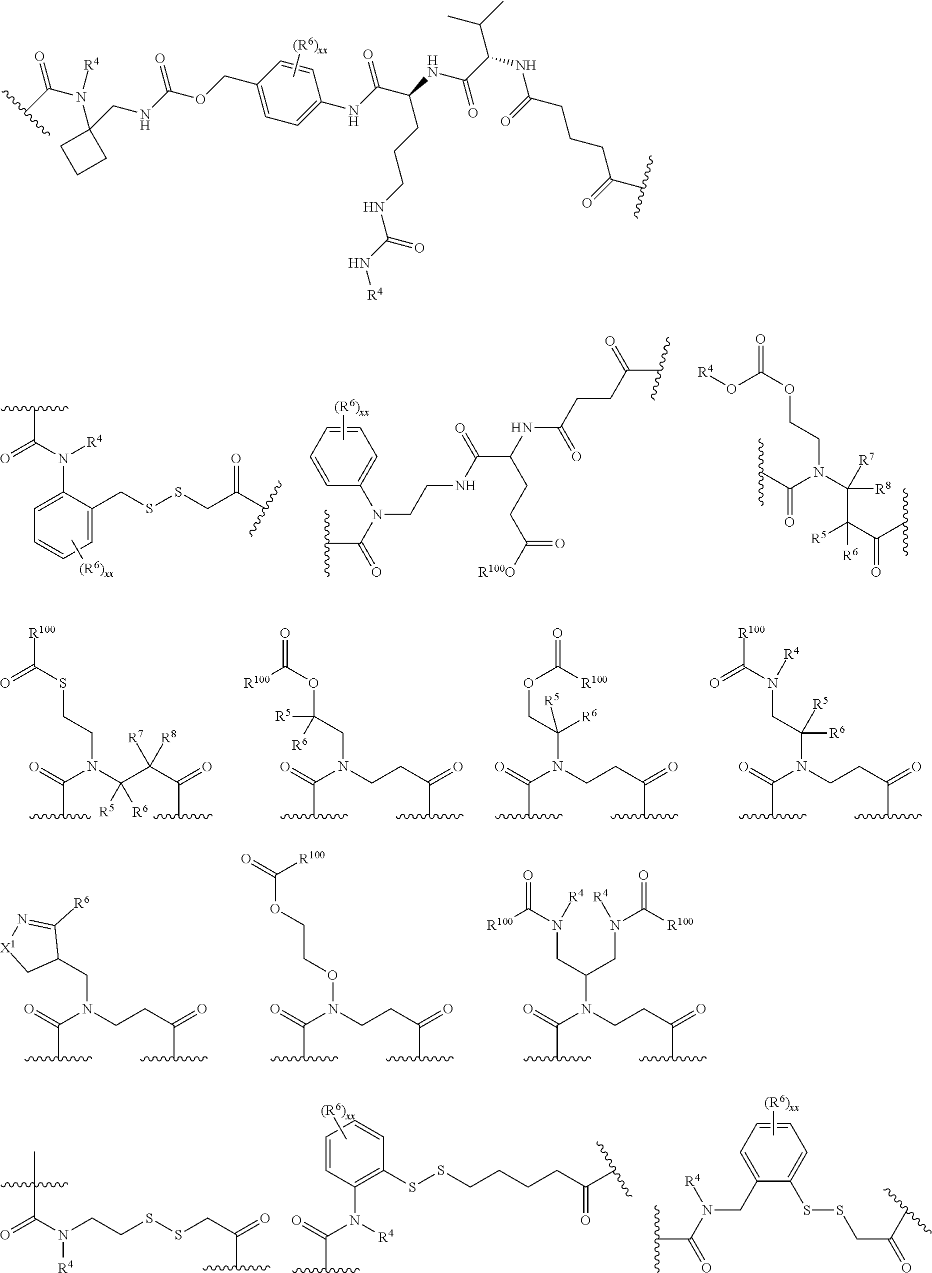

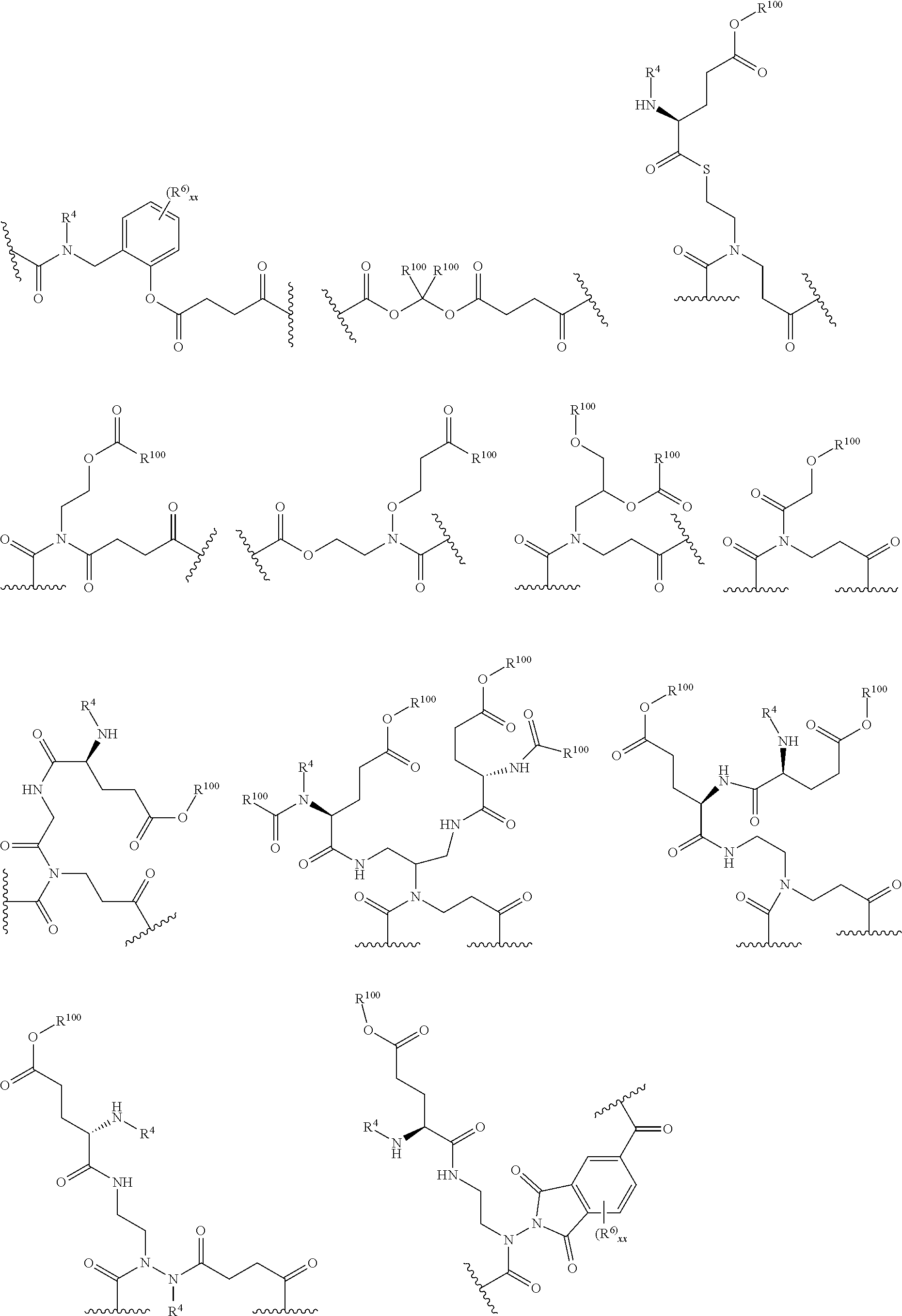

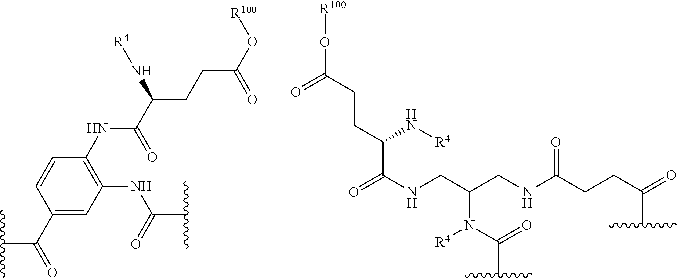

or any variant of the above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein
R⁶, R⁷, R⁸, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁵ and R⁶, ii) R⁶ and R⁷, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
xx is 1, 2, or 3;
R¹⁰⁰ and R¹⁰¹ are independently selected from hydrogen and alkyl;
X¹ and X² are independently selected from O, NH, and CR⁵R⁶;
integers p, r, and s are independently selected from 0, 1, and 2; and
wherein when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and
wherein when fragments (CR⁵R⁶)ₚ(CR⁷R⁸)ᵣ or (OCR⁵R⁶)ₚ(CR⁷R⁸)ᵣ are present, then [p+r]≥1; and wherein when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is optionally connected to one to two amino acid residue(s) A¹² or A¹³;
or any variant of the above X groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates one or more additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₄alkyl)C(═O), N(C₁₋₄alkyl)(CH₂)ₚO(CH₂)ᵣO(CH₂)ₛC(═O), N(C₁₋₄alkyl)(CH₂)ₚO(CH₂)ᵣO(CH₂)ₛOC(═O), N(C₁₋₄alkyl)(CH₂)ₚO(CH₂)ᵣO(CH₂)ₛNHC(═O), N(C₁₋₄alkyl)(CH₂)ₚO(CH₂)ᵣO(CH₂)ₛN(C₁₋₁₄alkyl)C(═O), and similar linear groups;
A¹ through A¹⁵ are independently optional and when present are independently selected from amino acid residues independently selected from unsubstituted or substituted at any N atom and where each of the amino acid residues, when present, is independently selected form alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid.

In one embodiment of Formula I, (H)ₙR¹ is independently selected from a nitrogen-containing heterocyclic structure comprised in the (H)ₙR¹ provided in (a) and (b) connected to X at one of the heterocyclic nitrogen atom(s) present within (H)ₙR¹; wherein the nitrogen atom becomes a nitrogen atom with a single positive charge, such as imidazolium, pyrazolium, pyridinium, or indazolium group.

In one aspect, provided is a compound of Formula I, Formula I-P-1, or Formula I-P-2 wherein the integers a through g are each 1; and wherein A¹ is Thr or Ser; A², A³A⁶, and A⁷ are independently selected from 2,3-diaminopropionic acid (Dap), 2,4-diaminobutyric acid (Dab), Ser, or Thr; A⁴ is Leu or Ile; and A⁵ is Phe, D-Phe, Bip, D-Bip, Val, and D-Val.

In additional aspect, provided is a compound of Formula I, Formula I-P-1, Formula I-P-2, Formula II-P, Formula III, or Formula IV-P, where group X, either at its left or right side therein, incorporates one or more additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₄alkyl)C(═O), N(C₁₋₄alkyl)(CH₂)ₚO(CH₂)ᵣO(CH₂)ₛC(═O), N(C₁₋₄alkyl)(CH₂)ₚO(CH₂)ᵣO(CH₂)ₛOC(═O), N(C₁₋₄alkyl)(CH₂)ₚO(CH₂)ᵣO(CH₂)ₛNHC(═O), N(C₁₋₄alkyl)(CH₂)ₚO(CH₂)ᵣO(CH₂)ₛN(C₁₋₄alkyl)C(═O), and similar linear groups. In an embodiment, 1, 2, 3, 4, or 5 additional divalent groups are incorporated and are independently selected.

In another aspect, provided is a compound of Formula I, Formula I-P-1, or Formula I-P-2, wherein the cyclic peptide structure comprised of optional amino acid residues A¹ through A⁷ is a cyclic peptide structure identical to that present in polymyxin B, polymyxin E, or octapeptin, or similar cyclic peptide structures. In another embodiment, provided is a compound of Formula I, Formula I-P-1, or Formula I-P-2, wherein the cyclic peptide structure comprised of optional amino acid residues A¹ through A⁷ is a cyclic peptide structure identical to that present in polymyxin B, polymyxin E, or octapeptin, or similar structures, including cyclic peptide structures. In another embodiment, provided is a compound of Formula I, Formula I-P-1, or Formula I-P-2, wherein the cyclic peptide structure comprised of optional amino acid residues A¹ through A⁷ is a cyclic peptide structure identical to that present in polymyxin A, polymyxin B, polymyxin B nonapeptide (H-Thr-Dab-cyclo[Dab-Dab-D-Phe-Leu-Dab-Dab-Thr]), polymyxin B heptapeptide (H-cyclo[Dab-Dab-D-Phe-Leu- Dab-Dab-Thr]), polymyxin E, or octapeptin, or similar structures, including cyclic peptide structures.

In an additional aspect, provided is a compound of any one of Formula I, Formula II-P, Formula III, Formula IV-P, Formula V-P, and Formula V, or of any embodiments as provided herein that exhibits a therapeutic effect after administration to a mammal by releasing molecule(s) (H)ₙR¹. In an additional aspect, provided is a compound of any one of Formula I-P-1, Formula I-P-2, and Formula III-P, or of any embodiments as provided herein, that exhibits a therapeutic effect after administration to a mammal by releasing molecule(s) (H)ₙR¹ and/or (H)ₒR².

In another aspect, provided is a compound of any one of Formula I, Formula I-P-1, Formula I-P-2, Formula II-P, Formula III, Formula III-P, Formula IV-P, Formula V-P, and Formula V, or of any embodiments as provided herein possessing anticancer activity against cancerous cells, as determined by inhibition or slowing of cancer(s) cells growth using in vitro cytotoxicity test(s) or assay(s), or by testing of said compounds in animal models for cancer(s).

In additional aspect, the aforementioned cancer is a renal cancer or a kidney cancer.

In another aspect, provided is a method for the treatment of a kidney cancer disease in a mammal comprising administering to the mammal a therapeutically effective amount of a compound of any one of Formula I, Formula I-P-1, Formula I-P-2, Formula II-P, Formula III, Formula III-P, Formula IV-P, Formula V-P, and Formula V, or of any embodiments as provided herein.

In another aspect, provided is a compound of any one of Formula I, Formula I-P-1, Formula I-P-2, Formula III, Formula IV-P, Formula V-P, and Formula V, or of any embodiments as provided herein and with a reduced cytotoxicity against non-cancerous mammalian cell(s), when compared to a related cytotoxic structure (compound, e.g. (H)ₙR¹) incorporated into said compound, as determined by in vitro cytotoxicity test(s) or assay(s). In another aspect, provided is a compound of any one of Formula I-P-1, Formula I-P-2, and Formula III-P, or of any embodiments as provided herein and with a reduced cytotoxicity against non-cancerous mammalian cell(s), when compared to a related cytotoxic structure (compound, e.g. (H)ₙR¹ and/or (H)ₒR²) incorporated into said compound, as determined by in vitro cytotoxicity test(s) or assay(s).

In another aspect, provided is a compound of any one of Formula I, Formula II-P, Formula III, Formula IV-P, Formula V-P, and Formula V, or of any embodiments as provided herein possessing an increased in vivo efficacy against cancer(s), when compared to a related (parent) cytotoxic structure (compound, e.g. (H)ₙR¹) incorporated into said compound, as determined by in vivo test(s) in animal model(s) of cancer(s), wherein said compound and a related cytotoxic structure (compound, e.g. (H)ₙR¹) are dosed to an animal at identical molar dose of a common cytotoxic structure within the test and the comparator compounds. In another aspect, provided is a compound of any one of Formula I-P-1, Formula I-P-2, and Formula III-P, or of any embodiments as provided herein possessing an increased in vivo efficacy against cancer(s), when compared to a related (parent) cytotoxic structure (compound, e.g. (H)ₙR¹ and/or (H)ₒR²) incorporated into said compound, as determined by in vivo test(s) in animal model(s) of cancer(s), wherein said compound and a related cytotoxic structure (compound, e.g. (H)ₙR¹ and/or (H)ₒR²) are dosed to an animal at identical molar dose of a common cytotoxic structure within the test and the comparator compounds.

In yet another aspect, provided is a compound of any one of Formula I, Formula I-P-1, Formula I-P-2, Formula II-P, Formula III, Formula III-P, Formula IV-P, Formula V-P, and Formula V, or of any embodiments as provided herein possessing at least 2-fold increase in vivo efficacy against cancer(s), when compared to a related cytotoxic structure (compound) incorporated into said compound.

In additional aspect is provided a pharmaceutical composition comprising a compound of any one of Formula I, Formula I-P-1, Formula I-P-2, Formula II-P, Formula III, Formula III-P, Formula IV-P, Formula V-P, and Formula V, or of any embodiments as provided herein, or a pharmaceutically acceptable salt, prodrug, solvate, or hydrate thereof, and a pharmaceutically acceptable carrier, excipient or diluent.

In an another aspect is provided a method for treating a kidney cancer in humans or other warm-blooded animals by administering to the subject in need thereof a therapeutically effective amount of a compound of any one of Formula I, Formula I-P-1, Formula I-P-2, Formula II-P, Formula III, Formula III-P, Formula IV-P, Formula V-P, and Formula V, or of any embodiments as provided herein or a pharmaceutically acceptable salt, prodrug, solvate, or hydrate thereof.

The compound of any one of Formula I, Formula I-P-1, Formula I-P-2, Formula II-P, Formula III, Formula III-P, Formula IV-P, Formula V-P, and Formula V, or of any embodiments as provided herein may be administered, for example, orally, parenterally, transdermally, topically, rectally, or intranasally, or via an intra-tumoral administration.

In yet another aspect is provided novel intermediates and processes for preparing compounds of any one of Formula I, Formula I-P-1, Formula I-P-2, Formula II-P, Formula III, Formula III-P, Formula IV-P, Formula V-P, and Formula V, or of any embodiments as provided herein.

### BRIEF DESCRIPTION OF FIGURES

FIG. 1 is a graph showing the efficacy of the compound of Example 1 vs axitinib in an orthotopic model. The compound of Example 1 was administered at a dose of 12 and 4 mg/kg (equal to 2.0 and 0.67 mg/kg axitinib free drug form, respectively, based on molecular weights of the compound of Example 1 and axitinib: 2303.0 Daltons (6 TFA salt) and 386.47, respectively). The compound of Example 1 was tested uncorrected for solvate, or water content. BLI stands for bioluminescence.

FIG. 2 is a graph showing body weight changes during the efficacy study of the compound of Example 1 compared to axitinib. The compound of Example 1 was dosed at 12 and 4 mg/kg and axitinib was dosed at 30 mg/kg. The compound of Example 1 was tested uncorrected for solvate, or water content.

### DETAILED DESCRIPTION

Unless otherwise stated, the following terms used in the specification and Claims have the meanings given below.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₁₄ alkyl refers to alkyl of one to fourteen carbon atoms, inclusive.

The term alkyl refers to both straight and branched saturated hydrocarbon groups. Reference to an individual radical such as “propyl” embraces only the straight chain radical, and a branched chain isomer such as “isopropyl” being specifically referred to. Unless specified otherwise “alkyl” contains 1-12 carbon atoms. In addition to any group specifically recited in any of the embodiments or claims, the alkyl group is optionally substituted with one, two, three, or four substituents selected from the group consisting of halo, hydroxy, cyano, C₁₋₁₂ alkyl, C₃₋₇cycloalkyl, aryl, biaryl, heterocyclic, and heteroaryl. In some embodiments, alkyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂-phenyl, biphenylmethyl, and the like. In some embodiments, alkyl is unsubstituted. Alkyl groups distinguished as “alkyl” and “alkyl¹” or “alkyl²” refer to independently selected alkyl groups that may be different from each other, or independently equal to each other. If the term “alkyl” is used more than once in the same group, then each “alkyl” is independent of another “alkyl”, at each appearance.

The term “Alk” refers to alkyl, as defined herein.

The term “alkylene” refers to a divalent alkyl group. Unless specified otherwise, “alkylene” contains 1-12 carbon atoms. In some embodiments, “alkylene” is lienar. The alkylene group is optionally substituted as described for alkyl. In some embodiments, alkylene is unsubstituted. Alkylene groups distinguished as “alkylene” and “alkylene¹” or “alkylene²” refer to independently selected alkylene groups that may be different from each other, or independently equal to each other.

The term “alkenyl” refers to both straight and branched hydrocarbon groups containing at least one double bond, and in some embodiments 1, 2, or 3 double bonds. Unless specified otherwise, “alkenyl” contains 2-12 carbon atoms. In addition to any group specifically recited in any of the embodiments or claims, the alkenyl is optionally substituted with one, two, or three substituents selected from the group consisting of halo, C₁₋₁₂ alkyl, C₃₋₇ cycloalkyl, aryl, biaryl, Het¹, and Het². In some embodiments, alkenyl is unsubstituted. In some embodiments, alkenyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂-phenyl, biphenylmethyl, and the like.

The term “alkenylene” refers to a divalent alkenyl group. Unless specified otherwise “alkenylene” contains 2-12 carbon atoms. The alkenylene group is optionally substituted as described for alkenyl. In some embodiments, the alkenylene group is unsubstituted.

The term “cycloalkyl” means a cyclic saturated, monovalent, monocyclic or bicyclic, saturated or unsaturated hydrocarbon group of three to 18 (in some embodiments, three to six) carbon atoms. In some embodiments, cycloalkyl includes, but is not limited to, cyclopropyl, cyclohexyl, cyclododecanoyl, and the like. In addition to any group specifically recited in any of the embodiments or claims, the cycloalkyl group is optionally substituted with one, two, or three substituents selected from the group consisting of halo, C₁₋₁₂ alkyl, C₃₋₇ cycloalkyl, aryl, Het¹, Het², and heteroaryl. In some embodiments, cycloalkyl is unsubstituted.

The term “cycloalkylene” means a divalent cycloalkyl group. In addition to any group specifically recited in any of the embodiments or claims, the cycloalkylene group is optionally substituted as described for cycloalkyl. In some embodiments, the cycloalkylene is unsubstituted. In some or any embodiments, the C₃₋₆cycloalkylene group formed by any two of R⁵ through R¹⁰ is optionally substituted with one or two groups independently selected from C₁₋₆alkyl and aryl.

The term “heteroalkyl” means an alkyl or cycloalkyl group, as defined above, having a substituent containing a heteroatom selected from N, O, and S(O)ₙ, where n is an integer selected from 0 to 2, where in some embodiments, the substituent includes, hydroxy (OH), C₁₋₄alkoxy, amino, thio (—SH), and the like. Said heteroatom may be incorporated in any part of the heteroalkyl group [e.g., heteroalkyl can be C₁₋₄alkylC(═O)O C₃₋₆ cycloalkylNH₂], or contain a heterocyclic substituent [e.g., heteroalkyl can be 2-(4-morpholino)ethyl]. In some embodiments, substituents include —NRₐR_{b}, —ORₐ, and —S(O)ₙR_{c}, wherein each Rₐ is independently hydrogen, C₁₋₄alkyl, C₃₋₆cycloalkyl, optionally substituted aryl, optionally substituted heterocyclic, or —C(O)R (where R is C₁₋₄alkyl); each R_{b} is independently hydrogen, C₁₋₄alkyl, —SO₂R (where R is C₁₋₄alkyl or C₁₋₄ hydroxyalkyl), —SO₂NRR′ (where R and R′ are independently of each other hydrogen or C₁₋₄ alkyl), or —CONR′R″ (where R′ and R″ are independently of each other hydrogen or C₁₋₄ alkyl); n is an integer from 0 to 2; and each R, is independently hydrogen, C₁₋₄alkyl, C₃₋₆ cycloalkyl, optionally substituted aryl, or NRₐR_{b} where Rₐ and R_{b} are as defined above. In some embodiments, heteroalkyl includes, but is not limited to 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to substituted or unsubstituted phenyl, biphenyl, triphenyl, or naphthyl. In addition to any group specifically recited in any of the embodiments or claims, the aryl is optionally substituted with 1 to 3 substituents independently selected from halo, —C₁₋₁₂alkyl (unsubstituted or substituted, in one embodiment with 1, 2, or 3 halo), aryl, —OH, —OC₁₋₁₂alkyl, —S(O)ₙC₁₋₄alkyl (wherein n is 0, 1, or 2), —C₁₋₄alkylNH₂, —NHC₁₋₄alkyl, —C(═O)H, C(═O)OR^{a}, OC(═O)R^{a}, OC(═O)NR^{a}R^{c}, OC(═O)heteroaryl, OC(═O)(heterocyclic ring) and —C═N—OR_{d} wherein R_{d} is hydrogen or —C₁₋₄alkyl. Two adjacent substituents in the aryl group may be connected to form a C₄₋₇cycloalkyl or 4- to 7-member heterocyclic group fused to said aryl group. Aryl groups distinguished as “aryl” and “aryl¹” or “ary²” refer to independently selected aryl groups that may be different from each other, or independently equal to each other. If the term “aryl” is used more than once in the same group, then each “aryl” is independent of another “aryl”, at each appearance.

The term “arylene” refers to a divalent aryl group, as defined herein.

The term “arylalkyl” refers to an alkyl group substituted with an aryl group, each as defined herein, including where the aryl and alkyl are optionally substituted as described in their respective definitions.

The term “arylheteroaryl” refers to an aryl group substituted with a heteroaryl group, each as defined herein, including where the aryl and heteroaryl are optionally substituted as described in their respective definitions.

The term “(heteroaryl)arylene” refers to a divalent aryl group, as defined herein, substituted with a heteroaryl group.

The term “heteroarylaryl” refers to a heteroaryl group substituted with an aryl group, each as defined herein, including where the aryl and heteroaryl are optionally substituted as described in their respective definitions.

The term “(aryl)heteroarylene” refers to a divalent heteroaryl group, as defined herein, substituted with an aryl group.

The term “biaryl” refers to an aryl group as defined herein substituted with another aryl group as defined herein, including where the aryl groups are independently optionally substituted as described in the definition.

The term “biarylene” refers to a divalent biaryl group, as defined herein.

The term “biarylalkyl” refers to an alkyl group substituted with an aryl group which is substituted with another aryl group, each as defined herein, including where each aryl independently and alkyl are optionally substituted as described in their respective

### Definitions

The terms “heterocyclic,” “heterocyclic ring,” “heterocyclyl,” and “heterocycle” refer to a monocyclic or bicyclic aromatic ring or a saturated or unsaturated, monocyclic or bicyclic ring that is not aromatic comprising 3 to 12 carbon atoms and 1 to 4 heteroatoms independently selected from the group consisting of oxygen, nitrogen, P(═O), and S(O)ₘ within the ring, wherein m is an integer from 0 to 2. In addition to any group specifically recited in any of the embodiments or claims, the heterocyclic ring is optionally substituted with one, two, or three halo, C(═O)OR^{a}, OC(═O)R^{a}, OC(═O)NR^{a}R_{b}, —C₁. 20alkyl, —OH, —NH₂, —OC₁₋₂₀alkyl, —S(O)ₘC₁₋₂₀alkyl (wherein m is 0, 1, or 2), —C₁₋₂₀alkyl-NH₂, —NHC₁₋₄alkyl, —C(═O)H, or —C═N—OR_{d} wherein each R^{a}, R^{b} and R_{d} is independently hydrogen or C₁₋₂₀alkyl. In some embodiments, the heterocyclic ring is unsubstituted. In some or any embodiments, the 4 to 7 or 5 to 7 membered ring formed by any two of R⁵ through R¹⁰ and/or formed by R⁶ and R⁸ optionally substituted as described herein for heterocycle. In some or any embodiments, the 5 to 7 membered ring formed by R¹¹ and R¹² and/or formed by R⁴ and R¹¹ and/or formed by R⁶ and R¹² is optionally substituted with one or two groups independently selected from C₁₋₆alkyl and aryl.

In some embodiments, heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one, azabicyclo[3.1.0]hexane and the like, and N-oxides of said nitrogen heterocycles. In addition to any group specifically recited in any of the embodiments or claims, heterocyclic rings include substituted and unsubstituted rings, including those substituted with groups selected from C(═O)OR^{a}, OC(═O)R^{a}, OC(═O)NR^{a}R^{b} where each R^{a} and R^{b} are independently hydrogen or C₁₋₆alkyl.

Hetⁱ at each occurrence is independently a C-linked 5- or 6-membered heterocyclic ring having 1 to 4 heteroatoms selected from the group consisting of oxygen, nitrogen, and sulfur within the ring.

Het² at each occurrence is independently a N-linked 5 or 6 membered heterocyclic ring having 1 to 4 nitrogen and optionally having one oxygen or sulfur within the ring.

The term “heterocyclylene” refers to a divalent heterocyclyl group, as defined herein.

The term “unsaturated” in the context of the term cycloalkyl, cycloalkylene, and heterocycle refers to a partially unsaturated, but not aromatic ring.

The term “heteroaryl” refers to a five-(5) or six-(6) membered C- or N-linked heterocyclic ring, optionally fused to a benzene or to another heterocyclic ring (wherein at least one of the heterocyclic rings is aromatic). Heterocyclic ring fused to a benzene ring is also referred to as benzo-heterocyclic group. In some embodiments, heteroaryl includes, but is not limited to, pyridine, thiophene, furan, pyrazole, indole, benzimidazole, quinoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, and 1,2,4-dithiazolone. In addition to any group specifically recited in any of the embodiments or claims, heteroaryl groups include substituted and unsubstituted rings, including those substituted with groups selected from C(═O)OR^{a}, OC(═O)R^{a}, and OC(═O)NR^{a}R^{b} where each R^{a} and R^{b} are independently hydrogen or C₁₋₆alkyl. In some embodiments, heteroaryl is unsubstituted. Heteroaryl groups distinguished as “heteroaryl” and “heteroaryl¹” or “heteroaryl²” refer to independently selected heteroaryl groups that may be different from each other, or independently equal to each other. If the term “heteroaryl” is used more than once in the same group, then each “heteroaryl” is independent of another “heteroaryl”, at each appearance.

The term “heteroarylalkyl” refers to an alkyl group substituted with an heteroaryl group, each as defined herein.

The term “mono-substituted” refers to a group having at least one substituent in said group, not counting the point of connection of this group to the main structure or general formula. The term “multiply-substituted” refers to a group having at least two substituents in said group, not counting the point of connection of this group to the main structure or general formula.

Unless specified otherwise, “carbon atom” means the atom of element carbon optionally substituted with H, halo, NR^{a}R^{b}, C₁₋₁₂alkyl, C₃₋₇ cycloalkyl, aryl, heteroaryl, or with a heterocyclic ring. Carbon atom comprises atoms with sp3, sp2, and sp electronic hybridization.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds provided herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include all individual enantiomers and any mixtures, racemic, partially racemic, or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H), carbon (C), or nitrogen (N) substitution for compounds of the Formulas I-V-P include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application, or stability enhancement. Optionally, a compound described herein may incorporate a known in the art radioactive isotope or radioisotope, such as any number of ³H, ¹⁵O, ¹²C, or ¹³N isotopes, to afford a respective radiolabeled compound of Formulas I-V-P.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating”, “treatment”, or “therapy” of a disease includes:
(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Leaving group” has the meaning conventionally associated with it in synthetic organic chemistry, i.e., an atom or group capable of being displaced by a nucleophile and includes halogen, C₁₋₄alkylsulfonyloxy, ester, or amino such as chloro, bromo, iodo, mesyloxy, tosyloxy, trifluorosulfonyloxy, methoxy, N,O-dimethylhydroxyl-amino, and the like.

“Prodrug” means any compound which releases an active parent drug according to a compound provided herein in vivo when such prodrug is administered to a mammalian subject. Prodrugs of a compound of provided herein are prepared by modifying functional groups present in a compound provided herein in such a way that the modifications may be cleaved in vivo to release the parent compound. Prodrugs include compounds provided herein wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl) of hydroxy functional groups in compounds provided herein, and the like. Prodrugs of compounds provided herein may be used for particular therapeutic application, such as for pulmonary delivery of an aerosol containing a prodrug of such a compound, or to improve tolerance to same agent. For example, a methanesulfonate prodrug form of polymyxin drug colistin (described, for example by Bergen et al. in Antimicrob. Agents Chemother. 2006, vol. 50, p. 1953) is used to reduce neurotoxic effects of colistin, and is used for aerosol administration of this drug. This and other known forms of prodrugs could be likewise used to further improve pharmaceutical properties of the compounds provided herein.

The term “mammal” refers to all mammals including humans, livestock, and companion animals.

The compounds described herein are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature).

### ILLUSTRATIVE EMBODIMENTS

In another aspect, provided herein is a compound of Formula I-P-2:

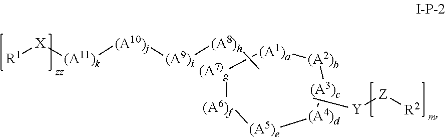

or a pharmaceutically acceptable salt, solvate, or hydrate thereof wherein:
R¹ and R² are optional groups, with at least one of the groups R¹ and R² being present in the Formula I-P-2; and
R¹ and R² are independently selected from alkyl, aryl, biaryl, heteroaryl, heteroarylaryl, and arylheteroaryl; or
R¹ and R² are residues independently derived by subtracting a single or multiple H atom(s) from respective parent (precursor) structure(s) (H)ₙR¹ and (H)ₒR² at any one of optional H-containing group(s) independently selected from NH, OH, SH, C(═O)OH, CONH, SO₂NH, and S(═O)NH present in said structures (H)ₙR¹ and (H)ₒR²; and wherein
a) the structures (H)ₙR¹ and (H)ₒR² are independently structures of compounds possessing biological or therapeutic activity; or
b) the structures (H)ₙR¹ and (H)ₒR² are structures of cytotoxic, antibody, or immunomodulating compounds possessing an activity or capable of inducing activity against cancer cells, including compounds with activity against renal cancer cells; or
c) the structures (H)ₙR¹ and (H)ₒR² are structures of mono- or multi-valent antibody(ies) with activity against cancer cells; or
d) the structures (H)ₙR¹ and (H)ₒR² are independently afatinib ((E)-N-[4-(3-chloro-4-fluoroanilino)-7-[(3S)-oxolan-3-yl]oxyquinazolin-6-yl]-4-(dimethylamino)but-2-enamide), ARS-1630, axitinib (N-methyl-2-[[3-[(E)-2-pyridin-2-ylethenyl]-1H-indazol-6-yl]sulfanyl]benzamide), BGB-324, BLU-554, brivanib ((S)—(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate), (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib (same as 4-amino-5-fluoro-3-(6-(4-methylpiperazin-1-yl)-1H-benzo[d]imidazol-2-yl)quinolin-2(1H)-one), E-7046, emtansine, englerin ((1R,3aR,4S,5R,7R,8S,8aR)-5-(glycoloyloxy)-7-isopropyl-1,4-dimethyldecahydro-4,7-epoxyazulen-8-yl (2E)-3-phenylacrylate), foretinib, lenvatinib (4-[3-chloro-4-(cyclopropylcarbamoylamino)phenoxy]-7-methoxyquinoline-6-carboxamide), monomethyl auristatin E ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide), irinotecan, maytansinoid, neratinib, nilotinib, nintedanib, ozogamicin, paclitaxel, pazopanib (same as 5-[[4-[(2,3-dimethylindazol-6-yl)-methylamino]pyrimidin-2-yl]amino]-2-methylbenzenesulfonamide), regorafenib, sacituzumab, selpercatinib, semaxanib ((Z)-3-((3,5-dimethyl-1H-pyrrol-2-yl)methylene)indolin-2-one), sorafenib (4-[4-[[4-chloro-3-(trifluoromethyl)phenyl]carbamoylamino]phenoxy]-N-methylpyridine-2-carboxamide), sunitinib ((Z)—N-(2-(diethylamino)ethyl)-5-((5-fluoro-2-oxoindolin-3-ylidene)methyl)-2,4-dimethyl-1H-pyrrole-3-carboxamide), SN-38 (7-ethyl-10-hydroxy-camptothecin), sorafenib (4-[4-[[4-chloro-3-(trifluoromethyl)phenyl]carbamoylamino], phenoxy]-N-methyl-pyridine-2-carboxamide), trastuzumab, tesirine (same as [4-[[(2S)-2-[[(2S)-2-[3-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]propanoyla mino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy]pentoxy]-6-hydroxy-2-methoxy-8-methyl-11-oxo-6a,7-dihydro-6H-pyrrolo[2,1-c][1,4]benzodiazepine-5-carboxylate), temsirolimus ([(1R,2R,4S)-4-[(2R)-2-[(1R,9S,12S,15R,16E,18R,19R,21R,23 S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl]3-hydroxy-2-(hydroxymethyl)-2-methylpropanoate), tivantinib, tivozanib (1-{2-chloro-4-[(6,7-dimethoxy-4-quinolinyl)oxy]phenyl}-3-(5-methyl-1,2-oxazol-3-yl)urea), vatalanib, veliparib, vinblastine; or variants thereof, or
the structures (H)ₙR¹ and (H)ₒR² are independently structures of compounds active against a kidney disease; and wherein
when an optional group R¹ is absent, then the fragment R¹X is replaced for group R^{11a}, wherein R^{11a}, is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇ cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is replaced for group R^{12a} wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇ cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroarylN(C₁₋₁₂alkyl)C(═O), heteroarylSO₂, and heteroarylNHSO₂; or
e) (H)ₙR¹ is heterocyclic structure(s) comprised in the (H)ₙR¹ provided in (a) and (b) connected to X at one of heterocyclic nitrogen atom(s) present within the structure (H)ₙR¹; wherein said nitrogen atom becomes a nitrogen atom with a single positive charge, imidazolium, pyrazolium, pyridinium, or indazolium group; and
integers n and o are independently selected from 0, 1, 2, 3, 4, 5, 6, and 7, such that [n+o]≥1; and
A¹ through A¹¹ are independently optional and when present are independently selected from amino acid residues independently selected from substituted or unsubstituted at any N atom and where each of the amino acid residues, when present, is independently selected form alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and
integers a through k, m, and zz are independently selected from 0, 1, and 2, and wherein
[m+zz]≥1; and wherein
when any of integers a through k is 0, then any of the two groups adjacent to a respective absent group (according to the integer 0 at said absent group) are connected to each other directly; and wherein
when both integers selected from a through k at adjacent groups A¹-A², A³-A⁴ A⁵-A⁶ or A¹-A⁷ are 0, then respective pairs of adjacent groups A¹-A², A³-A⁴, A⁵-A⁶, or A¹-A⁷ are absent, to result in acyclic structure of formula I-P; wherein one of remaining groups selected from A-A⁷ is connected to either group A⁸ or group Y, and the last amino acid residue of resulted peptide sequence (which is not connected to either group A⁸ or group Y) terminates with groups COOH, CH₂OH, or C(═O)NR³R⁴, wherein R³ and R⁴ are independently selected from H, alkyl, aryl, heteroaryl, and heterocyclyl; or wherein
when the integers a through g are all 0, then the groups A-A⁷ are absent, and the group A⁸ terminates with either COOH, CH₂OH, or C(═O)NR³R⁴; or the group A⁸ is directly connected to the group Y; and
optional divalent groups X, Y, and Z are independently selected from O, NH, N(C₁₋₆ alkyl), S, S—S, S—N, S(═O), SO₂, C(═O), OC(═O), C(═O)O, NHC(═O)NH, N(C₁₋₆ alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene, (C₁₋₁₂alkylene)C(═O)O, OC(═O)(C₁₋₁₂alkylene), (C₁₋₁₂alkylene)OC(═O), C(═O)O(C₁₋₁₂alkylene), (C₁₋₁₂alkylene)C(═O)N(R⁵), N(R⁵)C(═O)(C₁₋₁₂alkylene), (C₁₋₁₂alkylene)N(R⁵)C(═O), C(═O)N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R)C(═O), C(═O)CR⁵═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)OC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵(CR⁹R¹⁰)ₛN(C═O), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵(CR⁹R¹⁰)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂ C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O) C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), C(═O)N[CH₂CH₂NHC(═O)CH₂CH₂CH(NH₂)COOH](CR⁵R⁶)ₚ OC(═O)(CR⁷R⁸)ᵣ(CR⁹R¹⁰)ₛC(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂C(═O), C(═O)N[CH₂CH₂NHC(═O)CH₂CH₂CH(NH₂)COOH]CH₂CH₂OC(═O)CH(Me)—CH₂C(═O), C(═O)N[CH₂CH₂N(C₁₋₆alkyl)C(═O)CH₂CH₂CH(NH₂)COOH]CH₂CH₂C(═O), (S)—C(═O)N[CH₂CH₂NHC(═O)CH₂CH₂CH(NH₂)COOH]CH₂CH₂C(═O), (R)—C(═O)N[CH₂CH₂NHC(═O)CH₂CH₂CH(NH₂) COOH]CH₂CH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), and (R)—C(═O)N[CH₂CH₂NHC(═O)CHNH(CH₂CH₂NH₂)]CH₂CH₂C(═O);
or any variant of the above groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein
R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁶ and R⁷, and ii) R⁹ and R¹⁰, together with the atom to which they are attached form a C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r and s are independently selected from 0, 1, and 2; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
X, Y, and Z are independently selected from one to four amino acid residue(s) A¹²,
A¹³, A¹⁴, and A¹⁵ connected to each other with peptide bonds; wherein A¹, A³, A¹⁴, or A¹⁵ are substituted or unsubstituted at any N atom and are independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid, or 5-amino-4-arylpentanoic acid, and similar natural or unnatural amino acid residues; or
X is a group comprised of the following structures, additionally connected to one to two amino acid residue(s) A¹² or A¹³, at the right side of the following structures: (C₁₋₁₂alkylene)C(═O)O, OC(═O)(C₁₋₁₂alkylene), (C₁₋₁₂alkylene)OC(═O), C(═O)O(C₁₋₁₂alkylene), (C₁₋₁₂alkylene)C(═O)N(R⁵), N(R⁵)C(═O)(C₁₋₁₂alkylene), (C₁₋₁₂alkylene)N(R⁵)C(═O), C(═O) N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R)C(═O), C(═O)CR^{S}═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁹)ᵣN(C₁₋₆alkyl)C(═O)A¹⁴](CR⁹R^{o})ₛC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)NOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵](CR⁹R¹⁰)ₛN(C═O), C(═O)N[(CR⁵R⁶)ₚ(CR⁷R⁸)ᵣNHC(═O)A¹⁴A¹⁵](CR⁹R¹⁰)ₛO(C═O), C(═O)N[(CR⁵R⁶)ₚ(CR⁷R⁸)ᵣOC(═O)A¹⁴A¹⁵](CR⁹R¹⁰)ₛC(═O), C(═O)N[(CR⁵R⁶)ₚ(CR⁷R⁸)ᵣOC(═O)A¹⁴A¹⁵](CR⁹R¹⁰)ₛN(C═O), C(═O)N[(CR⁵R⁶)ₚ(CR⁷R⁸)ᵣOC(═O)A¹⁴A¹⁵](CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂ C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O), C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), C(═O)N[CH₂CH₂NHC(═O)CH₂CH₂CH(NH₂)COOH](CR⁵R⁶)ₚ OC(═O)(CR⁷HR⁸)ᵣ (CR⁹R¹⁰)ₛC(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂c(═O), C(═O)N[CH₂CH₂NHC(═O)CH₂CH₂CH(NH₂)COOH]CH₂CH₂OC(═O)CH(Me)—CH₂C(═O), C(═O)N[CH₂cH₂N(C₆alkyl(═O)(HCHCH₂CH(NH₂)COOH]CH₂CH₂C(═O), (S)—C(═O)N[CH₂CH₂NHC(═O)CH₂CH₂CH(NH₂CO)COOH]HCH₂CH((═O), (R)—C(═O)N[CH₂CH₂NHC(═O)CH₂CH₂CH(NH₂) COOH]CH₂OH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), or (R)—C(═O)N[CH₂CH₂NHC(═O)CHNH(CH₂CH₂NH₂)]CH₂CH₂C(═O);
or any variant of these groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² or A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
Z is a group comprised of the following structures, additionally connected to one to two amino acid residue(s) A¹² or A¹³, at the left side of the following structures: (C₁₋₁₂alkylene)C(═O)O, OC(═O)(C₁₋₁₂alkylene), (C₁₋₁₂alkylene)OC(═O), C(═O)O(C₁₋₁₂alkylene), (C₁₋₁₂alkylene)C(═O)N(R⁵), N(R⁵)C(═O)(C₁₋₁₂alkylene), (C₁₋₁₂alkylene)N(R⁵)C(═O), C(═O) N(R⁵)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O) C(═O)CR⁵═CR⁷—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N(R⁵)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁵)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R′)C(═O), C(═O)CR^{S}═CR⁷—S—S—(CR⁹R¹⁰)ₛC(═O), C(═O)OCR⁵═CR⁷—(CR⁹R¹⁰)ₛC(═O), C(═O)N(R⁵)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁵)C(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₆alkyl)C(═O)A¹⁴](CR⁹R¹⁰)ₛOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵(CR⁹R¹⁰)ₛN(C═O), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵(CR⁹R¹⁰)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂ C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene-C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O) C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂C(═O), C(═O)N[CH₂CH₂NHC(═O)CH₂CH₂CH(NH₂)COOH]CH₂CH₂OC(═O)CH(Me)—CH₂C(═O), C(═O)N[CH₂CH₂N(C₁₋₆alkyl)C(═O)CH₂CH₂CH(NH₂)COOH]CH₂CH₂C(═O), (S)—C(═O)N[CH₂CH₂NHC(═O)CH₂CH₂CH(NH₂)COOH]CH₂CH₂C(═O), (R)—C(═O)N[CH₂CH₂NHC(═O)CH₂CH₂CH(NH₂) COOH]CH₂CH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), or (R)—C(═O)N[CH₂CH₂NHC(═O)CHNH(CH₂CH₂NH₂)]CH₂CH₂C(═O);
or any variant of these groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues As or A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸.

In an alternative embodiment of Formula I-P-2, R⁵ and R⁶ together with the atom to which they are attached form a C₃₋₆cycloalkylene.

In an alternative embodiment of Formula I-P-2, fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present and [p+r+s]≥1.

In an alternative embodiment of Formula I-P-2, fragments (CR⁵R⁶)ₚ(CR⁷R⁸)ᵣ or (OCR⁵R⁶)ₚ(CR⁷R⁸)ᵣ are present and [p+r]≥1.

In an alternative embodiment of Formula I-P-2, optional divalent groups X, Y, and Z are independently selected from (C₁₋₁₂alkylene)C(═O)N(R⁴), N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), C(═O)CR⁵═CR—(CR⁹R¹⁰)ₛN(R⁴)C(═O), and or any variant of the above groups formed by straightforward repositioning, addition, or deletion of the fragment N(R⁴)C(═O) or C(═O)N(R⁴)SO₂; or

optional divalent groups X, Y, and Z are independently selected from C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛC(═O), C═)[C⁵R⁶ₚ(CR⁷R⁸)ᵣN(C₁₋₆aklyl)C(═O)A¹⁴](C ⁹R¹⁰)ₛNHC(═O) C(═O)N[(CR⁵R⁶)ₚ(CR⁷R⁸)ᵣN(C₁₋₆alkyl)C(═O)A¹⁴](CR⁹R¹⁰)ₛNC(═O), C(═O)N[(CR⁵R⁶)ₚ(CR⁷R⁸)ᵣN(C₁₋₆alkyl)C(═O)A¹⁴(CR⁹R¹⁰)OC(═O), C(═O)N[(CR⁵R⁶)ₚ(CR⁷R⁸)ᵣOC(═O)A¹⁴](CR⁹R¹⁰)ₛN(C═), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC═O), C(═O)N[(CR⁵R⁶)ₚ(CR⁷R⁸)ᵣNHC(═O)A¹⁴A¹⁵](CR⁹R¹⁰)S(C═), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵(CR⁹R¹⁰)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂c(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH₂CH₂CH(NH₂)COOH](CR⁵R⁶)ₚ(CR⁷R)ᵣ C(═O)O(CR⁹R¹⁰)ₛ( )C(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁹R¹⁰)ᵣ (CR⁹R¹⁰)ₛC(═O), C(═O)N[CH₂CH₂NHC(═O)CH₂CH₂CH(NH₂)COOH](CR⁵R⁶)ₚ OC(═O)(CR⁷R⁸)ᵣ(CR⁹R¹⁰)ₛC(═O);
wherein R⁸ is independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl; or
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle.

In an alternative embodiment of Formula I-P-2, each optional divalent group X is independently selected from (C₁₋₁₂alkylene)C(═O)N(R⁴), N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), and C(═O)CR^{S}═CR⁷—(CR⁹R¹⁰)ₛN(R⁴)C(═O), or any variant of the above groups formed by straightforward repositioning, addition, or deletion of the fragment N(R⁴)C(═O) or C(═O)N(R⁴)SO₂; wherein X is additionally connected to one to two amino acid residue(s) A¹² or A¹³; or
each optional divalent group X is independently selected from C(═O)(CR⁵R⁶)ₚ(CR⁷R⁸)ᵣ(CR⁹R¹⁰)ₛC(═O), C(═O)O(CR⁵R⁶)_{c}(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)SC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵(CR⁹R¹⁰)ₛN(C═O), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵(CR⁹R¹⁰)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)PC(═O)O(CR⁷R⁸)ᵣ (CR⁹R¹⁰)ₛC(═O), C(═O)N[CH₂CH₂NHC(═O)CH₂CH₂CH(NH₂)COOH](CR⁵R⁶)ₚ OC(═O)(CR⁷R⁸)ᵣ(CR⁹R¹⁰)ₛC(═O); wherein X is additionally connected to one to two amino acid residue(s) A¹² or A¹³.

In an alternative embodiment of Formula I-P-2, each optional divalent group X is S(CR⁵R⁶)ₚ(CR⁷R⁸)ᵣ(CR⁹R¹⁰)ₛOC(═O) or a group selected from

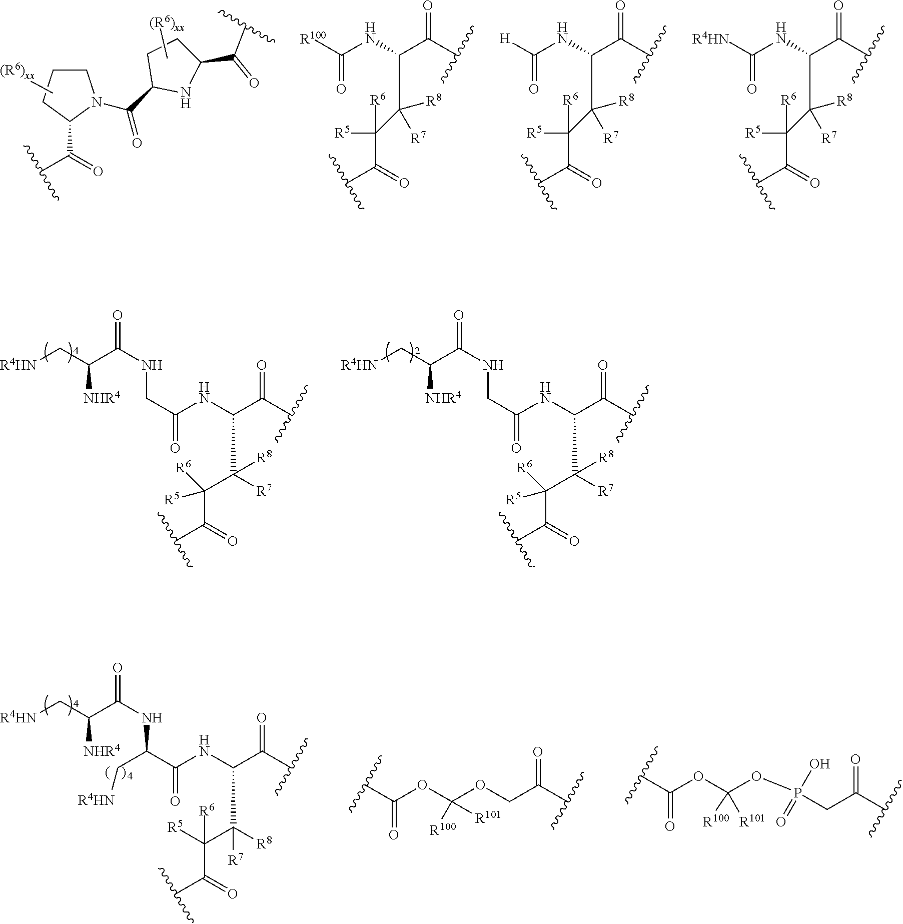

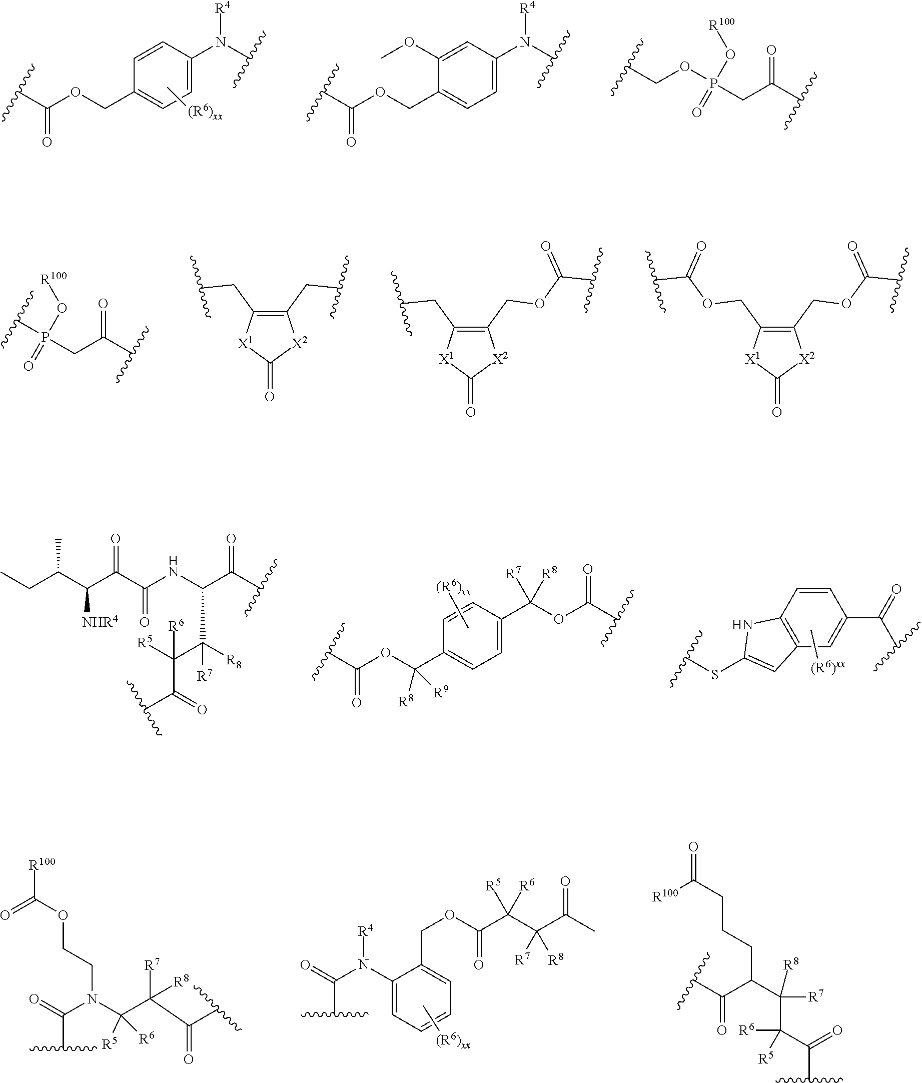

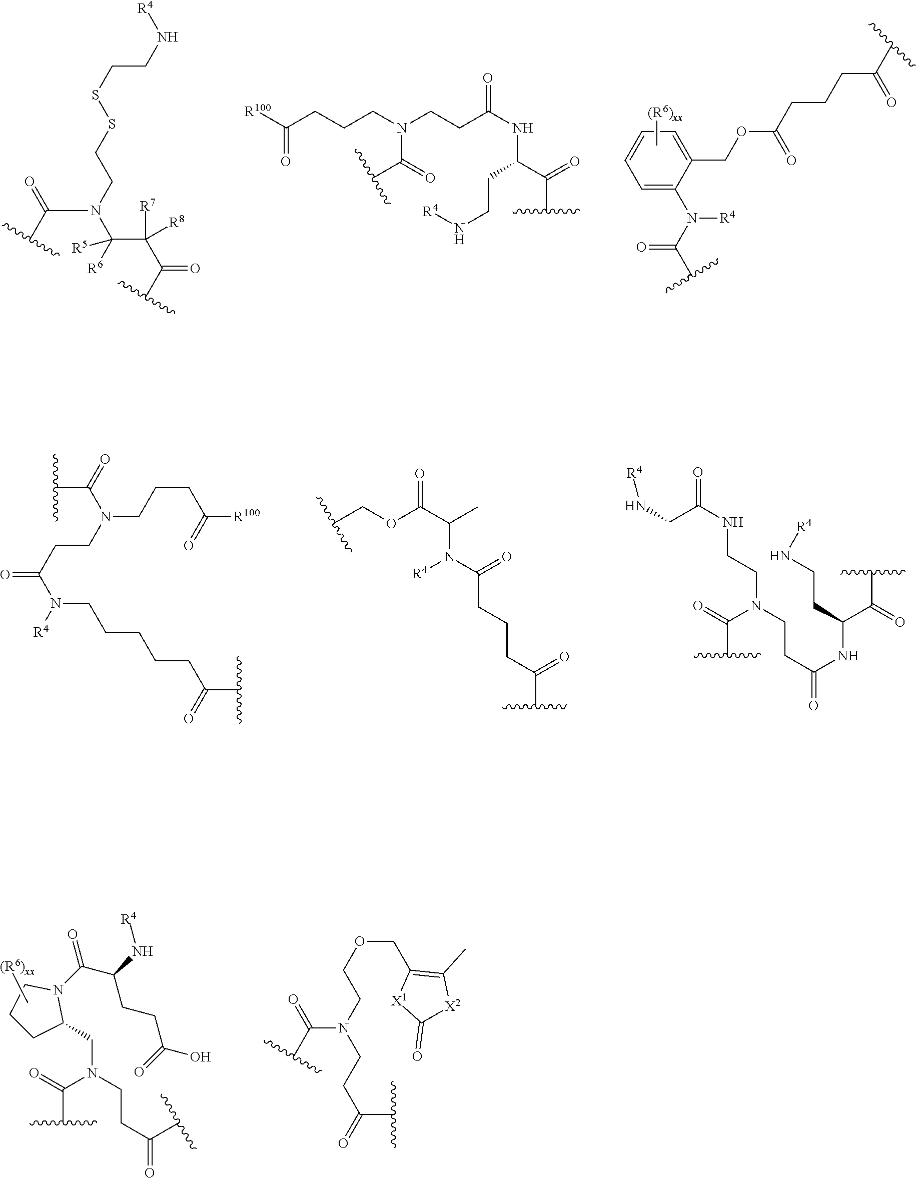

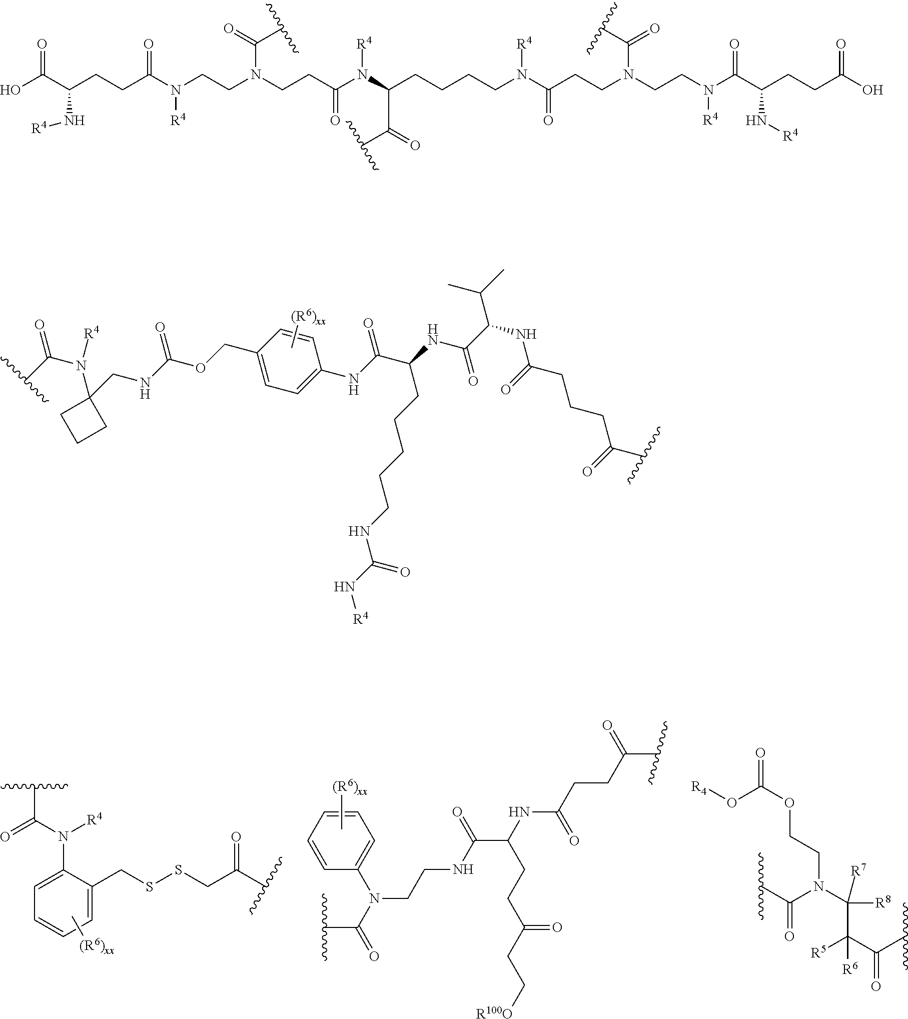

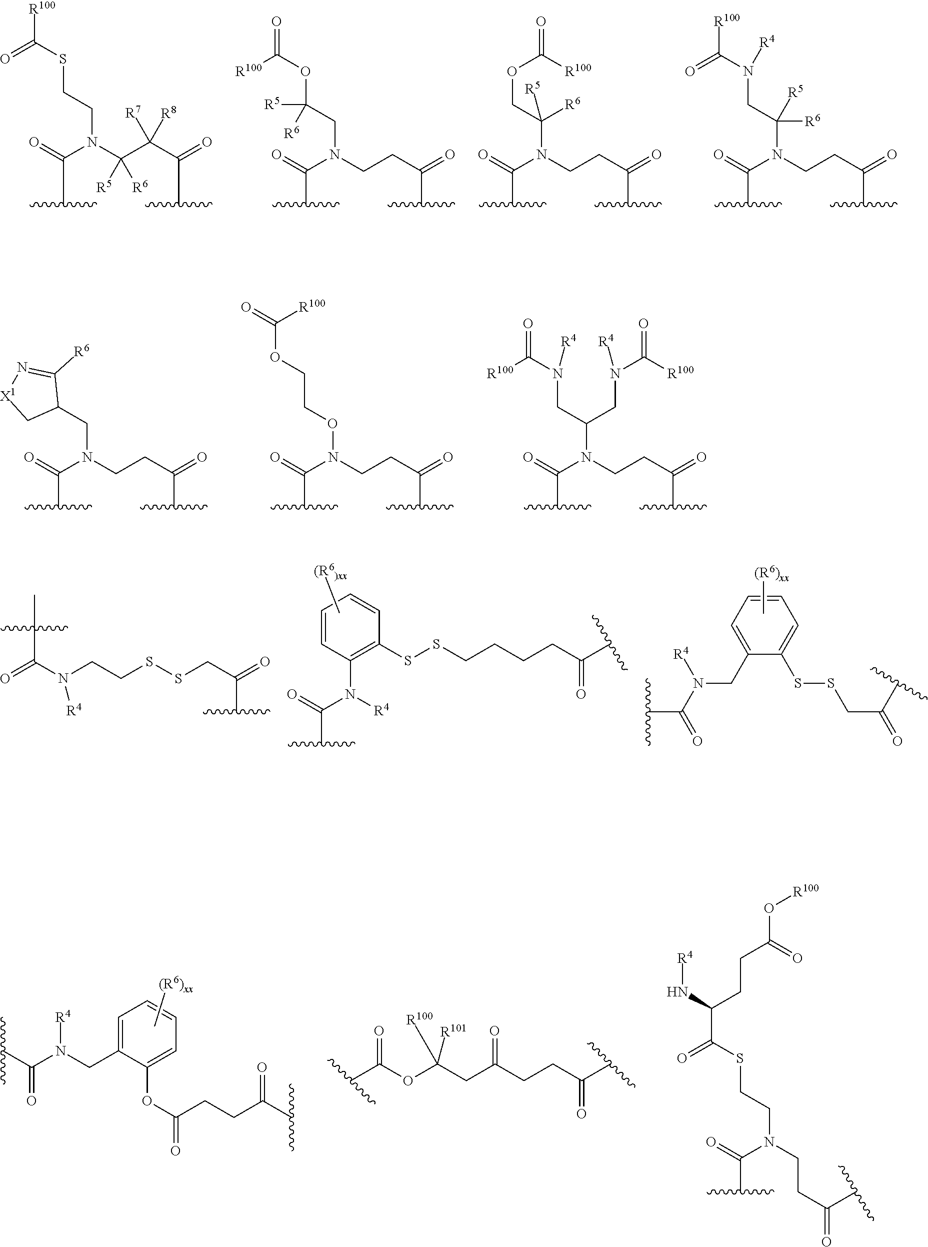

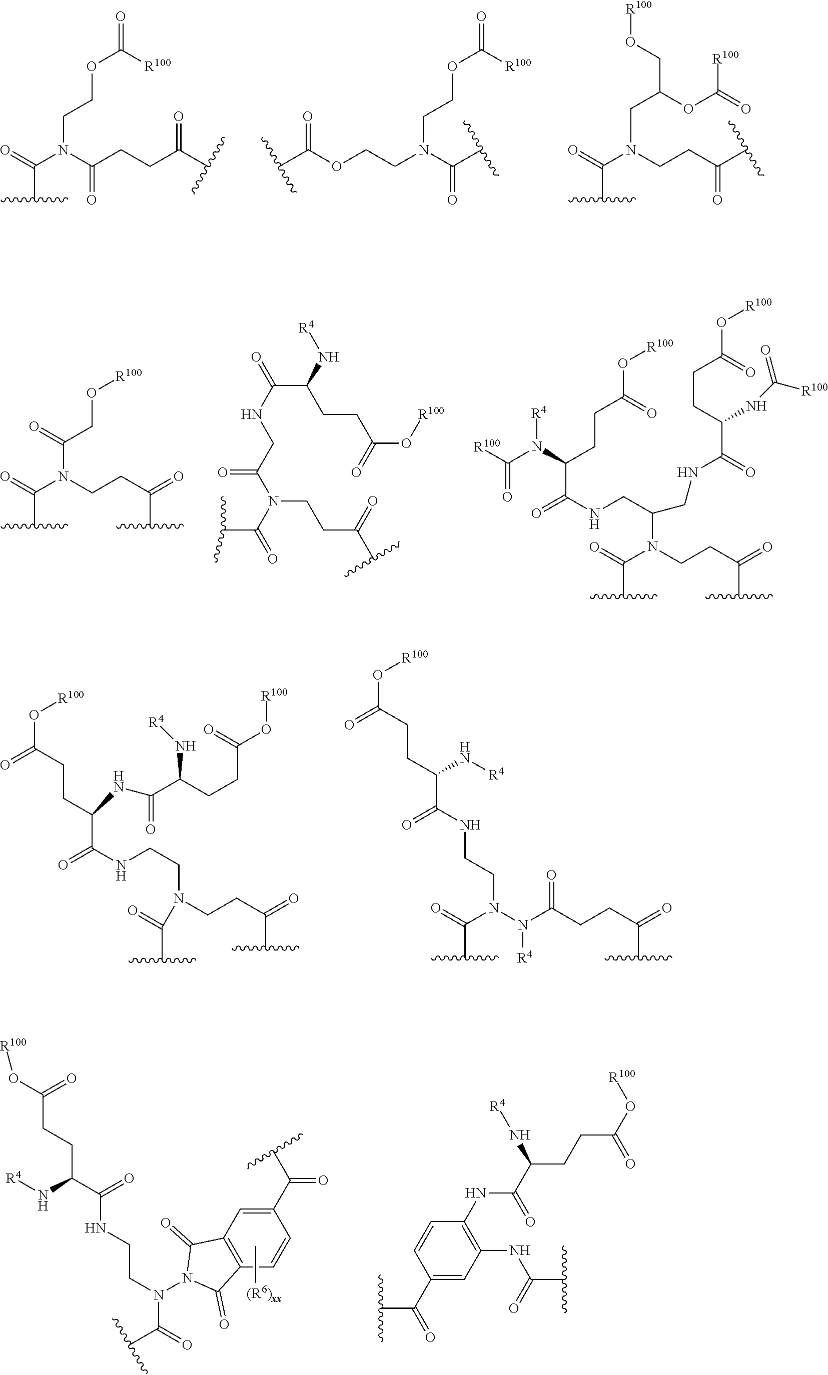

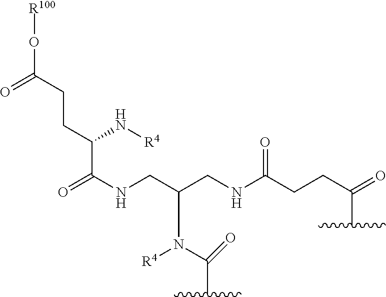

or any variant of the above X groups formed by the repositioning, addition, or deletion of the fragment N(R⁴)C(═O); or
wherein the group X is optionally connected to one to two amino acid residue(s) A¹² or A¹³;
wherein xx is 1, 2, or 3;
R¹⁰⁰ and R¹⁰¹ are independently selected from hydrogen and alkyl;
X¹ and X² are independently selected from O, NH, and CR⁵R⁶; and
all other variables are as defined herein in Formula I-P-2.

In an alternative embodiment of Formula I-P-2, each optional divalent group Z is independently selected from (C₁₋₁₂alkylene)C(═O)N(R⁴), N(R⁴)C(═O)(C₁₋₁₂alkylene), (C₁₋₁₂alkylene)N(R⁴)C(═O), C(═O)N(R⁴)(C₁₋₁₂alkylene), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)C(═O), C(═O)N(R⁴)CR⁵═CR⁷—(CR⁹R¹⁰)ₛC(═O), and C(═O)CR⁵═CR⁷—(CR⁹R¹⁰)ₛN(R⁴)C(═O),
or any variant of the above groups formed by straightforward repositioning, addition, or deletion of the fragment N(R⁴)C(═O) or C(═O)N(R⁴)SO₂;
wherein Z is additionally connected to one to two amino acid residue(s) A¹² or A¹³; or
each optional divalent group Z is independently selected from C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(═O), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O) C(═O)N(R⁵)SO₂(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)(CR⁵R⁶)ₚS—S(CR⁷R⁸)ᵣS—S(CR⁹R¹⁰)ₛOC(═O), C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁰R⁶)ₚ(CR⁷R⁸)ᵣN(C₁₋₆alkyl)C(═O)A¹⁴](CR⁹R¹⁰)ₛC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵(CR⁹R¹⁰)ₛC(═O), C(═O)N[(CR⁵R⁶)ₚ(CR⁷R⁸)ᵣNHC(═O)A¹⁴A¹⁵(CR⁹R¹⁰)ₛN(C═O), C(═O)N[(CR⁵R⁶)ₚ(CR⁷R⁸)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A](CR⁹R 1)ₛC(═O), C(═O)N[(CR⁵R⁶)ₚ(CR⁷R⁸)ᵣOC(═O)A¹⁴A¹⁵(CR⁹R¹⁰)ₛN(C═O), C(═O)N[(CR⁵R⁶)ₚ(CR⁷R⁸)ᵣOC(═O)A¹⁴A¹⁵(CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂c(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚ(CR⁷R⁸)ᵣOC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), and C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O); wherein Z is additionally connected to one to two amino acid residue(s) A¹² or A¹³.

Within the broadest definition, certain compounds of Formula I-P-1, Formula I-P-2, or Formula I may be preferred. Specific and preferred values listed below for radicals, substituents, and ranges, are for illustration only; they do not exclude other defined values or other values within defined ranges for the radicals and substituents.

In some preferred compounds described herein C₁₋₁₄alkyl can be methyl, ethyl, propyl, isopropyl, butyl, iso-butyl, sec-butyl, octyl, nonyl, decyl, and isomeric forms thereof.

In some preferred compounds described herein C₂₋₂alkenyl can be vinyl, propenyl, allyl, butenyl, and isomeric forms thereof (including cis and trans isomers).

In some preferred compounds described herein C₃₋₇cycloalkyl can be cyclopropyl, cyclobutyl, cyclopentyl, cyclohexyl, and isomeric forms thereof.

In some preferred compounds described herein C₁₋₁₄heteroalkyl can be hydroxymethyl, hydroxyethyl, 2-(N,N-dimethylamino)ethyl, 2-(4-morpholino)ethyl, and 2-methoxyethyl.

In some preferred compounds described herein halo can be fluoro (F) or chloro (Cl).

It will also be appreciated by those skilled in the art that compounds described herein may have additional chiral centers and be isolated in optically active and racemic forms. The compounds described herein can be racemic, optically active, tautomeric, geometric, in a stereoisomeric form, or a mixture thereof.

Any embodiment described herein can be combined with any other embodiment described herein.

Embodiment 1: The compound of Formula I, Formula I-P-1, or Formula I-P-2 as provided above, and wherein
the integers a through g are each 1; and wherein
A¹ is Thr or Ser; A², A³ A⁶, and A⁷ are independently selected from Dab, Dap, Ser, and Thr; A⁴ is Leu or Ile; and A⁵ is Phe, D-Phe, Bip, D-Bip, Val, or D-Val.

Embodiment 2: The compound of Formula I, Formula I-P-2, or Formula I-P-2 or of Embodiment 1, and wherein the cyclic peptide structure in the Formula I, Formula I-P-1, or Formula I-P-2 is comprised of optional amino acid residues A¹ through A⁷ and is a cyclic peptide structure identical to that present in polymyxin A, polymyxin B, polymyxin B nonapeptide (H-Thr-Dab-cyclo[Dab-Dab-D-Phe-Leu- Dab-Dab-Thr]), polymyxin B heptapeptide (H-cyclo[Dab-Dab-D-Phe-Leu- Dab-Dab-Thr]), polymyxin E, or octapeptin, or similar structures.

Embodiment 3: The compound of Formula I-P-1 or of Embodiments 1 or 2 according to Formula II-P:

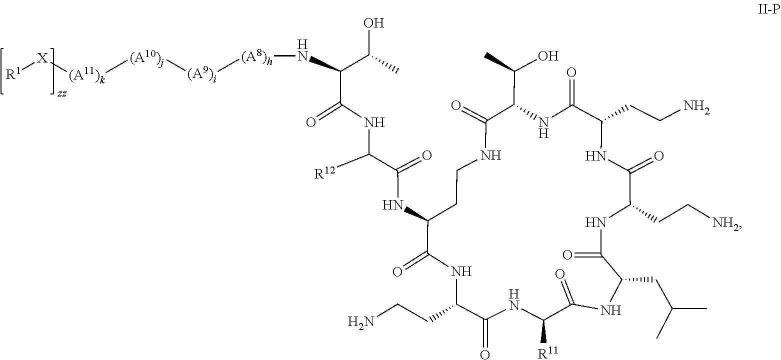

or a pharmaceutically acceptable salt, solvate, or hydrate thereof, wherein:
R¹¹ is CH₂CH(CH₃)₂ or CH₂Ph; and
R² is CH₂NH₂ or CH₂CH₂NH₂.

Embodiment 4: The compound of Formula I or of Embodiments 1 or 2 according to Formula III:

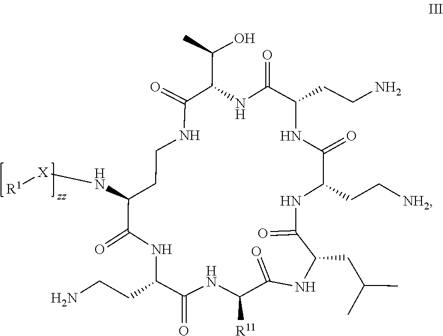

or a pharmaceutically acceptable salt, solvate, or hydrate thereof, wherein:
the integer zz is equal to 1 or 2;
R¹ is independently selected from a residue formed by subtracting a single H atom at one of the following H-containing groups independently selected from NH, OH, SH, C(═O)OH, CONH, SO₂NH, and S(═O)NH present in afatinib, ARS-1630, axitinib, BGB-324, BLU-554, brivanib, cabozantinib, cediranib, ceritinib, ciforadenant, derazantinib, dovitinib, foretinib, lenvatinib, monomethyl auristatin E, irinotecan, maytansinoid, neratinib, nilotinib, nintedanib, ozogamicin, paclitaxel, pazopanib, regorafenib, sacituzumab, selpercatinib, semaxanib, sorafenib, sunitinib, SN-38, trastuzumab, tesirine, temsirolimus, and tivantinib; or variants of aforementioned structures derived from these by chemical modification(s) of said structure(s); and
X is a structure selected from the group consisting of C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), and C(═O)N[CH₂CH₂OC(═O)CH(NH₂)CH₂CH₂COOH]CH₂CH₂C(═O).

Embodiment 5: The compound of Formula I, Formula I-P-1, Formula I-P-2, Formula II-P, or Formula III of Embodiment 3, wherein
group X is selected from the structures below, wherein either the left side or the right side of group X is connected to R¹:

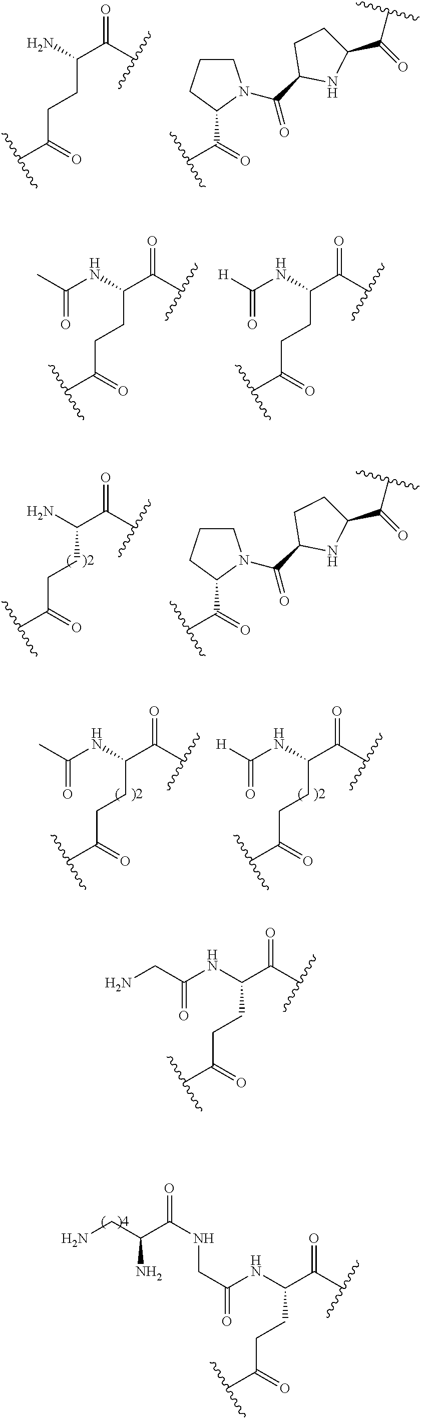

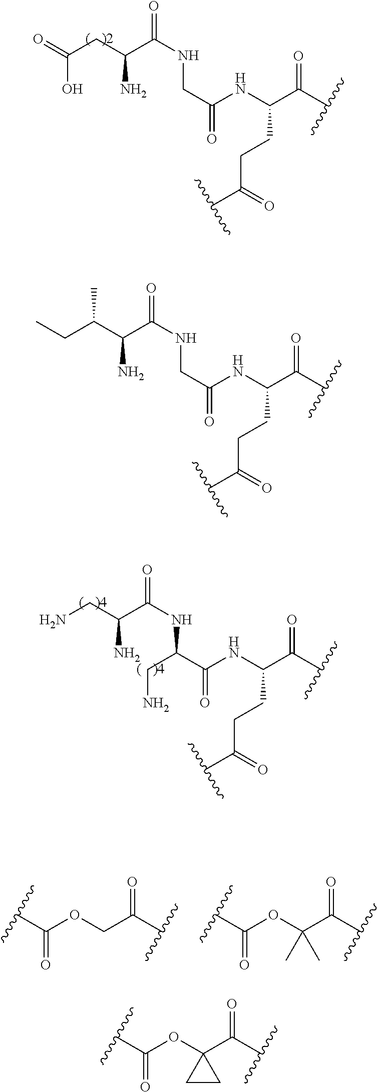

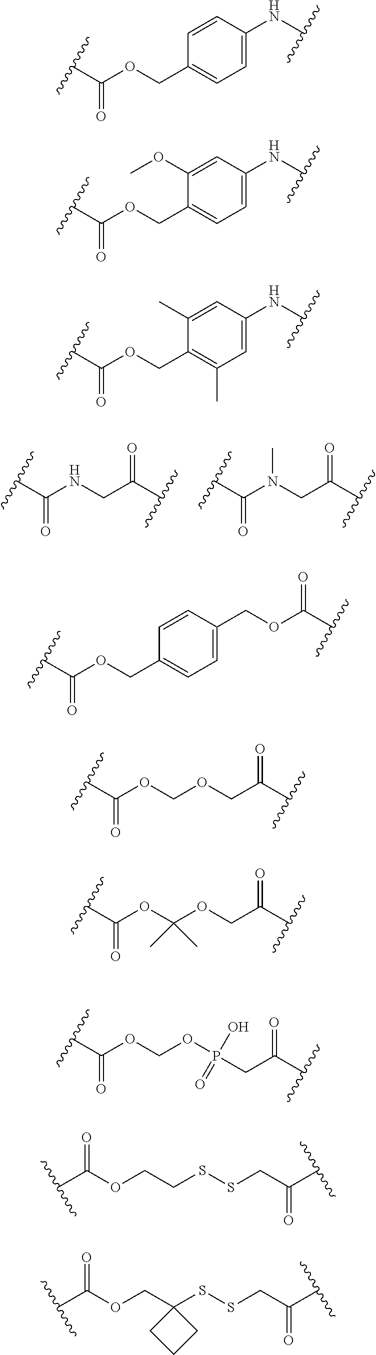

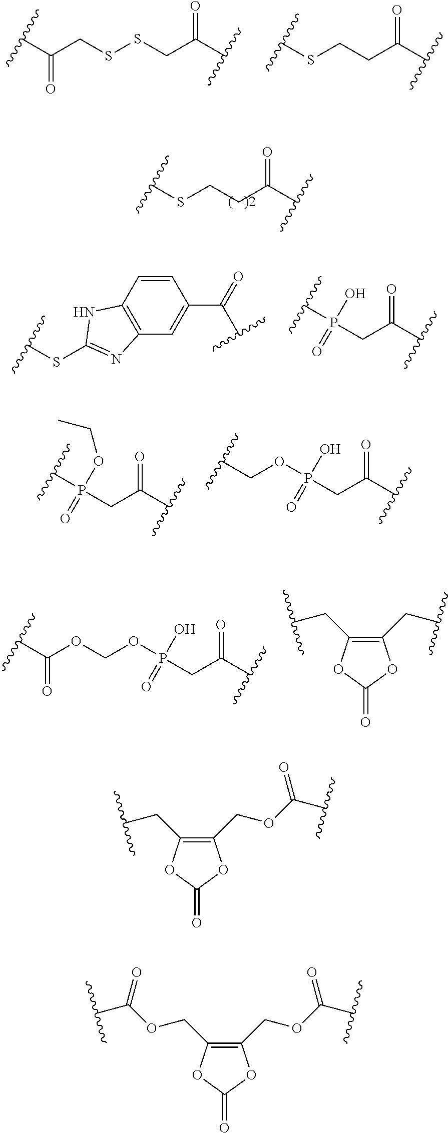

Embodiment 6: The compound of Formula I, Formula I-P-1, Formula I-P-2, Formula II-P, or Formula III of Embodiment 4, wherein group X, either at its left or right side therein, incorporates one or more additional divalent groups selected from C₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O)ᵣN(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups. In an Embodiment, 1, 2, 3, 4, or 5, additional divalent groups are incorporated and are independently selected.

Embodiment 7: The compound of Formula I-P-2 or of Embodiments 1 or 2 according to Formula III-P:

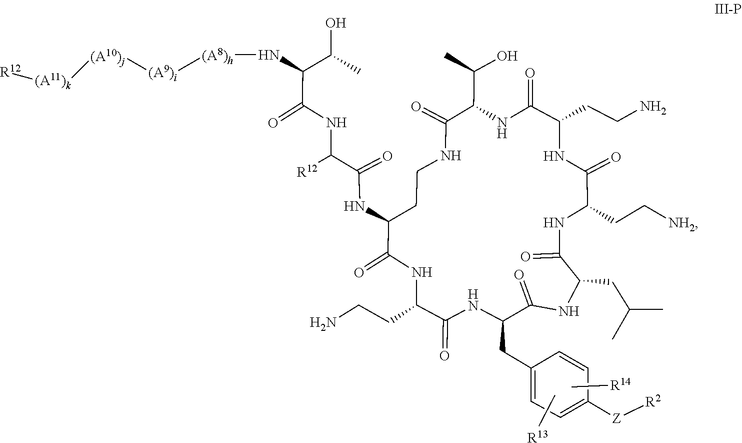

wherein groups R¹³ and R¹⁴ are independently selected from H, halo, NH₂, CN, OH, OC₁₋₁₄alkyl, O-aryl, NH(C₁₋₆alkyl), NH(OC₁₋₆alkyl), C₁₋₄alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄alkyl.

Embodiment 8: The compound of the formula III-P of Embodiment 7, wherein group Z is selected from the structures below, wherein the right side of group Z is connected to R²:

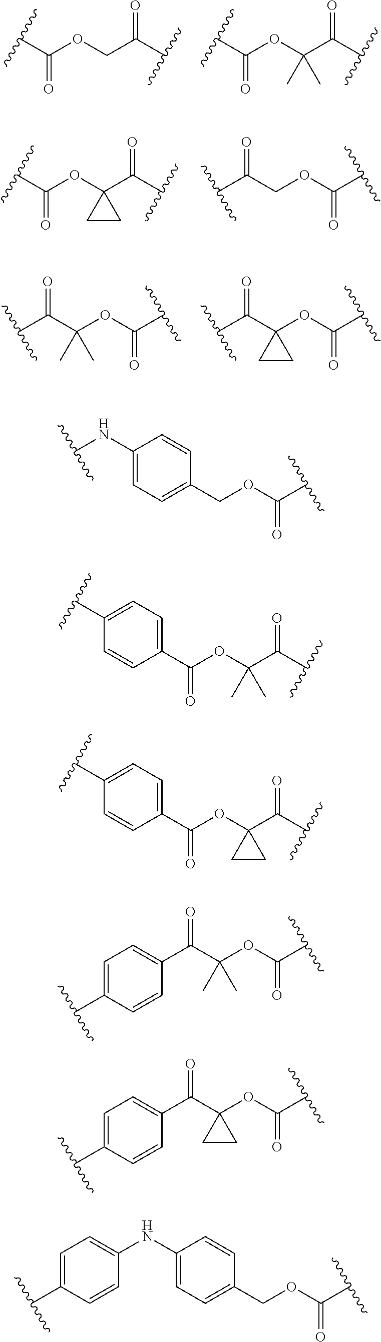

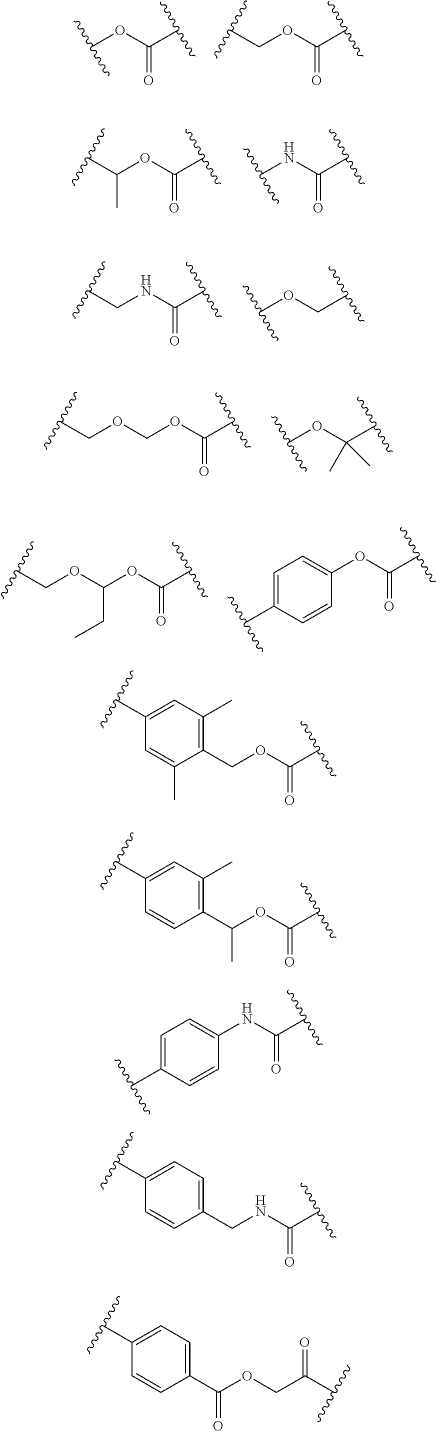

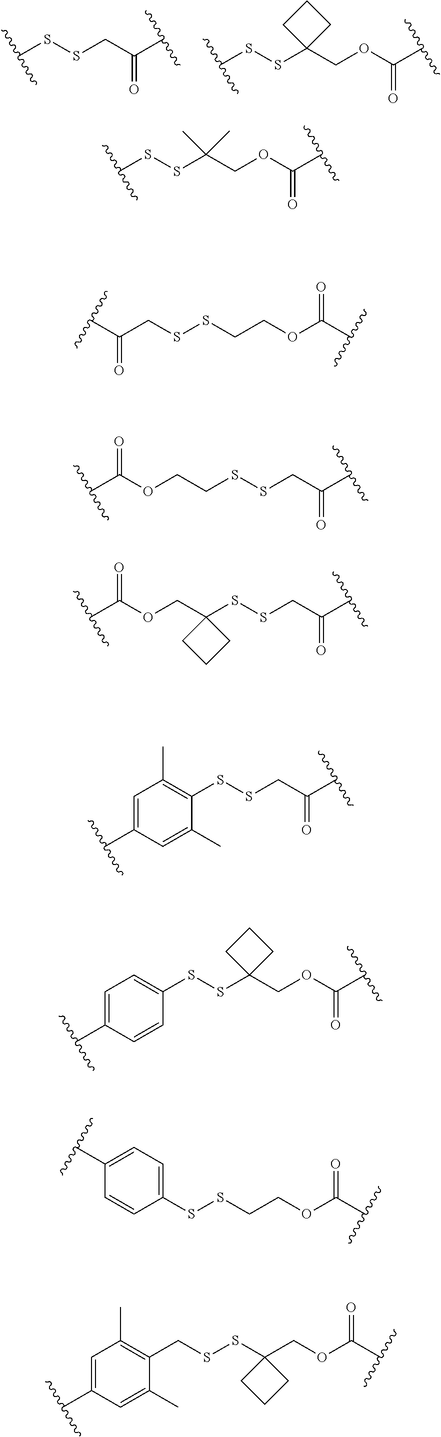

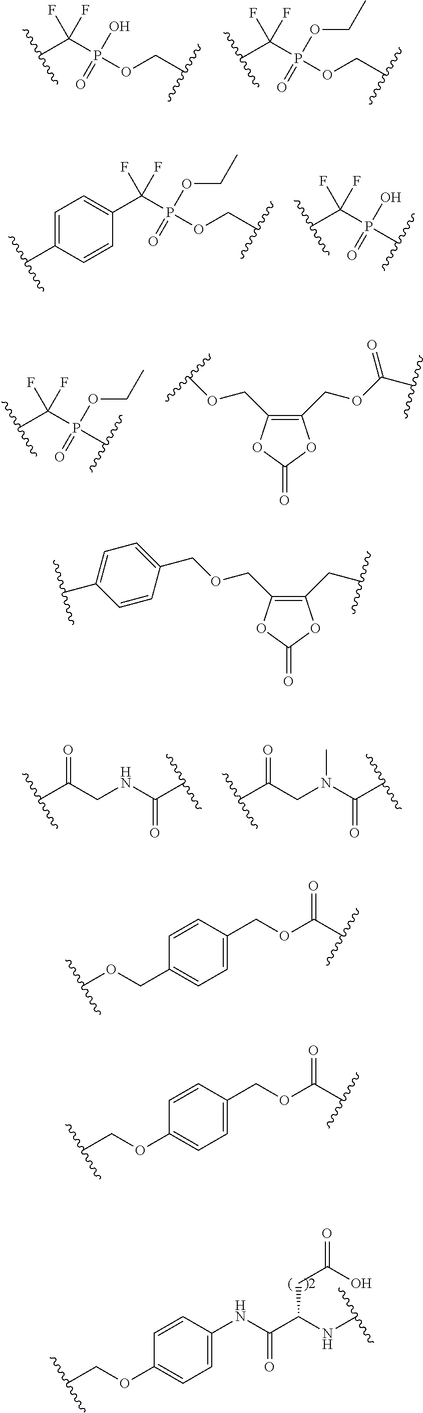

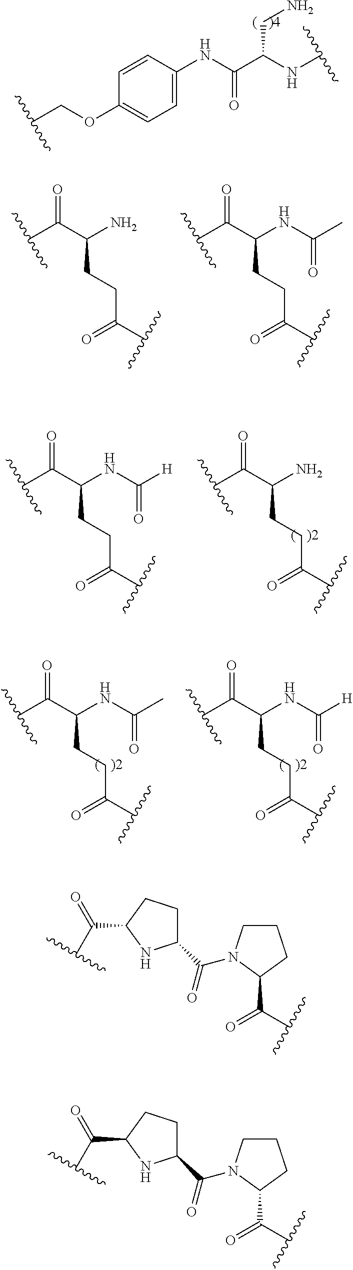

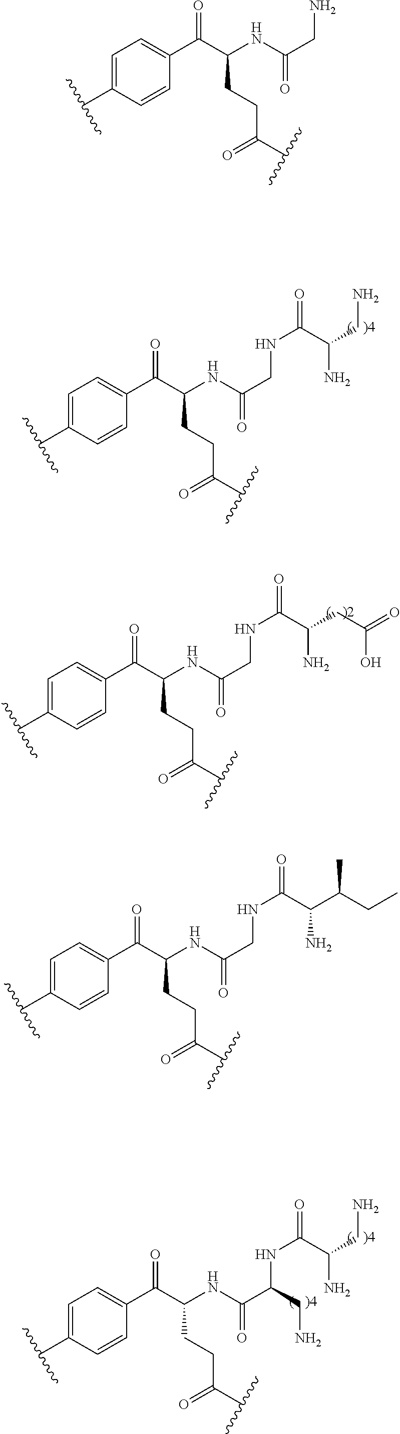

In one embodiment the compound of Formula I-P−1 is a compound of Formula II-P:

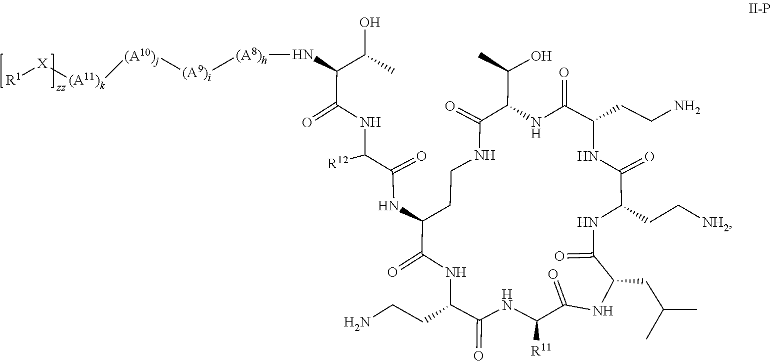

or a pharmaceutically acceptable salt, solvate, or hydrate thereof, wherein
R¹¹ is CH₂CH(CH₃)₂ or CH₂Ph;
R¹² is CH₂NH₂ or CH₂CH₂NH₂; and
other groups and integers in the compound of Formula II-P are selected as defined above for the compound of Formula I-P-1, or any embodiment thereof.

One preferred group of compounds of Formula I, Formula I-P-1, Formula I-P-2, Formula II, Formula II-P, Formula III, or Formula III-P is illustrated below, wherein each X is independently selected from the structures below, wherein either the left side or the right side of X is connected to RU.

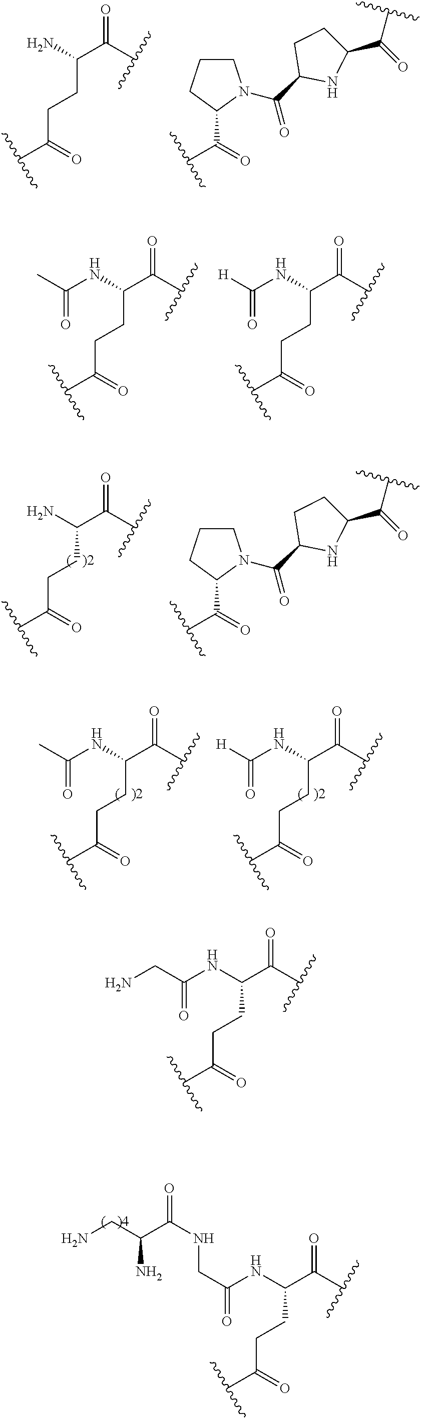

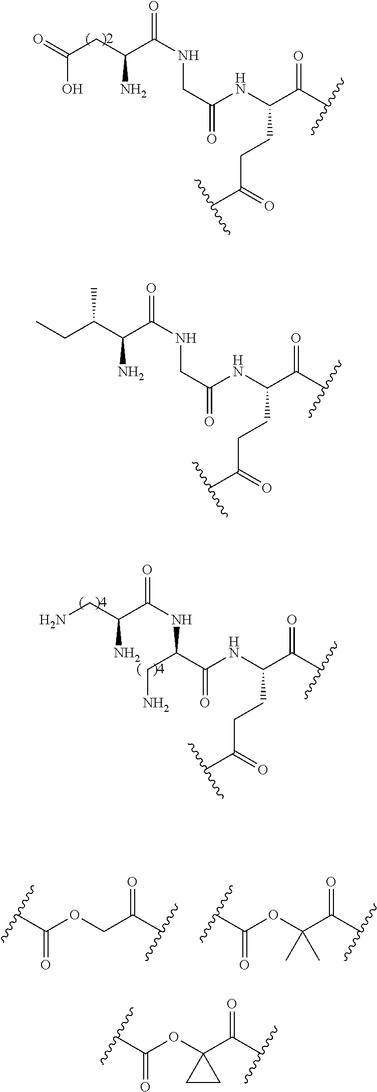

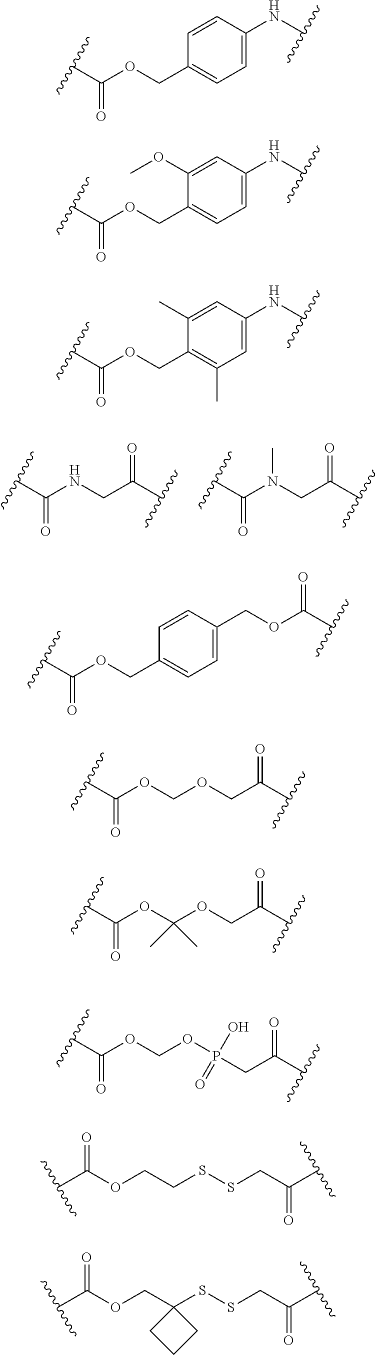

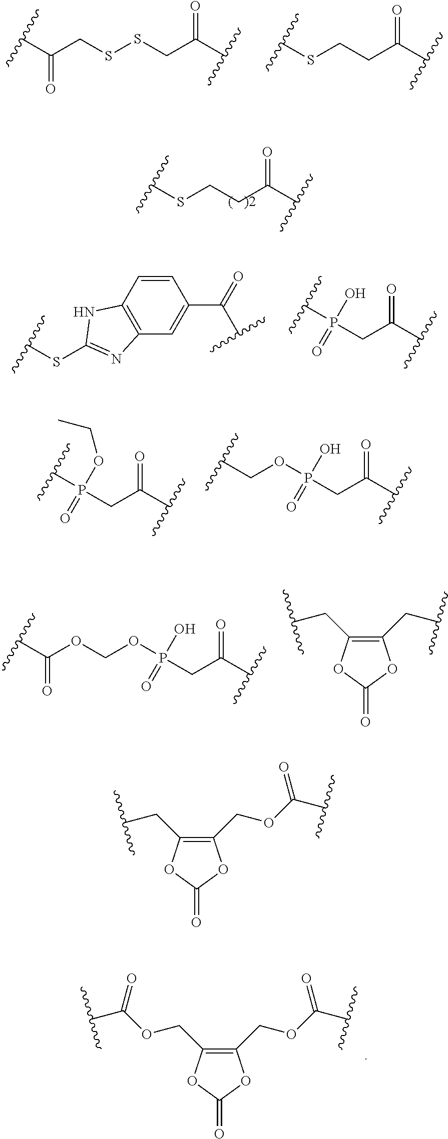

One preferred group of compounds of Formula I, Formula I-P-1, Formula I-P-2, Formula II, Formula II-P, Formula III, or Formula III-P is illustrated below, wherein each X is independently selected from the structures below, wherein either the left side or the right side of X is connected to RU:

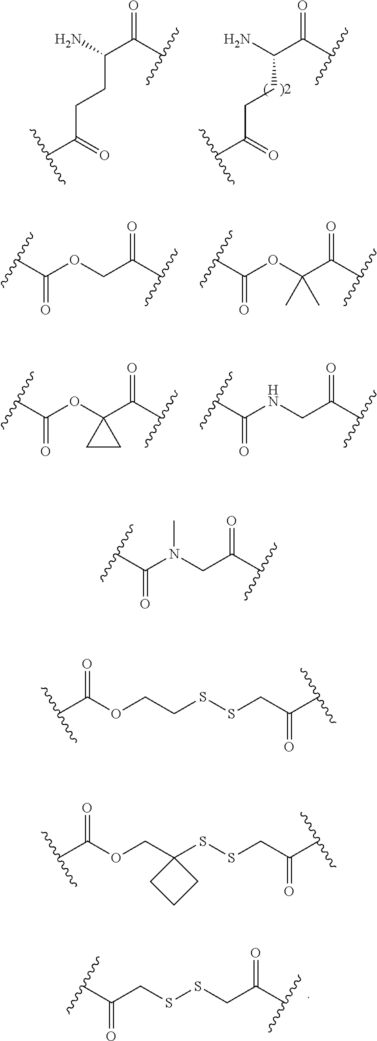

In another embodiment, provided is a compound of Formula I, Formula I-P-1, Formula I-P-2, Formula II-P, Formula II, Formula III-P, or Formula III, wherein each X is independently selected from the following structures, connected to R¹ at the left side of X:

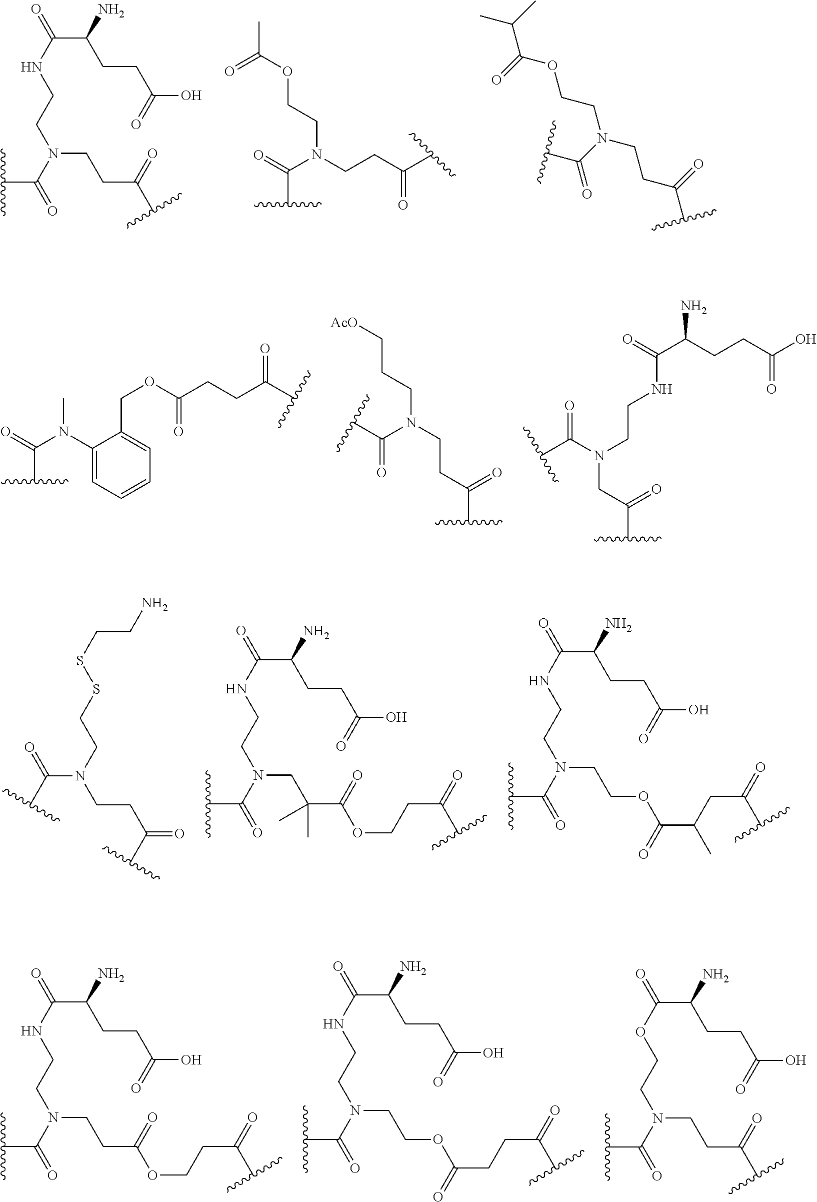

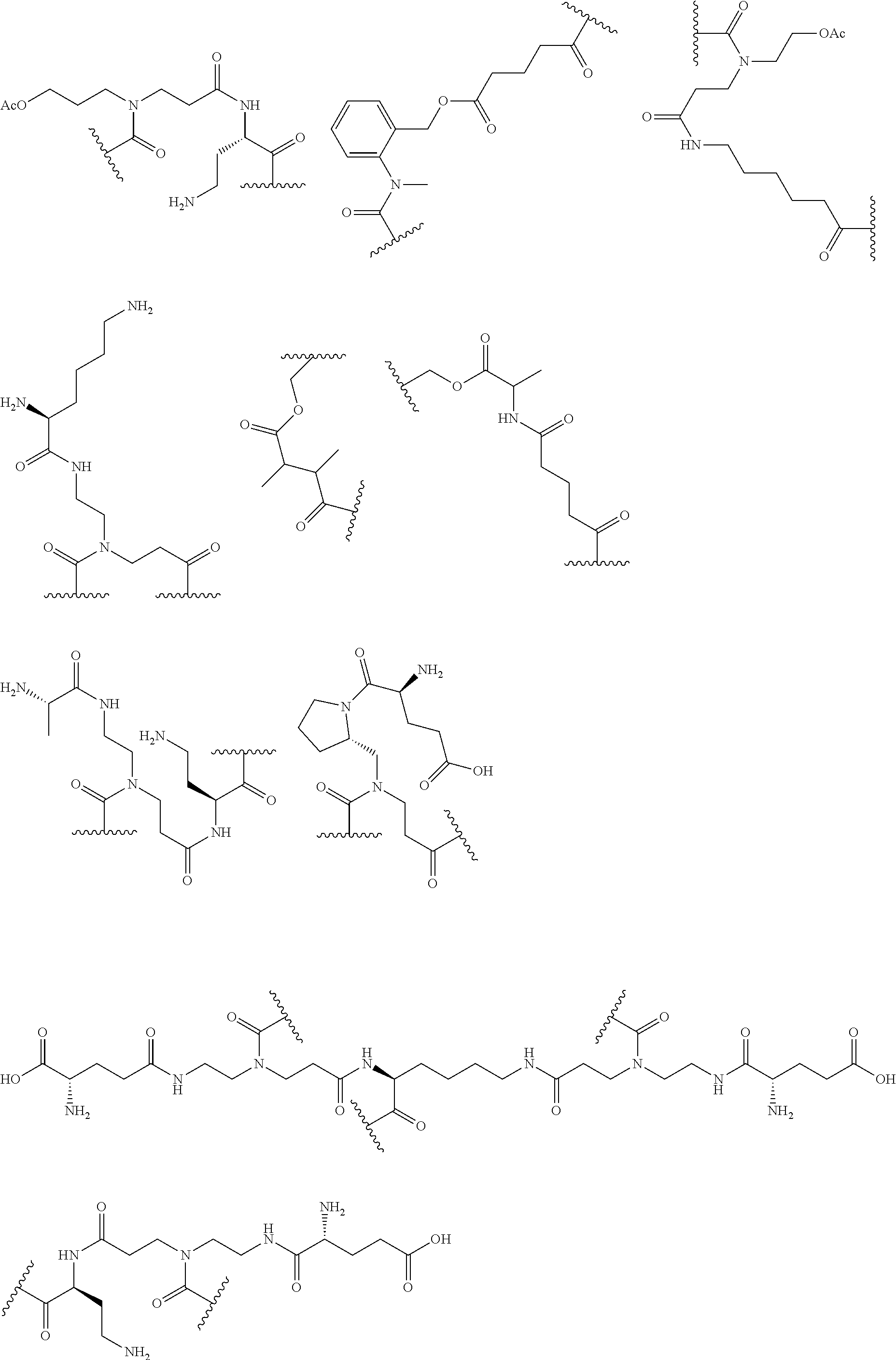

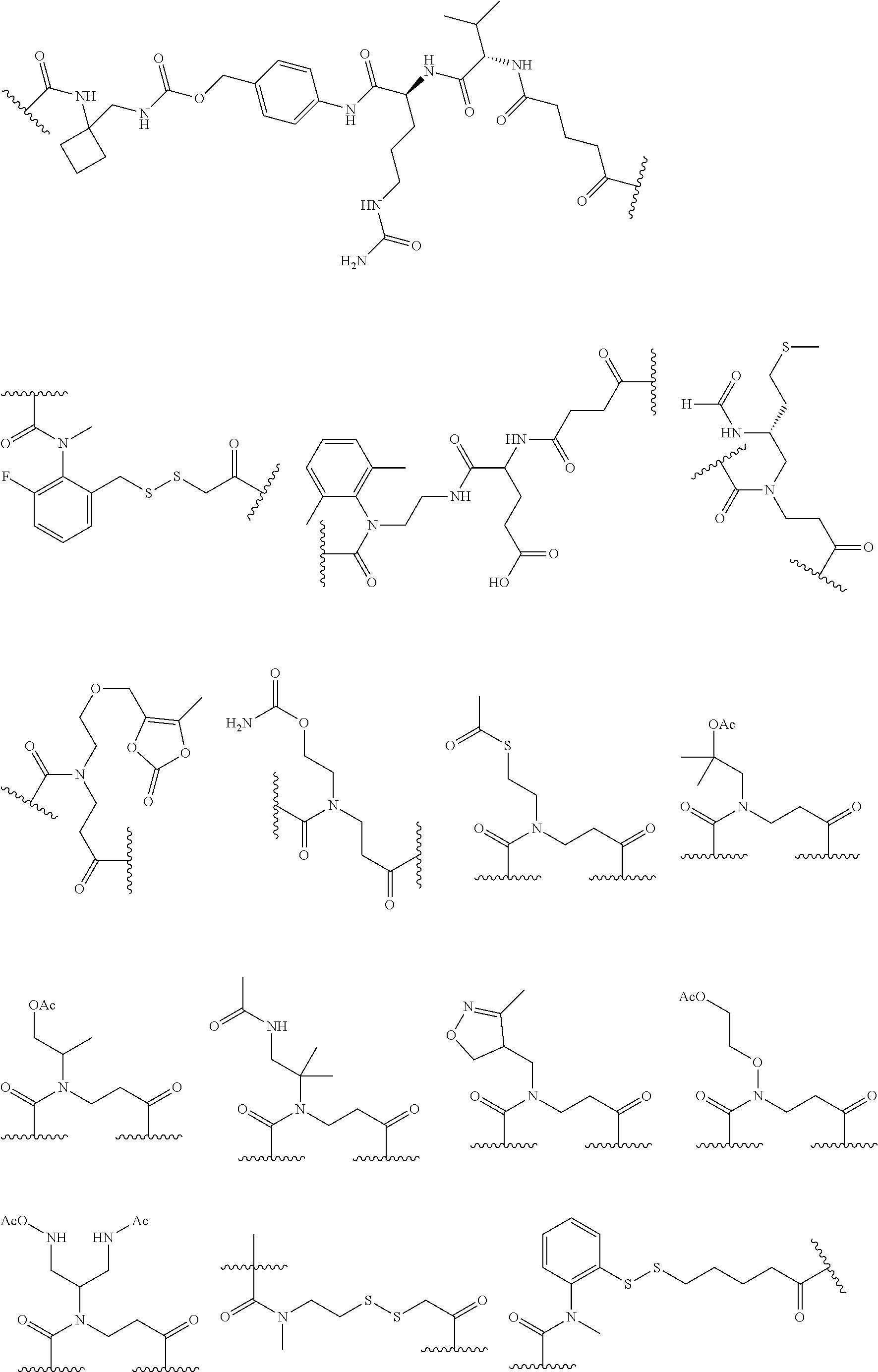

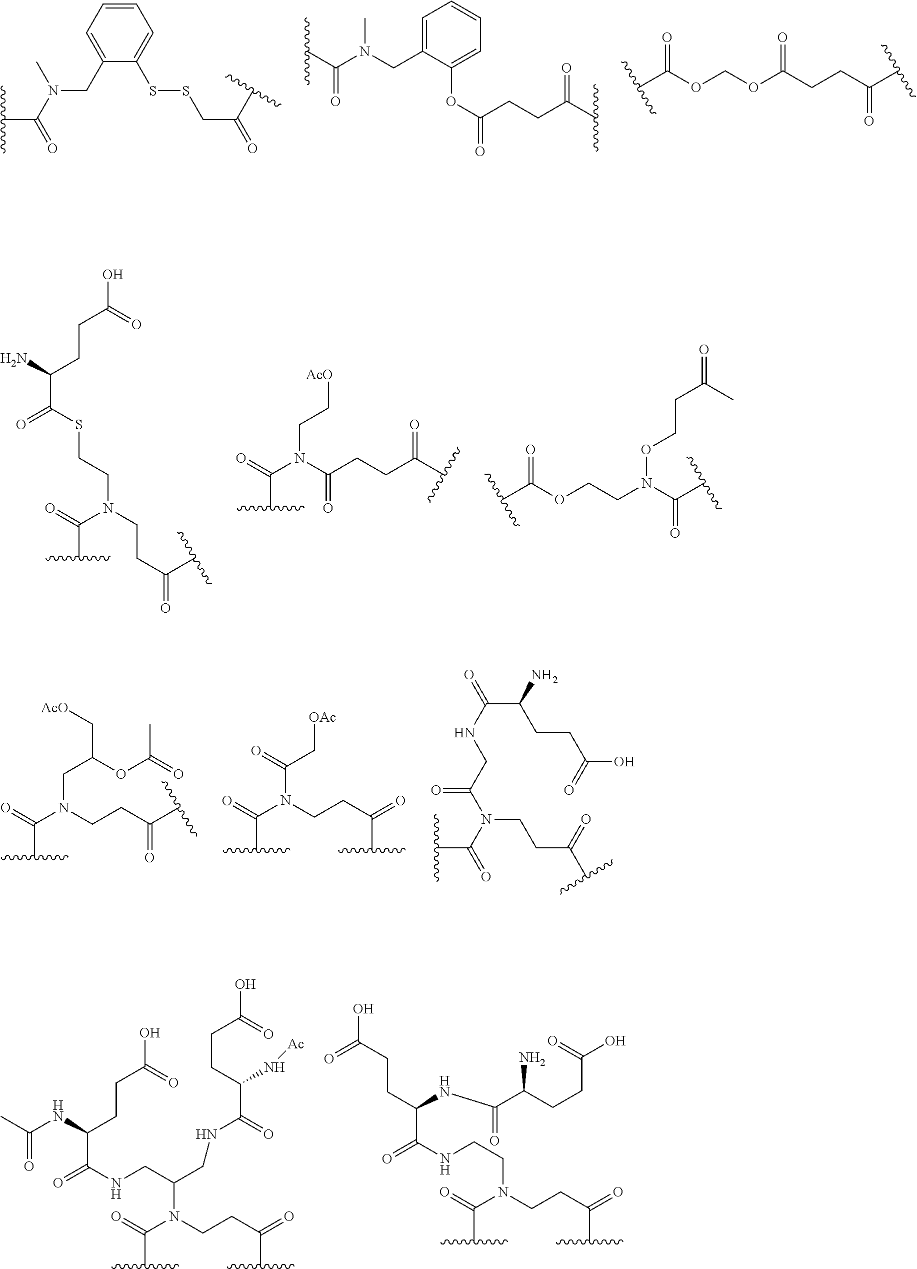

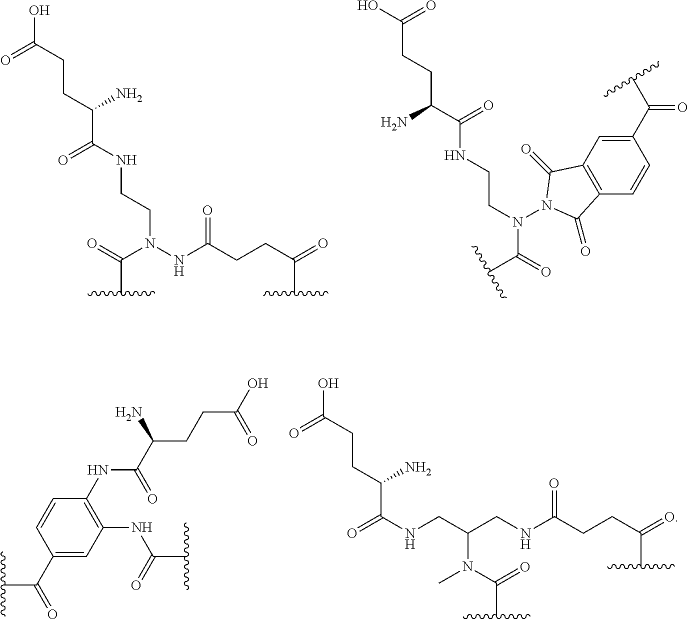

In another embodiment, provided is a compound of Formula I, Formula I-P-1, Formula I-P-2, Formula II-P, Formula II, Formula III-P, or Formula III, wherein each X is independently selected from the following structures, connected to R at the left side of X:

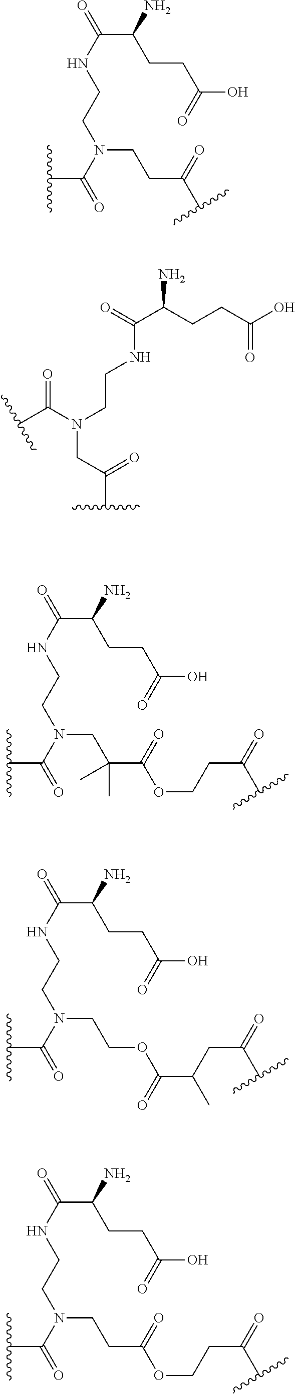 [SMILES: *C(=O)CCN(CCNC(=O)[C@@H](N)CCC(=O)O)C(*)=O ; *C(=O)CN(CCNC(=O)[C@@H](N)CCC(=O)O)C(*)=O ; *C(=O)CCOC(=O)C(C)(C)CN(CCNC(=O)[C@@H](N)CCC(=O)O)C(*)=O ; *C(=O)CC(C)C(=O)OCCN(CCNC(=O)[C@@H](N)CCC(=O)O)C(*)=O ; *C(=O)CCOC(=O)CCN(CCNC(=O)[C@@H](N)CCC(=O)O)C(*)=O]

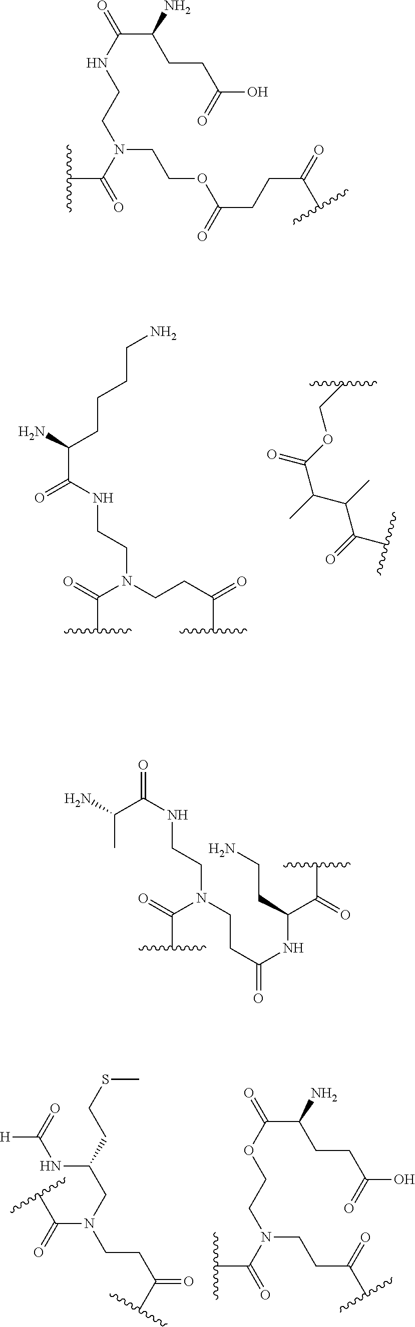

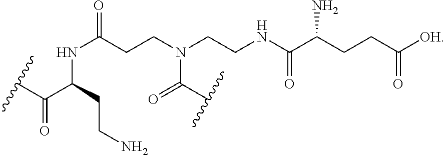 [SMILES: *C(=O)[C@H](CCN)NC(=O)CCN(CCNC(=O)[C@H](N)CCC(=O)O)C(*)=O]

In another preferred embodiment, each X illustrated in the two paragraphs above, either at its left or right side therein, independently incorporates one or more additional divalent groups independently selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₄alkyl)C(═O), N(C₁₋₄alkyl)(CH₂)ₚO(CH₂)ᵣO(CH₂)ₛC(═O), N(C₁₋₄alkyl)(CH₂)ₚO(CH₂)ᵣO(CH₂)ₛOC(═O), N(C₁₋₄alkyl)(CH₂)ₚO(CH₂)ᵣO(CH₂)ₛNHC(═O), N(C₁₋₄alkyl)(CH₂)ₚO(CH₂)ᵣO(CH₂)ₛN(C₁₋₁₄alkyl)C(═O), and similar linear groups. In an Embodiment, 1, 2, 3, 4, or 5, additional divalent groups are incorporated and are independently selected. In one embodiment, the compound of Formula I-P-1 is a compound of Formula III-P:

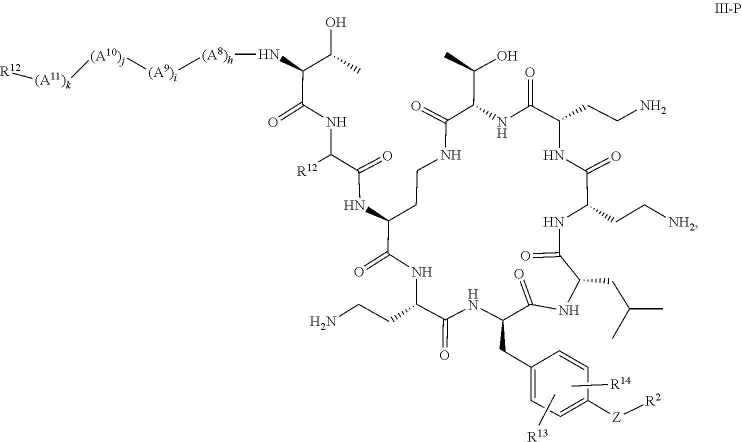

wherein R¹³ and R¹⁴ are independently selected from H, halo, NH₂, CN, OH, OC₁₋₁₄alkyl, O-aryl, NH(C₁₋₆alkyl), NH(OC₁₋₆alkyl), C₁₋₁₄alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄alkyl; and
wherein other groups and integers in the compound of Formula III-P are selected as defined above for the compound of Formula I-P-1, or any embodiment thereof.

One preferred group of compounds of Formula III-P is that wherein Z is selected from the structures below, wherein the right side of Z is connected to R².

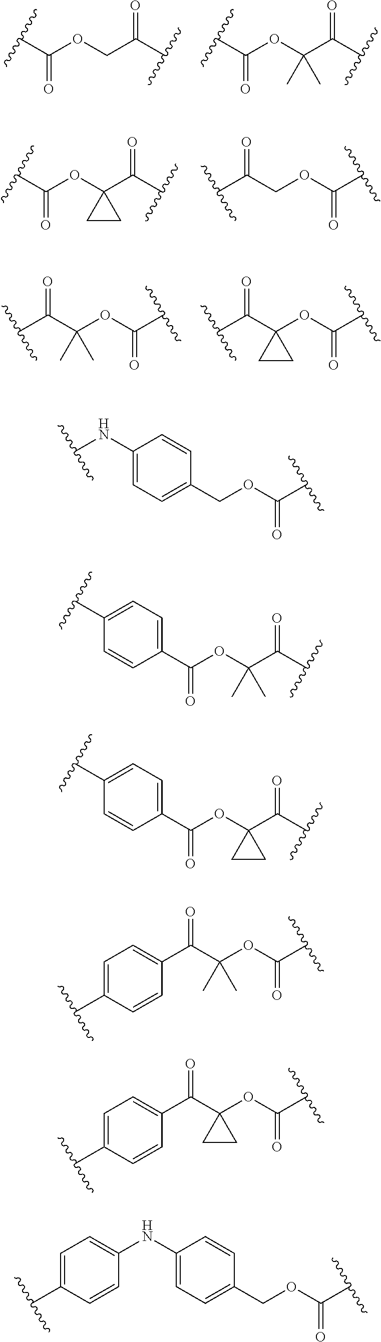

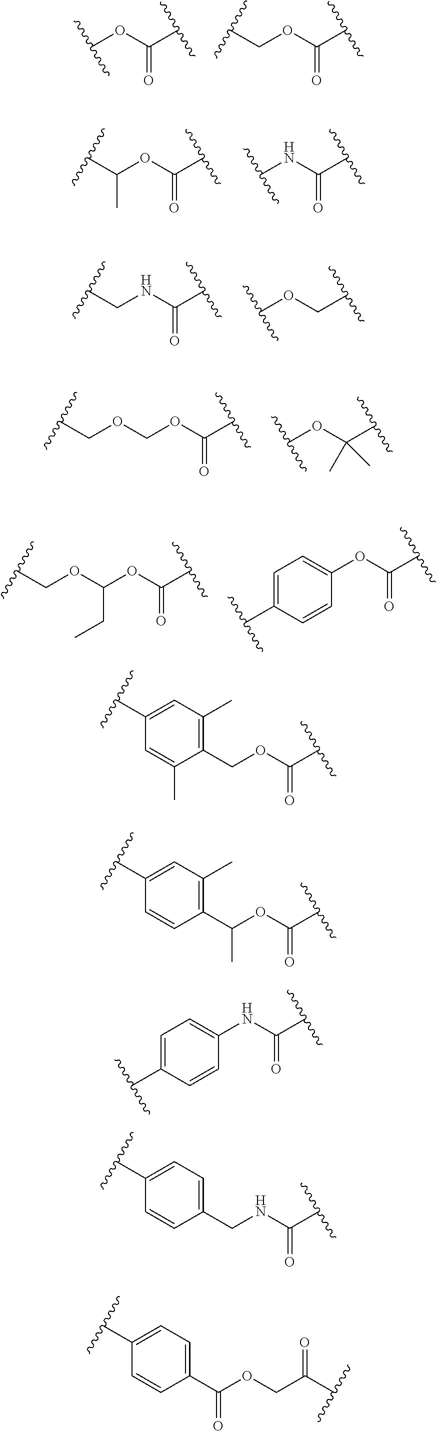

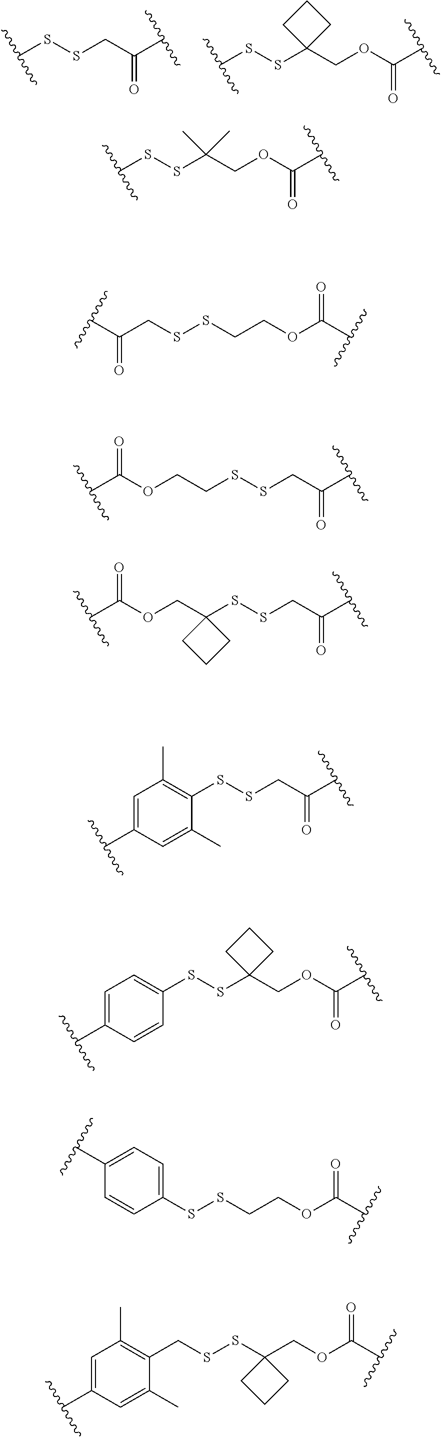

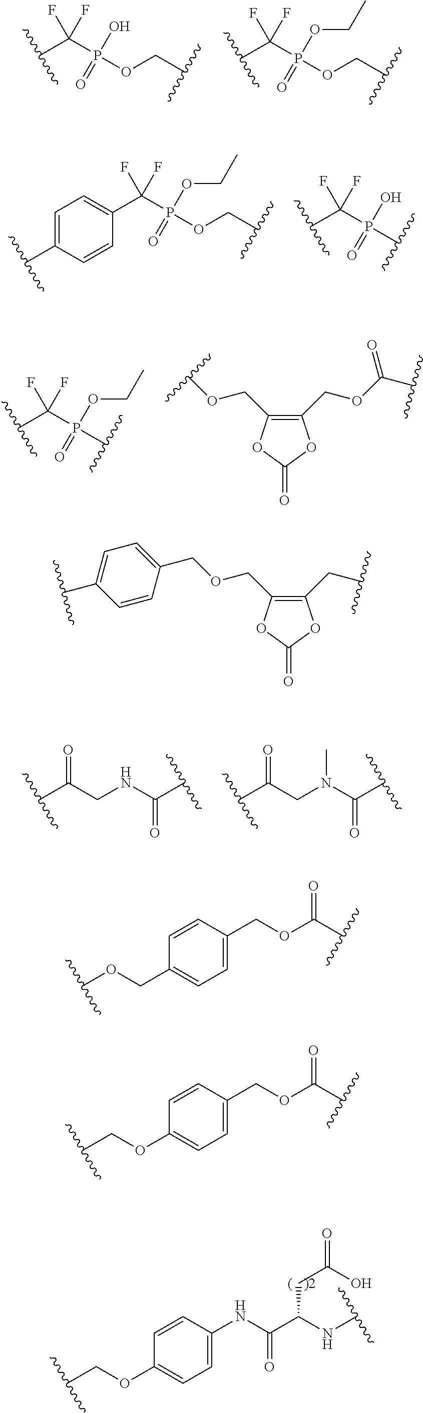

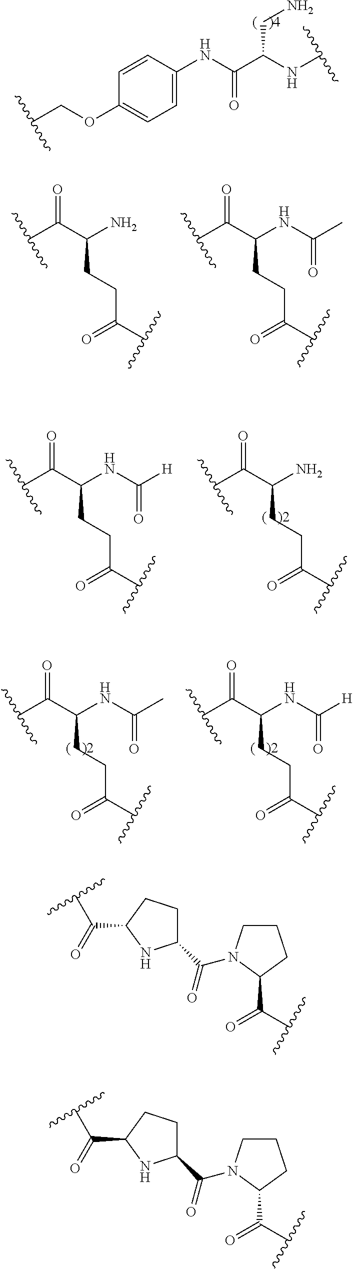

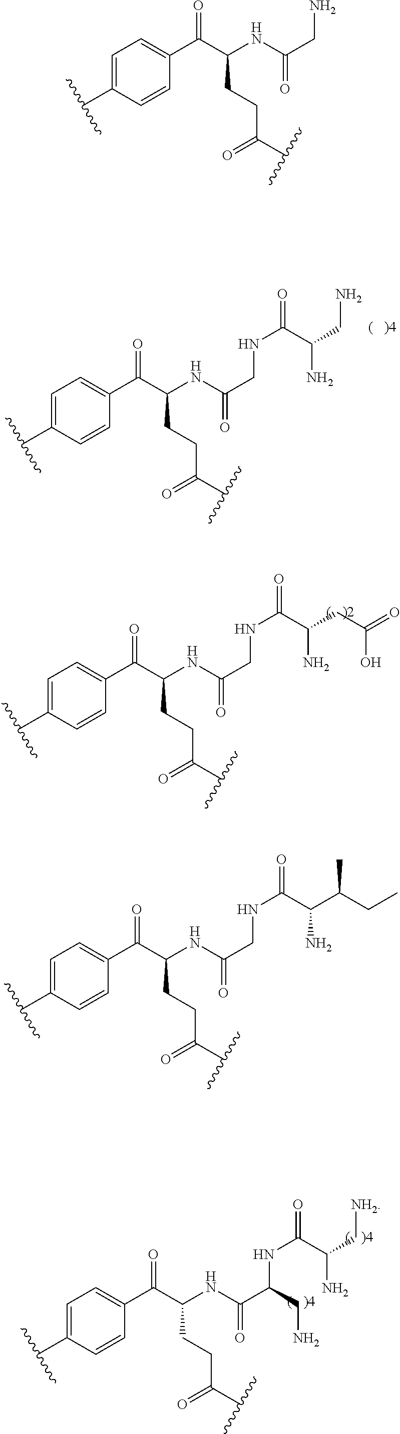

In another preferred embodiment, each Z illustrated above, at its left side therein, incorporates one or more additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups. In an Embodiment, 1, 2, 3, 4, or 5, additional divalent groups are incorporated and are independently selected.

In another preferred embodiment, the compound of Formula I-P-1 is a compound of Formula IV-P:

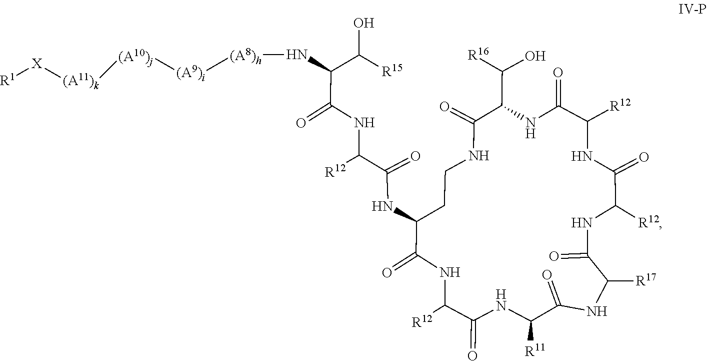

or a pharmaceutically acceptable salt, solvate, or hydrate thereof, wherein
X is selected from the following structures and is connected to R at the left side of X: C(═O)N[(CR⁵R⁶)ₚ(CR⁷R⁸)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR¹R⁹)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛC(═O), C═)[C⁵R⁶)ₚCR⁷R⁸)ᵣ(c₁₋₆aklyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛNC(═O), C(═O)N[(CR⁵R⁶)ₚ(CR⁷R)ᵣN(C₁₋₆alkyl)C(═O)A¹⁴(CR R °)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹)ₛO(C⁰), C(═O)N[(CR⁵R⁶)ₚ(CR⁷R⁸)ᵣNHC(═O)A¹⁴A⁵](CR⁹R¹⁰)(C═O), C(═O)N[(CR⁵R⁶)ₚ(CR⁷R⁸)ᵣNHC(═O)A¹⁴A¹⁵](CR⁹R¹⁰)S(C═), C(═O)N[N(CR⁵R⁶)ₚ(CR⁷R)ᵣNHC(═O)A¹⁴A¹⁵(CR⁹R¹⁰)ₛO(C═O), C(═O)N[(CR⁵R⁶)ₚ(CR⁷R⁸)ᵣOC(═O)A¹⁴A¹⁵](CR⁹R¹⁰)ₛC(═O), C(═O)N[(CR⁵R⁶)ₚ(CR⁷R⁸)ᵣOC(═O)A¹⁴A⁵](CR⁹R¹⁰)ₛN(C═O), C(═O)N[(CR⁵R⁶)ₚ(CR⁷R⁸)ᵣOC(═O)A¹⁴A⁵](CR⁹R¹⁰)ₛO(C═O), C(═O)N[(CR⁵R⁶)ₚ(CR⁷R⁸)ᵣNHC(═O) (CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)(CR⁹R¹⁰)ₛNCH(NH₂)COOH]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O), C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂C(═O), C(═O)N[CH₂CH₂NHC(═O)CH₂CH₂CH(NH₂)COOH]CH₂CH₂OC(═O)CH(Me)CH₂C(═O), C(═O)N[CH₂CH₂N(C₁₋₆alkyl)C(═O)CH₂CH₂CH(NH₂)COOH]CH₂CH₂C(═O), (S)—C(═O)N[CH₂CH₂NHC(═O)CH₂CH₂CH(NH₂)COOH]CH₂CH₂C(═O), (R)—C(═O)N[CH₂CH₂NHC(═O)CH₂CH₂CH(NH₂) COOH]CH₂CH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), or (R)—C(═O)N[CH₂CH₂NHC(═O)CHNH(CH₂CH₂NH₂)]CH₂CH₂C(═O);
R¹¹ is C₁₋₁₂alkyl, CH(CH₃)₂, CH₂aryl, or CH₂Ph;
R¹² is CH₂NH₂, CH₂CH₂NH₂, or CH₂CH₂CH₂CH₂NH₂;
R¹⁵, R¹⁷ and R¹⁷ are independently H, Me, or C₁₋₁₂alkyl; and other groups and integers are defined as for the compound of the Formula I-P-1.

In another preferred embodiment, the compound of Formula I-P-1 is a compound of Formula V-P:

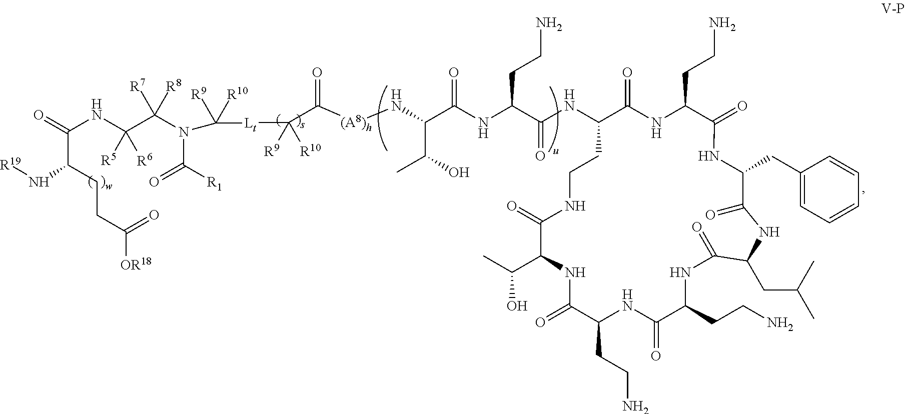

or pharmaceutically acceptable salt, solvate, or hydrate thereof, wherein
R¹⁸ is H or C₁₋₁₂alkyl;
R¹⁹ is H, C₁₋₁₂alkyl, C(═O)H, C(═O)C₁₋₁₂alkyl, C(═O)OC₁₋₁₂alkyl, C(═O)OC₁₋₁₂alkyl, C(═O)NHC₁₋₁₂alkyl, SO₂C₁₋₁₂alkyl, SO₂aryl, C(═O)C₃₋₇cycloalkyl, C(═O)OC₃₋₇cycloalkyl, C(═O)NHC₃₋₇cycloalkyl, C(═O)NHC₁₋₁₂alkyl, SO₂C₃₋₇cycloalkyl, or A¹;
each optional group L is selected from alkyl, C²R²¹OC(═O)CR²²R²³ and CR²⁰R²¹C(═O)OCR²²R²³;
R²⁰ through R²³ are independently selected from H, C₁₋₁₂alkyl, and C₃₋₇cycloalkyl; or any of the two adjacent groups R²⁰ and R²¹ or R²² and R²³ independently taken together form a C₃₋₇cycloalkyl group;
integer t is 0, 1, or 2;
integers u and w are independently 0 or 1; and
other groups and integers are defined as for the compound of the Formula I-P-1.

In another preferred embodiment, the compound of Formula I-P-1 is a compound of Formula V:

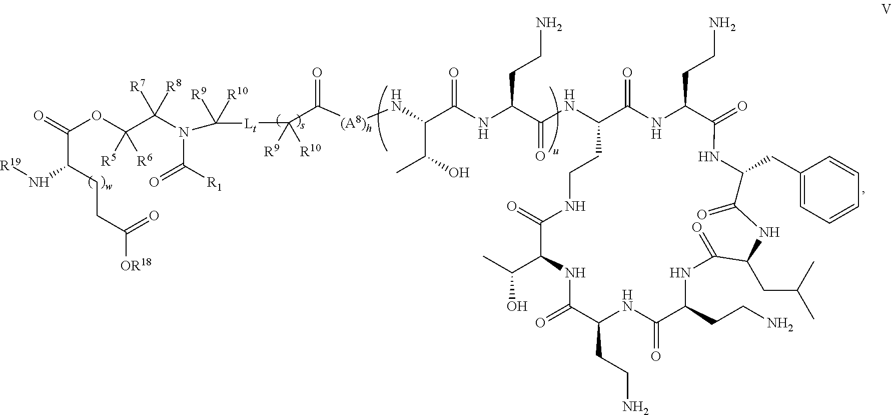

or pharmaceutically acceptable salt, solvate, or hydrate thereof, wherein
R¹⁸ is H or C₁₋₁₂alkyl;
R¹⁹ is H, C₁₋₁₂alkyl, C(═O)H, C(═O)C₁₋₁₂alkyl, C(═O)OC₁₋₁₂alkyl, C(═O)OC₁₋₁₂alkyl, C(═O)NHC₁₋₁₂alkyl, SO₂C₁₋₁₂alkyl, SO₂aryl, C(═O)C₃₋₇cycloalkyl, C(═O)OC₃₋₇cycloalkyl, C(═O)NHC₃₋₇cycloalkyl, C(═O)NHC₁₋₁₂alkyl, SO₂C₃₋₇cycloalkyl, or A¹;
each optional group L is selected from alkyl, C²R²¹OC(═O)CR²²R²³ and CR²⁰R²¹C(═O)OCR²²R²³;
R²⁰ through R²³ are independently selected from H, C₁₋₁₂alkyl, and C₃₋₇cycloalkyl; or any of the two adjacent groups R²⁰ and R²¹ or R²² and R²³ independently taken together form a C₃₋₇cycloalkyl group;
integer t is 0, 1, or 2;
integers u and w are independently 0 or 1; and
other groups and integers are defined as for the compound of the Formula I-P-1.

In one embodiment of Formula V-P or Formula V, R¹⁸ is H or C₁₋₁₂alkyl; R¹⁹ is H, C₁₋₁₂alkyl, C(═O)H, C(═O)C₁₋₁₂alkyl, C(═O)OC₁₋₁₂alkyl, C(═O)OC₁₋₁₂alkyl, C(═O)NHC₁₋₁₂alkyl, C(═O)C₃₋₇cycloalkyl, C(═O)OC₃₋₇cycloalkyl, C(═O)NHC₃₋₇cycloalkyl, or C(═O)NHC₁₋₁₂alkyl; each optional group L is selected from alkyl, CR²⁰R²¹OC(═O)CR²²R²³ and CR²⁰R²¹C(═O)OCR²²R²³; R²⁰ through R²³ are independently selected from H, C₁₋₁₂alkyl, and C₃₋₇cycloalkyl; the integer t is 0, 1, or 2; integer n is 0 or 1; and integers u and w are 1.

In a preferred embodiment, provided is a compound of Formula V-P or Formula V, wherein
R¹ is derived by subtracting H from any NH group in R¹(H), and wherein R¹(H) is axitinib (N-methyl-2-[[3-[(E)-2-pyridin-2-ylethenyl]-1H-indazol-6-yl]sulfanyl]benzamide) or tivozanib (1-{2-chloro-4-[(6,7-dimethoxy-4-quinolinyl)oxy]phenyl}-3-(5-methyl-1,2-oxazol-3-yl)urea);
R⁵ through R⁸ are all H; and
integers h and t are both 0, such that optional groups A⁸ and L are absent and the atoms adjacent to the absent groups A⁸ and L are connected directly to each other.

Non-limiting examples of R¹ include:

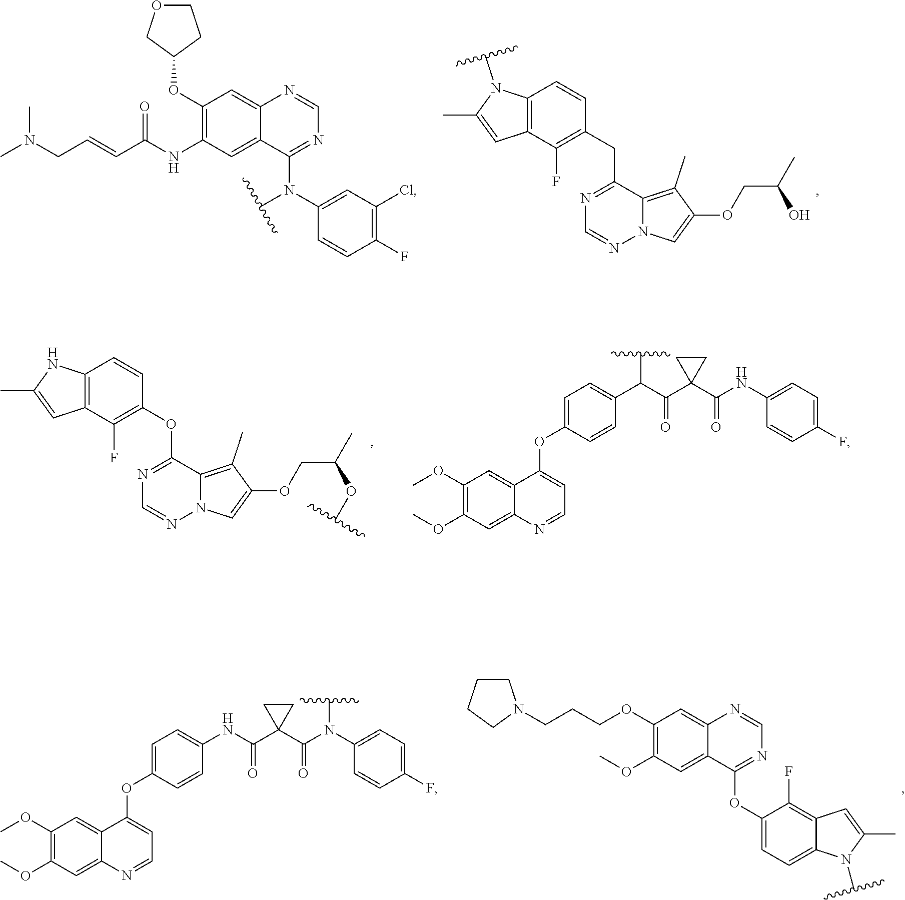 [SMILES: *N(c1ccc(F)c(Cl)c1)c1ncnc2cc(O[C@H]3CCOC3)c(NC(=O)/C=C/CN(C)C)cc12 ; *n1c(C)cc2c(F)c(Cc3ncnn4cc(OC[C@@H](C)O)c(C)c34)ccc21 ; *O[C@H](C)COc1cn2ncnc(Oc3ccc4[nH]c(C)cc4c3F)c2c1C ; *C(C(=O)C1(C(=O)Nc2ccc(F)cc2)CC1)c1ccc(Oc2ccnc3cc(OC)c(OC)cc23)cc1 ; *N(C(=O)C1(C(=O)Nc2ccc(Oc3ccnc4cc(OC)c(OC)cc34)cc2)CC1)c1ccc(F)cc1 ; *n1c(C)cc2c(F)c(Oc3ncnc4cc(OCCCN5CCCC5)c(OC)cc34)ccc21]

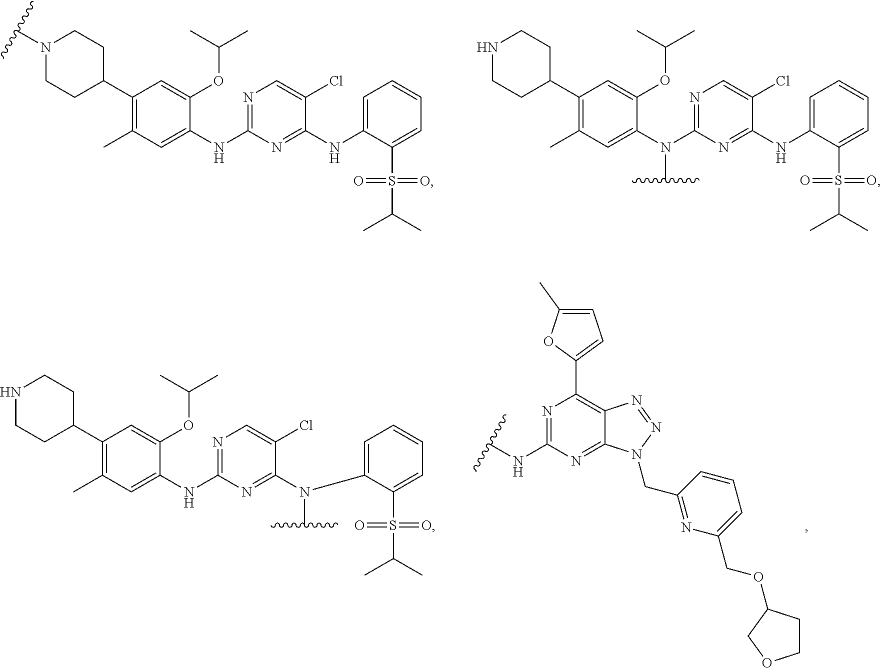 [SMILES: *N1CCC(c2cc(OC(C)C)c(Nc3ncc(Cl)c(Nc4ccccc4S(=O)(=O)C(C)C)n3)cc2C)CC1 ; *N(c1ncc(Cl)c(Nc2ccccc2S(=O)(=O)C(C)C)n1)c1cc(C)c(C2CCNCC2)cc1OC(C)C ; *N(c1ccccc1S(=O)(=O)C(C)C)c1nc(Nc2cc(C)c(C3CCNCC3)cc2OC(C)C)ncc1Cl ; *Nc1nc(-c2ccc(C)o2)c2nnn(Cc3cccc(COC4CCOC4)n3)c2n1]

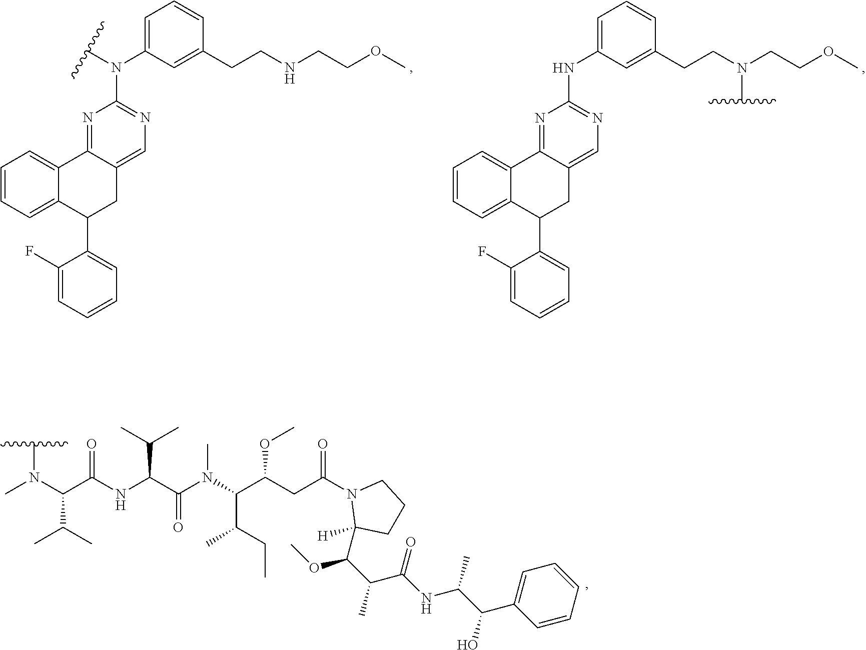 [SMILES: *N(c1cccc(CCNCCOC)c1)c1ncc2c(n1)-c1ccccc1C(c1ccccc1F)C2 ; *N(CCOC)CCc1cccc(Nc2ncc3c(n2)-c2ccccc2C(c2ccccc2F)C3)c1 ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

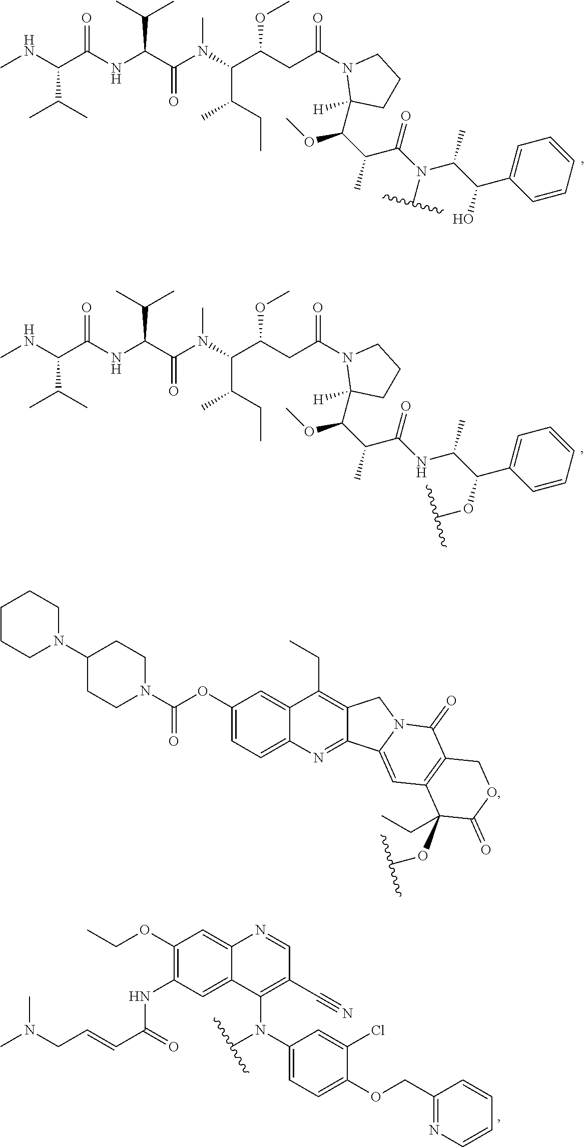 [SMILES: *N(C(=O)[C@H](C)[C@@H](OC)[C@@H]1CCCN1C(=O)C[C@@H](OC)[C@H]([C@@H](C)CC)N(C)C(=O)[C@@H](NC(=O)[C@@H](NC)C(C)C)C(C)C)[C@H](C)[C@@H](O)c1ccccc1 ; *O[C@@H](c1ccccc1)[C@@H](C)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@@H](NC)C(C)C)C(C)C ; *O[C@]1(CC)C(=O)OCc2c1cc1n(c2=O)Cc2c-1nc1ccc(OC(=O)N3CCC(N4CCCCC4)CC3)cc1c2CC ; *N(c1ccc(OCc2ccccn2)c(Cl)c1)c1c(C#N)cnc2cc(OCC)c(NC(=O)/C=C/CN(C)C)cc12]

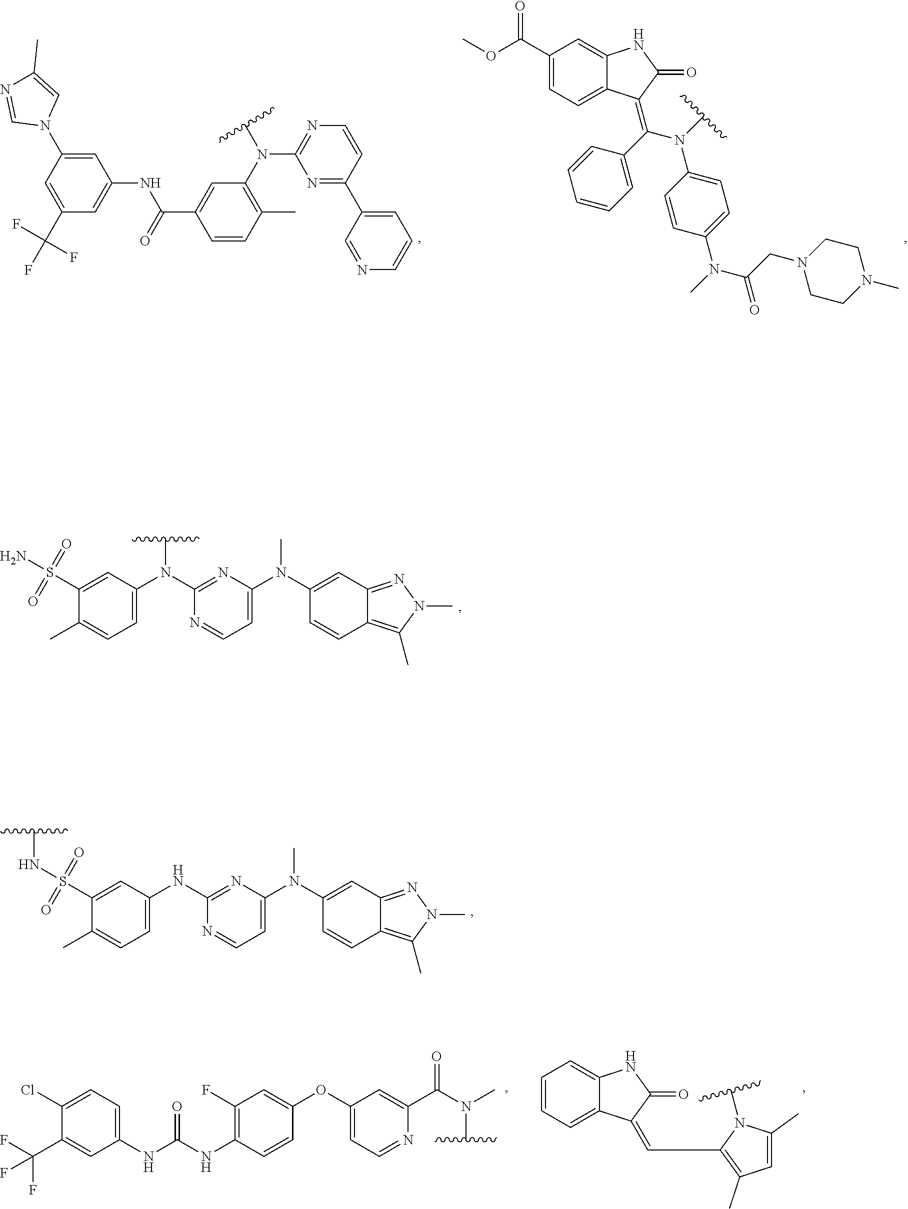

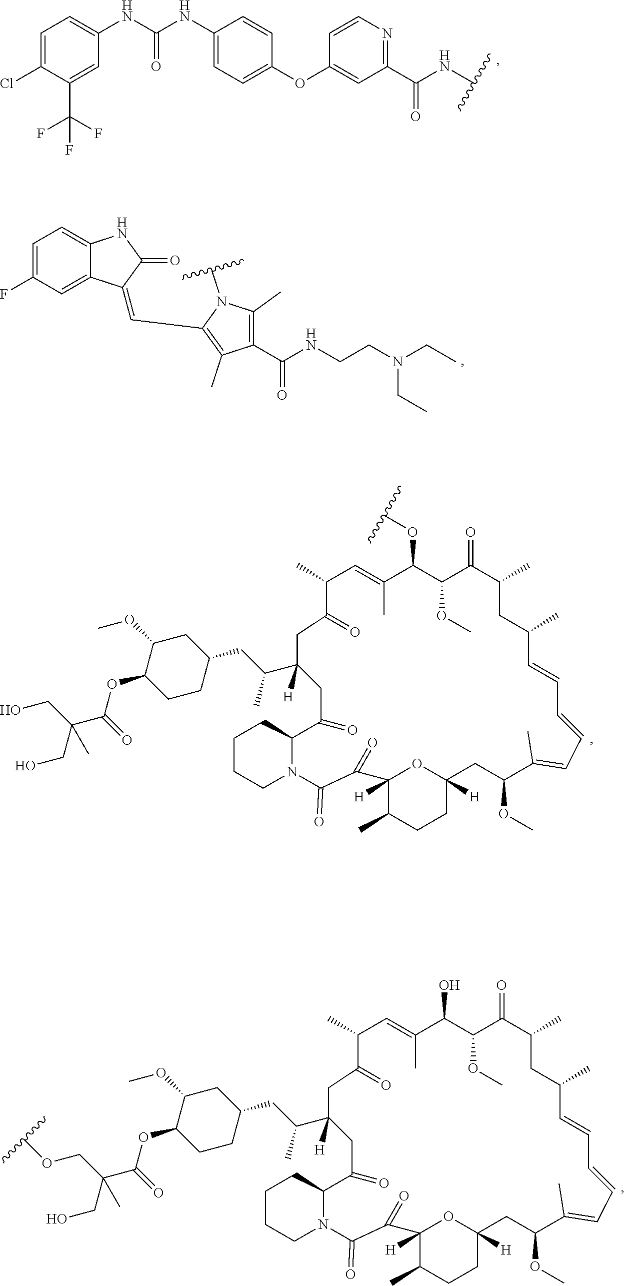

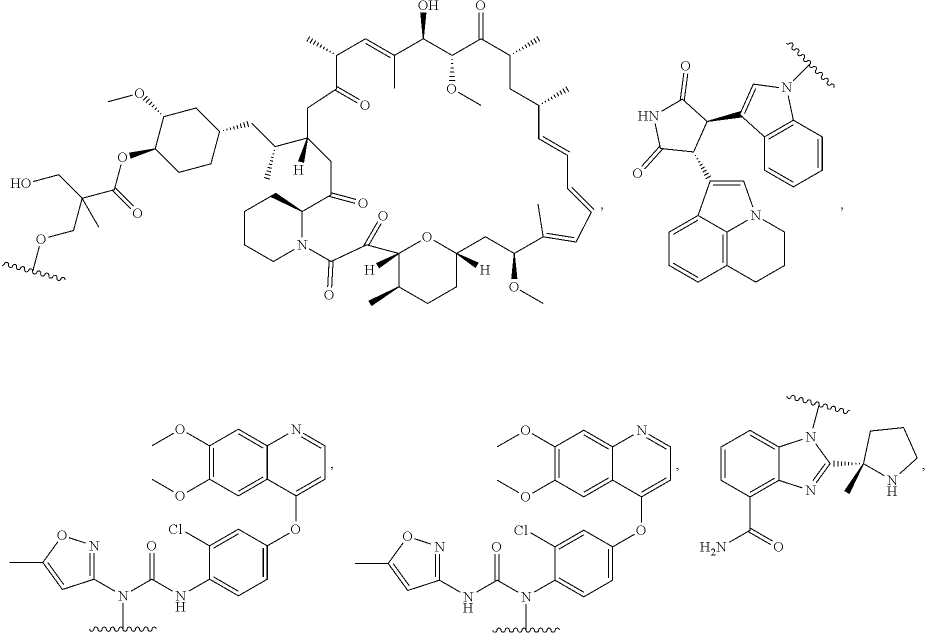 [SMILES: *OCC(C)(CO)C(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@H](O3)C(=O)C(=O)N3CCCC[C@H]3C(=O)C2)C[C@H]1OC ; *n1cc([C@@H]2C(=O)NC(=O)[C@H]2c2cn3c4c(cccc24)CCC3)c2ccccc21 ; *N(C(=O)Nc1ccc(Oc2ccnc3cc(OC)c(OC)cc23)cc1Cl)c1cc(C)on1 ; *N(C(=O)Nc1cc(C)on1)c1ccc(Oc2ccnc3cc(OC)c(OC)cc23)cc1Cl ; *n1c([C@]2(C)CCCN2)nc2c(C(N)=O)cccc21]

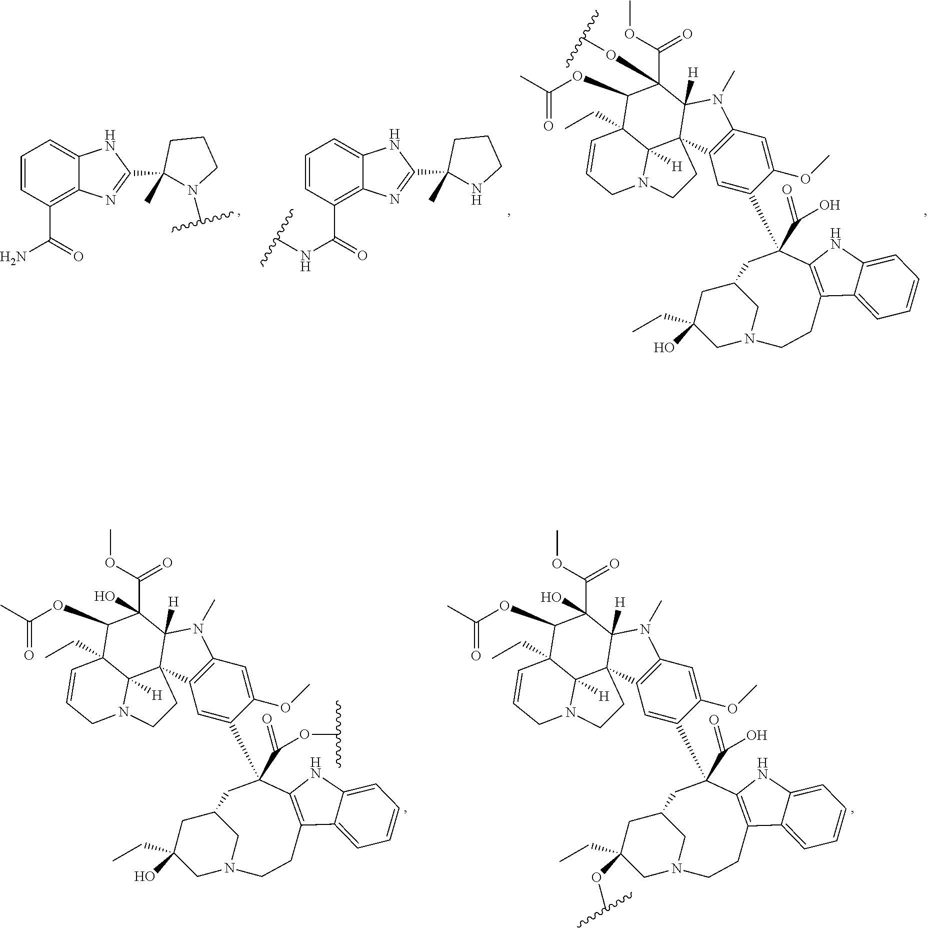

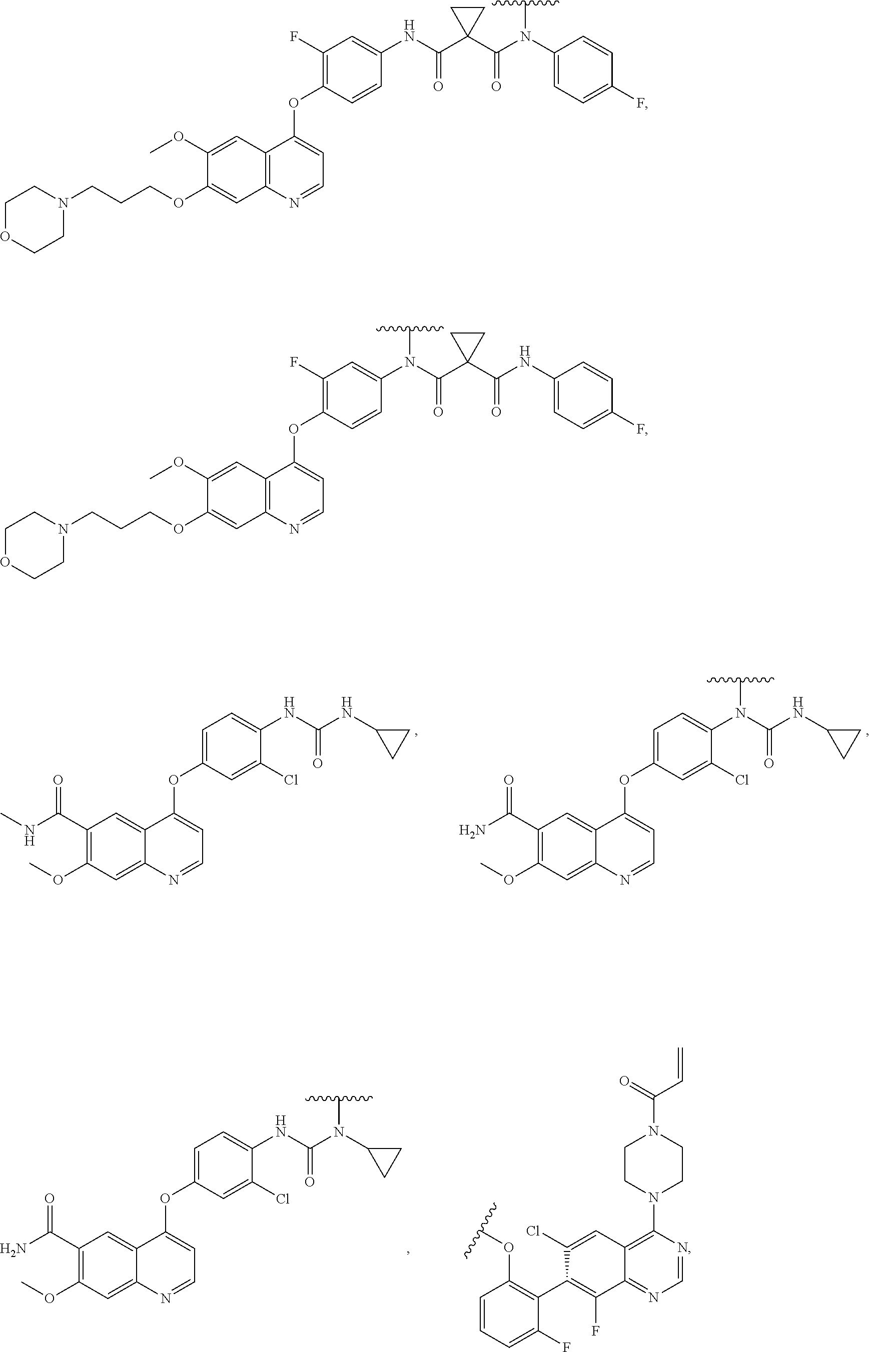

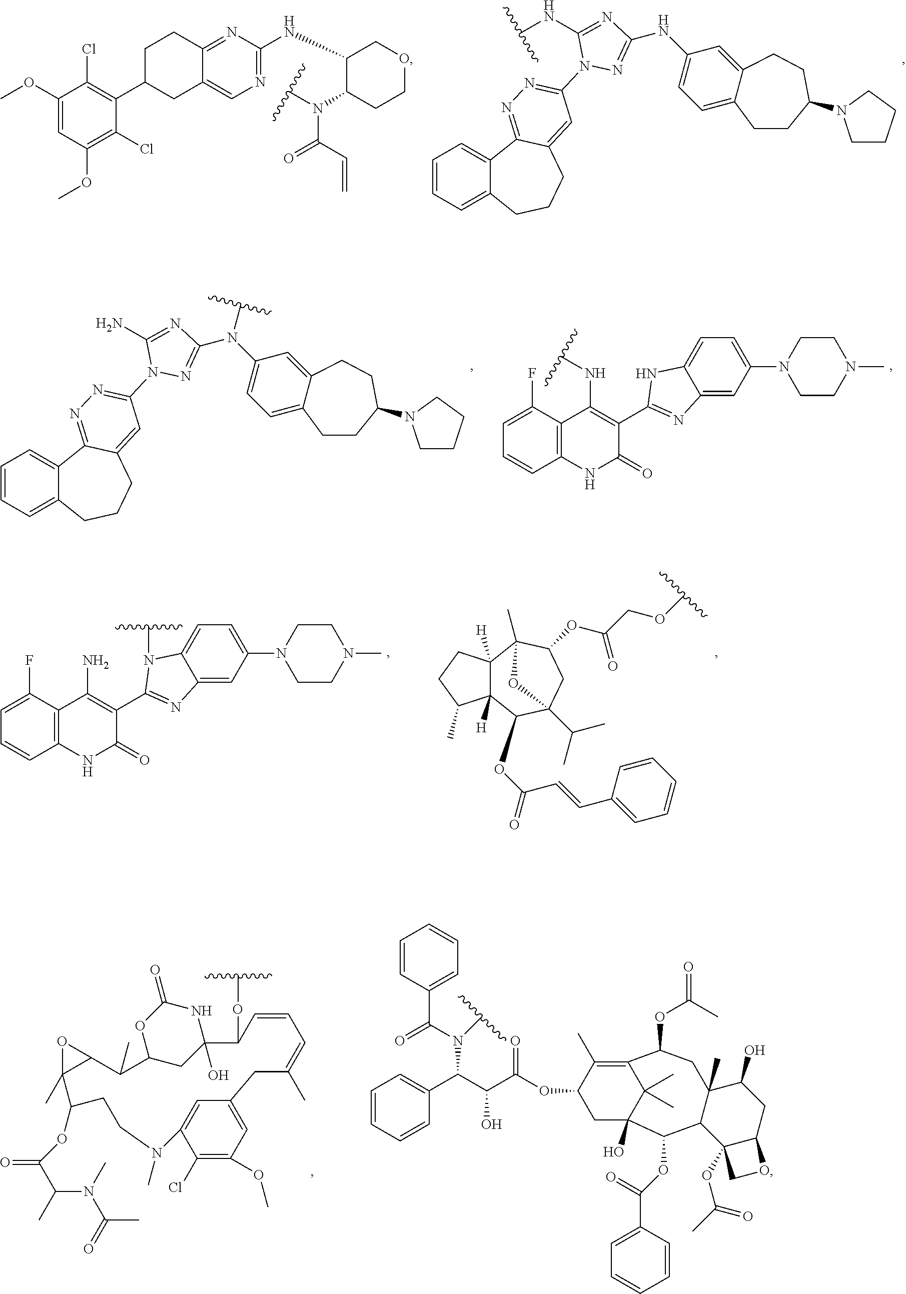

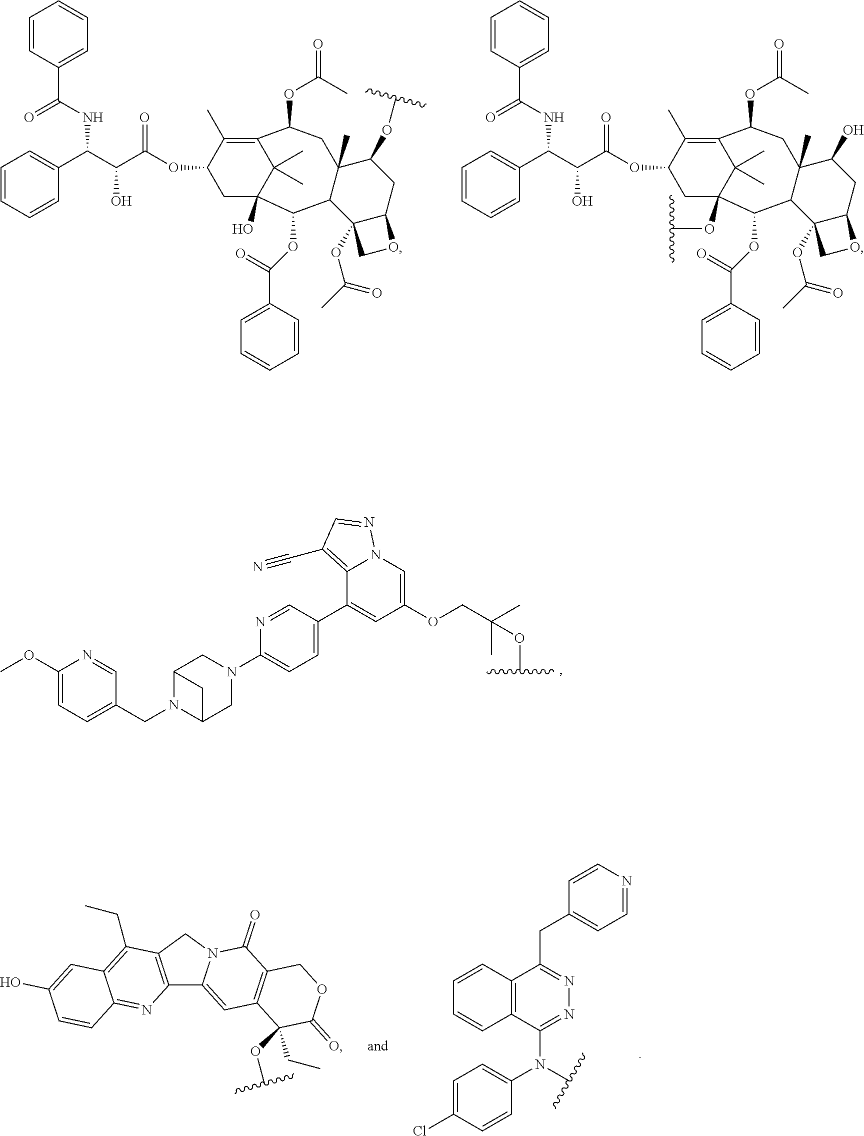 [SMILES: *O[C@H]1C[C@H]2OC[C@@]2(OC(C)=O)C2[C@H](OC(=O)c3ccccc3)[C@]3(O)C[C@H](OC(=O)[C@H](O)[C@@H](NC(=O)c4ccccc4)c4ccccc4)C(C)=C([C@@H](OC(C)=O)C[C@@]21C)C3(C)C ; *O[C@]12C[C@H](OC(=O)[C@H](O)[C@@H](NC(=O)c3ccccc3)c3ccccc3)C(C)=C([C@@H](OC(C)=O)C[C@@]3(C)C([C@@H]1OC(=O)c1ccccc1)[C@]1(OC(C)=O)CO[C@@H]1C[C@@H]3O)C2(C)C ; *OC(C)(C)COc1cc(-c2ccc(N3CC4CC(C3)N4Cc3ccc(OC)nc3)nc2)c2c(C#N)cnn2c1 ; *O[C@]1(CC)C(=O)OCc2c1cc1n(c2=O)Cc2c-1nc1ccc(O)cc1c2CC ; *N(c1ccc(Cl)cc1)c1nnc(Cc2ccncc2)c2ccccc12]

In a preferred embodiment, provided is a compound of any of Formula I, Formula I-P-1, Formula I-P-2, Formula II-P, Formula III, Formula III-P, Formula IV-P, Formula V-P, and Formula V, wherein R¹ is selected from the structures below:

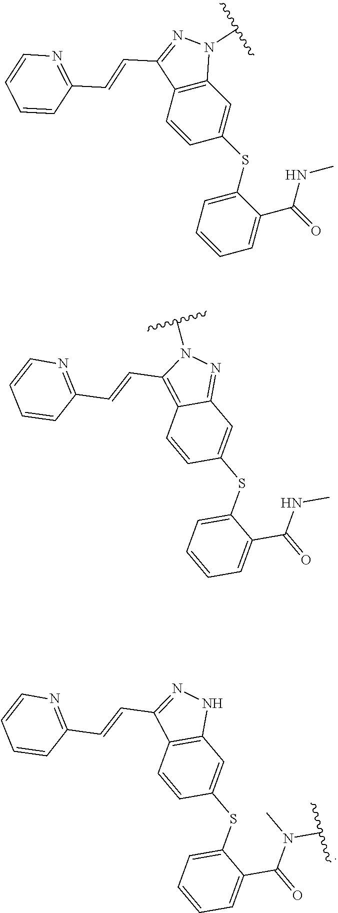 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1 ; *N(C)C(=O)c1ccccc1Sc1ccc2c(/C=C/c3ccccn3)n[nH]c2c1]

In one preferred embodiment, the compound is selected from the structures below, or a pharmaceutically acceptable salt, solvate, or hydrate thereof:

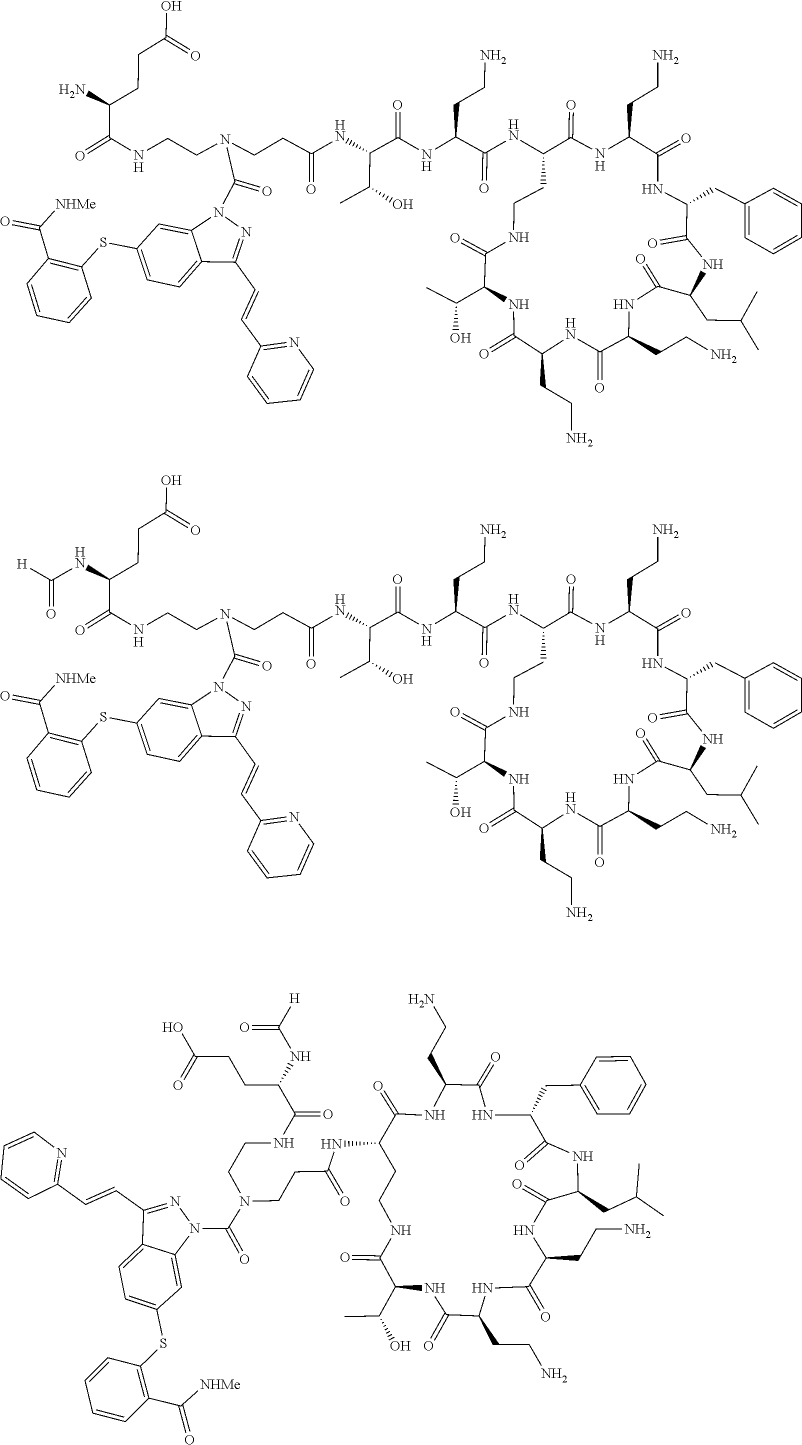

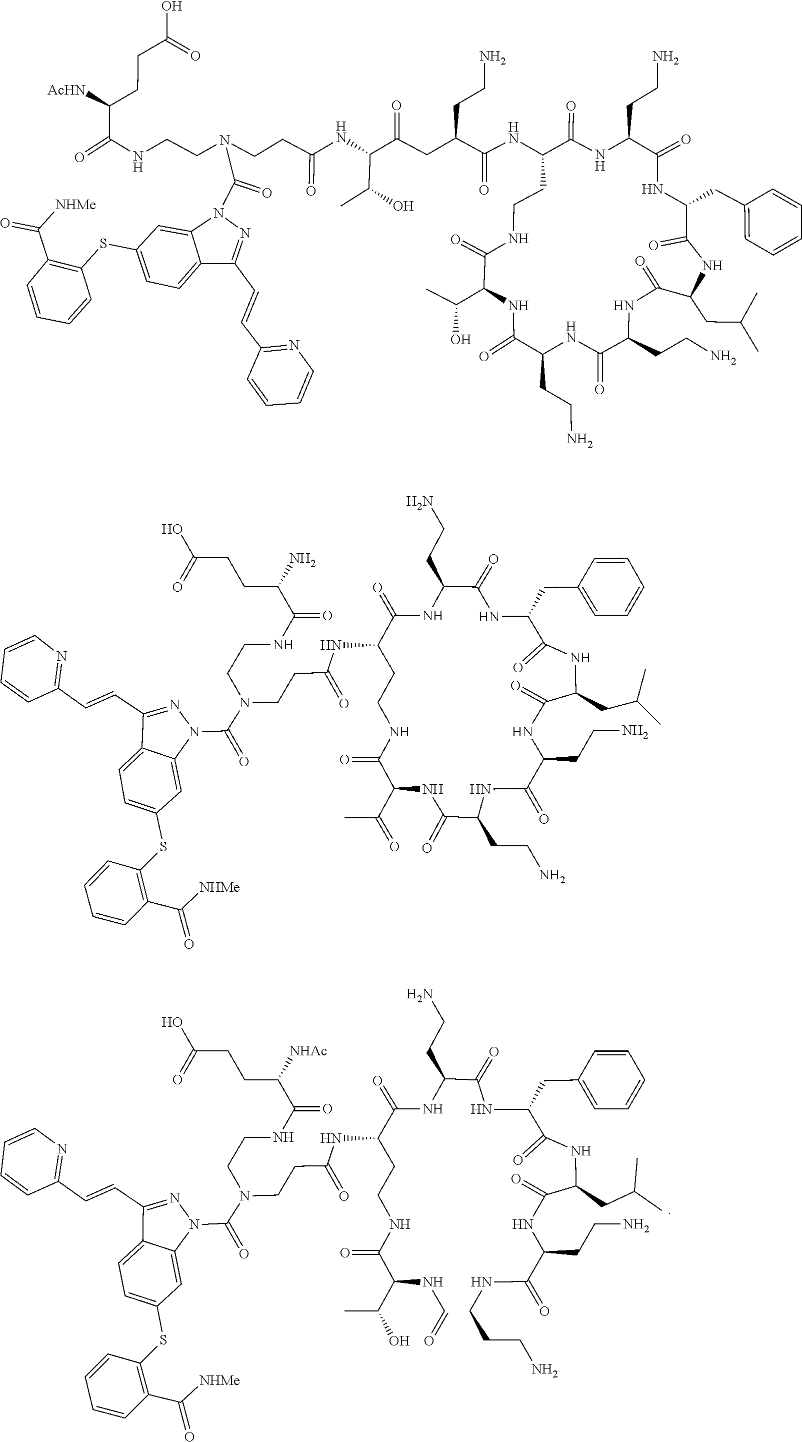

In one preferred embodiment, the compound is selected from the structures below, or a pharmaceutically acceptable salt, solvate, or hydrate thereof:

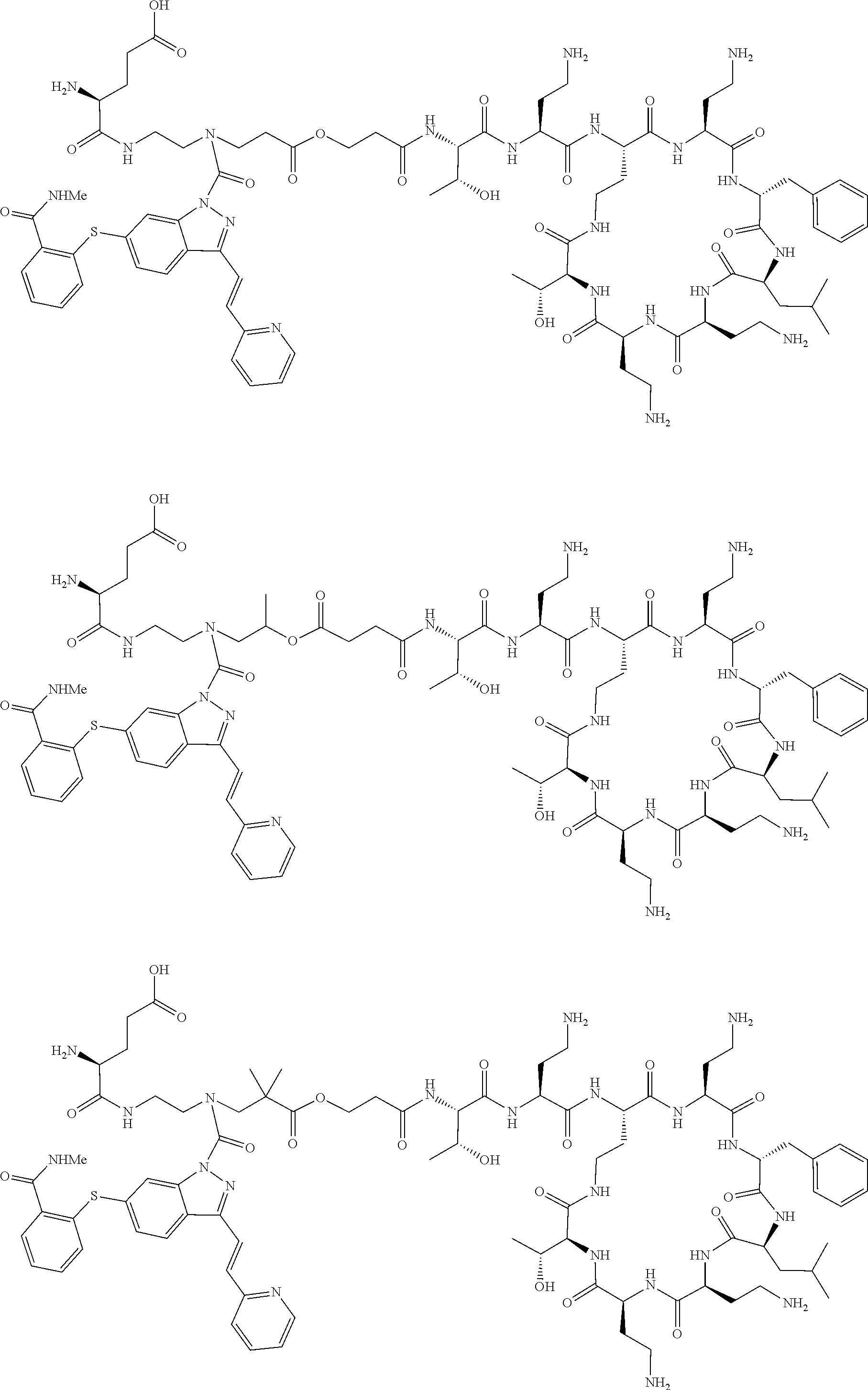

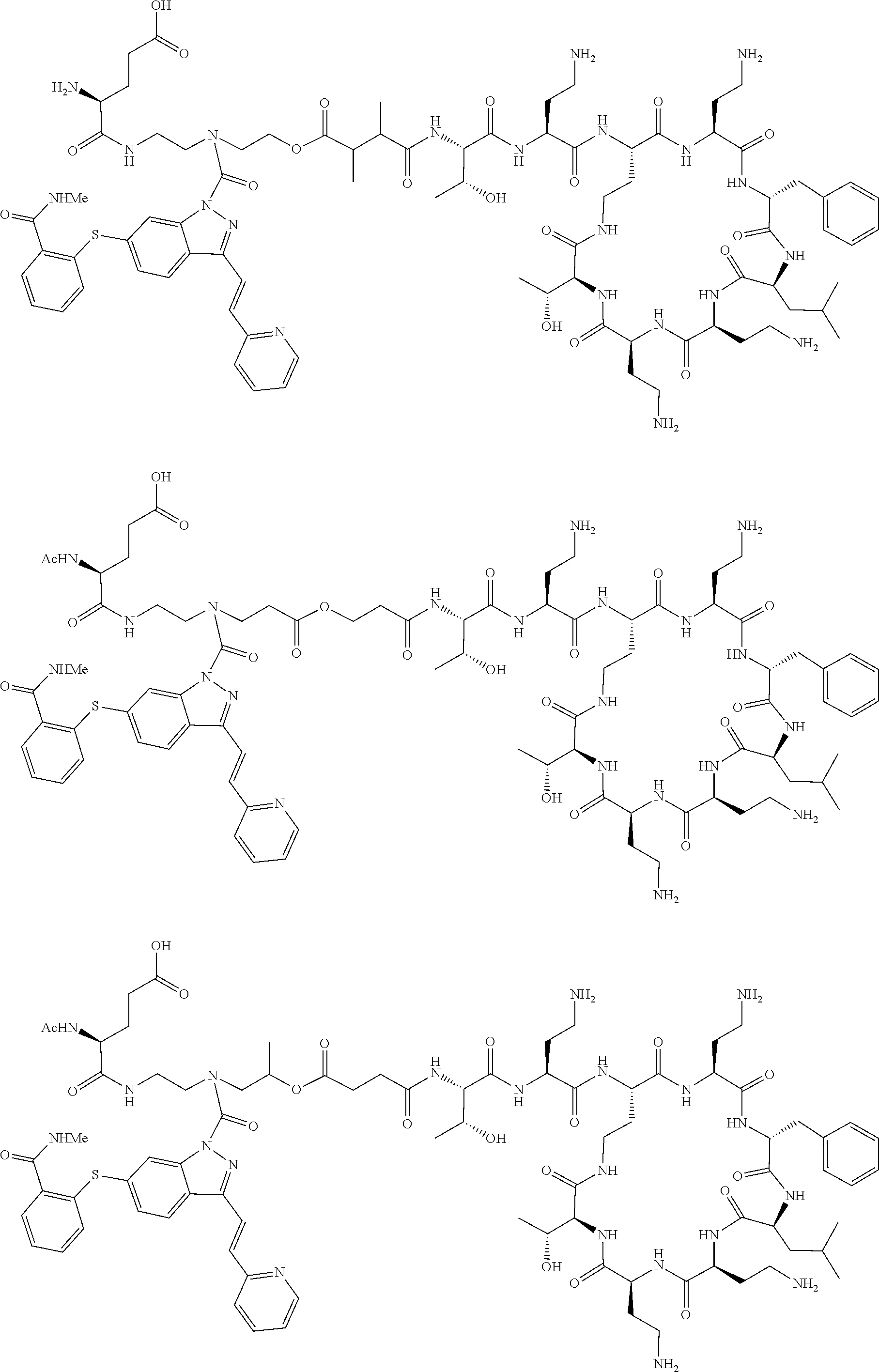

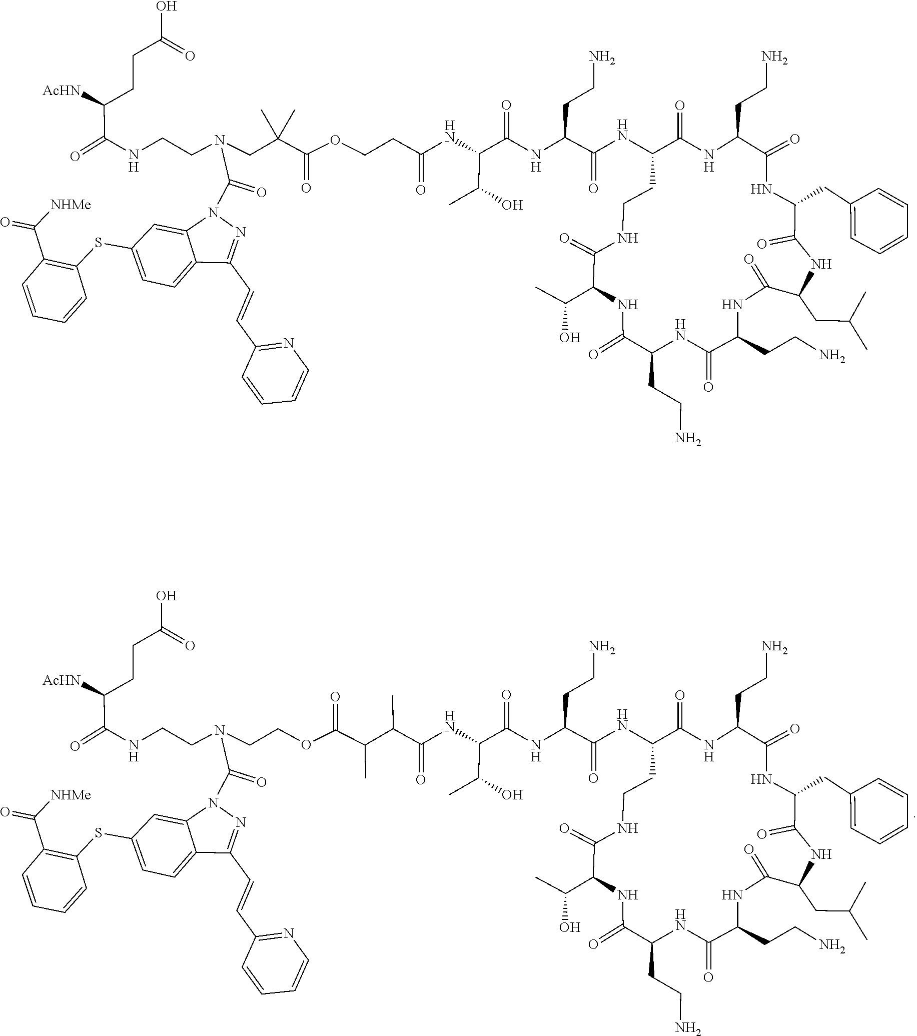

In one preferred embodiment, the compound is selected from the structures below, or a pharmaceutically acceptable salt, solvate, or hydrate thereof:

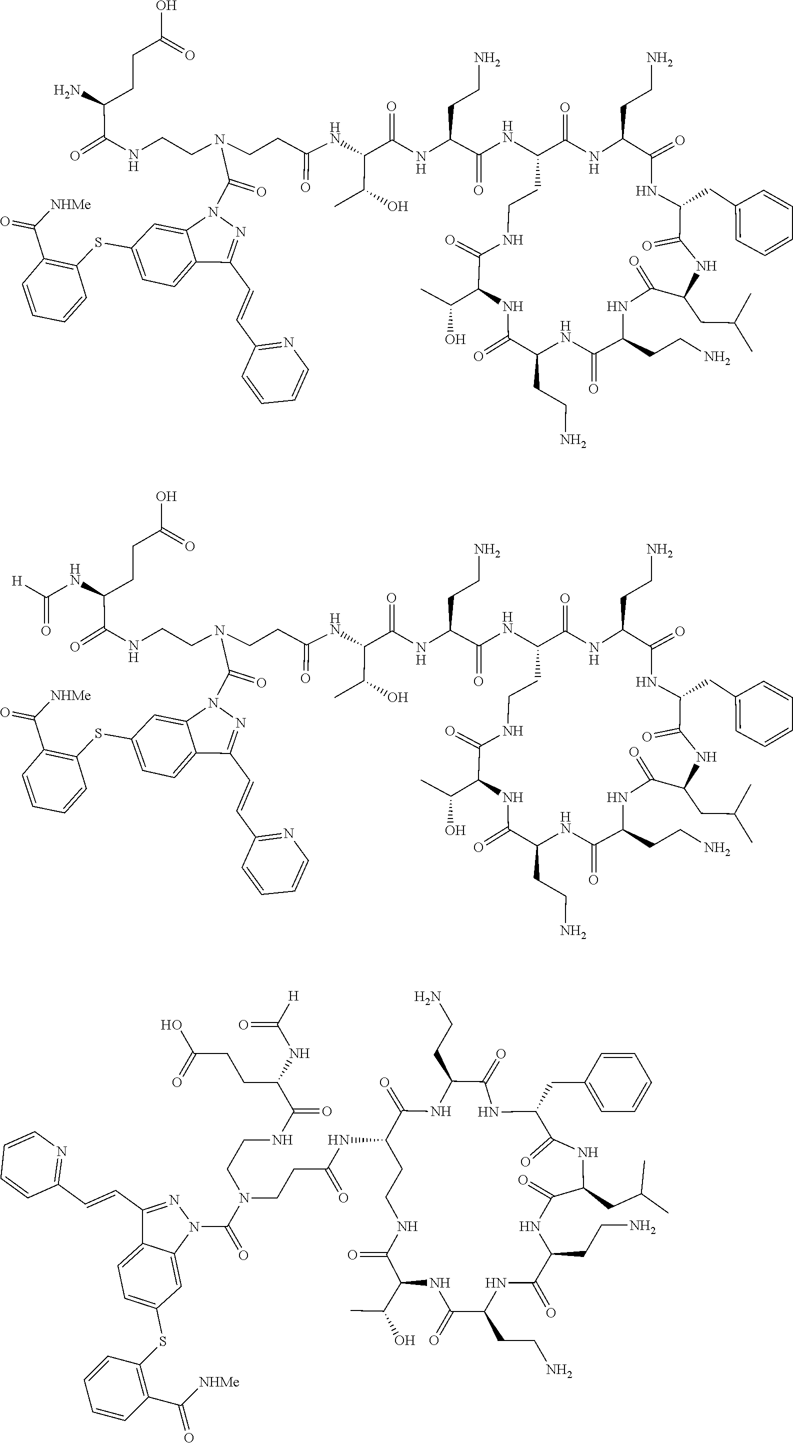

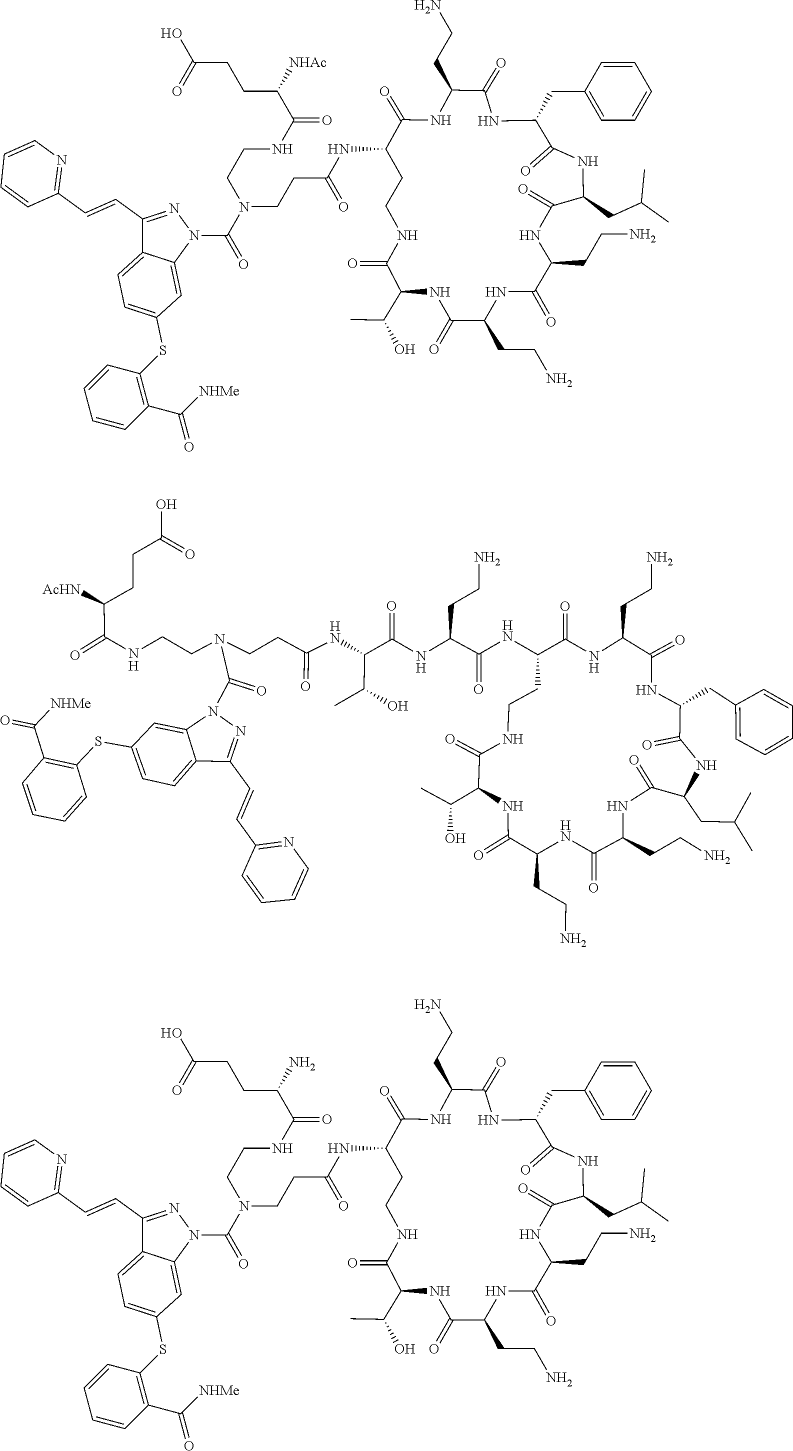

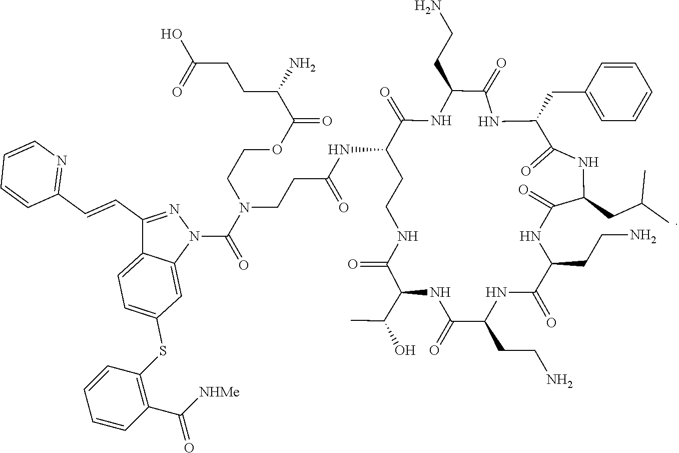 [SMILES: CNC(=O)c1ccccc1Sc1ccc2c(/C=C/c3ccccn3)nn(C(=O)N(CCOC(=O)[C@@H](N)CCC(=O)O)CCC(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)c2c1]

In one preferred embodiment, the compound is selected from the structures below, or a pharmaceutically acceptable salt, solvate, or hydrate thereof:

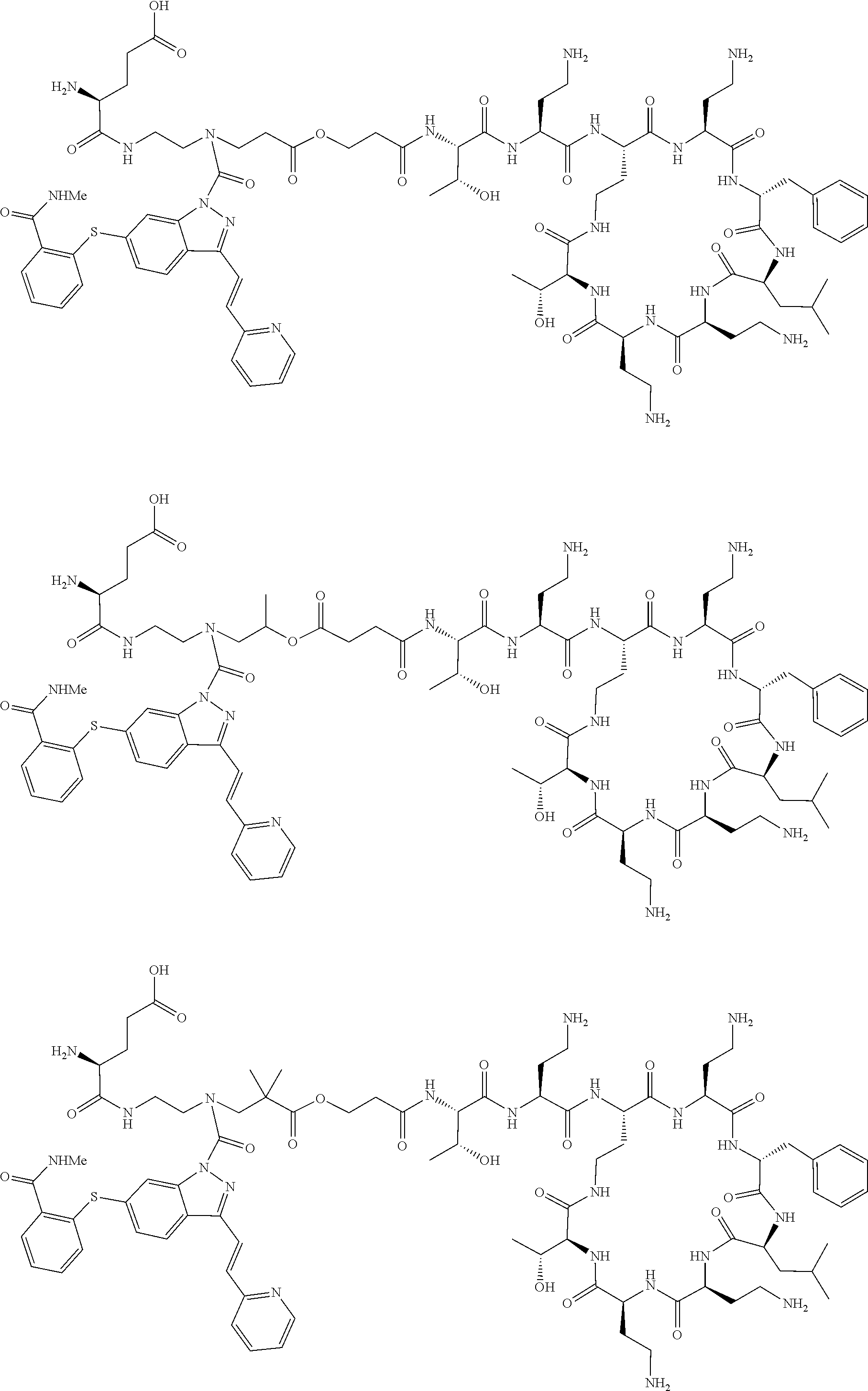

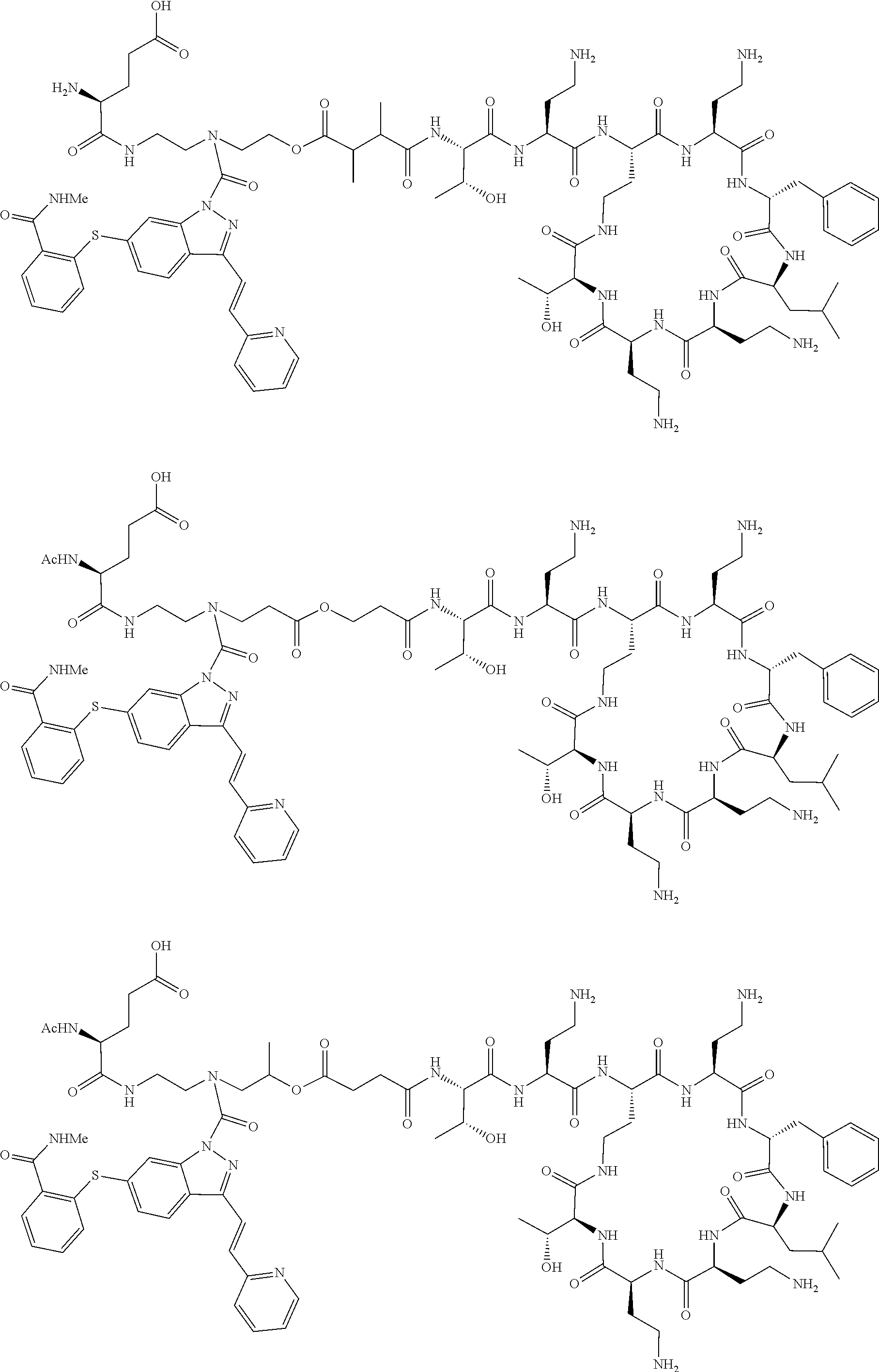

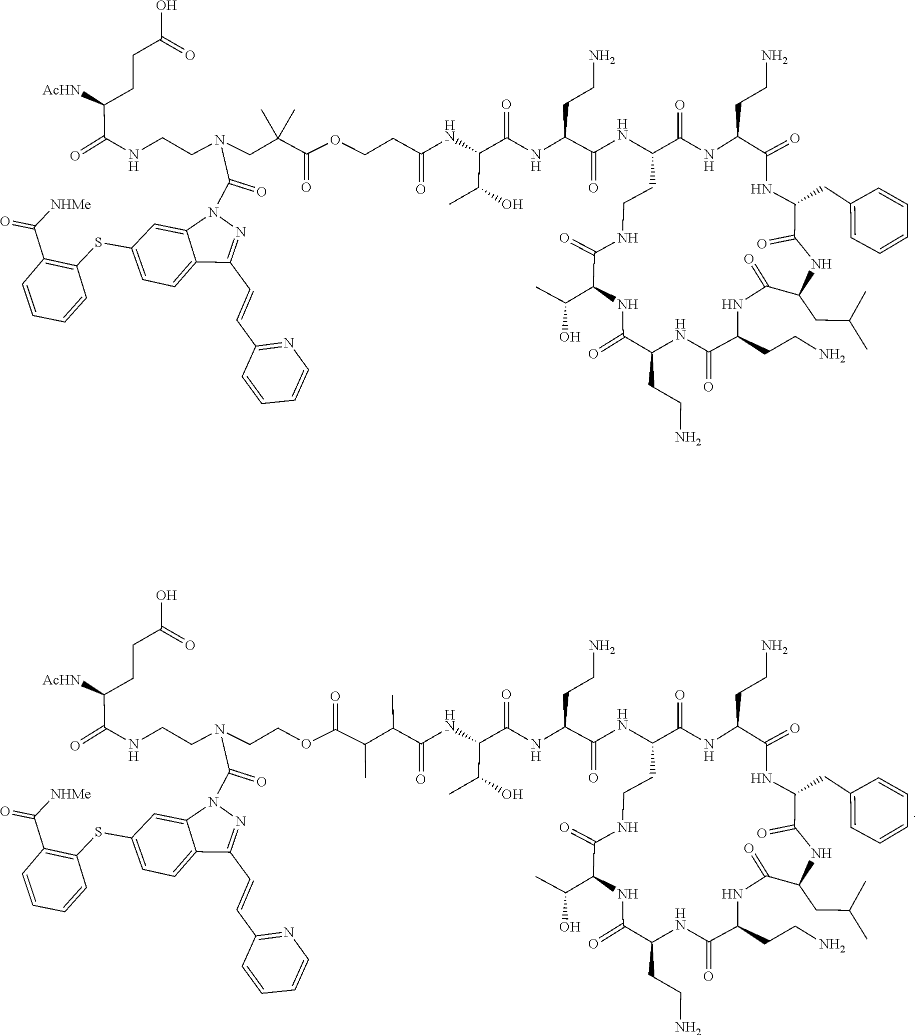

In one preferred embodiment, the compound is selected from the structures below, or a pharmaceutically acceptable salt, solvate, or hydrate thereof:

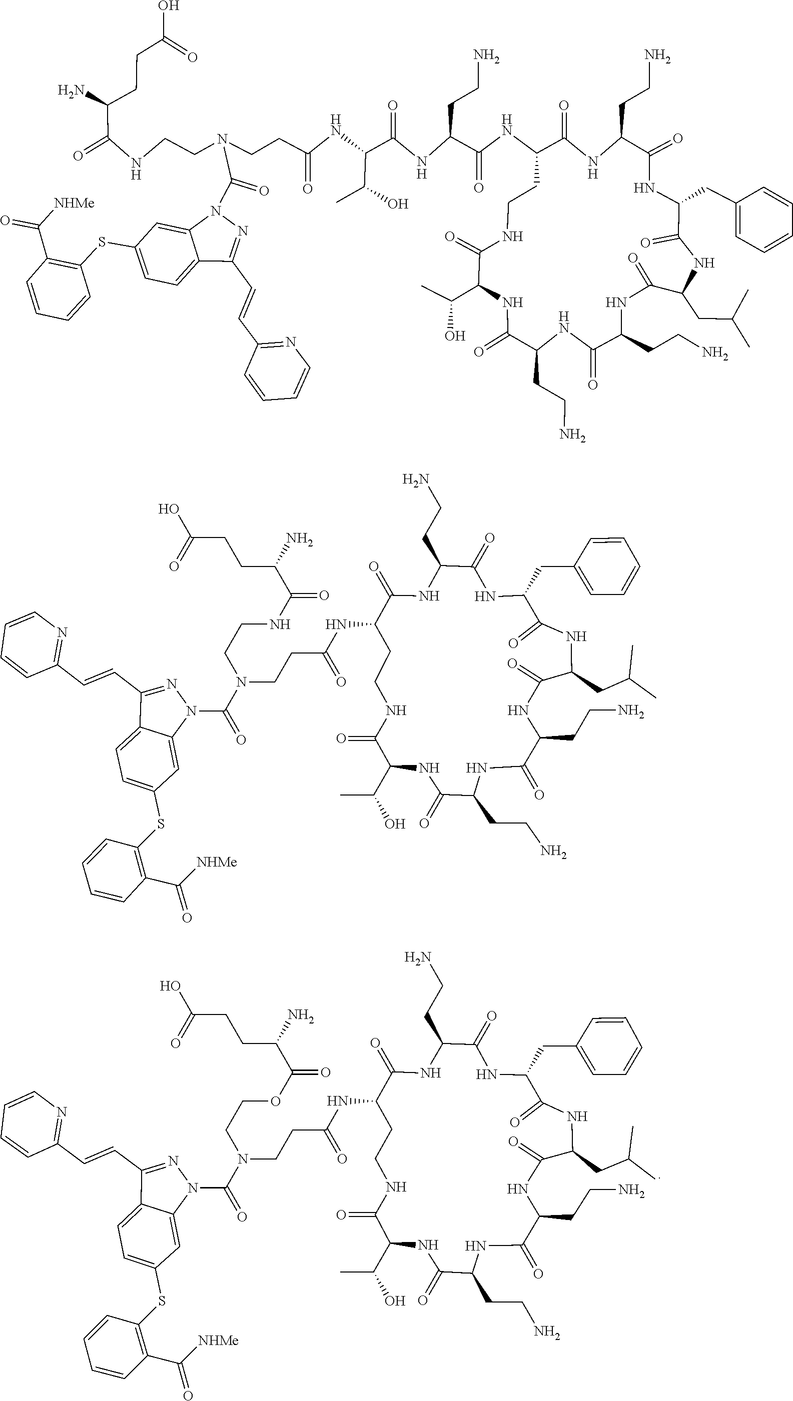

In some or any embodiments, the compound is according to any one of Formula I, Formula II-P, Formula III, Formula IV-P, Formula V-P, and Formula V, or of any one of embodiments as provided herein, wherein said compound exerts a therapeutic effect after administration into a mammal by releasing a bioactive or cytotoxic agent (H)ₙR¹. In some or any embodiments, the compound is according to any one of Formula I-P-1, Formula I-P-2, and Formula III-P, or of any one of embodiments as provided herein, wherein said compound exerts a therapeutic effect after administration into a mammal by releasing a bioactive or cytotoxic agent(s) (H)ₙR¹ and/or (H)ₒR².

In some or any embodiments, the compound is according to any one of Formula I, Formula II-P, Formula III, Formula IV-P, Formula V-P, and Formula V, or of any one of embodiments as provided herein, wherein said compound possesses a reduced cytotoxicity against non-cancerous mammalian cell(s), when compared to an agent or drug (H)ₙR¹ incorporated into said compound, as determined by in vitro cytotoxicity test(s), such as cell growth inhibition test(s). In some or any embodiments, the compound is according to any one of Formula I-P-1, Formula I-P-2, and Formula III-P, or of any one of embodiments as provided herein, wherein said compound possesses a reduced cytotoxicity against non-cancerous mammalian cell(s), when compared to an agent(s) or drug(s) (H)ₙR¹ and/or (H)ₒR² incorporated into said compound, as determined by in vitro cytotoxicity test(s), such as cell growth inhibition test(s).

In some or any embodiments, the compound is according to any one of Formula I, Formula II-P, Formula III, Formula IV-P, Formula V-P, and Formula V, or of any one of embodiments as provided herein, wherein said compound possesses at least about 50% reduced cytotoxicity against non-cancerous mammalian cell(s), when compared to the corresponding agent or drug of formula (H)ₙR¹, as determined by in vitro cytotoxicity test(s), such as cell growth inhibition test(s). In some or any embodiments, the compound is according to any one of Formula I-P-1, Formula I-P-2, and Formula III-P, or of any one of embodiments as provided herein, wherein said compound possesses at least about 50% reduced cytotoxicity against non-cancerous mammalian cell(s), when compared to the corresponding agent(s) or drug(s) of formula (H)ₙR¹ and/or (H)ₒR², as determined by in vitro cytotoxicity test(s), such as cell growth inhibition test(s).

In some or any embodiments, the compound is according to any one of Formula I, Formula I-P-1, Formula I-P-2, Formula II-P, Formula III, Formula III-P, Formula IV-P, Formula V-P, and Formula V, or of any one of embodiments as provided herein, wherein said compound when administered to a mammal, exhibit preferential accumulation in kidneys, with a ratio for its concentration in kidneys compared to that in blood of between about 10 and 500.

In some or any embodiments the compound is according to any one of Formula I, Formula I-P-1, Formula I-P-2, Formula II-P, Formula III, Formula III-P, Formula IV-P, Formula V-P, and Formula V, or of any embodiments as provided herein, wherein when administered to a mammal, exhibits preferential accumulation in kidneys, with a ratio for its concentration in kidneys compared to that in blood of at least 20.

In some or any embodiments the compound is according to any one of Formula I, Formula II-P, Formula III, Formula IV-P, Formula V-P, and Formula V, or of any embodiments as provided herein, wherein when administered to a mammal at a dosing (expressed in molar amount) equal to a standard therapeutic dosing (in molar amount) of an agent (H)ₙR¹, said compound exhibits about 1.5- to 15-fold higher loading (tissue concentration) of agent (H)ₙR¹ in kidneys, as compared to the standard therapeutic dosing of (H)ₙR¹. In some or any embodiments the compound is according to any one of Formula I-P-1, Formula I-P-2, and Formula III-P, or of any embodiments as provided herein, wherein when administered to a mammal at a dosing (expressed in molar amount) equal to a standard therapeutic dosing (in molar amount) of an agent(s) (H)ₙR¹ and/or (H)ₒR², said compound exhibits about 1.5- to 15-fold higher loading (tissue concentration) of agent (H)ₙR¹ and/or (H)ₒR² in kidneys, as compared to the standard therapeutic dosing of (H)ₙR¹ and/or (H)ₒR².

In some or any embodiments the compound is according to any one of Formula I, Formula II-P, Formula III, Formula IV-P, Formula V-P, and Formula V, or of any embodiments as provided herein, wherein when administered to a mammal at a dosing (expressed in molar amount) equal to a standard therapeutic dosing (in molar amount) of an agent (H)ₙR¹, said compound exhibits at least 2-fold higher loading (tissue concentration) of agent (H)ₙR¹ in kidneys, as compared to the standard therapeutic dosing of (H)ₙR¹. In some or any embodiments the compound is according to any one of Formula I-P-1, Formula I-P-2, and Formula III-P, or of any embodiments as provided herein, wherein when administered to a mammal at a dosing (expressed in molar amount) equal to a standard therapeutic dosing (in molar amount) of an agent(s) (H)ₙR¹ and/or (H)ₒR², said compound exhibits at least 2-fold higher loading (tissue concentration) of agent(s) (H)ₙR¹ and/or (H)ₒR² in kidneys, as compared to the standard therapeutic dosing of (H)ₙR¹ and/or (H)ₒR².

In some or any embodiments the compound is according to any one of Formula I, Formula II-P, Formula III, Formula IV-P, Formula V-P, and Formula V, or of any embodiments as provided herein, wherein when administered to a mammal at a dosing (expressed in molar amount) equal to a standard therapeutic dosing (in molar amount) of agent (H)ₙR¹, exhibits about 1.5- to 15-fold higher efficacy, as compared to the standard therapeutic dosing of agent (H)ₙR¹, with said therapeutic effect determined as a slowed, stopped, or reversed progression of cancer (as determined per changes in a cancer tumor size, and/or by using biochemical biomarkers for cancer monitoring, or similar methods). In some or any embodiments the compound is according to any one of Formula I-P-1, Formula I-P-2, and Formula III-P, or of any embodiments as provided herein, wherein when administered to a mammal at a dosing (expressed in molar amount) equal to a standard therapeutic dosing (in molar amount) of agent(s) (H)ₙR¹ and/or (H)ₒR², exhibits about 1.5- to 15-fold higher efficacy, as compared to the standard therapeutic dosing of agent(s) (H)ₙR¹ and/or (H)ₒR², with said therapeutic effect determined as a slowed, stopped, or reversed progression of cancer (as determined per changes in a cancer tumor size, and/or by using biochemical biomarkers for cancer monitoring, or similar methods).

In some or any embodiments the compound is according to any one of Formula I, Formula II-P, Formula III, Formula IV-P, Formula V-P, and Formula V, or of any embodiments as provided herein, when administered wherein when administered to a mammal at a dosing (expressed in molar amount) equal to a standard therapeutic dosing (in molar amount) of agent (H)ₙR¹, exhibits at least 2-fold higher efficacy, as compared to the standard therapeutic dosing of agent (H)ₙR¹, with said therapeutic effect determined as a slowed, stopped, or reversed progression of cancer (as determined per changes in a cancer tumor size, and/or by using biochemical biomarkers for cancer monitoring, or similar methods). In some or any embodiments the compound is according to any one of Formula I-P-1, Formula I-P-2, and Formula III-P, or of any embodiments as provided herein, when administered wherein when administered to a mammal at a dosing (expressed in molar amount) equal to a standard therapeutic dosing (in molar amount) of agent(s) (H)ₙR¹ and/or (H)ₒR², exhibits at least 2-fold higher efficacy, as compared to the standard therapeutic dosing of agent(s) (H)ₙR¹ and/or (H)ₒR², with said therapeutic effect determined as a slowed, stopped, or reversed progression of cancer (as determined per changes in a cancer tumor size, and/or by using biochemical biomarkers for cancer monitoring, or similar methods).

In some or any embodiments the compound is according to any one of Formula I, Formula II-P, Formula III, Formula IV-P, Formula V-P, and Formula V, or of any embodiments as provided herein, when administered to a mammal at a dosing (expressed in molar amount) equal to a standard therapeutic dosing (in molar amount) of an agent (H)ₙR¹, exhibits at least 2-fold reduced rate of adverse effects and/or off-target toxicity manifestation, as compared to the standard therapeutic dosing of (H)ₙR¹, as determined by gross observations of a mammal under therapy, a blood cells count, a tissue biopsy, and/or by analysis of biochemical biomarkers, or similar method. In some or any embodiments the compound is according to any one of Formula I-P-1, Formula I-P-2, and Formula III-P, or of any embodiments as provided herein, when administered to a mammal at a dosing (expressed in molar amount) equal to a standard therapeutic dosing (in molar amount) of an agent(s) (H)ₙR¹ and/or (H)ₒR², exhibits at least 2-fold reduced rate of adverse effects and/or off-target toxicity manifestation, as compared to the standard therapeutic dosing of (H)ₙR¹ and/or (H)ₒR², as determined by gross observations of a mammal under therapy, a blood cells count, a tissue biopsy, and/or by analysis of biochemical biomarkers, or similar method.

In some or any embodiments, provided is a method for the treatment of a cancer disease, such as kidney or renal cancer disease, in a mammal comprising administering to the mammal a therapeutically effective amount of a compound is according to any one of Formula I, Formula I-P-1, Formula I-P-2, Formula II-P, Formula III, Formula III-P, Formula IV-P, Formula V-P, and Formula V, or as defined in any of the embodiments described herein.

In some or any embodiments, provided is a method for the treatment of a cancer disease in a mammal comprising administering to the mammal a therapeutically effective amount of a compound is according to any one of Formula I, Formula I-P-1, Formula I-P-2, Formula II-P, Formula III, Formula III-P, Formula IV-P, Formula V-P, and Formula V, or as defined in any of the embodiments described herein wherein the compound is administered to the mammal orally, parenterally, transdermally, topically, rectally, intranasally, or by intra-tumoral administration (such as injection) in a pharmaceutical composition, including an aerosol form. In some or any embodiments, the method is that wherein the cancer is a renal cancer carcinoma (RCC) or metastatic renal cell carcinoma (mRCC) diseases.

In some embodiments and aspects, a compound provided herein may be used in a combination with an adjunct agent, to act synergistically and/or enhance therapeutic effects of said compound itself, or of an adjunct agent, or both. Such adjunct agents include other anticancer or immunomodulating agent(s), such as a monoclonal antibody agent, or another cytotoxic agent(s), or another oncology (cancer) agent, or humanized antibody such as pembrolizimab.

Such combinations of the compounds provided herein are useful for the prevention, treatment, and alleviation of symptoms of cancer diseases, in particular, kidney cancers.

In one such aspect, a compound provided herein has modest or no anticancer activity in vitro, but exhibits high anticancer efficacy when administered to a mammal in need of a cancer therapy.

In some or any embodiments is provided a pharmaceutical composition comprising a therapeutically effective amount of a compound according to any one of Formula I, Formula I-P-1, Formula I-P-2, Formula II-P, Formula III, Formula III-P, Formula IV-P, Formula V-P, and Formula V, or as defined in any of the embodiments described herein, and a pharmaceutically acceptable carrier.

In another aspect, provided is a method treating a cancer in a mammal comprising administering to the mammal a therapeutically effective amount of a compound according to any one of Formula I, Formula I-P-1, Formula I-P-2, Formula II-P, Formula III, Formula III-P, Formula IV-P, Formula V-P, and Formula V, or as defined in any of the embodiments described herein or a pharmaceutical composition thereof (i.e. the compound and a pharmaceutically acceptable carrier). In some or any embodiments, the compound is administered to the mammal parenterally, transdermally, orally, intranasally, topically, rectally, or via an intra-tumoral administration, optionally, in a pharmaceutical composition. In some or any embodiments, the cancer is renal cancer, including renal cell carcinoma (RCC) and metastatic RCC (mRCC).

### General Synthetic Methods

The compounds described herein can be prepared in accordance with one or more of methods described, for example, in references below. General syntheses of certain related starting materials have been described in the literature. For example, the preparation of Boc-protected polymyxin nonapeptide was described by O'Dowd et al. in Tetrahedron Lett. 2007, vol. 48, p. 2003. Additional protected polymyxin B nonapeptide and colistin nonapeptide derivatives can be prepared as described by Okimura et al. in Chem. Pharm. Bull. 2007, vol. 55, pp. 1724-1730. Likewise, the general peptide acylation chemistry was described in the ref. Tetrahedron Lett. 2007, vol. 48, pp. 2003-2005.

Additional general methods suitable for preparation of compounds of Formula I, Formula I-P-1, Formula I-P-2, Formula II-P, Formula III, Formula III-P, Formula IV-P, Formula V-P, or Formula V have been described in publications WO 2021/150792, WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687.

Methods suitable for incorporation of suitable enzymatically and/or chemically cleavable groups X, Y, and Z (and additional spacers/linkers in same) in compounds of Formula I, Formula I-P-1, Formula I-P-2, Formula II-P, Formula III, Formula III-P, Formula IV-P, Formula V-P, or Formula V have been described in generally related synthetic art for preparation of ADCs and other agents, such as reported, for example, in publications US 20170355769; J. Am. Chem. Soc. 2018, vol. 140, p. 1617; Bioconjugate Chem. 2016, vol. 27, p. 1606; Bioconjugate Chem. 2016, vol. 27, p. 1645; Bioconjugate Chem. 2015, vol. 26, p. 919; Mol. Pharmaceutics 2015, vol. 12, p. 1813; ACS Med. Chem. Lett. 2017, vol. 8, p. 1037; ACS Med. Chem. Lett. 2016, vol. 7, p. 983; Org. Process Res. Dev. 2019, vol. 23, p. 2647; Bioconjugate Chem. 2016, vol. 27, p. 1880; Bioconjugate Chem. 2017, vol. 28, p. 620; Org. Process Res. Dev. 2018, vol. 22, p. 286; Bioconjugate Chem. 2015, vol. 26, p. 2216; J. Med. Chem. 2014, vol. 57, p. 6949; Bioconjugate Chem. 2018, vol. 29, p. 1155; J. Am. Chem. Soc. 2015, vol. 137, p. 3229; Mol. Pharmaceutics 2018, vol. 15, p. 2384; ACS Med. Chem. Lett. 2016, vol. 7, p. 988; Chem. Biodiversity 2019, vol. 16, e1800520; Nature Commun. 2018, vol. 9, p. 2512; Mol. Pharmaceutics 2011, vol. 8, p. 901; ACS Med. Chem. Lett. 2019, vol. 10, p. 1393; J. Nat. Prod. 2017, vol. 80, p. 2447; ACS Med. Chem. Lett. 2019, vol. 10, p. 1674; Pharmaceutics 2013, vol. 5, p. 220; and other references cited in these publications.

Specific methods, amino acid reagents, as well as linker/spacer structures described in above literature are directly adaptable to prepare compounds of Formula I, Formula I-P-1, Formula I-P-2, Formula II-P, Formula III, Formula III-P, Formula IV-P, Formula V-P, or Formula V, by straightforward variations in specific reagents and protection/deprotection schemes, obvious to one skilled in synthetic organic chemistry.

Additional syntheses of specific compounds described herein are illustrated by various synthetic Schemes for Examples below, likewise adaptable to preparation of additional compounds provided herein.

### EXAMPLES

Embodiments are described in the following examples, which are meant to illustrate and not limit the scope of this disclosure. Common abbreviations well-known to those with ordinary skills in the synthetic art. used throughout. NMR means 400 MHz H NMR spectra (delta, ppm) recorded in D₂O unless specified otherwise. LCMS means liquid chromatography mass-spectroscopy analysis. MS means mass-spectroscopy data (m/z) for a positive ionization method. Chromatography means silica gel chromatography using common organic solvents unless specified otherwise. TLC means thin-layer chromatography. HPLC means reverse-phase high-performance chromatography using commercial C18 phase columns. CDI means carbonyldiimidazole. DCM means dichloromethane. TES means Et₃SiH, TFA means CF₃COOH, EA means EtOAc or ethyl acetate, ACN means MeCN, DMF means N,N-dimethylformamide, DCC means N,N′-dicyclohexylcarbodiimide, DCE means 1,2-dichloroethane, NMP means N-methyl pyrrolidinone, and PE means hexanes or light petroleum ether. MeOH means methanol and t-BuOH means tert-Butyl alcohol. THE means tetrahydrofuran. Cs₂CO₃ means cesium carbonate, NaHCO₃ means sodium carbonate, and Na₂SO₄ means sodium sulfate. HCl means hydrochloric acid. T3P means propanephosphonic acid anhydride. DIEA means N,N-Diisopropylethylamine and DMAP means 4-dimethylaminopyridine. HATU means (1-[bis(dimethylamino)methylene]-1H-1,2,3-triazolo[4,5-b]pyridinium 3-oxide hexafluorophosphate, hexafluorophosphate azabenzotriazole tetramethyl uronium. Boc means tert-Butyloxycarbonyl and Cbz means benzyloxycarbonyl. Pd means palladium. r.t or RT means room temperature. Ar means argon. C18 chromatography means reverse phase chromatography using a gradient of water and acetonitrile (ACN), or of same and containing 0.05% to 1% of TFA. The reagent PMBN(Boc)₄ is H-Thr-Dab(Boc)-cyclo[Dab(Boc)-Dab(Boc)-D-Phe-Leu- Dab(Boc)-Dab(Boc)-Thr]. The reagent Dab(Boc)PMBN(Boc)₄[same as Dab(Boc)-PMBN(Boc)₄] is H-Dab(Boc)-Thr-Dab(Boc)-cyclo[Dab(Boc)-Dab(Boc)-D-Phe-Leu- Dab(Boc)-Dab(Boc)-Thr]{same as Dab(Boc)-Thr-Dab(Boc)-cyclo[Dab(Bofc)-Dab(Boc)-D-Phe-Leu- Dab(Boc)-Dab(Boc)-Thr]}. Axitinib-Pnp is (E)-4-nitrophenyl 6-((2-(methylcarbamoyl)phenyl)thio)-3-(2-(pyridin-2-yl)vinyl)-1H-indazole-1-carboxylate. Other reagent abbreviations are just as employed in common synthetic literature, including the American Chemical Society list of abbreviations, such as found, for example, in the Journal of Organic Chemistry; or in the Journal of Peptide Chemistry. Unless specified otherwise, all reagents were either from commercial sources, or made by conventional methods described in available literature.

### Example 1

### Synthesis of the Compound of Example 1

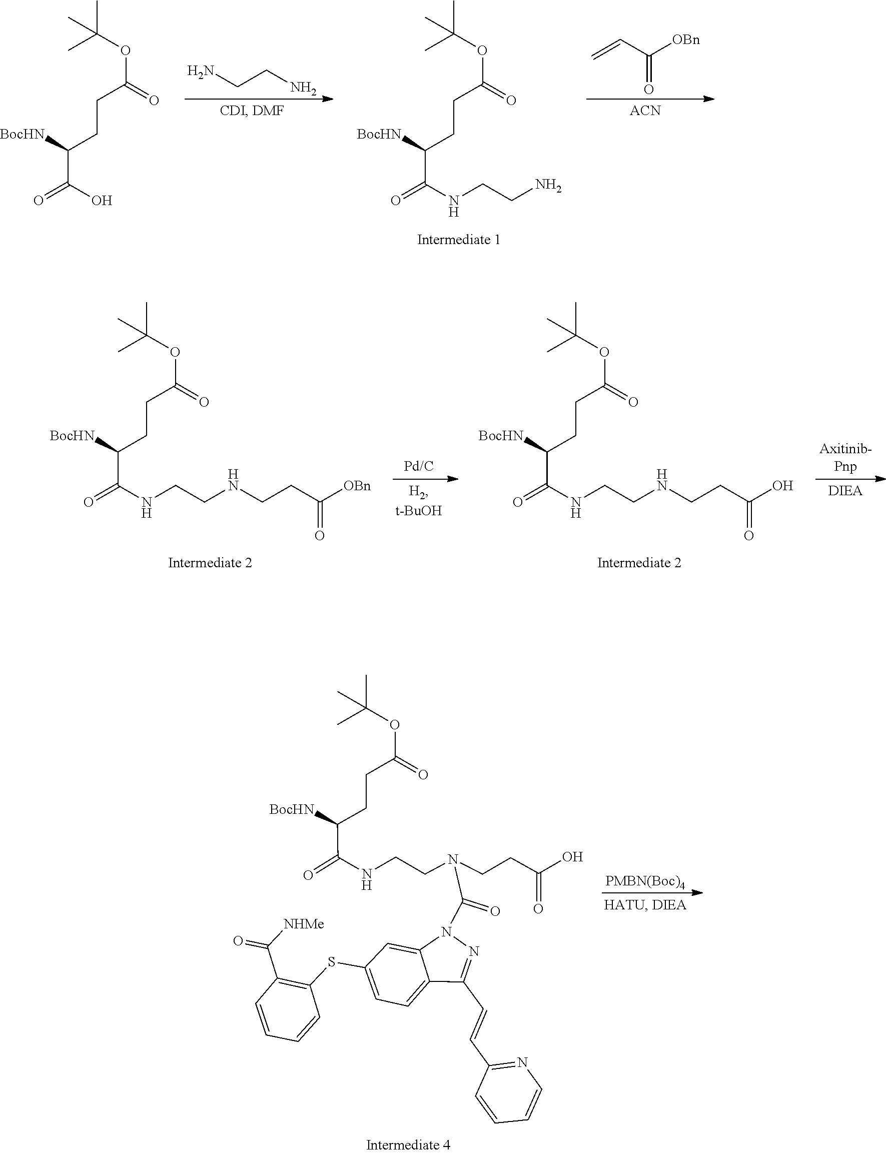

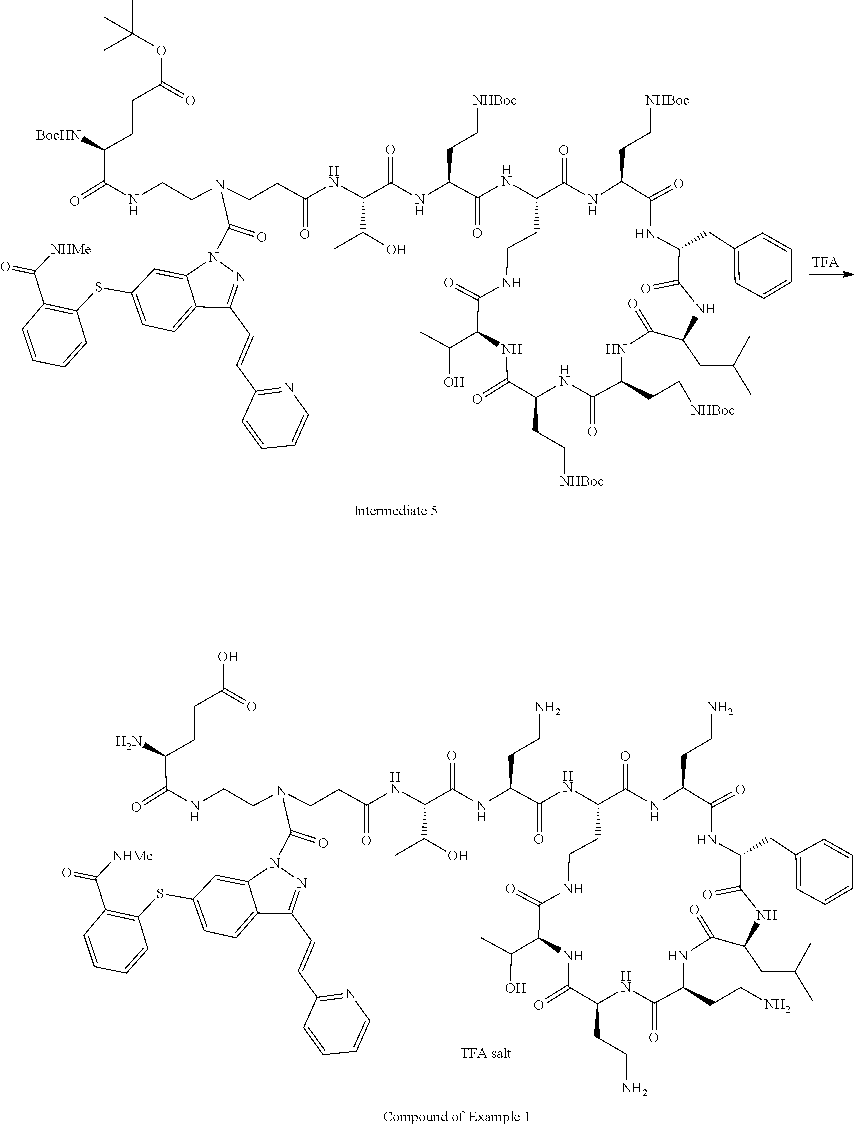

Intermediate 1. The mixture of (S)-5-(tert-butoxy)-2-((tert-butoxycarbonyl)amino)-5-oxopentanoic acid (505 mg, 1.67 mmol) and CDI (324 mg, 2 mmol) in DMF (4 mL) was stirred at r.t. for 1.5 hr, then ethane-1,2-diamine (1.1 mL, 16.7 mmol) was added to the reaction. The reaction was stirred at r.t. for 1.5 hr. It was diluted with water (20 mL), extracted with DCM (15 mL×4), washed with H₂O (10 mL*2) and brine (10 mL). Dried and evaporated to afford crude Intermediate 1 (0.62 g). MS: 346.16 [M+H]+.

Intermediate 2. The mixture of Intermediate 1 (0.62 g, 1.67 mmol) and benzyl acrylate (0.24 g, 1.5 mmol) in ACN (4 mL) was stirred at 45° C. under Ar for 36 hrs. The volatiles were removed, and the residue was purified by C18 column (ACN/H₂O=0˜80%) to give Intermediate 2 (0.5 g). MS: 508.31 [M+H]+.

Intermediate 3. The suspension of Intermediate 2 (0.5 g, 0.99 mmol) and 10% Pd/C (0.15 g) in t-BuOH (5 mL) was degassed with H₂ for 5 times, and then stirred at rt under H₂ for 4.5 hrs. Filtered and dried to give crude Intermediate 3 (0.42 g). MS: 418.25 [M+H]⁺.

Intermediate 4. The mixture of Intermediate 3 (0.313 mg, 0.75 mmol), Axitinib-Pnp (0.413 g, 0.75 mmol) and DIEA (0.2 mL, 1.13 mmol) in NMP (5 mL) was stirred at r.t. under Ar overnight, then was diluted and extracted with EA (50 mL×3), washed with H₂O (5 mL*2) and brine (5 mL). Dried and evaporated. Purified by C18 column (ACN/H₂O=0˜90%) to afford Intermediate 4 (0.35 g). MS: 830.16 [M+H]⁺.

Intermediate 5. The reaction of Intermediate 4 (0.34 g, 0.41 mmol), PMBN(Boc)₄ (0.56 g, 0.41 mmol), HATU (0.187 g, 0.49 mmol) and DIEA (0.145 mL, 0.82 mmol) in DMF (3 mL) was stirred at r.t. for 4 hrs. The mixture was extracted with EA (60 mL), washed with H₂O (10 mL*2) and brine (10 mL). Dried and evaporated. Purified by C¹⁸ column (ACN/H₂O=0˜80%) to provide Intermediate 5 (0.45 g). MS: 1087.95 [M+2H]²⁺.

Compound of Example 1. The mixture Intermediate 5 (110 mg, 0.05 mmol) in TFA/DCM (0.5 mL/1.5 mL) was stirred at r.t. for 3 hrs. The volatiles were evaporated, and the crude product was purified by HPLC (ACN/H₂O=0˜50%) to afford the Compound of Example 1 (43 mg). NMR: 8.45 (t, J=7.9 Hz, 1H), 8.25 (d, J=7.2 Hz, 1H), 7.97-7.86 (m, 2H), 7.82 (t, J=6.6 Hz, 1H), 7.65 (s, 2H), 7.50-7.38 (m, 4H), 7.25 (dq, J=14.9, 7.3 Hz, 4H), 7.14 (d, J=7.4 Hz, 2H), 4.47 (dt, J=11.4, 5.9 Hz, 1H), 4.42-4.31 (m, 2H), 4.22-4.00 (m, 8H), 3.88 (s, 2H), 3.74 (s, 3H), 3.38 (s, 1H), 3.21 (s, 1H), 3.09-2.87 (m, 9H), 2.83-2.55 (m, 7H), 2.26 (d, J=6.1 Hz, 2H), 2.20-2.03 (m, 5H), 1.92 (s, 4H), 1.87-1.66 (m, 3H), 1.39 (ddd, J=14.0, 9.6, 4.3 Hz, 1H), 1.30 (ddd, J=14.4, 10.6, 4.1 Hz, 1H), 1.07 (t, J=5.2 Hz, 3H), 0.98 (s, 3H), 0.78-0.64 (m, 4H), 0.59 (t, J=5.0 Hz, 3H). MS: 1619.68 [M+H]⁺.

### Example 2

### Synthesis of the Compound of Example 2

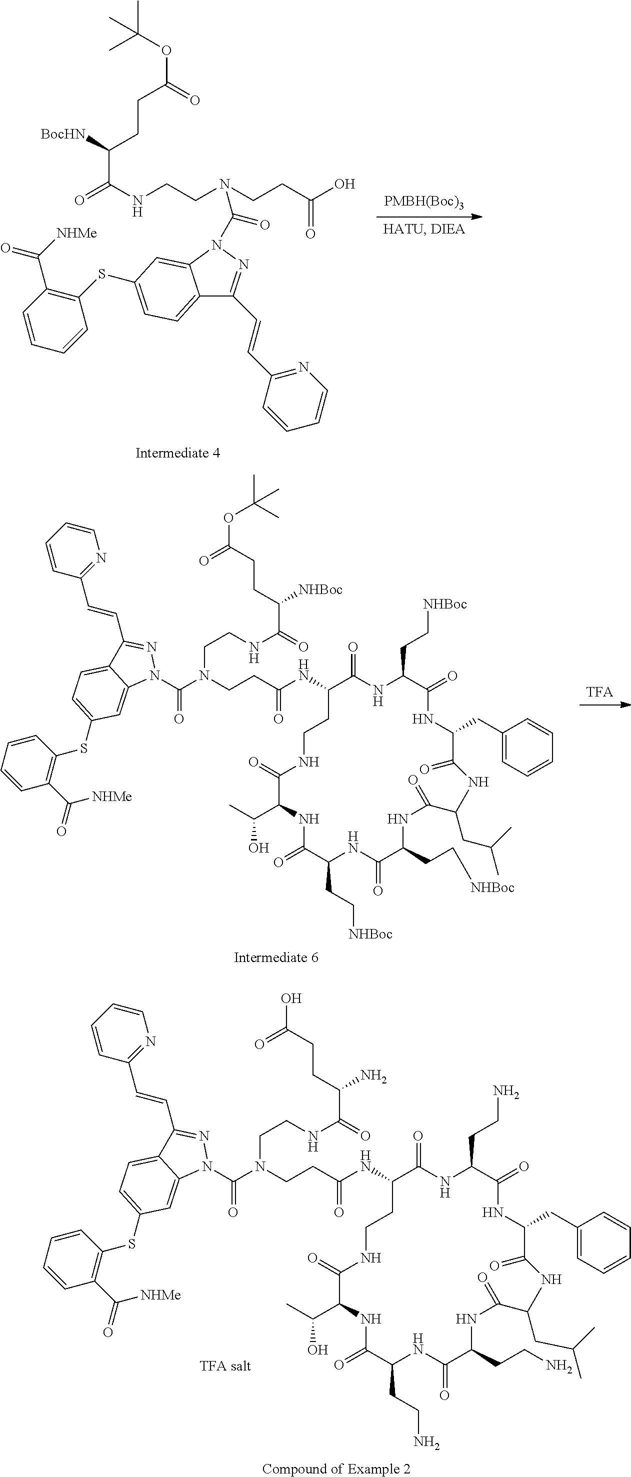

Intermediate 6. The Intermediate 6 was made according to the similar procedure to the Intermediate 5 except that Intermediate 4 was coupled with PMBH(Boc)₃ instead of PMBN(Boc)₄. MS: 887.7 [M-99]²⁺.

Compound of Example 2. The Compound of Example 2 was made according to the similar procedure to the Compound of Example 1 except that Intermediate 4 was coupled with PMBH(Boc)₃ instead of PMBN(Boc)₄. NMR (600 MHz, Deuterium Oxide) δ 8.55 (dt, J=6.1, 2.7 Hz, 1H), 8.46-8.37 (m, 1H), 8.18 (s, 1H), 7.90-7.84 (m, 1H), 7.78 (ddd, J=7.3, 5.9, 1.2 Hz, 1H), 7.65 (s, 1H), 7.42-7.32 (m, 4H), 7.22-7.14 (m, 3H), 7.09-7.03 (m, 2H), 4.33 (s, 1H), 4.18-3.56 (m, 13H), 3.30 (s, 1H), 3.07 (dt, J=13.9, 7.3 Hz, 1H), 2.93 (dddd, J=37.3, 15.9, 10.9, 6.4 Hz, 5H), 2.83-2.68 (m, 3H), 2.58 (d, J=6.9 Hz, 6H), 2.18 (t, J=7.5 Hz, 2H), 2.14-1.72 (m, 9H), 1.64 (d, J=44.5 Hz, 2H), 1.31 (d, J=9.8 Hz, 2H), 0.96 (s, 3H), 0.62 (d, J=6.5 Hz, 3H), 0.58-0.51 (m, 3H). MS: 1417.62 [M+H]⁺.

### Example 3A

### Synthesis of the Compound of Example 3

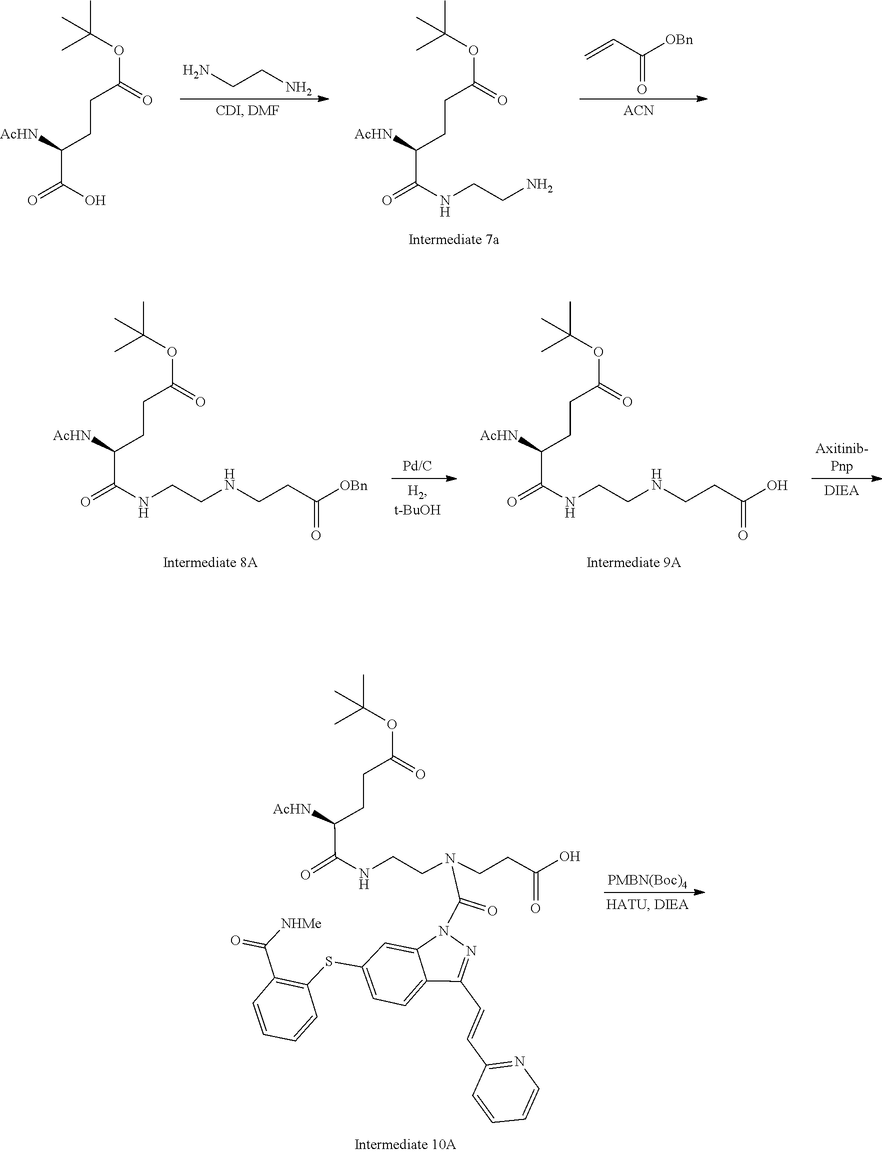

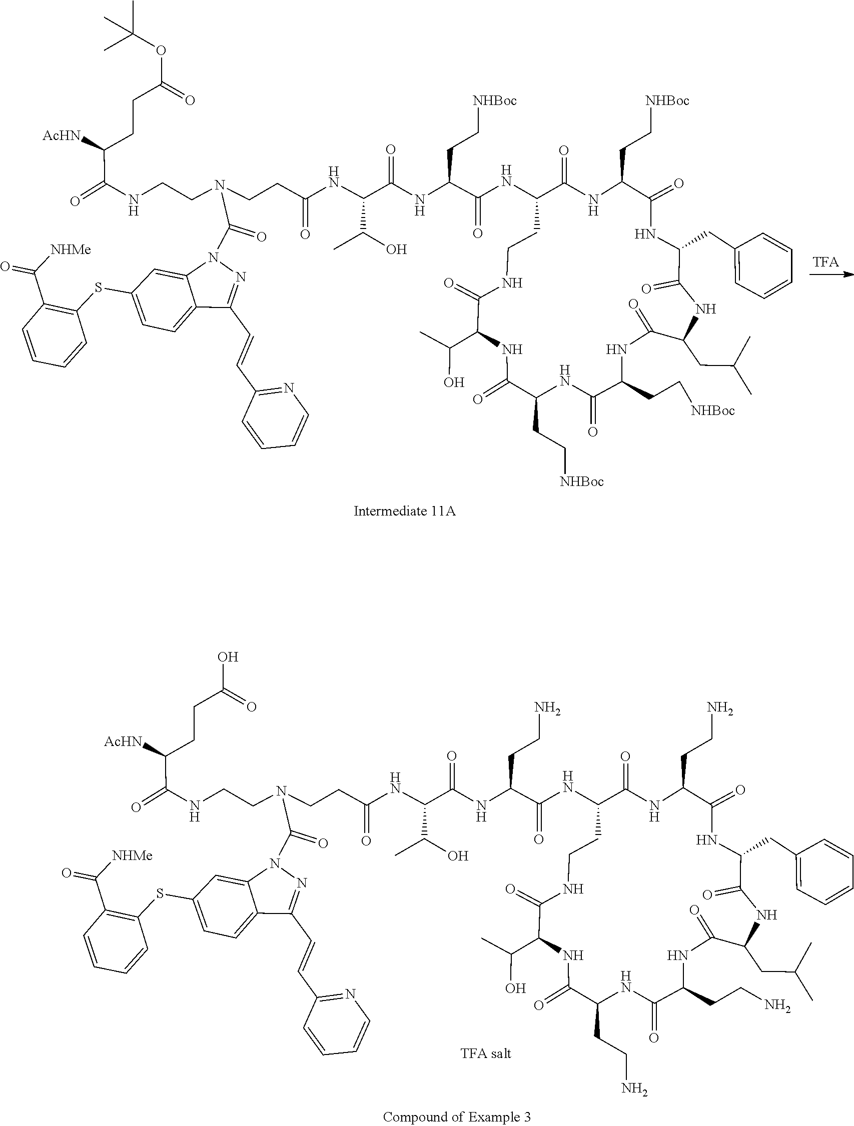

Compound of Example 3. The Compound of Example 3 is made according to a similar procedure to the Compound of Example 1 except that starting from (S)-2-acetamido-5-(tert-butoxy)-5-oxopentanoic acid instead of (S)-5-(tert-butoxy)-2-((tert-butoxycarbonyl)amino)-5-oxopentanoic acid.

### Example 3B

### Alternative Synthesis of the Compound of Example 3

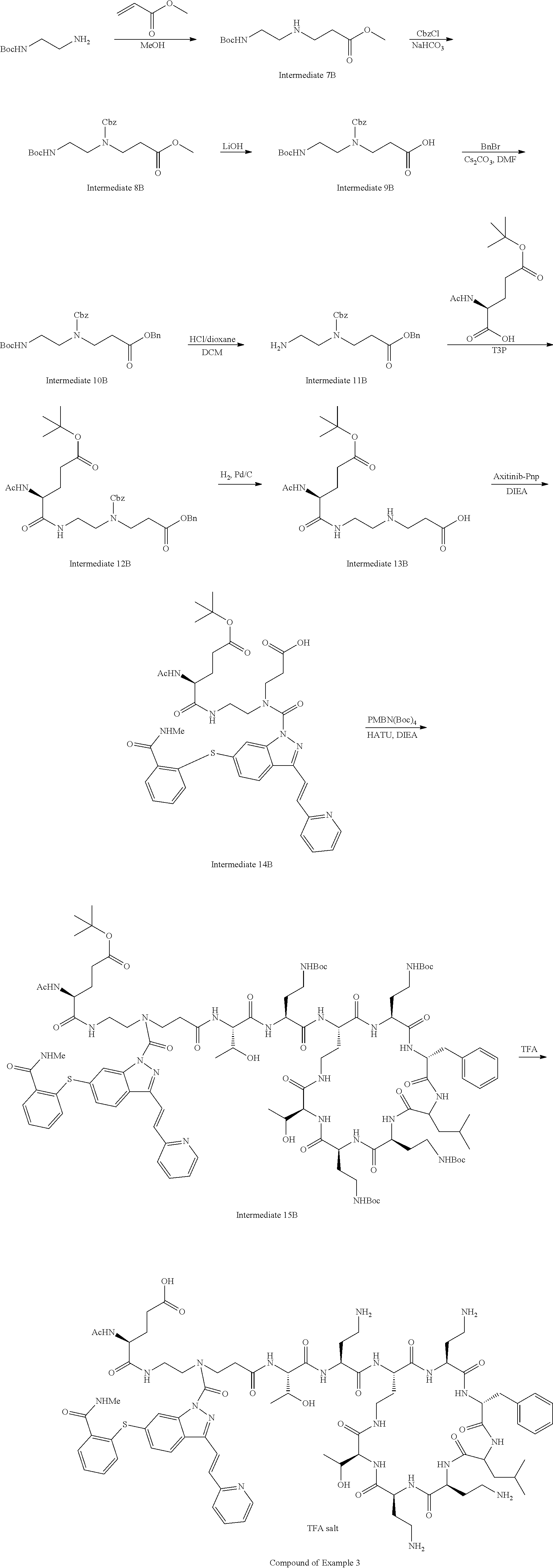

Intermediate 7B. The mixture of tert-butyl (2-aminoethyl)carbamate (25 g, 156 mmol) in MeOH (250 mL) was added methyl prop-2-enoate (10.8 g, 125 mmol) drop-wised at 0° C. under N₂, then stirred at r.t. for 12 hrs. It was evaporated and purified by silica gel chromatography to afford Intermediate 7B (25.0 g, 65.1% yield). NMR (400 MHz, CDCl₃) δ 4.98 (br s, 1H), 3.68 (s, 3H), 3.20 (q, J=5.7 Hz, 2H), 2.88 (t, J=6.5 Hz, 2H), 2.73 (t, J=5.8 Hz, 2H), 2.50 (t, J=6.5 Hz, 2H), 1.43 (s, 9H).

Intermediate 8B. The mixture of Intermediate 7B (25.0 g, 102 mmol) and NaHCO₃ (17.1 g, 203 mmol) in dioxane (125 mL) and H₂O (125 mL) was added CbzCl (22.5 g, 132 mmol) drop-wised at 0° C. The reaction mixture was warmed to 25° C. slowly and stirred for 4 hrs. The mixture was diluted with H₂O (300 mL), extracted with EtOAc (100 mL×3). The combined organic layers were washed with brine (10 mL×2), dried over Na₂SO₄, filtered and concentrated to afford Intermediate 8B as a yellow oil (40 g crude). NMR (400 MHz, CDCl₃) δ 7.39-7.35 (m, 3H), 7.34-7.27 (m, 2H), 5.12 (s, 2H), 3.64 (d, J=10.7 Hz, 3H), 3.56 (t, J=7.1 Hz, 2H), 3.40 (br s, 2H), 3.26 (d, J=11.6 Hz, 2H), 2.70-2.47 (m, 2H), 1.42 (s, 9H).

Intermediate 9B. The mixture of Intermediate 8B (10.0 g, 26.3 mmol) in THE (90.0 mL) was added LiOH (1.43 g, 34.2 mmol) dissolved in H₂O (30.0 mL) at 0° C. The reaction mixture was warmed to 25° C. and stirred for 2.5 hrs. The reaction mixture was concentrated under reduced pressure to remove THF. The aqueous phase was acidified by 2 M HCl until pH=4 and extracted with DCM 150 mL (50 mL*3). The combined organic layers were washed with brine (10 mL), dried over Na₂SO₄, filtered and concentrated to afford Intermediate 9B as a light-yellow oil (13.0 g crude). MS: 267.1 [M-99]⁺.

Intermediate 10B. The mixture of Intermediate 9B (11.0 g, 30 mmol) in DMF (100 mL) was added Cs₂CO₃ (29.3 g, 90.1 mmol), followed by addition of (bromomethyl) benzene (7.70 g, 45.0 mmol) drop-wised at rt. The reaction mixture was stirred for 12 hrs. The mixture was poured into H₂O (30 mL), extracted with EtOAc (40 mL×3). The combined organic layers were dried over Na₂SO₄, filtered and concentrated. The residue was purified by silica gel column chromatography to afford Intermediate 10 (9.00 g, 65.7% yield) as brown oil. MS: 357.2 [M−99]⁺.

Intermediate 11B. The mixture of Intermediate 10B (9.00 g, 19.7 mmol) in DCM (90 mL) was added HCl (4 M in dioxane, 29.6 mL, 118 mmol). The reaction mixture was stirred at rt for 4 hrs. The mixture was concentrated to afford Intermediate 11B (7.00 g crude) as white solid.

Intermediate 12. The mixture of (S)-2-acetamido-5-(tert-butoxy)-5-oxopentanoic acid (2.00 g, 8.15 mmol), Intermediate 11B (3.84 g, 9.79 mmol) in DCM (10 mL) was added TEA (3.40 mL, 24.5 mmol) and T3P (7.78 g, 12.2 mmol) at 0° C. The reaction mixture was stirred for 12 hrs. The mixture was washed with brine (30 mL×2), dried over Na₂SO₄, filtered and concentrated. The residue was purified by silica gel column chromatography to afford Intermediate 12B (1.80 g, 37.8% yield) as light yellow solid. MS: 528.2 [M−55]⁺.

Intermediate 13B. The mixture of Intermediate 12B (1.75 g, 3.00 mmol) in 2,2,2-trifluoroethan-1-ol (20.0 mL) was added Pd/C 10% (0.350 g, 3.29 mmol), the mixture was stirred at rt for 12 hrs. The suspension was filtered through a pad of silica gel and filter cake was washed with 2,2,2-trifluoroethan-1-ol (20.0 mL×3). The combined filtrates were concentrated under reduced pressure to afford Intermediate 13B (1.00 g, 92.6% yield) as yellow oil. MS: 360.2 [M+H]⁺.

Intermediate 14B. The Intermediate 14B was made according to the similar procedure to the Intermediate 4, yield 58.2%. MS: 772.3 [M+H]⁺.

Intermediate 15B. The Intermediate 15B was made according to the similar procedure to the Intermediate 5, yield 55%. MS: 1059.7 [M+2H]²⁺.

Compound of Example 3. The Compound of Example 3 was made according to the similar procedure to the Compound of Example 1. NMR (400 MHz, Deuterium Oxide) δ 8.67 (d, J=5.9 Hz, 1H), 8.57-8.50 (m, 1H), 8.34 (d, J=8.4 Hz, 1H), 8.07-7.98 (m, 2H), 7.90 (t, J=6.6 Hz, 1H), 7.80-7.68 (m, 2H), 7.58-7.45 (m, 4H), 7.36-7.26 (m, 4H), 7.23-7.15 (m, 2H), 4.28-4.05 (m, 10H), 3.98-3.72 (m, 5H), 3.61-3.41 (m, 3H), 3.33-3.20 (m, 2H), 3.17-2.94 (m, 10H), 2.89-2.61 (m, 8H), 2.30-2.11 (m, 7H), 2.02 (s, 1H), 1.94-1.78 (m, 7H), 1.53-1.29 (m, 3H), 1.21-0.99 (m, 6H), 0.78-0.62 (m, 6H). MS: 831.4 [M+2H]²⁺.

### Example 4A

### Synthesis of the Compound of Example 4

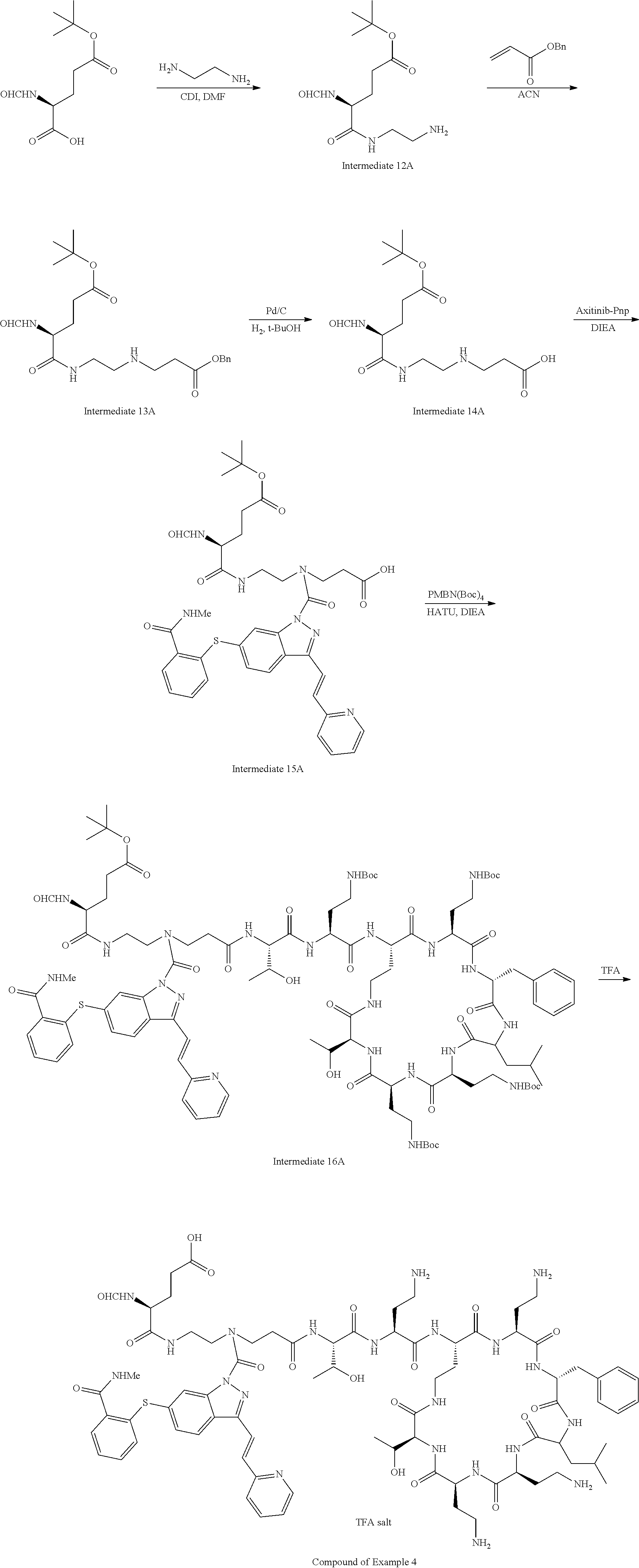

Compound of Example 4. The Compound of Example 4 is made according to a similar procedure to the Compound of Example 1 except that starting from (S)-2-formamido-5-(tert-butoxy)-5-oxopentanoic acid instead of (S)-5-(tert-butoxy)-2-((tert-butoxycarbonyl)amino)-5-oxopentanoic acid.

### Example 4B

### Alternative Synthesis of the Compound of Example 4

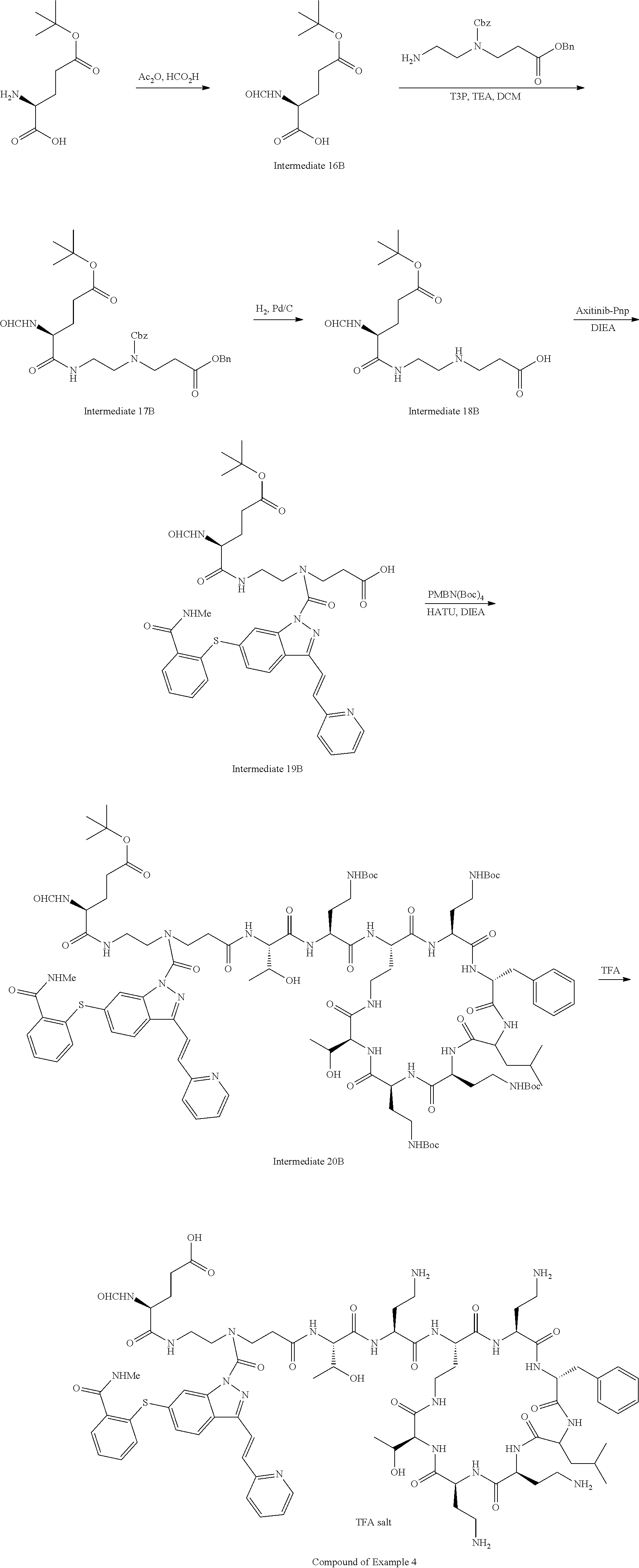

Intermediate 16B. To a solution of (S)-2-amino-5-(tert-butoxy)-5-oxopentanoic acid (5.00 g, 24.6 mmol) in formic acid (3.17 mL, 246 mmol) was added Ac₂O (1.01 mL, 10.8 mmol) slowly. The mixture was stirred at rt for 0.5 hr. The reaction mixture was quenched by addition of H₂O (20.0 mL) at rt, filtered and concentrated under reduced pressure to afford Intermediate 16B (2.60 g, 45.7% yield) as a white solid. MS: 230.0 [M−H]⁻.

Intermediate 17B. The Intermediate 17B was made according to the similar procedure to the Intermediate 12B, yield 19.5% as yellow oil. MS: 514.3 [M−55]⁺.

Intermediate 18B. The Intermediate 18 was made according to the similar procedure to the Intermediate 13B, white solid. MS: 346.2 [M+H]⁺.

Intermediate 19B. The Intermediate 19 was made according to the similar procedure to the Intermediate 4, white solid. MS: 758.4 [M+H]⁺.

Intermediate 20B. The Intermediate 20B was made according to the similar procedure to the Intermediate 5, 28.8% yield as white solid. MS: 1002.6 [M+2H]²⁺.

Compound of Example 4. The Compound of Example 4 was made according to the similar procedure to the Compound of Example 1. NMR (400 MHz, Deuterium Oxide) δ 8.67 (d, J=5.6 Hz, 1H), 8.58-8.51 (m, 1H), 8.35 (d, J=8.4 Hz, 1H), 8.05-7.95 (m, 3H), 7.94-7.87 (m, 1H), 7.76-7.68 (m, 2H), 7.57-7.44 (m, 5H), 7.37-7.24 (m, 5H), 7.19 (d, J=7.3 Hz, 2H), 4.53 (t, J=8.0 Hz, 1H), 4.41 (td, J=9.1, 4.7 Hz, 2H), 4.30-4.10 (m, 11H), 3.98-3.69 (m, 5H), 3.49 (br s, 2H), 3.32-3.23 (m, 1H), 3.13-2.97 (m, 11H), 2.89-2.69 (m, 5H), 2.67 (s, 3H), 2.32-2.13 (m, 8H), 2.10-1.95 (m, 3H), 1.93-1.83 (m, 3H), 1.77 (br s, 3H), 1.50-1.31 (m, 2H), 1.17-1.02 (m, 7H), 0.86-0.75 (m, 2H), 0.75-0.71 (m, 3H), 0.65 (d, J=5.9 Hz, 3H). MS: 824.2 [M+2H]²⁺.

### Example 5A

### Synthesis of the Compound of Example 5

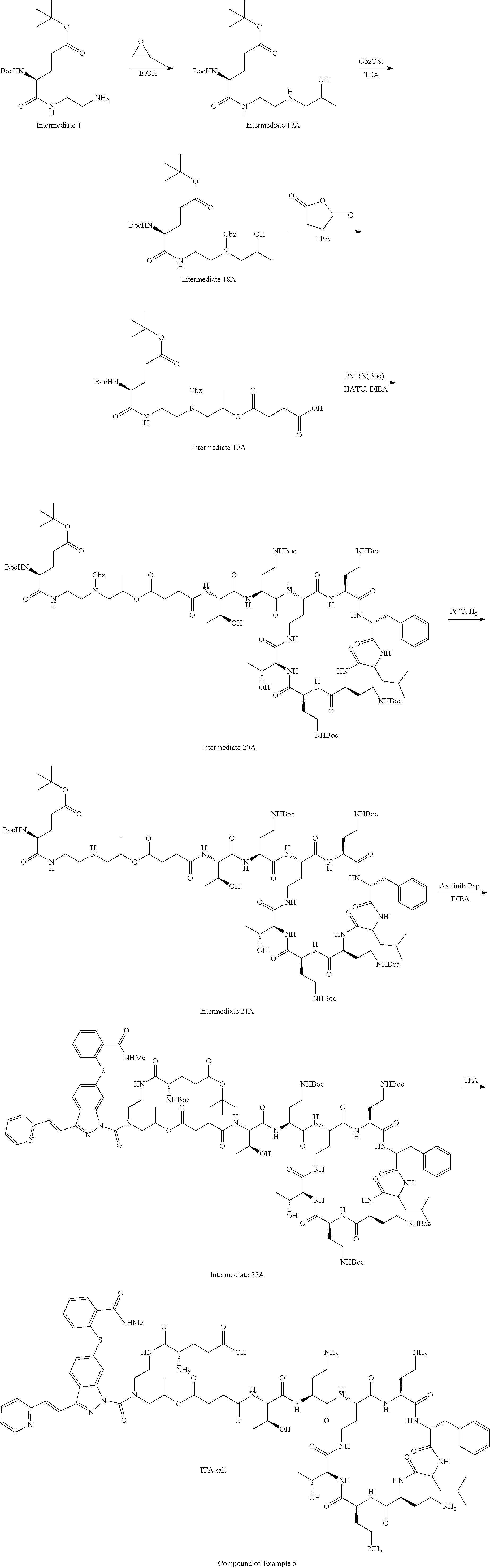

Compound of Example 5. The Compound of Example 5 is made according to a procedure as above starting from Intermediate 1.

### Example 5B

### Alternative Synthesis of the Compound of Example 5

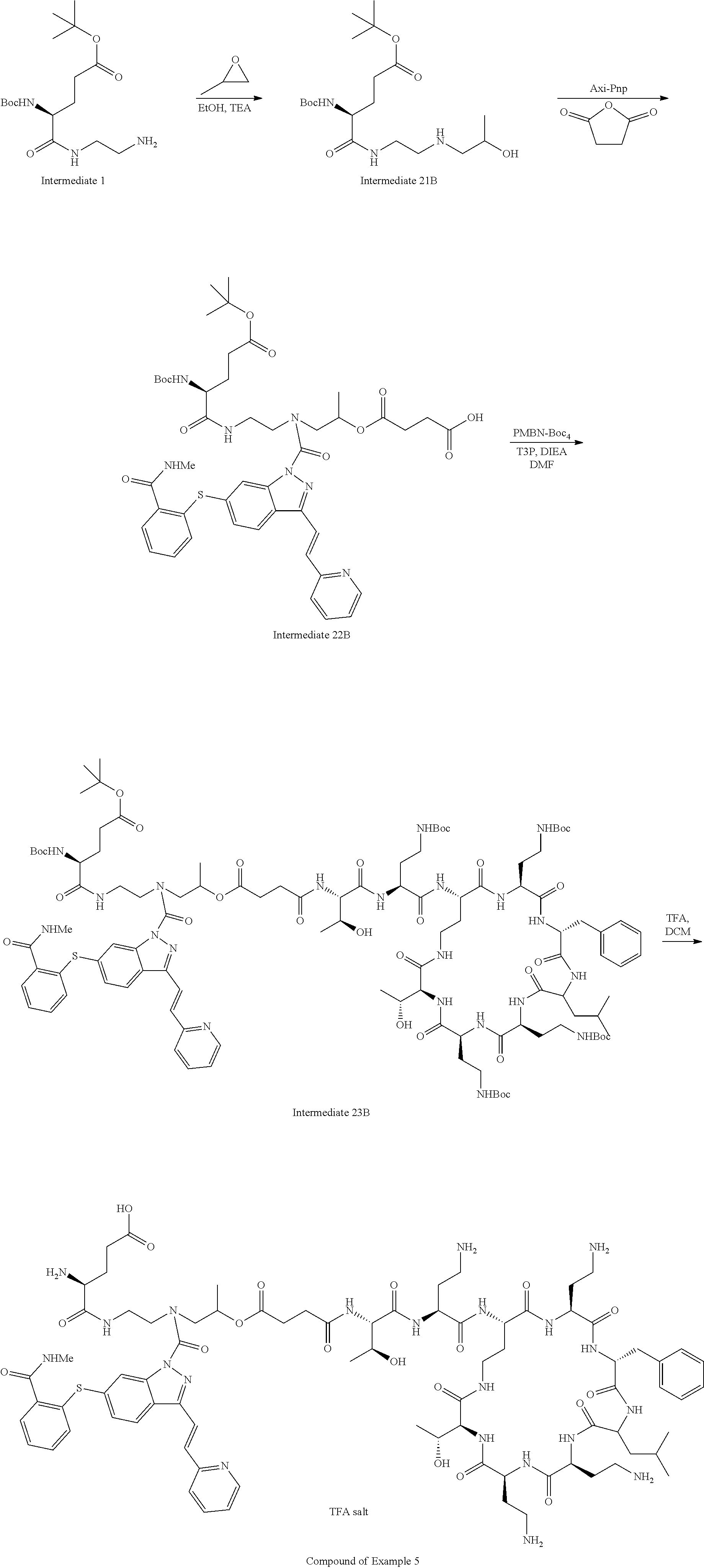

Intermediate 21B. 2-Methyloxirane (0.164 mL, 2.34 mmol) was added to a mixture of Intermediate 1 (810 mg, 2.34 mmol), TEA (0.418 mL, 3 mmol) in 10 mL of EtOH. The mixture was stirred at rt overnight. The mixture was concentrated and purified by silica gel chromatography to afford Intermediate 21B (220 mg) as colorless oil. MS: 404.3 [M+H]⁺.

Intermediate 22B. Intermediate 21B (112 mg, 0.28 mmol), Axi-PNP.HCl (127 mg, 0.22 mmol), TEA (0.2 mL, 1.4 mmol) in 2 mL of DMF was stirred at rt for 3 h. 113 mg of succinic anhydride and 38.6 mg of DMAP was added. The mixture was stirred at rt overnight. The mixture was partitioned between H₂O (10 mL), 0.5 M HCl (5 mL) and EtOAc (10 mL), the water phase was extracted with EtOAc (5 mL), the combined organic layer was washed with brine (10 mL), dried over Na₂SO₄, filtered and concentrated. The residue was purified by reversed C18 pre. HPLC to afford Intermediate 22B (43 mg). MS: 916.2 [M+H]⁺.

Intermediate 23B. The Intermediate 23B was made according to the similar procedure to the Intermediate 5, 28.8% yield as white solid. MS: 1131.4 [M+2H]²⁺.

Compound of Example 5. The Compound of Example 5 was made according to the similar procedure to the Compound of Example 1. NMR (600 MHz, Deuterium Oxide) δ 8.63 (d, J=6.0 Hz, 1H), 8.58-8.51 (m, 1H), 8.35 (d, J=8.4 Hz, 1H), 8.45 (t, J=8.4 Hz, 1H), 8.25 (d, J=7.8 Hz, 1H), 7.30-7.21 (m, 4H), 7.16 (d, J=7.8 Hz, 2H), 4.51 (t, J=7.8 Hz, 1H), 4.38 (m, 2H), 4.20-4.11 (m, 11H), 3.91-3.69 (m, 5H), 3.37-3.22 (m, 3H), 3.03-2.94 (m, 11H), 2.80 (m, 2H), 2.72-2.61 (m, 5H), 2.46-2.42 (m, 2H), 2.29-2.25 (m, 2H), 2.21-1.76 (m, 13H), 1.44-1.32 (m, 2H), 1.10-1.09 (m, 9H), 0.80-0.75 (m, 2H), 0.70-0.68 (m, 3H), 0.63-0.61 (m, 3H). MS: 1705.3 [M+H]⁺.

### Example 6

### Synthesis of the Compound of Example 6

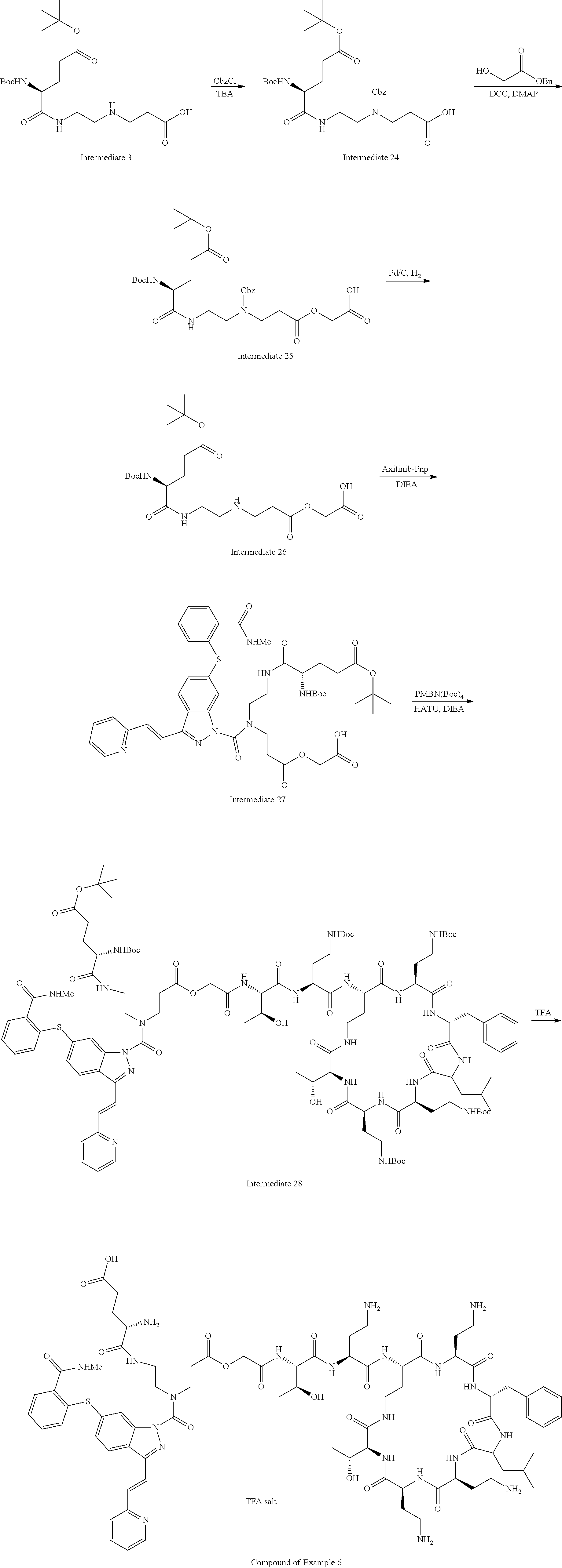

Intermediate 24. The mixture of Intermediate 3 (1.2 g, 2.87 mmol) and TEA (0.8 mL, 5.75 mmol) in DCM (15 mL) was stirred at 0° C., then CbzCl (0.45 mL, 3.16 mmol) was added dropwise to the reaction. The reaction was stirred at r.t. for 3 hrs. Extracted with DCM (80 mL), washed with H₂O (30 mL) and brine (30 mL). Dried and evaporated. Purified by C18 column (ACN/H₂O=0-80%) to give Intermediate 24 (635 mg). MS: 552.15 [M+H]⁺.

Intermediate 25. The reaction of Intermediate 24 (600 mg, 1.09 mmol), benzyl 2-hydroxyacetate (451 mg, 2.72 mmol), DCC (671 mg, 3.26 mmol) and DMAP (27 mg, 0.22 mmol) in DCM (20 mL) was stirred at r.t under Ar for 18 hrs. Extracted with DCM (80 mL) washed with H₂O (30 mL) and brine (30 mL). Dried and evaporated. Purified by C18 column (ACN/H₂O=0˜90%) to give Intermediate 25 (407 mg). MS: 700.16 [M+H]⁺.

Intermediate 26. The suspension of Intermediate 25 (400 mg, 0.57 mmol) and Pd/C (56% H₂O, 200 mg) in t-BuOH (10 mL) was degassed with H₂ for 5 times, then stirred at r.t under H₂ for 6 hrs. Filtered and evaporated to provide Intermediate 26 (250 mg). MS: 476.26 [M+H]⁺.

Intermediate 27. The mixture of Intermediate 26 (250 mg, 0.53 mmol), Axi-Pnp (319 mg, 0.57 mmol) and DIEA (0.23 mL, 1.31 mmol) in NMP (5 mL) was stirred at r.t for 6 hrs. Extracted with EA (20 mL*3), washed with H₂O (10 mL*2) and brine (10 mL). Dried and evaporated. Purified by C18 column (ACN/H₂O=0˜80%) to give Intermediate 27 (280 mg). MS: 888.89 [M+H]⁺.

Intermediate 28. The reaction of Intermediate 27 (240 mg, 0.27 mmol), PMBN(Boc)₄ (368 mg, 0.27 mmol), HATU (113 mg, 0.30 mmol) and DIEA (0.096 mL, 0.54 mmol) in DMF (6 mL) was stirred at 25° C. under Ar for 5 hrs. Extracted with EA (50 mL), washed with H₂O (5 mL*2) and brine (5 mL). Dried and evaporated. Purified by C18 column (ACN/H₂O=0˜90%) to provide Intermediate 28 (85 mg). MS: 1117.00 [M+2H]²⁺.

Compound of Example 6. The mixture of Intermediate 28 (83 mg, 0.037 mmol) in TFA/DCM (1 mL/3 mL) was stirred at rt for 1 hr. The volatiles were removed and the residue was purified by C18 column (ACN/H₂O=0˜70%) to afford the Compound of Example 6 (23 mg). NMR: δ 8.56-8.51 (m, 1H), 8.34 (t, J=8.1 Hz, 1H), 8.15 (d, J=8.3 Hz, 1H), 7.90 (d, J=8.5 Hz, 1H), 7.84 (dd, J=16.7, 1.9 Hz, 1H), 7.73 (t, J=6.7 Hz, 1H), 7.63-7.55 (m, 2H), 7.46-7.42 (m, 1H), 7.40-7.35 (m, 3H), 7.20 (dt, J=6.9, 4.7 Hz, 3H), 7.18-7.14 (m, 1H), 7.09-7.05 (m, 2H), 4.42 (t, J=8.2 Hz, 1H), 4.31 (ddd, J=9.6, 5.2, 2.3 Hz, 2H), 4.18-4.11 (m, 3H), 4.09-4.00 (m, 6H), 3.90 (s, 1H), 3.67 (s, 2H), 3.33 (s, 1H), 3.16 (dq, J=14.6, 7.4 Hz, 1H), 3.00-2.86 (m, 10H), 2.76 (s, 2H), 2.73-2.68 (m, 1H), 2.62 (q, J=11.8, 10.2 Hz, 1H), 2.55 (s, 3H), 2.18 (t, J=7.3 Hz, 2H), 2.13-1.59 (m, 15H), 1.33 (ddd, J=14.0, 9.9, 4.2 Hz, 1H), 1.29-1.20 (m, 1H), 1.00 (dd, J=15.3, 6.4 Hz, 6H), 0.61 (d, J=6.5 Hz, 3H), 0.54 (d, J=6.4 Hz, 3H). MS: 1677.92 [M+H]⁺.

### Example 7

### Synthesis of the Compound of Example 7

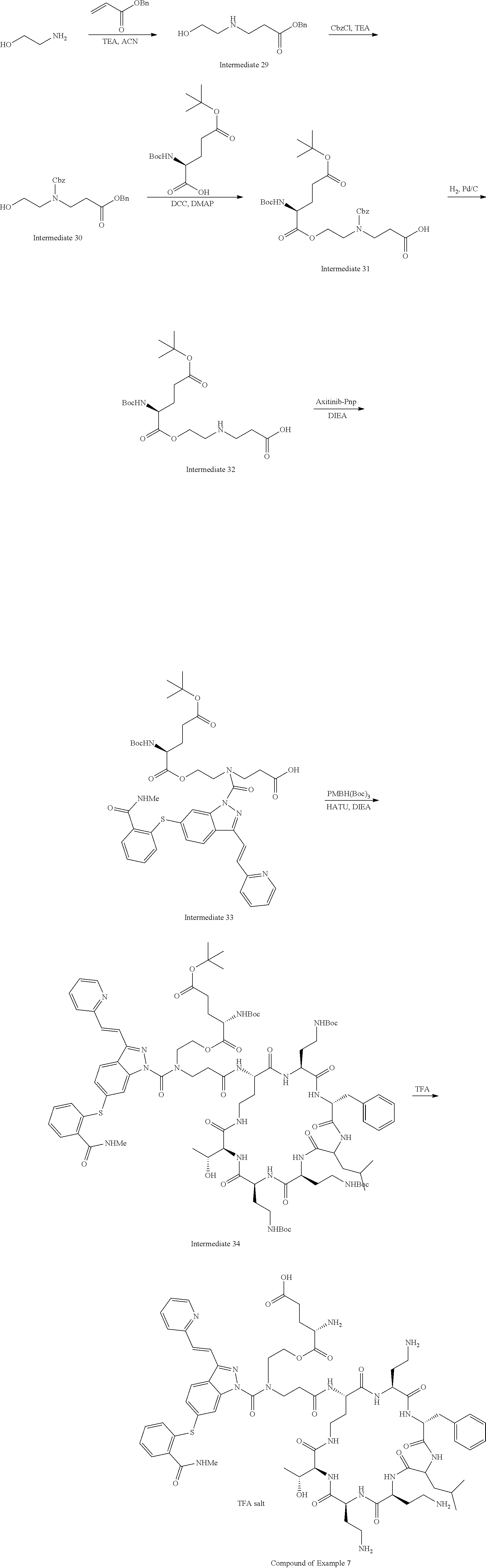

Intermediate 29. The mixture of 2-aminoethan-1-ol (47.8 g, 295 mmol) and TEA (41.0 mL, 295 mmol) in ACN (500 mL) was stirred at 50° C. under N₂ for 4 hrs. The reaction mixture was concentrated under reduced pressure to afford Intermediate 29 (71.3 g crude) as a yellow solid.

Intermediate 30. To a solution of Intermediate 29 (73.1 g, 327 mmol) in ACN/MeOH=2/1 (600 mL) was added benzyl chloroformate (92.2 mL, 655 mmol) and TEA (50.1 mL, 360 mmol). The mixture was stirred at rt for 12 hrs. The reaction mixture was concentrated under reduced pressure. The residue was purified by silica gel chromatography to afford Intermediate 30 (103 g, 88.0% yield) as yellow oil.

Intermediate 31. To a solution of Intermediate 30 (10.0 g, 27.9 mmol) in DCM (100 mL) was added (S)-5-(tert-butoxy)-2-((tert-butoxycarbonyl)amino)-5-oxopentanoic acid (8.49 g, 27.9 mmol), DMAP (4.10 g, 33.5 mmol) and DCC (6.93 g, 33.5 mmol). The mixture was stirred at rt for 12 hrs. The reaction mixture was quenched by addition H₂O (100 mL), extracted with DCM (50 mL*2). The combined organic layers were washed with brine (50 mL) dried over Na₂SO₄, filtered and concentrated under reduced pressure to give a residue. The residue was purified by silica gel chromatography to afford Intermediate 31 (13.0 g, 72.2% yield) as yellow oil. MS: 543.2 [M−99]⁺.

Intermediate 32. To a solution of Intermediate 31 (5.00 g, 7.77 mmol) in 2,2,2-trifluoroethanol (100 mL) was added Pd/C 10% (0.83 g, 7.77 mmol) under N₂ atmosphere. The suspension was degassed and purged with H₂ for 2 times. The mixture was stirred under H₂ (15 psi) at rt for 12 hrs. The reaction mixture was filtered and concentrated under reduced pressure to afford Intermediate 32 (3.00 g, 92% yield) as a white solid. MS: 419.2 [M+H]⁺.

Intermediate 33. To a solution of Intermediate 32 (700 mg, 1.67 mmol) in NMP (7 mL) was added Axi-PNP (590 mg, 1.00 mmol) and DIEA (648 mg, 5.01 mmol). The mixture was stirred at rt for 12 hrs. The crude product was purified by prep-IPLC (TFA) to afford Intermediate 33 (600 mg, 43% yield). MS: 831.5 [M+H]⁺.

Intermediate 34. The reaction of Intermediate 33 (900 mg, 1.08 mmol), PMBH (Boc)₃ (1.15 g, 1.08 mmol), DIEA (419 mg, 3.24 mmol) and HATU (617 mg, 1.625 mmol) in THE (8 mL) was stirred at rt for 12 hrs. The reaction mixture was concentrated under reduced pressure. The residue was purified by prep-HPLC (TFA condition) to afford Intermediate 33 (1.30 g, 64% yield). MS: 888.5 [M+2H]²⁺.

Compound of Example 7. The mixture of Intermediate 34 (1.30 g, 0.693 mmol) in TFA/DCM (3 mL/15 mL) was stirred at rt for 12 hrs. The volatiles were removed and the residue was purified by reversed C18 column to afford the Compound of Example 7 (1126 mg, 0.601 mmol, 86.7% purity). NMR (400 MHz, Deuterium Oxide): δ 8.60-8.40 (m, 2H), 8.22 (d, J=7.6 Hz, 1H), 7.92-7.78 (m, 3H), 7.66 (br s, 1H), 7.57 (d, J=16.3 Hz, 1H), 7.46-7.34 (m, 4H), 7.20 (d, J=7.3 Hz, 3H), 7.13 (br s, 1H), 7.08 (d, J=7.1 Hz, 2H), 4.45-4.21 (m, 6H), 4.13 (d, J=5.1 Hz, 3H), 4.05 (br s, 2H), 3.92 (br s, 6H), 3.08 (dd, J=13.8, 7.3 Hz, 2H), 3.03-2.88 (m, 5H), 2.87-2.69 (m, 5H), 2.60 (s, 6H), 2.23-1.96 (m, 7H), 1.94-1.59 (m, 7H), 1.32 (br s, 2H), 0.95 (br s, 3H), 0.76 (br s, 1H), 0.63 (d, J=5.9 Hz, 3H), 0.55 (d, J=5.5 Hz, 3H). MS: 710.2 [M+2H]²⁺.

### Example 8

### Synthesis of the Compound of Example 8

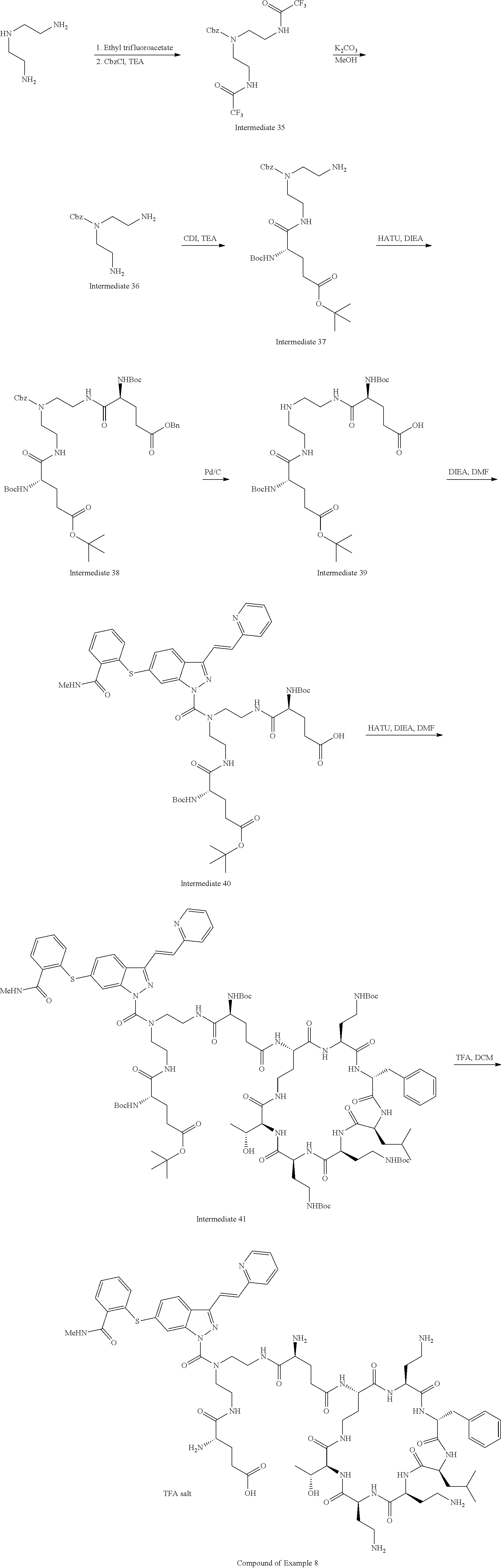

Intermediate 35. N1-(2-aminoethyl)ethane-1,2-diamine (3.35 g, 32 mmol) was dissolved in DCM (30 mL) and cooled to 0° C. Ethyl trifluoroacetate (9.69 g, 68 mmol) in DCM (10 mL) was slowly added. After 1 h, TEA (3.87 g, 38.4 mmol) was added and then CbzCl (6.0 g, 35.2 mmol) in DCM (10 mL) was added at 0° C. The mixture was stirred at rt for 16 hrs. Diluted with water, and the DCM solution was separated and concentrated, the residue was purified by silica gel chromatography to afford Intermediate 35 (2.9 g) as white solid, yield 21%. MS: 430.0 [M+H]⁺.

Intermediate 36. To a solution of Intermediate 35 (200 mg, 0.47 mmol) in MeOH (5 mL)/H₂O (1 ml) was added K₂CO₃ (129 mg, 0.93 mmol). The mixture was stirred at rt for overnight. The solvent was removed and purified by pre-HPLC (TFA) to afford Intermediate 36 (90 mg, 81% yield) as colorless oil. MS: 238.2 [M+H]⁺.

Intermediate 37. To a solution of (S)-5-(tert-butoxy)-2-((tert-butoxycarbonyl)amino)-5-oxopentanoic acid (115 mg, 0.38 mmol) in DMF (1 mL) was added CDI (58 mg, 0.36 mmol) at 0° C., followed by addition of Intermediate 36 (90 mg, 0.38 mmol). The resulting mixture was stirred at room temperature for 4 hrs. The mixture was diluted with 10 mL of EtOAc and washed with 10 mL of water, the organic layer was separated and concentrated. The residue was purified by pre-HPLC (CH₃CN (0.1% TFA)/H₂O, 0-60%) to afford Intermediate 37 (93 mg, 47% yield) as colorless oil. MS: 523.3 [M+H]⁺.

Intermediate 38. To a solution of (S)-5-(benzyloxy)-2-((tert-butoxycarbonyl)amino)-5-oxopentanoic acid (72 mg, 0.21 mmol) in DMF (2 mL) cooled at 0° C. were added HATU (88 mg, 0.23 mmol) and DIEA (68.9 mg, 0.53 mmol), the mixture was stirred at 0° C. for 20 mins. Then Intermediate 37 (93 mg, 0.18 mmol) was added. The resulting mixture was stirred at room temperature for overnight. The mixture was diluted with 10 mL of EtOAc and washed with 10 mL of water, the organic layer was separated and concentrated. The residue was purified by pre-HPLC (CH₃CN (0.1% TFA)/H₂O, 0-90%) to afford Intermediate 38 (110 mg, 73% yield) as colorless oil. MS: 842.2 [M+H]⁺.

Intermediate 39. To a solution of Intermediate 38 (110 mg, 0.13 mmol) in EtOAc (20 ml) was added 10% Pd/C (50% content, 10 mg), the mixture was then stirred at rt overnight, under H₂ atmosphere. The mixture was filtered through Celite and concentrated to afford Intermediate 39 (94 mg crude) as colorless oil. MS: 618.4 [M+H]⁺.

Intermediate 40. DIEA (53 mg, 0.41 mmol) was added to a solution of Intermediate 39 (84 mg, 0.13 mmol) and 4-nitrophenyl (E)-6-((2-(methylcarbamoyl)phenyl)thio)-3-(2-(pyridin-2-yl)vinyl)-1H-indazole-1-carboxylate (75 mg, 0.136 mmol) dissolved in DMF (2 mL). The mixture was stirred at rt for overnight. The mixture was diluted with 10 mL of EtOAc and washed with 10 mL of water, the organic layer was separated and concentrated. The residue was purified by pre-HPLC (CH₃CN (0.1% TFA)/H₂O, 0-80%) to afford Intermediate 40 (24 mg, 17% yield) as white solid. MS: 1030.3 [M+H]⁺.

Intermediate 41. To a solution of Intermediate 40 (21 mg, 0.02 mmol) in DMF (2 mL) was added HATU (9.8 mg, 0.024 mmol), followed by DIEA (19.2 mg, 0.14 mmol) and PMBH-Boc₃ (25 mg, 0.022 mmol). The mixture was stirred at rt for 3 hrs. The mixture was diluted with 10 mL of EtOAc and washed with 10 mL of water, the organic layer was separated and concentrated. The residue was purified by pre-HPLC (CH₃CN (0.1% TFA)/H₂O, 0-80%) to afford Intermediate 41 (17 mg, 41% yield) as white solid. MS: 1037.6 [M+2H]²⁺.

Compound of Example 8. To a solution of Intermediate 41 (17 mg, 0.008 mmol) in DCM (2 mL) was added TFA (0.5 mL). The resulting mixture was stirred at room temperature for 16 h. The mixture was concentrated and purified by reversed C18 column (0.05% TFA in CH₃CN: H₂O=5˜30%) to afford Compound of Example 8 (6.2 mg, 32% yield) as white solid. NMR (400 MHz, Deuterium Oxide): δ 8.66-8.55 (m, 1H), 8.38-8.36 (m, 1H), 8.19-8.14 (m, 1H), 8.05-7.88 (m, 3H), 7.80-7.68 (m, 3H), 7.56-7.47 (m, 4H), 7.32-7.29 (m, 3H), 7.21-7.16 (m, 2H), 4.48-4.41 (m, 6H), 4.20-4.11 (m, 4H), 3.85-3.77 (m, 8H), 3.08-2.97 (m, 10H), 2.77-2.60 (m, 6H), 2.25-2.12 (m, 14H), 1.98-1.36 (m, 16H), 1.09-1.08 (m, 3H), 0.72-0.70 (m, 3H), 0.65-0.63 (m, 3H). MS: 1517.7 [M+H]⁺.

### Example 9

### Synthesis of the compound of Example 9

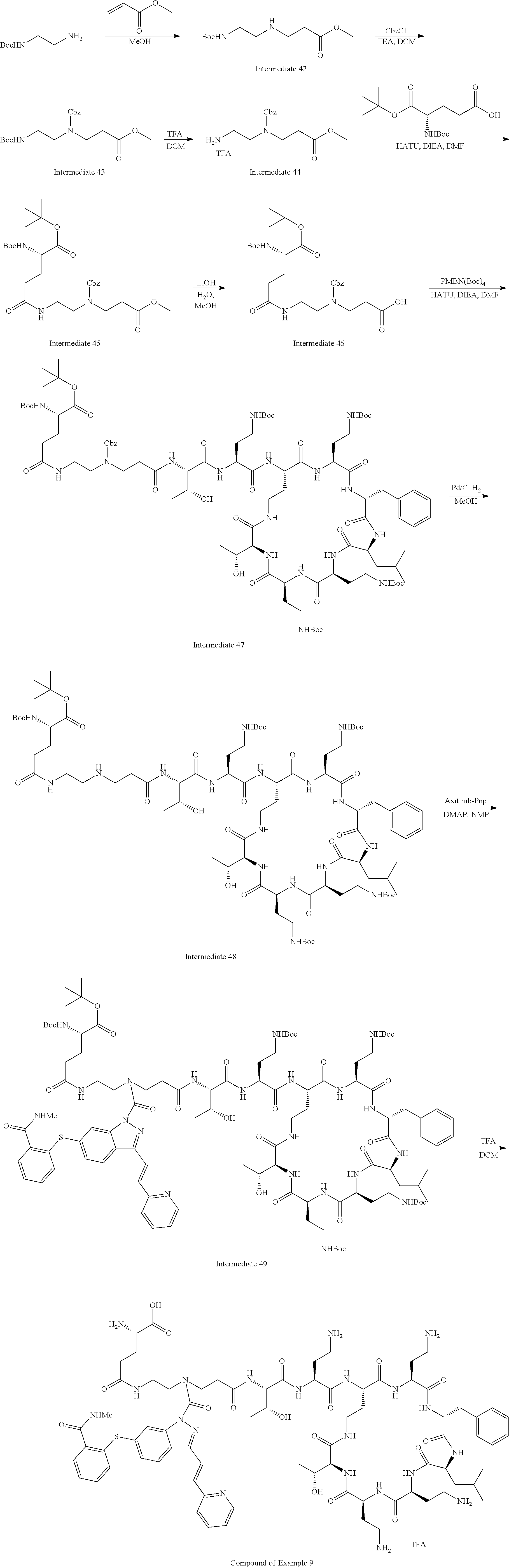

Intermediate 42. The mixture of N-Boc-ethylenediamine (2.4 g, 15 mmol) and methyl acrylate (0.86 g, 10 mmol) in MeOH (4 mL) was stirred at 0-4° C. under Ar for 4 h. Volatiles were evaporated under vacuum, and the residue was purified by silica gel chromatography (gradient 0.1% TEA EA/0.1% TEA PE 0˜100%) to give Intermediate 42 (1.3 g). MS: 247.2 [M+H]⁺.

Intermediate 43. The mixture of Intermediate 42 (1.3 g, 5.3 mmol), CbzCl (1 g, 5.8 mmol) and TEA (1.5 mL, 10.6 mmol) in DCM (10 mL) was stirred at 5° C. under Ar for 4 h. The mixture was removed volatiles, and extracted with EA (50 mL), washed with H₂O (5 mL×2) and brine (5 mL). The EA layer was dried (Na₂SO₄), filtered, and evaporated. The crude material was purified by silica gel chromatography (EA/PE 0˜60%) to give Intermediate 43 (2.0 g). MS: 381.2 [M+H]⁺.

Intermediate 44. Intermediate 43 (2.0 g, 5.26 mmol) in TFA/DCM (2 mL/15 mL) was stirred at r.t. for 1.5 h. Volatiles were removed under vacuum to give Intermediate 44 (2.1 g), used directly at the next step. MS: 281.2 [M+H]⁺.

Intermediate 45. The mixture of Intermediate 44 (2.1 g, 5.26 mmol), (S)-5-(tert-butoxy)-4-((tert-butoxycarbonyl)amino)-5-oxopentanoic acid (1.33 g, 4.38 mmol), HATU (2.33 g, 6.14 mmol) and DIEA (1.55 mL, 8.77 mmol) in DMF (12 mL) was stirred at 40° C. for 4 h. The mixture was cooled to r.t., extracted with EA (100 mL), washed with H₂O (15 mL×2) and brine (15 mL). The EA layer was dried (Na₂SO₄) and solvent evaporated under vacuum. The product was purified by silica gel chromatography (EA/PE 0˜80%) to give Intermediate 45 (2.6 g). MS: 566.1 [M+H]⁺.

Intermediate 46. Intermediate 45 (0.4 g, 0.7 mmol), was added to a solution of LiOH—H₂O (45 mg, 1.1 mmol) in MeOH/H₂O (2 mL/1 mL). The reaction was stirred at r.t. for 6.5 h, acidified, and then extracted with EA (50 mL), washed with H₂O (5 mL×2) and brine (5 mL). The EA layer was dried and evaporated. The product was purified by silica gel chromatography (EA/PE 0˜90%) to give Intermediate 46 (0.22 g). MS: 552.0 [M+H]⁺.

Intermediate 47. The mixture of Intermediate 46 (0.22 g, 0.4 mmol), PMBN(Boc)₄; 0.544 g, 0.4 mmol), HATU (0.182 g, 0.48 mmol) and DIEA (0.172 mL, 0.8 mmol) in DMF (6 mL) was stirred at 40° C. for 4 h. The reaction mixture was cooled to r.t., then quenched with water and extracted with EA (100 mL). Organic layer was washed with H₂O (15 mL×2), brine (15 mL), dried (Na₂SO₄) and evaporated under vacuum. The product was purified by C18 column chromatography ACN/H₂O 0˜100%) to give Intermediate 47 (0.49 g). MS: 1896.5 [M+H]⁺.

Intermediate 48. The mixture of Intermediate 47 (0.49 g, 0.28 mmol) and 10% Pd/C (0.1 g) in MeOH (10 mL) was stirred at r.t. under H₂ for 4.5 h, filtered, evaporated, and dried to afford Intermediate 48 (0.385 g). MS: 1762.8 [M+H]⁺.

Intermediate 49. The mixture of Intermediate 48 (0.35 g, 0.2 mmol), Axitinib-Pnp (0.175 g, 0.35 mmol) and DMAP (0.049 g, 0.4 mmol) in NMP (5 mL) was stirred at 50° C. under Ar for 5 h. The mixture was cooled to r.t., extracted with EA (50 mL), washed with H₂O (5 mL×2) and brine (5 mL). The EA layer was dried (Na₂SO₄) and evaporated. The product was purified by C18 column chromatography (ACN/H₂O 0˜90%) to give Intermediate 49 (0.208 g).

Compound of Example 9. Intermediate 49 (0.27 g, 0.124 mmol) in TFA (1 mL) and DCM (6 mL) was stirred at r.t. for about 1 h. Volatiles were removed under vacuum and the residue was purified by C18 column chromatography (ACN/H₂O 0˜40%) to afford the compound of Example 9 (125 mg) as a TFA salt. MS: 1618.9 [M+H]⁺. NMR: 8.44 (d, J 8.2 Hz, 1H), 8.25 (d, J 8.0 Hz, 1H), 7.95 (s, 2H), 7.82 (t, J 6.7 Hz, 1H), 7.66 (s, 2H), 7.49 (s, 1H), 7.47-7.40 (m, 3H), 7.31-7.20 (m, 5H), 7.14 (d, J 7.6 Hz, 2H), 4.48 (t, J 8.1 Hz, 1H), 4.36 (d, J 7.0 Hz, 2H), 4.23-4.16 (m, 3H), 4.14-4.00 (m, 7H), 3.87 (s, 2H), 3.75 (s, 3H), 3.37 (s, 1H), 3.20 (d, J 13.0 Hz, 1H), 3.00 (d, J 43.2 Hz, 12H), 2.72 (d, J 53.7 Hz, 4H), 2.62 (s, 4H), 2.24 (t, J 7.6 Hz, 2H), 2.13 (s, 7H), 2.02-1.65 (m, 10H), 1.08-1.04 (m, 3H), 0.97 (s, 3H), 0.73 (s, 1H), 0.67 (d, J 6.5 Hz, 3H), 0.60 (d, J 6.4 Hz, 3H). Optionally, the TFA salt of the compound of Example 9 is converted into an HCl salt, a H₂SO₄ salt, a citric acid salt, a lactic acid salt, a mandelic acid salt, or another pharmaceutically acceptable salt. As generally applicable, this can be accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Utility and Testing

The compounds provided herein exhibit a pronounced therapeutic effect (efficacy) against a variety of kidney cancers, including RCCs and mRCCs. Therefore, these agents are useful for a targeted therapy of kidney-associated cancers.

The novel compounds provided herein comprise an anticancer-bioactive molecule generally conjugated with a carrier peptidic fragment(s) (for example, a polymyxin cyclopeptide derivative). The latter serves as a carrier for a delivery of such a compound into kidneys, due to the unique ability of said peptidic fragment to bind kidney tissues.

First, some of the compounds provided herein exhibit innate activity against cancer cells (or anticancer cytotoxicity) as intact molecular structures. Such intrinsic activity is inherent in said molecules, and this activity does not rely on a metabolic release of an anticancer agent conjugated within the structures (in other words, covalently connected to a peptidic fragment that serves as a carrier for delivery of a compound into kidney, due to the propensity of said peptidic part to bind kidney tissues).

Secondly, certain compounds provided herein exhibit modest or no innate anticancer cytotoxicity as intact molecules. Upon administration, these accumulate in kidneys and then are metabolized in the organ affected by renal cancers. This metabolism leads to a release of an anticancer drug (or a cytotoxic agent), which is incorporated within the administered compound to result in anticancer therapeutic effect at the site of a cancer (manifested, for example, in a cancer tumor size reduction, or a stopped tumor growth). Importantly, this metabolic degradation occurs selectively; the active entity is released in the requisite drug form (without metabolic alteration that could reduce the required anticancer activity).

Thirdly, certain compounds provided herein combine the innate anticancer activity as intact molecules with an anticancer effect resulting from a metabolic release of active anticancer drug (a cytotoxic agent) that was incorporated within the administered molecule. This combined effect can be either additive or synergistic in nature. This modality comprises a dual-action mode: the innate activity of the intact conjugate compound, beneficially coupled with the activity of a metabolically-released drug (bioactive) incorporated within such administered conjugate.

Important, all the therapeutic modalities described above are exhibited upon a selective or targeted renal delivery of the compounds provided herein. In other words, the compounds administered to a mammal in the need of a therapy rapidly accumulate in the kidneys affected by a renal cancer.

Preferential accumulation of compounds provided herein in kidneys (or near the site of a kidney cancer) may be evaluated by pharmacokinetic (PK) tests, such as in a standard rat PK test. PK data are generally used to establish the key parameters predictive of the therapy outcome, such as drug concentration (C) at given time points, drug concentration at the target tissue(s) (C_{Target}), area under the curve (AUC) for a plot monitoring the change in the systemic drug concentration over time, and other parameters. Thus, drug concentration in organ (or body compartment) affected by cancer is important for effective action of anticancer agents (as described, for example, by Zhang et al. in Drug Metabolism and Disposition. 2019, vol. 47, p. 1122).

Representative compounds provided herein have been tested in a rodent PK model of intravenous administration performed analogously to methods described in the monograph Current Protocols in Pharmacology, 2005, 7.1.1-7.1.26, John Wiley & Sons, Inc.

In PK studies, the level (concentration) of a therapeutic drug is determined in key body compartments, such as blood and select organ tissues, over a given time course. The levels of an active compound in the organ affected by a disease is of particular importance, since such a compound is intended to target the disease therein. For therapy of renal cancers, the targeted organ is kidney.

Anticancer efficacy (in vivo activity) depends on and is directly tracked to certain required levels of an anticancer drug in the mammal in the need of therapy (as reviewed, for example, by Fogli et al. in Cancer Treatment Reviews. 2020, vol. 84, 101966; by Hu-Lowe et al. in Clin. Cancer Research. 2008, vol. 14, p. 7272; and by Zhang et al. in Drug Metabolism and Disposition. 2019, vol. 47, p. 1122). This concentration-therapy relationship is rooted in a mode of action of anticancer drugs, generally based on a concentration-dependent inhibition of the cancer cell growth (for example, cancer cells manifested as a tumor).

Thus, incomplete or no inhibition of cancer cells (or tumor growth) could result, if a drug concentration is too low to achieve said inhibition. This generally leads to ineffective therapy, often aggravated with an increased risk of developing a cancer drug-resistance, with latter rendering the disease non-responsive to a drug (as reviewed, for example, by Komarova et al. in PNAS. 2005, vol 102, p. 9714). Conversely, if a drug concentration at a targeted organ is higher, then an enhanced anticancer efficacy is generally observed, and with a minimized risk of developing cancer drug-resistance.

For example, an efficacy effect of the renal cancer drug axitinib is reliably forecasted from its concentration in the blood, with the blood circulation encompassing kidneys affected by the disease (as reviewed by Hu-Lowe et al. in Clin. Cancer Research. 2008, vol. 14, p. 7272). Specifically, the total axitinib blood concentration of about 40 ng/mL was reported as the marker (predictor) of high therapeutic efficacy against renal carcinomas. Therefore, if the drug concentration falls below 40 ng/mL, a reduced therapeutic efficacy is anticipated (Hu-Lowe et al. in Clin. Cancer Research. 2008, vol. 14, p. 7272). Upon axitinib administration, kidney drug concentrations generally do not exceed the levels of this drug in blood (and often are lower for kidney, relative to that in blood; see, for example, Table 2 below).

Therefore, axitinib kidney concentrations of 40 ng/mL or about 39 ng/g (per kidney tissue density of 1.03 g/mL) is generally predictive of an effective inhibition of renal cancers, needed for a successful therapy of the mammal in the need thereof.

Illustrative PK data for the compound of Example 1 is summarized in the Table 1 below. As clear from the data for the mouse PK model, this compound exhibits an effective ability to target kidneys, as confirmed by high levels of preferential accumulation in kidney tissues, the targeted organ.

TABLE 1
Targeted delivery of an illustrative compound
of Example 1 to kidneys, as compared to blood.

| | Blood Plasma | Kidney Tissue | Kidney-to-Blood |
|---|---|---|---|
| EXAMPLES | Conc.,^{a} ng/mL | Conc.,^{a,b} ng/mL | Conc. Ratio |
| Example 1 | 9.0 | 563.1 | 63 |

^{a}At T = 3 h after administration by injection into a mouse, 3 mg/kg.
^{b}Corrected for kidney tissue density of about 1.03 mg/mL.

Additional PK data in rodents for compounds of Example 1, 2, and 7 and the reference compound of Example 9 provided herein are illustrated in the Table 2 below. The data indicated a targeted delivery of anticancer drug axitinib, preferentially released from these compounds into kidneys.

TABLE 2
Targeted delivery of renal cancer drug axitinib using the compound
of Example 1: the drug concentration in kidneys compared to
blood, at test time (T, h) points post-administration.

| | Blood Plasma | Kidney Tissue |
|---|---|---|
| EXAMPLES | Conc.,^{a} ng/mL | Conc.,^{a.c} ng/mL |
| Axitinib^{b,c} | T = 3 h: 397.3 | T = 3 h: 301.0 |
| | T = 6 h: 75.6 | T = 6 h: 96.6 |
| | T = 12 h: 11.4 | T = 12 h: 16.2 |
| | T = 24 h: BLQ^{d} | T = 24 h: BLQ |
| Example 1 | T = 3 h: 9.7 | T = 3 h: 1955.3 |
| | T = 6 h: 4.0 | T = 6 h: 1448.5 |
| | T = 12 h: BLQ | T = 12 h: 1437.9 |
| | T = 24 h: BLQ | T = 24 h: 707.8 |
| Example 2 | T = 3 h: 3.3 | T = 3 h: 5140 |
| | T = 6 h: BLQ | T = 6 h: 4630 |
| Example 7 | T = 3 h: 14.5 | T = 3 h: 4280 |
| | T = 6 h: BLQ | T = 6 h: 4860 |
| Example 9^{e} | T = 3 h: 14.9 | T = 3 h: 371.6 |
| | T = 6 h: BLQ | T = 6 h: 28.8 |

^{a}Agent administered though an injection into a mouse, as a single dose of 3 mg/kg.
^{b}Agent administered through an oral gavage, as a single dose of 30 mg/kg.
^{c}Corrected for kidney tissue density of about 1.03 mg/mL.
^{d}BLQ: observed value below the level of quantitation.
^{d}ND: not determined.
^{e}Reference compound.

As clear from the mouse PK data, the compounds of Example 1, 2, and 7 and reference compound Example 9 dosed at 3 mg/kg exhibit an effective ability to target kidneys (>40 ng/g). In fact, the levels of active drug in the kidneys is far superior to that of axitinib dosed at 30 mg/kg. This suggests that compounds described herein would be at least as efficacious for the anticancer therapy as the active agent axitinib.

It is surprising to see by Table 2 that high levels of axitinib were detected in kidney tissues released from the compound of Example 1, for example, >6-fold from the compound of Example 1 vs from axitinib itself at 3 h (1955.3/301.0=6.50), and about 15-fold at 6 h. High levels of axitinib from the reference compound of Example 9 were also detected at a level that is much higher from axitinib by itself, but in a much less significant amount vs the compound of Example 1. More strikingly surprising, compounds of Examples 2 and 7 detected significantly higher levels of axitinib in the kidney than that from compound of Example 1. These data indicate a targeted delivery of the anticancer drug axitinib, preferentially released into the kidneys from the compounds described herein. Specifically, administration of the compounds provided herein allows for beneficially higher levels of the therapeutic agent in kidneys, as opposed to administration of axitinib in its standard free drug form.

In contrast, the administration of axitinib itself resulted in a high level in blood, which is the key reason for off-target adverse effects of axitinib and other drugs in therapy of renal cancers (as reviewed, for example, by Fogli et al. in Cancer Treatment Reviews. 2020, vol. 84, 101966).

As noted already, the axitinib efficacy is predicted from the total drug concentration in plasma of at least 40 ng/mL (as reviewed by Hu-Lowe et al. in Clin. Cancer Research. 2008, vol. 14, p. 7272). According to experimental data in Table 2, the compound of Example 1 effectively and selectively delivered axitinib into kidneys, with this drug released from said compound at levels well over 40 ng/mL. The data are indicative of an effective therapy of kidney cancers with the compounds provided herein, such as the compound of Example 1.

Also apparent from above data, the exemplary compound provided herein selectively delivers the renal cancer drug into kidneys (the disease site), and at levels markedly exceeding the kidney levels achieved using axitinib itself. For example, only trace (BLQ, below levels of quantitation) axitinib concentration was detected in rodent kidneys at 24 h post-administration of the drug (Table 2). In contrast, above-therapeutic>40 ng/mL levels of the drug was delivered into kidneys by administration of the compound of Example 1 throughout representative PK time points with surprisingly high drug levels of about 708 ng/mL at T=24 h.

Remarkably and surprisingly, the axitinib kidney levels achieved though administration of the compound of Example 1 at a modest dose of just 3 mg/kg was 707.8 mg/mL at the time point of 24 h. In contrast, the kidney levels of oral axitinib observed even at very high dose of 30 mg/kg (exceeding standard therapeutic dosing of about 0.17 mg/kg in human use) were too low to be quantified (BLQ), as determined 24 h after administration of the drug in its standard free (unconjugated) form. These data conclusively indicate a greatly increased efficacy potential for the compounds provided herein, as compared to the standard axitinib therapy of renal cancers.

The markedly improved drug levels at the site of kidney cancers (achieved with for exemplary compounds) are indicative of the improved efficacy (in vivo activity) of said compounds, as compared to a standard administration of axitinib. Importantly, this could be achieved with a reduced amount of axitinib administered in the form of a conjugate compound provided here (such as the compound of Example 1).

In addition, these favorable and surprising PK data indicate an option of less frequent administration and/or reduced dosing of compounds provided herein, as compared to axitinib. For example, a standard twice-daily administration of axitinib could be substituted by a once-daily or once-weekly administration of a compound provided herein. This provides a significant convenience to a patient under therapy for a kidney cancer.

Separately, the selective (or targeted) kidney delivery of compounds provided herein comprises a significant safety benefit. A standard therapy with cytotoxic cancer drugs is generally accompanied by significant adverse effects. For example, axitinib therapy suffers from multiple adverse effects as noted as Warnings in the prescribing information for the drug (marketed as Inlyta). In particular, hypertensive adverse effects were reported (for example, by Fogli et al. in Cancer Treatment Reviews. 2020, vol. 84, 101966), with an incidence of 40-64%, including a hypertensive crisis. Principally, these adverse effects results from high levels of axitinib circulating in the blood, and thus distributed into vital organs not affected by renal cancers.

Indeed, the PK data of Table 1 indicate similar levels of the drug in blood as compared to kidneys, after a standard administration of free axitinib to a mouse. As a result, the drug may exert adverse cytotoxic effects—often referred to as “off-target activity”—in healthy organs not intended for such cancer therapy, just as observed in the human use of axitinib (reviewed, for example, by Fogli et al. in Cancer Treatment Reviews. 2020, vol. 84, 101966).

In contrast, the administration of exemplary compounds provided herein results in greatly minimized amounts of a (released) active drug in blood, and beneficially concomitant with a selective (targeted) delivery of the drug into kidneys. Therefore, a greatly reduced off-target activity (toxicity) is anticipated for therapy with a compound provided herein, as compared to a standard drug with anticancer agent, such as axitinib.

In vitro activity of compounds provided herein may be assessed by standard testing procedures using various cancer cell lines (alongside normal cell comparators), such as described, for example, in ACS Pharmacol. Transl. Sci. 2019, vol. 2, p. 18; J. Med. Chem. 2018, vol. 61, p. 5304); and methods in references cited therein.

It is important to distinguish between in vitro activity (potency) from in vivo activity (efficacy). In vitro tests allow for the tested compound to interact with cancer cells directly, typically, by introducing the test compound into cancer cells suspended in a solution of nutrients that allow for the cell growth.

In contrast to in vitro tests, in vivo evaluation entails administration of a compound to a mammal (such as a rodent), for example, intravenously. The compound is then circulated in the blood and is distributed in organs and tissues. This distribution can occur with varying efficiency for different organs, and may result in drug accumulation in some organs, concomitant with low levels of same drug in other organs. Importantly, in this process, the compound is exposed to numerous proteins and enzymes (such as esterases and peptidases) that may metabolize (degrade) the compound during in vivo test.

For example, some compounds provided herein are metabolized in vivo to release the active drug molecule conjugated within said compounds using metabolically cleavable linkers. As a result, such compound that has no or modest innate or intrinsic activity (potency) when tested in vitro may exhibit a high in vivo activity (efficacy) when tested in vivo.

The in vitro anticancer activity of certain compounds provided herein is surprising. While these incorporate an anticancer drug structure within the new chemical composition, the anticancer moiety connected therein is dramatically altered, as compared to the (highly optimized) original anticancer drug structure when not conjugated, e.g. axitinib.

The innate anticancer activity of novel axitinib-polypeptide conjugates provided herein is especially surprising in face of the highly restrictive structure-activity relationships (SAR) for its close analogs. As reported, for example, in Molecules. 2018, 23, 747, multiple isosteric designs closely mimicking axitinib structure have failed to replicate the activity of this drug, and exhibited many-fold reduced inhibition of the targeted cancer enzymes (such as VEGFR-2 kinase), as compared to axitinib itself. Specifically, precluding the NH hydrogen-binding by way of replacing these groups with NMe was found to be detrimental for inhibition of the cancer enzyme, such as VEGFR-2 enzyme. Thus, the in vitro anticancer activity indicative of an intrinsic ability of compounds provided herein to inhibit cancer cells is not at all anticipated.

As stated above, certain compounds described herein possess a reduced in vitro cytotoxicity against cancer cells, resulting in a beneficially reduced off-target activity(ies) against healthy organs (with said off-target effect being responsible for most adverse effects in the standard cancer therapy). While exhibiting a reduced cytotoxicity of the intact conjugate molecule in vitro, such compounds exert therapeutic anticancer effect in vivo after being metabolized with release of the active drug (conjugated within the administered molecule) at the target organ of a cancer. Thus, the administration of such a compound (which may be inactive in vitro) to a mammal in the need of cancer therapy, results in a selective targeted delivery therapy, with a pronounced anticancer activity observed in vivo.

Surprisingly, while the compound of Example 1 incorporates a polymyxin structure, it also exhibits a minimized antibacterial activity (measured in vitro as MIC, minimum inhibitory concentration) typical for the polymyxin antibiotics such as polymyxin B and colistin.

In vivo activity of compounds provided herein may be assessed by testing procedures such described, for example, in J. Vis. Exp. 2014, (86), e51485; Experimental & Molecular Medicine. 2018, vol. 50, p. 30; and methods in references cited therein.

Surprisingly, certain compounds provided herein, when tested in rodent kidney cancer model with intravenous (IV) administration at a dosing (molar amount) equal to a standard therapeutic dosing (molar amount) of axitinib, brivanib, pazopanib or sunitinib, exhibit 2-fold or higher efficacy, as compared to the standard therapeutic dosing of, for example, axitinib, brivanib, pazopanib, sunitinib, or tivozanib, with said therapeutic effect determined as a slowed, stopped, or reversed progression of cancer (for example, determined per changes in a cancer tumor size, and/or by using biochemical biomarkers for cancer monitoring, or similar methods such described in such described, for example, in J. Vis. Exp. 2014, (86), e51485; Experimental & Molecular Medicine. 2018, vol. 50, p. 30).

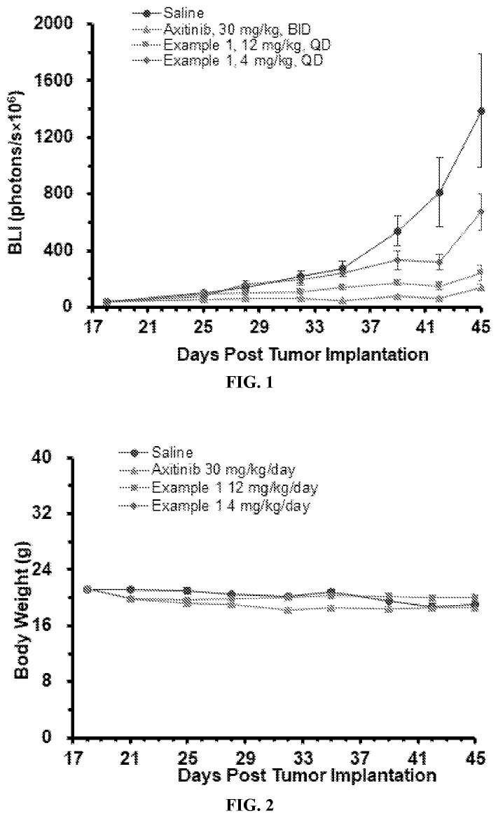

An illustrative efficacy study for the compounds described herein was investigated in an orthotopic mouse model. In this model, a luciferase-expressing A498 renal cancer cell line (A498-luc) was used. The cancer cells were implanted into a kidney capsule and the tumor growth and effect were monitored by the image size using IVIS Lumina III (Perkin Elmer) and mouse body weight changes. Specifically, mice were anesthetized by SC injection of Zolet™ m 50 (Virbac S. A.) after 24 h fasting. The skin of anesthetized mice was sterilized with iodine followed by alcohol. 4×10⁶ luciferase-expressing A498-luc cells were implanted in the left kidney by insulin syringe. The skin incisions were closed with autosuture clip. The tumor growths were monitored by image analysis. Mice were randomized into four groups (10 mice/group), two doses compound of Example 1 at 4 and 12 mg/kg, control group with saline, dosed intraperitoneally (ip), once a day (QD) and control group with axitinib, dosed orally (PO), twice a day (BID). Mice were injected with 15 mg/mL (at 5 mL/g body weight) of D-luciferin (Pharmaron) and anesthetized with 1-2% isofluorane inhalation. At 10 mins after the luciferin injection, the mice were imaged (once a week). Living image software (Perkin Elmer) is used to compute regions of interest (ROI) and integrate the total bioluminescence signal in each ROI. Bioluminescent signals (photos/s) from ROI were quantified and used as an indicator of tumor growth and antitumor activity. Body weight of all mice were measured twice a week throughout the study. Exemplary efficacy results for the compound of Example 1 are summarized in FIGS. 1 and 2.

As noted from FIG. 1, the compound of Example 1 achieved high efficacy (82.5% and 52%) of tumor growth inhibition (TGI) at the doses of 12 and 4 mg/kg (2.0 and 0.67 mg/kg of axitinib), QD, vs axitinib 89.7% at the dose of 30 mg/kg/day, BID. These data revealed that at about one 15^{th} of the dose of axitinib, axitinib released from the compound of Example 1 can achieve>80% TGI. Importantly, the compound of Example 1 was also dosed less frequently (QD) than axitinib (BID).

While the compound of Example 1 demonstrated high efficacy at 12 mg/kg/day (equal to 2.0 mg/kg axitinib free drug) with intraperitoneal injection, it also showed a better safety profile—minimized loss in weight vs axitinib at 30 mg/kg/day as indicated by data in FIG. 2. The loss in body weight is an established marker of toxicity for many cancer drugs, commonly associated with other adverse effects. The data herein indicate that the compounds described herein possess significantly improved safety margin (therapeutic index) while effectively inhibiting the tumor growth in the cancer therapy.

Surprisingly, while possessing a high anticancer efficacy in mammals, the compounds provided herein exhibit limited toxicity to non-cancerous kidney cells. This is demonstrated in the following assay. The cell toxicity of the compound of Example 1 was assessed in an in vitro assay using the HK-2 cell line, an immortalized proximal tubules cell line derived from a normal human kidney. The assay is analogously to that described, for example, by Keirstead et al. in Toxicol. Sci. 2014, vol. 137, pp. 278-291. The results were summarized in Table 3.

TABLE 3
Cell toxicity of compound of Example 1
vs axitinb by a HK-2 cytotoxicity assay

| | In vitro human HK-2 |
|---|---|
| EXAMPLES | assay IC₅₀, μg/mL |
| Axitinib | 36.1 |
| Example 1 | >100 |

As indicated by Table 3, the compound of Example 1 (axitinib in a conjugated form) is at least 2-3-fold less active, indicating that it would be safer than axitinib (the free drug form).

The illustrative compound of Example 1 was also well-tolerated in a 14-days repeat-dose mouse tolerability test, when administered to the test animals at a dose of at least 18 mg/kg/day.

An improved safety profile of the compounds described herein is further established in biomarker assays predictive of nephotoxicity. Several such assays (including NGAL assay) have been described, for example, by Keirstead et al. in Toxicol. Sci. 2014, vol. 137, pp. 278-291.

Surprisingly, certain compounds provided herein, when administered to a mammal at a dosing (expressed in molar amount) equal to a standard therapeutic dosing (molar amount) of axitinib, brivanib, pazopanib, sunitinib, tivozanib exhibit at least 2-fold reduced rate (frequency or incidence) of adverse effects and/or off-target toxicity manifestation (such as myelosuppression or bone marrow toxicity), as compared to the standard therapeutic dosing of axitinib, brivanib, pazopanib, or sunitinib (for example, as determined by the platelet and/or other blood cells count for myelosuppression or bone marrow toxicity).

Thus, certain compounds described herein exhibit high anticancer efficacy, but do not suffer from excessive off-target toxicity affecting organs not affected by kidney cancers, and exhibit little or no nephotoxicity against normal kidney cells.

Therefore, unprecedented types of novel compounds and compositions provided herein potentially provide a long sought-after, safer and effective targeted therapy for kidney cancers, including metastatic renal cancer carcinomas.

### Administration and Pharmaceutical Formulations

In general, the compounds provided herein can be administered in a therapeutically effective amount by any of the accepted modes of administration for agents that serve similar utilities. By way of example, compounds provided herein may be administered orally, parenterally, transdermally, topically, rectally, or intranasally, or by way of intra-tumoral administration directly into a cancerous tumor. The actual amount of a compound provided herein, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

The data obtained from the cell culture assays and animal studies can be used in formulating a range of dosage for use in humans. The dosage of such compounds lies preferably within a range of circulating concentrations that include therapeutic efficacy with little or no toxicity. The dosage may vary within this range depending upon the dosage form employed and the route of administration utilized. For any compound used in the method provided herein, the therapeutically effective dose can be estimated initially from animal models. A dose may be formulated in animal models to achieve a circulating plasma concentration range which includes the IC₅₀(i.e., the concentration of the test compound which achieves a half-maximal inhibition of symptoms) as determined in cell culture. Such information can be used to more accurately determine useful doses in humans.

When employed as pharmaceuticals, the compounds provided herein are usually administered in the form of pharmaceutical compositions. These compounds can be administered by a variety of routes including oral, parenteral, transdermal, topical, rectal, and intranasal.

Compounds provided herein are effective as injectable, oral, inhalable, topical, or intra-tumor administration compositions. Such compositions are prepared in a manner well known in the pharmaceutical art and comprise at least one active compound.

Also described herein are pharmaceutical compositions which contain, as the active ingredient, one or more of the compounds provided herein associated with pharmaceutically acceptable carriers. In making the compositions described herein, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions are preferably formulated in a unit dosage form, each dosage containing from about 0.1 to about 2000 mg, more usually about 1 to about 900 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound provided herein above is employed at no more than about 20 weight percent of the pharmaceutical composition, more preferably no more than about 15 weight percent, with the balance being pharmaceutically inert carrier(s).

An active compound is effective over a wide dosage range and is generally administered in a pharmaceutically or therapeutically effective amount. It, will be understood, however, that the amount of the compound actually administered can be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 mg/kg to about 250 mg/kg, more preferably about 1.0 mg/kg to about 50 mg/kg of body weight/day.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient described herein.

The tablets or pills described herein may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions described herein may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Additionally, liposomal formulations of the compounds described herein may be used, for example, to enhance therapeutic effect against certain infections, such as pneumonia or ling infections.

Intra-tumoral administration of compounds provided herein employs solutions or gels thereof prepared in suitable aqueous solutions containing appropriate excipient additives, such as dextrose, polyethylene glycol, cremophore, cyclodextrin, and similar excipient additives.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

Other suitable formulations for use can be found in Remington's Pharmaceutical Sciences, Mace Publishing Company, Philadelphia, PA, 17th ed. (1985).

Optionally, the compounds described herein may be co-administered with additional agents, including antioxidants, such as ascorbic acid, or megalin-receptor inhibitors generally known to attenuate adverse effects of polymyxin drugs.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference. Optionally, the compounds described herein could be administered as nanomicells, or nanomaterials-encapsulated compositions, prepared as described, for example, by Taki et al. in Pharmaceut., 2012, vol. 3, p. 1092.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the disclosure has been described herein in conjunction with a preferred aspect, a person with ordinary skills in the art, after reading the foregoing specification, can affect changes, substitutions of equivalents and other types of alterations to the disclosure as set forth herein. Each aspect described above can also have included or incorporated therewith such variations or aspects as disclosed in regard to any or all of the other aspects. The present disclosure is also not to be limited in terms of the particular aspects described herein, which are intended as single illustrations of individual aspects provided herein. Many modifications and variations of this idisclosure can be made without departing from its spirit and scope, as will be apparent to those skilled in the art. Functionally equivalent methods within the scope of this disclosure, in addition to those enumerated herein, will be apparent to those skilled in the art from the foregoing descriptions. It is to be understood that this disclosure is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A compound of Formula V-P or Formula V: 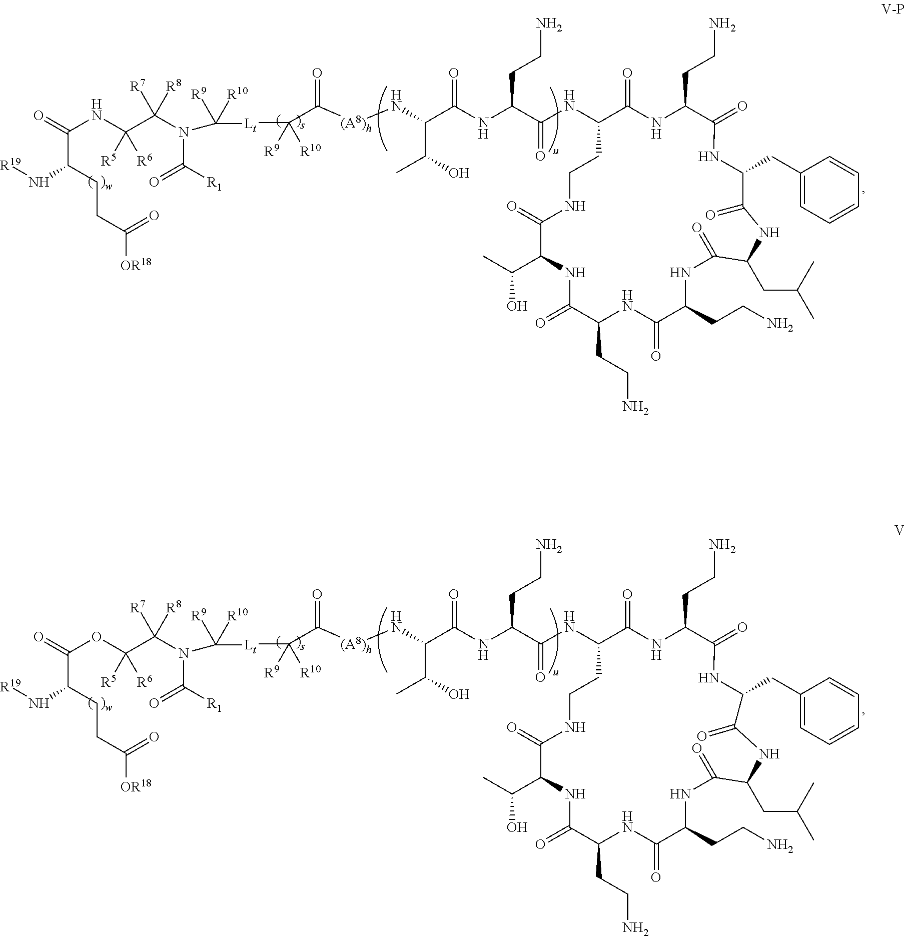
or pharmaceutically acceptable salt, solvate, or hydrate thereof,
wherein
R¹ is a residue formed by subtracting a single H atom from a precursor structure (H)ₙR¹ at any one of the following H-containing groups independently selected from NH, OH, SH, C(═O)OH, CONH, SO₂NH, and S(═O)NH when present in (H)ₙR¹; and wherein each (H)ₙR¹ is independently selected from:
a) a cytotoxic compound and an immunomodulating compound that possesses activity or is capable of inducing activity against one or more cancer cells;
b) afatinib, ARS-1630, axitinib, BGB-324, BLU-554, brivanib, (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib, emtansine, englerin, monomethyl auristatin E, irinotecan, maytansinoid, neratinib, nilotinib, nintedanib, ozogamicin, paclitaxel, pazopanib, regorafenib, sacituzumab, selpercatinib, semaxanib, sorafenib, sunitinib, SN-38, temsirolimus, tivantinib, tivozanib, vatalanib, veliparib, and vinblastine; or variants thereof, and
c) a nitrogen-containing heterocyclic structure comprised in the (H)ₙR¹ provided in (a) and (b) connected at a heterocyclic nitrogen atom present within (H)ₙR¹; wherein the nitrogen atom becomes a nitrogen atom with a single positive charge, selected from imidazolium, pyrazolium, pyridinium, or indazolium group;
R⁶, R⁷, R⁸, R⁹ and R¹⁰ are independently selected from H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, and heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein the remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁵ and R⁶, ii) R⁶ and R⁷, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R¹ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and
R¹⁸ is H or C₁₋₁₂alkyl;
R¹⁹ is H, C₁₋₁₂alkyl, C(═O)H, C(═O)C₁₋₁₂alkyl, C(═O)OC₁₋₁₂alkyl, C(═O)OC₁₋₁₂-alkyl, C(═O)NHC₁₋₁₂alkyl, SO₂C₁₋₁₂alkyl, SO₂aryl, C(═O)C₃₋₇cycloalkyl, C(═O)OC₃₋₇cycloalkyl, C(═O)NHC₃₋₇cycloalkyl, C(═O)NHC₁₋₁₂alkyl, SO₂C₃₋₇cycloalkyl, or A¹;
each optional group L is selected from alkyl, CR²⁰R²¹OC(═O)CR²²R²³ and CR²⁰R²¹C(═O)OCR²²R²³;
R²⁰ through R²³ are independently selected from H, C₁₋₁₂alkyl, or C₃₋₇cycloalkyl; or any of the two adjacent groups R²⁰ and R²¹ or R²² and R²³ independently taken together form a C₃₋₇cycloalkyl group;
A⁸ is optional and when present is selected from amino acid residue independently selected from unsubstituted or substituted at any N atom and where each of the amino acid residues, when present, is selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid;
integer h is 0, 1, or 2;
integer t is 0, 1, or 2; and
integers u and w are independently 0 or 1.

2. A compound of Formula I 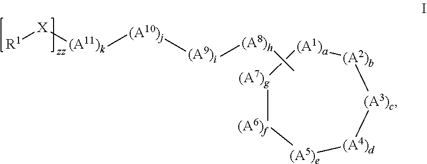
or a pharmaceutically acceptable salt, solvate, or hydrate thereof wherein:
R¹ is a residue formed by subtracting a single H atom from respective parent (precursor) structure (H)ₙR¹ at any one of the following H-containing group(s) independently selected from NH, OH, SH, C(═O)OH, CONH, SO₂NH, and S(═O)NH when present in (H)ₙR¹; and wherein a) (H)ₙR¹ is a cytotoxic compound, or an immunomodulating compound possessing an activity or capable of inducing an activity against one or more cancer cells; or
b) (H)ₙR¹ is selected from afatinib, ARS-1630, axitinib, BGB-324, BLU-554, brivanib, (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib, emtansine, englerin, monomethyl auristatin E, irinotecan, maytansinoid, neratinib, nilotinib, nintedanib, ozogamicin, paclitaxel, pazopanib, regorafenib, sacituzumab, selpercatinib, semaxanib, sorafenib, sunitinib, SN-38, temsirolimus, tivantinib, tivozanib, vatalanib, veliparib, and vinblastine; or a variantthereof; or
c) (H)ₙR¹ is a heterocyclic structure comprised in the (H)ₙR¹ provided in (a) and (b) connected to X at one of heterocyclic nitrogen atom(s) present within the structure (H)ₙR¹; wherein said nitrogen atom becomes a nitrogen atom with a single positive charge, selected from imidazolium, pyrazolium, pyridinium, or indazolium group;
integer n is independently selected from 1, 2, and 3;
integers a through k are independently selected from 0, 1, and 2; and
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, and heterocyclyl; and
integer zz is independently selected from 1, 2, and 3;
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene, (C₁₋₁₂alkylene)C(═O)O, OC(═O)(C₁₋₁₂alkylene), (C₁₋₁₂alkylene)OC(═O), C(═O)O(C₁₋₁₂alkylene), (C₁₋₁₂alkylene)C(═O)N(R⁴), N(R⁴)C(═O)(C₁₋₁₂alkylene), (C₁₋₁₂alkylene)N(R⁴)C(═O), C(═O) N(R⁴)(C₁₋₁₂alkylene), C(═O)(CR⁵R⁶)ₚ(CR⁷R⁸)ᵣ(CR⁹R¹⁰)ₛC(═O), C(═O)O(CR⁵R⁶)O(CR⁷R⁸)ᵣ(CR⁹R¹⁰)ₛC(═O), P(═O)(OCR⁵R⁶)ₚ(CR⁷R⁸)ᵣ(CR⁹R¹⁰)ₛC(═O), C(═O)(CR⁷R⁸)ₚ(CR⁹R¹⁰)ᵣP(═O)(OCR⁵R⁶)ₘ, P(═O)(NHCR⁵R⁶)ₚ(CR⁷R⁸)ᵣ(CR⁹R¹⁰)ₛC(═O), P(═O)(OCR⁵R⁶)CF₂, P(═O)(OCR⁵R⁶)CF₂(CR⁷R⁸)ᵣC(═O), P(═O)(OH)CF₂, P(═O)(OH)CF₂(CR⁷R⁸)ᵣC(0), C(═O)(CR⁷R⁸)ᵣ(CR⁹R¹⁰)ₛP(═O)(NHCR⁵R⁶)ₚ, C(═O)O(CR⁵R⁶)ₚ(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹)ₛOC(═O), C(═O)(CR⁵R⁶)ₚ(CR⁷R⁸)ᵣ(CR⁹R¹⁰)ₛN(R⁴)C(═O), C(═O)CR⁵═CR⁷—(CR⁹R¹⁰)ₛC(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁴)C(═O), C(═O)N(R⁴)SO₂(CR⁵R⁶)ₚ(CR⁷R⁸)ᵣ(CR⁹R¹⁰)ₛC(═O), C(═O)(CR⁵R⁶)ₚ(CR⁷R⁸)ᵣ(CR⁹R¹⁰)ₛN(R⁴)SO₂C(═O), C(═O)(CR⁵R⁶)ₚS—S(CR⁷R⁸)ᵣ(CR⁹R¹⁰)ₛC(═O), C(═O)O(CR⁵R⁶)ₚS—S(CR⁷R⁸)ᵣ(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁴)C(═O), C(═O)(CR⁵R⁶)ₚ(CR⁷R⁸)ᵣS—S(CR⁹R¹⁰)ₛC(═O), C(═O)O(CR⁵R⁶)ₚ(CR⁷R⁸)ᵣS—S(CR⁹R¹⁰)ₛC(═O), C(═O)N(R⁴)(CR⁵R⁶)ₚ(CR⁷R⁸)ᵣ(CR⁹R¹⁰)ₛC(═O), C(═O)(CR⁵R⁶)ₚS—S(CR⁷R⁸)ᵣS—S(CR⁹R¹⁰)ₛOC(═O), C(═O)(CR⁵R⁶)ₚ(CR⁷R⁸)ᵣ(CR⁹R¹⁰)ₛN(R⁴)C(═O) C(═O)CR⁵═CR⁷—S—S—(CR⁹R¹⁰)ₛC(═O), C(═O)OCR⁵═CR⁷—(CR⁹R¹⁰)ₛC(═O), C(═O)N(R⁴)CR⁵═CR⁷—(CR⁹R¹⁰)ₛC(═O), C(═O)CR⁵═CR⁷—(CR⁹R¹⁰)ₛOC(═O), C(═O)CR⁵═CR⁷—(CR⁹R¹⁰)ₛN(R⁴)C(═O), C(═O)N[(CR⁵R⁶)ₚ(CR⁷R)ᵣNHC(═O)A¹⁴](CR⁹R¹⁰)ₛC(═O), C(═O)N[(CR⁵R⁶)ₚ(CR⁷R⁸)ᵣNHC(═O)A¹⁴](CR⁹R¹⁰)ₛNHC(═O), C(═O)N[(CR⁵R⁶)ₚ(CR⁷R⁸)ᵣNHC(═O)A¹⁴](CR⁹R¹⁰)ₛOC(═O), C(═O)N[(CR⁵R⁶)ₚ(CR⁷R⁸)ᵣN(C₁₋₆alkyl)C(═O)A¹⁴](CR⁹R¹⁰)ₛNHC(═O), C(═O)N[(CR⁵R⁶)ₚ(CR⁷R⁸)ᵣN(C₁₋₆alkyl)C(═O)A¹⁴](CR⁹R¹⁰)ₛC(═O), C(═O)N[(CR⁵R⁶)ₚ(CR⁷R⁸)ᵣN(C₁₋₆alkyl)C(═O)A¹⁴](CR⁹R¹⁰)ₛOC(═O), C(═O)N[(CR⁵R⁶)ₚ(CR⁷R⁸)ᵣOC(═O)A¹⁴](CR⁹R¹⁰)ₛC(═O), C(═O)N[(CR⁵R⁶)ₚ(CR⁷R⁸)ᵣOC(═O)A¹⁴](CR⁹R¹⁰)ₛN(C═O), C(═O)N[(CR⁵R⁶)ₚ(CR⁷R⁸)ᵣOC(═O)A¹⁴](CR⁹R¹⁰)ₛO(C═O), C(═O)N[(CR⁵R⁶)ₚ(CR⁷R⁸)ᵣNHC(═O)A¹⁴A¹⁵](CR⁹R¹⁰)ₛC(═O), C(═O)N[(CR⁵R⁶)ₚ(CR⁷R⁸)ᵣNHC(═O)A¹⁴A¹⁵](CR⁹R¹⁰)ₛN(C═O), C(═O)N[(CR⁵R⁶)ₚ(CR⁷R⁸)ᵣNHC(═O)A¹⁴A¹⁵](CR⁹R¹⁰)ₛO(C═O), C(═O)N[(CR⁵R⁶)ₚ(CR⁷R⁸)ᵣOC(═O)A¹⁴A¹⁵](CR⁹R¹⁰)ₛC(═O), C(═O)N[(CR⁵R⁶)ₚ(CR⁷R⁸)ᵣOC(═O)A¹⁴A¹⁵](CR⁹R¹⁰)ₛN(C═O), C(═O)N[(CR⁵R⁶)ₚ(CR⁷R⁸)ᵣOC(═O)A¹⁴A¹⁵](CR⁹R¹⁰)ₛO(C═O), C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O), C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH(NH₂)CH₂CH₂COOH]CH₂CH₂C(═O)OCH₂CH₂C(═O), C(═O)N[CH₂CH₂NHC(═O)CH(NH₂)CH₂CH₂COOH]CH₂CH₂C(═O)OCH(Me) CH(Me)C(═O), C(═O)N[CH₂CH₂NHC(═O)CH(NH₂)CH₂CH₂COOH]CH₂CH₂C(═O)OC(Me)₂C(Me)₂C(═O), C(═O)N[CH₂CH₂NHC(═O)CH(NH₂)CH₂CH₂COOH]CH₂CH₂C(═O)O—C₃₋₆cycloalkylene- C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N[CH₂CH₂NHC(═O)CH(NH₂)CH₂CH₂COOH]CH₂CH(Me)OC(═O)CH₂CH₂C(═O), C(═O)N[CH₂CH₂NHC(═O)CH(NH₂)CH₂CH₂COOH]CH₂ C(Me)₂OC(═O)CH₂CH₂C(═O), C(═O)N[CH₂CH₂NHC(═O)CH(NH₂)CH₂CH₂COOH]CH₂CH₂OC(═O)—C₃₋₆cycloalkylene- C(═O), C(═O)N{CH₂CH₂NHC(═O)CH[NH(A¹)]CH₂CH₂COOH}CH₂CH₂OC(═O)CH₂CH₂C(═O), C(═O)N[CH₂CH₂NHC(═O)CH(NH₂)CH₂CH₂COOH]CH₂CH₂OC(═O)C(Me)CH(Me)C(═O), C(═O)N[CH₂CH₂NHC(═O)CH(NH₂)CH₂CH₂COOH]CH₂CH₂OC(═O)C(Me)₂C(Me)₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N{CH₂CH₂NHC(═O)CH[NHC(═O)C₁₋₆alkyl]CH₂CH₂COOH}CH₂CH₂C(═O), C(═O)N{CH₂CH₂NHC(═O)CH[NH(A¹)]CH₂CH₂COOH}CH₂CH₂C(═O), C(═O)N[CH₂CH₂NHC(═O)CH(NH₂)CH₂CH₂COOC₁₋₆alkyl]CH₂CH₂C(═O), C(═O)N[CH₂CH₂NHC(═O)CH₂CH₂CH(NH₂)COOH]CH₂CH₂C(═O), C(═O)N[CH₂CH₂NHC(═O)CH₂CH₂CH(NH₂)COOC₁₋₆alkyl]CH₂CH₂C(═O) C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O), C(═O)N[CH₂CH₂NHC(═O)CH₂CH₂CH(NH₂)COOH](CR⁵R⁶)ₚCR⁷R⁸)ᵣ OC(═O) (CR⁹R¹⁰)ₛC(═O), C(═O)N[CH₂CH₂NHC(═O)CH₂CH₂CH(NH₂)COOH](CR⁵R⁶)ₚC(═O)O(CR⁷R⁸)ᵣ(CR⁹R¹⁰)ₛC(═O), C(═O)N[CH₂CH₂NHC(═O)CH₂CH₂CH(NH₂)COOH](CR⁵R⁶)ₚOC(═O)(CR⁷R⁸)ᵣ(CR⁹R¹⁰)ₛC(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), C(═O)N[CH₂CH₂NHC(═O)CH₂CH₂CH(NH₂)COOH]CH₂CMe₂C(═O)OCH₂CH₂C(═O), C(═O)N[CH₂CH₂NHC(═O)CH₂CH₂CH(NH₂)COOH]CH₂CH₂OC(═O)CH(Me)-CH₂C(═O), C(═O)N[CH₂CH₂N(C₁₋₆alkyl)C(═O)CH₂CH₂CH(NH₂)COOH]CH₂CH₂C(═O), (S)—C(═O)N[CH₂CH₂NHC(═O)CH₂CH₂CH(NH₂)COOH]CH₂CH₂C(═O), (R)—C(═O)N[CH₂CH₂NHC(═O)CH₂CH₂CH(NH₂) COOH]CH₂CH₂C(═O), (S)—C(═O)N[CH₂CH₂NHC(═O)CH₂CH₂CH(NH(C═O)R⁷)COOH]CH₂CH₂C(═O), (R)—C(═O)N[CH₂CH₂NHC(═O)CH₂CH₂CH(NH(C═O)R⁷)COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), and (R)—C(═O)N[CH₂CH₂NHC(═O)CHNH(CH₂CH₂NH₂)]CH₂CH₂C(═O);
and any variant of above groups formed by repositioning(s), addition(s), or deletion(s) of the fragments C(═O), OC(═O), N(R⁴)C(═O), P(═O)(OCR⁵R⁶)CF₂, P(═O)(OH)CF₂, or C(═O)N(R⁴)SO₂ therein;
R⁶, R⁷, R⁸, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein the remaining atoms are carbon; or wherein
any two of R⁵ through R¹⁰, together with the carbon atom(s) to which they are attached form a 4 to 7-member saturated or unsaturated C₃₋₆cycloalkylene; or any of i) R⁵ and R⁶, ii) R⁶ and R⁷, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R¹ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and wherein
when fragments (CR⁵R⁶)ₚ(CR⁷R⁸)ᵣ or (OCR⁵R⁶)ₚ(CR⁶R⁷)ᵣ are present, then [p+r]≥1; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is optionally connected to one to two amino acid residue(s) A¹² or A¹³;
or any variant of the above X groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates one or more additional divalent groups selected from C₁₋₁₂alkylene, C₂₋₁₂alkenylene, C₂₋₁₂alkynylene, (CH₂—)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C¹⁻¹⁴alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C¹-14alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C¹-14alkyl)C(═O), and similar linear groups; and
A¹ through A¹⁵ are independently optional and when present are independently selected from amino acid residues independently selected from unsubstituted or substituted at any N atom and where each of the amino acid residues, when present, is independently selected from alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid.

3. The compound of claim 2 according to Formula I, or a pharmaceutically acceptable salt, solvate, or hydrate thereof, wherein
the integers a through g are each 1; and
A¹ is Thr or Ser; A², A³ A⁶, and A⁷ are independently selected from Dab, Dap, Ser, and Thr; A⁴ is Leu or Ile; and A⁵ is Phe, D-Phe, Bip, D-Bip, Val, or D-Val.

4. The compound of claim 2, according to Formula I, or a pharmaceutically acceptable salt, solvate, or hydrate thereof, wherein
the cyclic peptide structure in the compound of Formula I is comprised of optional amino acid residues A¹ through A⁷ and is a cyclic peptide structure identical to that present in polymyxin A, polymyxin B, polymyxin E, or octapeptin, or similar structures.

5. The compound of claim 2, according to Formula II-P: 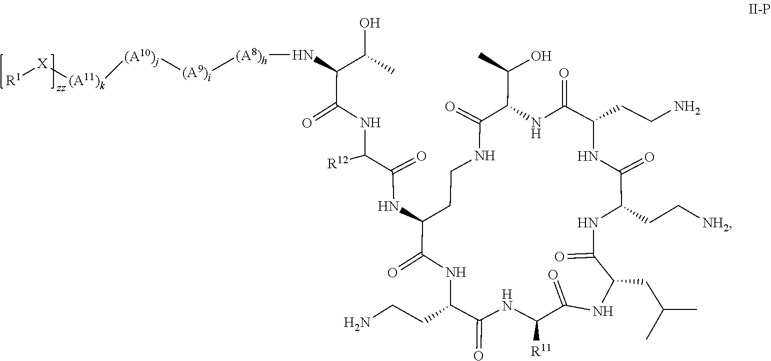
or a pharmaceutically acceptable salt, solvate, or hydrate thereof, wherein:
R¹¹ is CH₂CH(CH₃)₂ or CH₂Ph; and
R¹² is CH₂NH₂ or CH₂CH₂NH₂.

6. The compound of claim 2, according to Formula III: 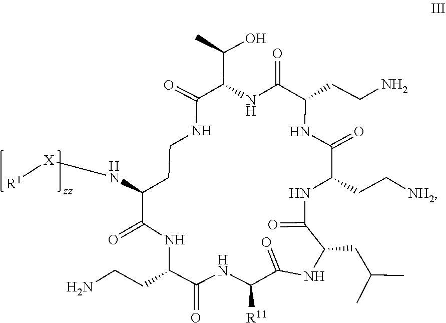
or a pharmaceutically acceptable salt, solvate, or hydrate thereof, wherein:
the integer zz is equal to 1 or 2;
R¹ is independently selected from a residue formed by subtracting a single H atom at one of the following H-containing groups independently selected from NH, OH, SH, C(═O)OH, CONH, SO₂NH, and S(═O)NH present in afatinib, ARS-1630, axitinib, BGB-324, BLU-554, brivanib, cabozantinib, cediranib, ceritinib, ciforadenant, derazantinib, dovitinib, foretinib, lenvatinib, monomethyl auristatin E, irinotecan, maytansinoid, neratinib, nilotinib, nintedanib, ozogamicin, paclitaxel, pazopanib, regorafenib, sacituzumab, selpercatinib, semaxanib, sorafenib, sunitinib, SN-38, trastuzumab, tesirine, temsirolimus, tivantinib; and a variant thereof;
X is selected from a structure below:
C(═O)N[CH₂CH₂NHC(═O)CH(NH₂)CH₂CH₂COOH]CH₂CH₂C(═O), C(═O)N{CH₂CH₂NHC(═O)—[(S)—CH(NH₂)]CH₂CH₂COOH}CH₂CH₂C(═O), C(═O)N{CH₂CH₂NHC(═O)—[(R)—CH(NH₂)]CH₂CH₂COOH}CH₂CH₂C(═O), C(═O)N[CH₂CH₂OC(═O)CH(NH₂)CH₂CH₂COOH]CH₂CH₂C(═O), C(═O)N[CH₂CH₂OC(═O)CH(S)—CH(NH₂)CH₂CH₂COOH]CH₂CH₂C(═O), C(═O)N[CH₂CH₂NHC(═O)CH(OH)CH₂CH₂COOH]CH₂CH₂C(═O), C(═O)N[CH₂CH₂NHC(═O)CH(NH₂)CH₂COOH]CH₂CH₂C(═O), C(═O)N{CH₂CH₂NHC(═O)—[(S)—CH(NH₂)]CH₂COOH}CH₂CH₂C(═O), C(═O)N{CH₂CH₂NHC(═O)—[(R)—CH(NH₂)]CH₂COOH}CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)—{(S)—CH[NHC(═O)H]}CH₂CH₂COOH]CH₂CH₂C(═O), C(═O)N{CH₂CH₂NHC(═O)—{(S)—CH[NHC(═O)Me]}CH₂CH₂COOH}CH₂CH₂C(═O), C(═O)N{CH₂CH₂NHC(═O)CH[NH(A¹)]CH₂CH₂COOH}CH₂CH₂C(═O)OCH₂CH₂C(═O), C(═O)N[CH₂CH₂NHC(═O)CH(NH₂)CH₂CH₂COOH]CH₂CH₂OC(═O)CH₂CH₂C(═O), C(═O)N{CH₂CH₂NHC(═O)CH[NHC(═O)H]CH₂CH₂COOH}CH₂CH₂C(═O), C(═O)N{CH₂CH₂NHC(═O)CH[NHC(═O)Me]CH₂CH₂COOH}CH₂CH₂C(═O), C(═O)N[CH₂CH₂NHC(═O)CH₂CH₂CH(NH₂)COOH]CH₂CH₂C(═O), C(═O)N[CH₂CH₂OC(═O)CH₂CH₂CH(NH₂)COOH]CH₂CH₂C(═O), C(═O)N[CH₂CH₂NHC(═O)CH₂CH₂CH(NH₂)COOH]CH₂CH₂C(═O)OCH₂CH₂C(═O), C(═O)N[CH₂CH₂NHC(═O)CH₂CH₂CH(NH₂)COOH]CH₂CH₂OC(═O)CH₂CH₂C(═O), (S)—C(═O)N[CH₂CH₂NHC(═O)CH₂CH₂CH(NH₂)COOH]CH₂CH₂C(═O), (R)—C(═O)N[CH₂CH₂NHC(═O)CH₂CH₂CH(NH₂) COOH]CH₂CH₂C(═O), (S)—C(═O)N[CH₂CH₂NHC(═O)CHNH(Me)]CH₂CH₂C(═O), (R)—C(═O)N[CH₂CH₂NHC(═O)CHNH(Me)]CH₂CH₂C(═O), (S)—C(═O)N[CH₂CH₂NHC(═O)CHNH(CH₂CH₂CH₂CH₂NH₂)]CH₂CH₂C(═O), (R)—C(═O)N[CH₂CH₂NHC(═O)CHNH(CH₂CH₂CH₂CH₂NH₂)]CH₂CH₂C(═O), (S)—C(═O)N[CH₂CH₂NHC(═O)CHNH(CH₂CH₂NH₂)]CH₂CH₂C(═O), (R)—C(═O)N[CH₂CH₂NHC(═O)CHNH(CH₂CH₂NH₂)]CH₂CH₂C(═O); and
R¹¹ is CH₂CH(CH₃)₂ or CH₂Ph.

7. The compound of claim 2, or a pharmaceutically acceptable salt, solvate, or hydrate thereof, wherein each X, either at its left or right side, independently incorporates one or more additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₄alkyl)(CH₂)ₚO(CH₂)ᵣO(CH₂)ₛC(═O), N(C₁₋₄alkyl)(CH₂)ₚO(CH₂)ᵣO(CH₂)ₛOC(═O), N(C₁₋₄alkyl)(CH₂)ₚO(CH₂)ᵣO(CH₂)ₛNHC(═O), and N(C₁₋₁₄alkyl)(CH₂)ₚO(CH₂)ᵣO(CH₂)ₛN(C₁₋₁₄alkyl)C(═O).

8. The compound of claim 2 or a pharmaceutically acceptable salt, solvate, or hydrate thereof, wherein each X is independently selected from the structures below, wherein either the left side or the right side of X is connected to R¹:

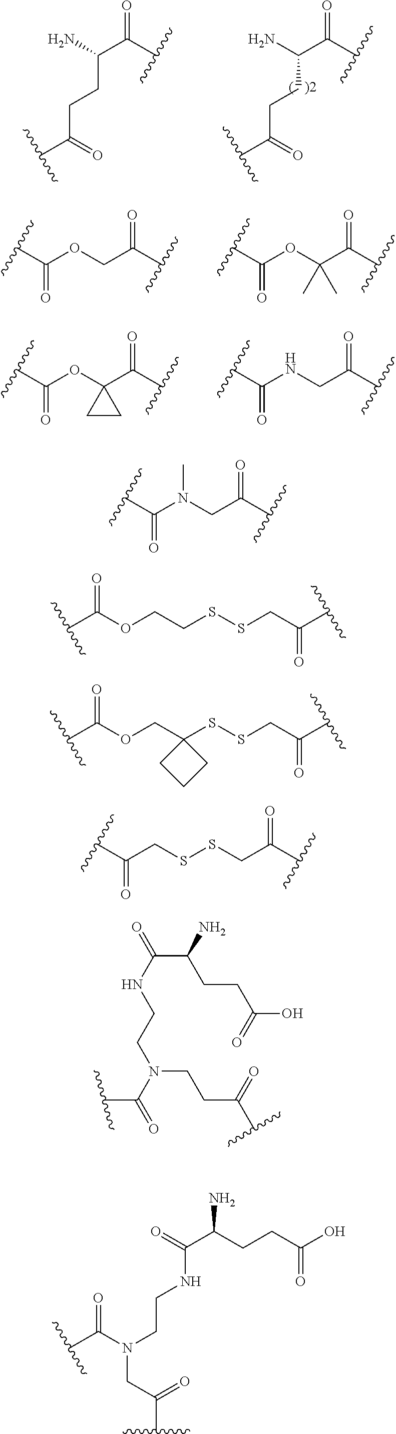

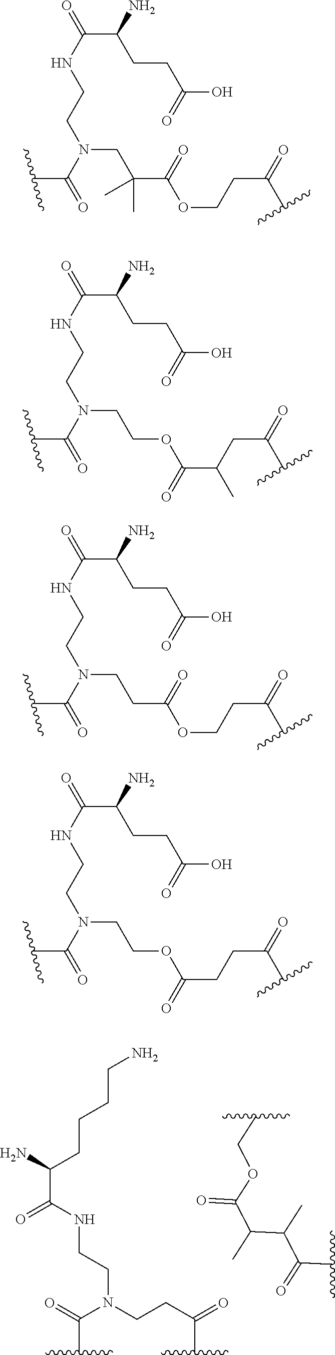

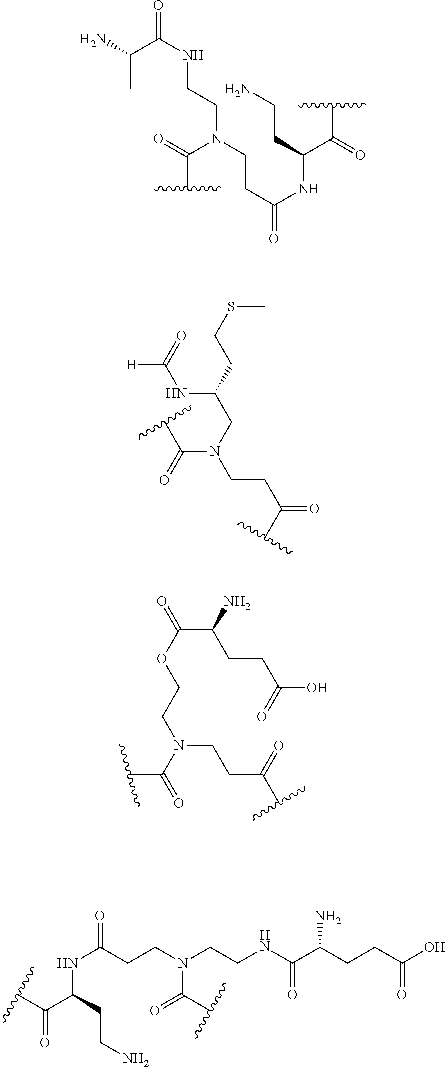 [SMILES: *C(=O)[C@H](CCN)NC(=O)CCN(CCNC(=O)[C@H](C)N)C(*)=O ; *C(=O)CCN(C[C@@H](CCSC)NC=O)C(*)=O ; *C(=O)CCN(CCOC(=O)[C@@H](N)CCC(=O)O)C(*)=O ; *C(=O)[C@H](CCN)NC(=O)CCN(CCNC(=O)[C@H](N)CCC(=O)O)C(*)=O]

9. (canceled)

10. The compound of claim 1, according to Formula V-P or Formula V: 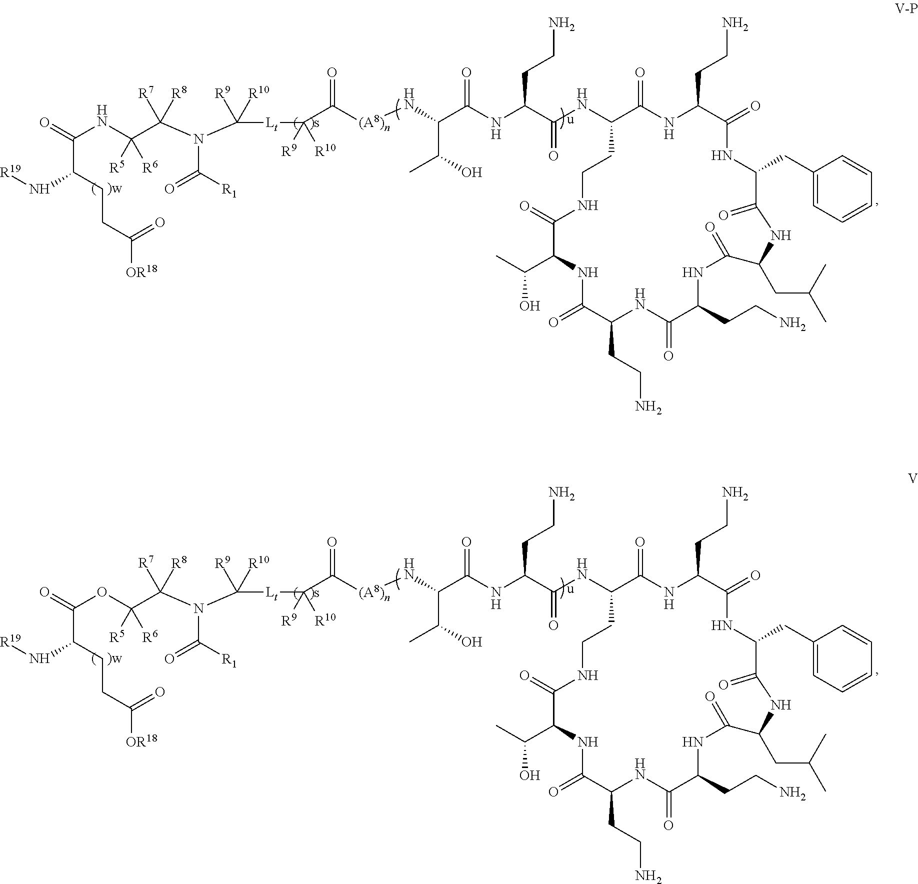
or a pharmaceutically acceptable salt, solvate, or hydrate thereof, wherein
R¹⁸ is H or C₁₋₁₂alkyl;
R¹⁹ is H, C₁₋₁₂alkyl, C(═O)H, C(═O)C₁₋₁₂alkyl, C(═O)OC₁₋₁₂alkyl, C(═O)OC₁₋₁₂alkyl, C(═O)NHC₁₋₁₂alkyl, C(═O)C₃₋₇cycloalkyl, C(═O)OC₃₋₇cycloalkyl, C(═O)NHC₃₋₇cycloalkyl, or C(═O)NHC₁₋₁₂alkyl;
each optional group L is selected from alkyl, CR²⁰R²¹OC(═O)CR²²R²³ and CR²⁰R²¹C(═O)OCR²²R²³;
R²⁰ through R²³ are independently selected from H, C₁₋₁₂alkyl, and C₃₋₇cycloalkyl;
the integer t is 0, 1, or 2;
integer h is 0, 1, or 2; and
integers u and w are 1.

11. (canceled)

12. The compound of claim 10, wherein
R¹ is derived by subtracting H from one of a NH group in (H)R¹, wherein (H)R¹ is axitinib or tivozanib;
R⁵ through R⁸ are all H; and
integers h and t are both 0, such that optional groups A⁸ and L are absent and the atoms adjacent to the absent groups A⁸ and L are connected directly to each other.

13. The compound of claim 1, or a pharmaceutically acceptable salt, solvate, or hydrate thereof, wherein R¹ is selected from the structures below:

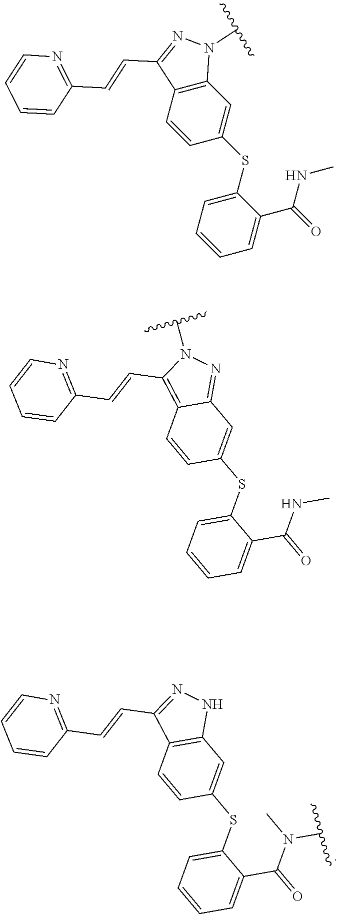 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1 ; *N(C)C(=O)c1ccccc1Sc1ccc2c(/C=C/c3ccccn3)n[nH]c2c1]

14. The compound of claim 2, or a pharmaceutically acceptable salt, solvate, or hydrate thereof, selected from the structures below:

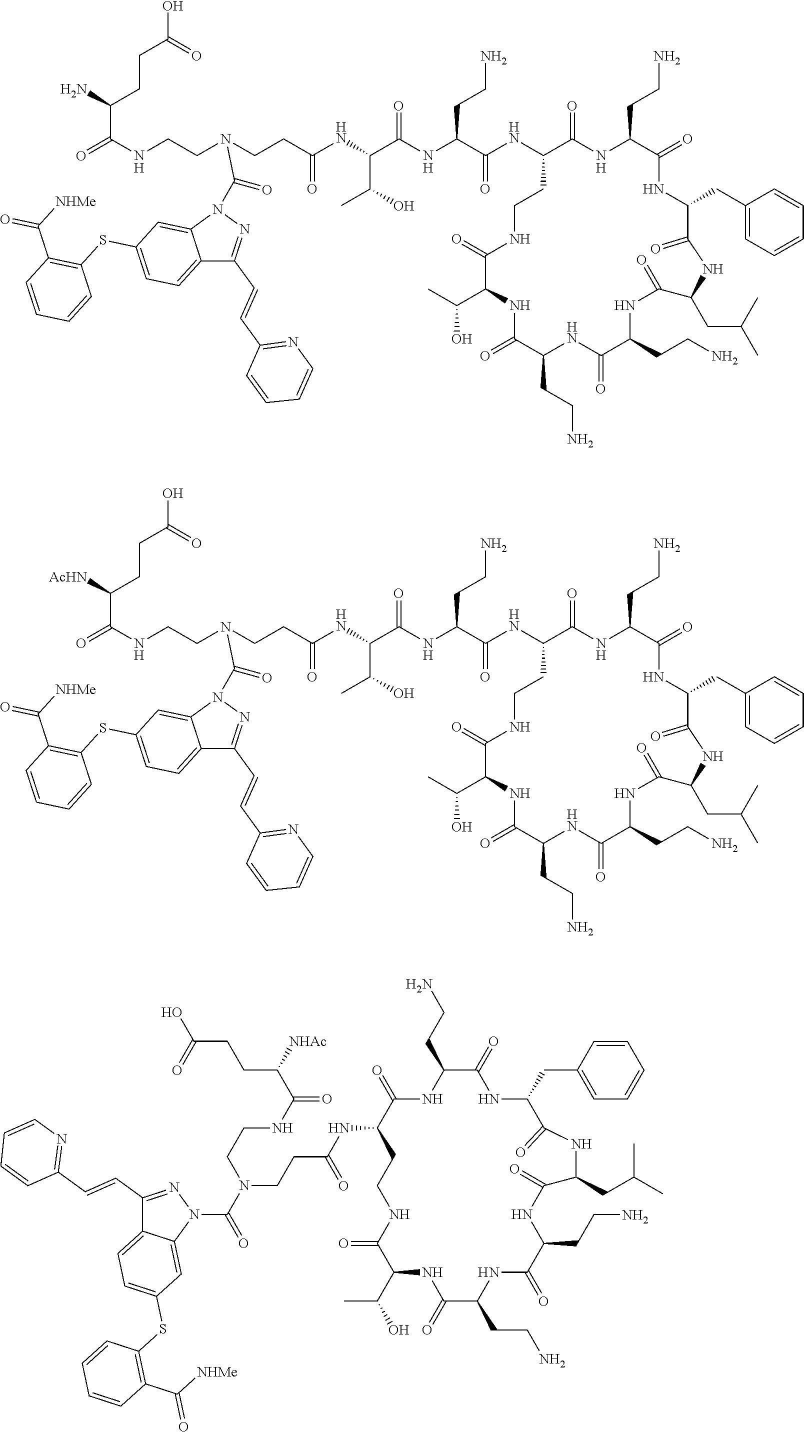

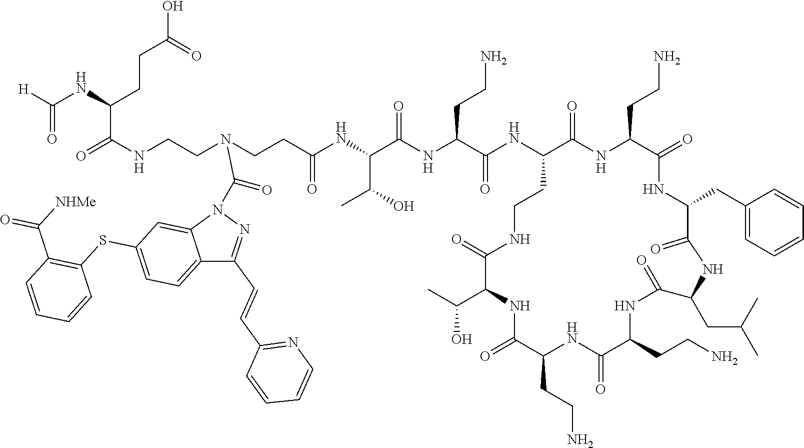 [SMILES: CNC(=O)c1ccccc1Sc1ccc2c(/C=C/c3ccccn3)nn(C(=O)N(CCNC(=O)[C@H](CCC(=O)O)NC=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

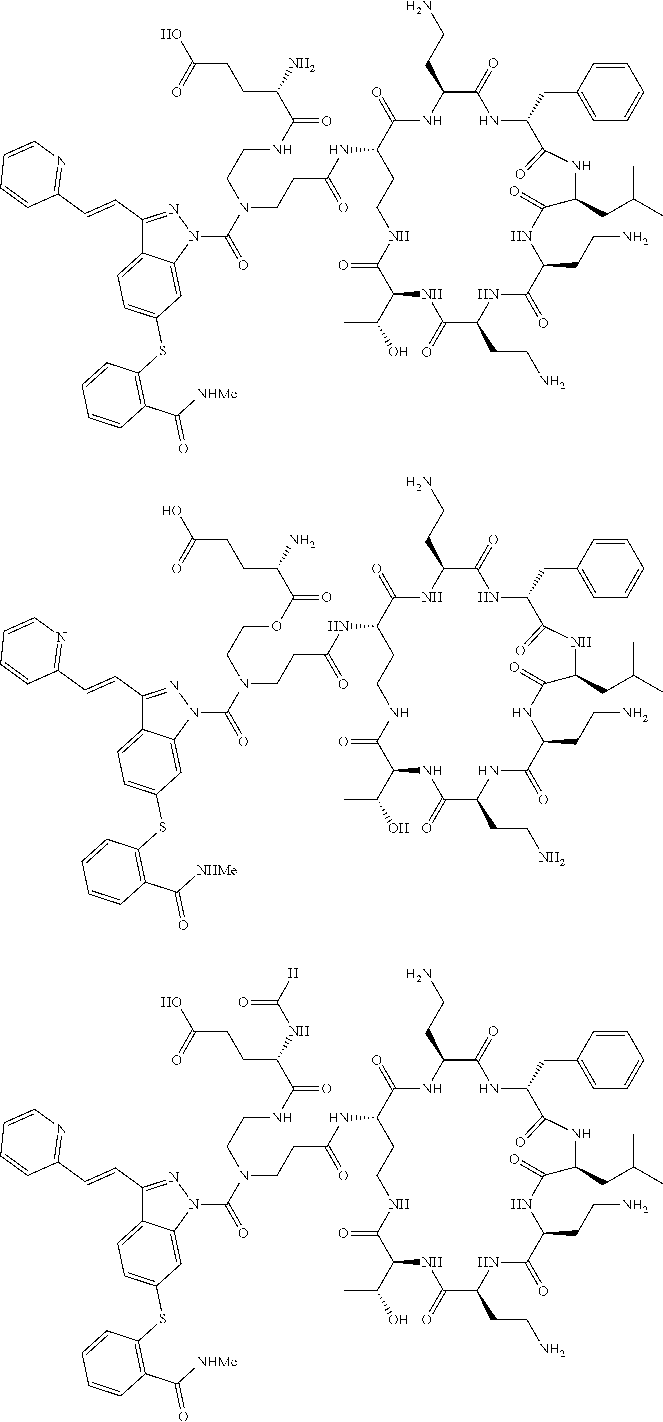

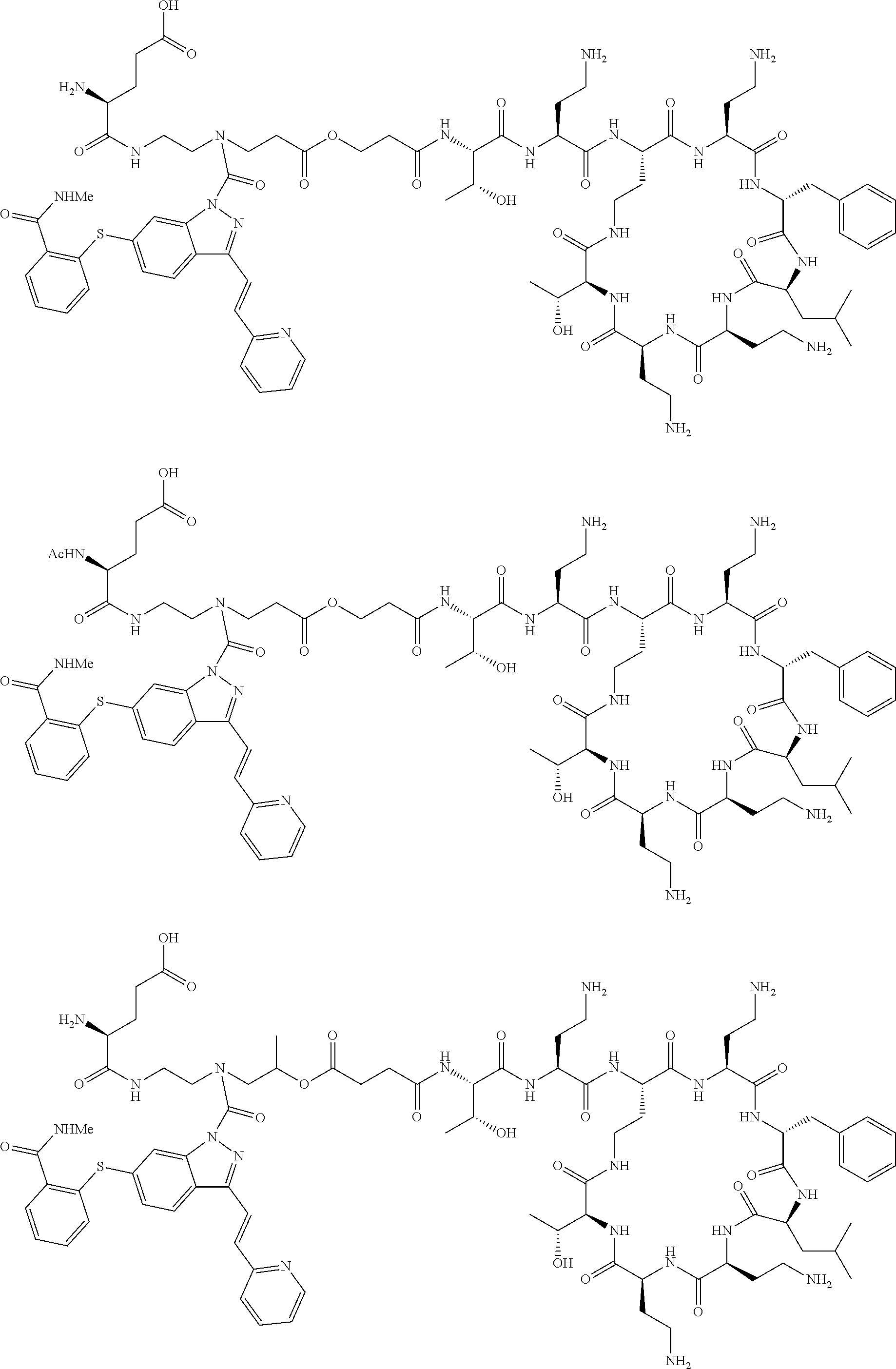

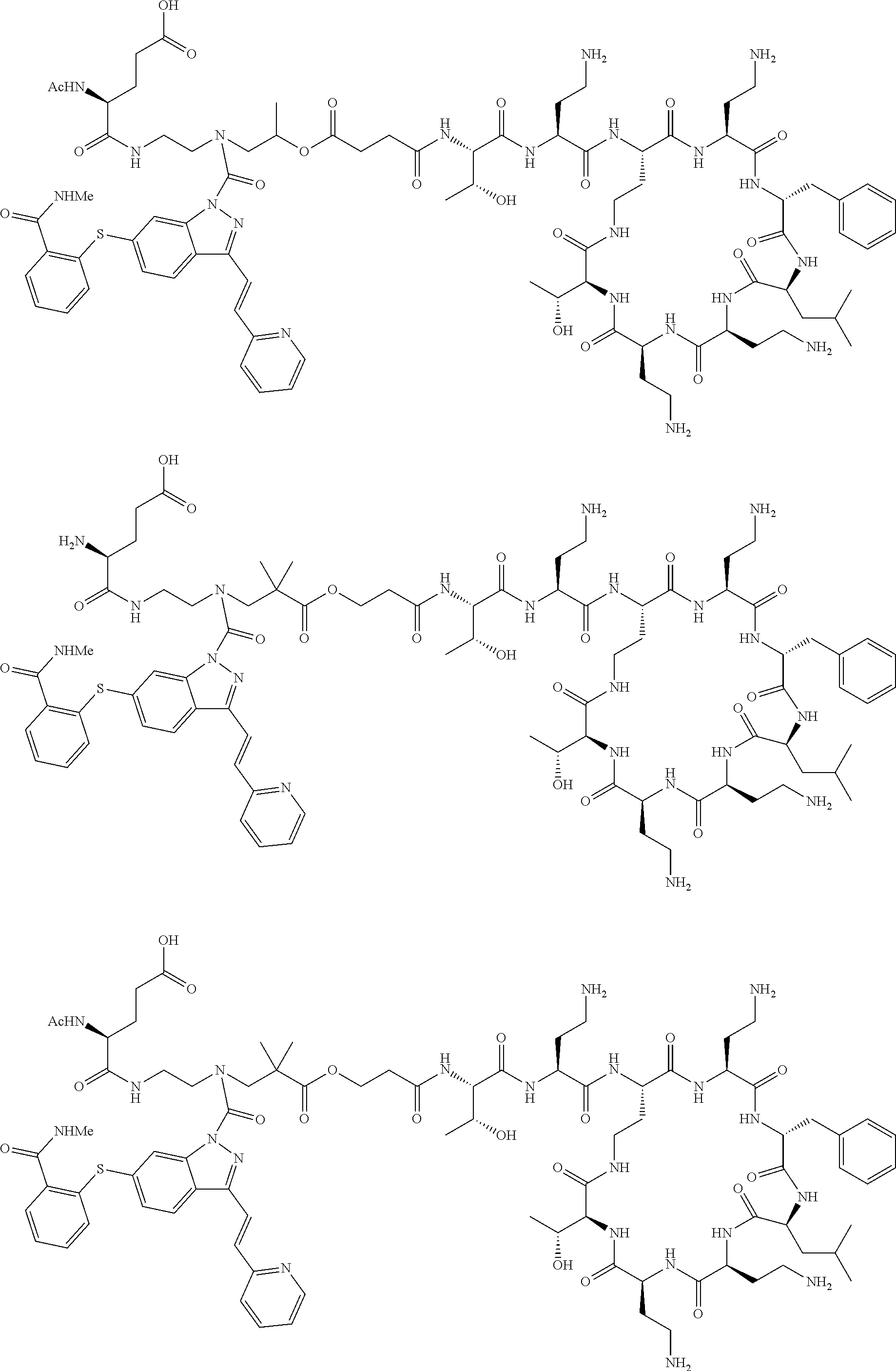

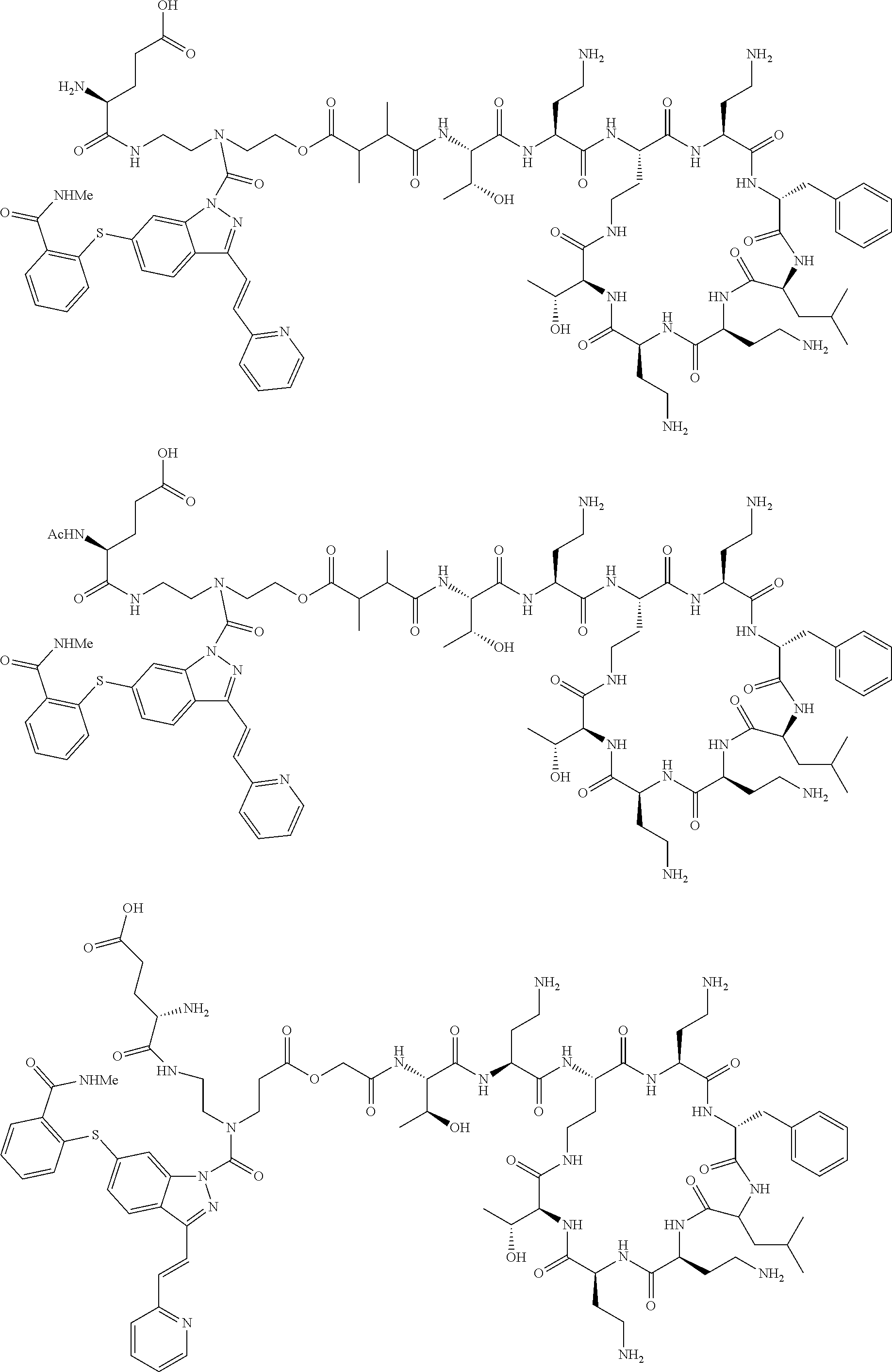

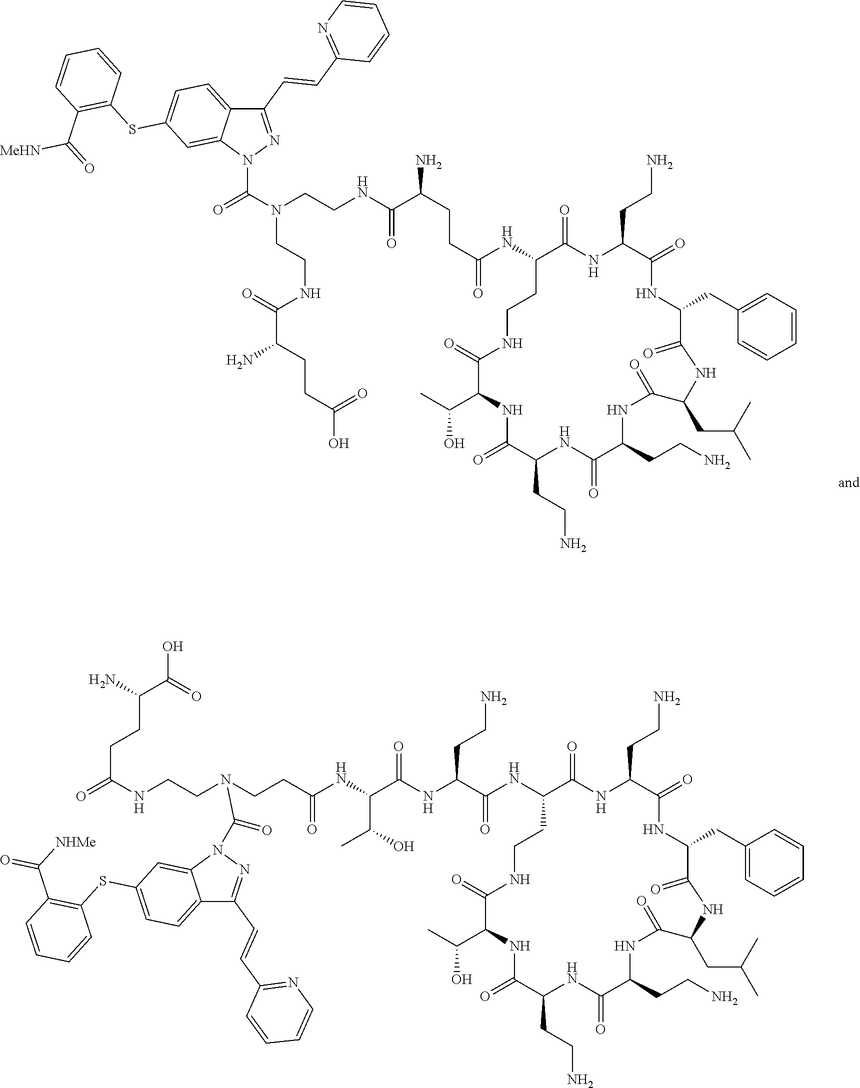 [SMILES: CNC(=O)c1ccccc1Sc1ccc2c(/C=C/c3ccccn3)nn(C(=O)N(CCNC(=O)[C@@H](N)CCC(=O)O)CCNC(=O)[C@@H](N)CCC(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)c2c1 ; CNC(=O)c1ccccc1Sc1ccc2c(/C=C/c3ccccn3)nn(C(=O)N(CCNC(=O)CC[C@H](N)C(=O)O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

15. (canceled)

16. (canceled)

17. (canceled)

18. The compound of claim 2, or a pharmaceutically acceptable salt, solvate, or hydrate thereof, possessing in vitro and/or in vivo anticancer activity against cancerous cells, as determined by inhibition or slowing of cancer cell growth using in vitro cytotoxicity test(s) or assay(s), or by testing of said compounds in animal models for cancer(s); optionally, wherein the cancer is a renal cancer or a kidney cancer.

19. (canceled)

20. The compound of claim 2, or a pharmaceutically acceptable salt, solvate, or hydrate thereof, possessing a reduced cytotoxicity against non-cancerous mammalian cell(s), when compared to an agent or drug (H)ₙR¹ incorporated into said compound, as determined by in vitro cytotoxicity test(s), wherein the in vitro cytotoxicity test(s) is a cell growth inhibition test(s); optionally, possessing at least about 50% reduced cytotoxicity against non-cancerous mammalian cell(s), when compared to the corresponding agent or drug of formula (H)ₙR¹, as determined by in vitro cytotoxicity test(s), wherein the in vitro cytotoxicity test(s) is a cell growth inhibition test(s).

21. (canceled)

22. The compound of claim 2, or a pharmaceutically acceptable salt, solvate, or hydrate thereof, wherein when administered to a mammal, said compound exhibits preferential accumulation in kidneys, with a ratio for its molar concentration in kidneys compared to that in blood of between about 10 and 500.

23. (canceled)

24. The compound of claim 2, or a pharmaceutically acceptable salt, solvate, or hydrate thereof, wherein when administered to a mammal at a dosing (expressed in molar amount) equal to a standard therapeutic dosing (in molar amount) of an agent (H)ₙR¹, said compound exhibits about 1.5- to 15-fold higher loading (tissue concentration) of agent (H)ₙR¹ in kidneys, as compared to the standard therapeutic dosing of (H)ₙR¹.

25. (canceled)

26. (canceled)

27. A pharmaceutical composition comprising a therapeutically effective amount of a compound of claim 1, or a pharmaceutically acceptable salt, solvate, or hydrate thereof, and a pharmaceutically acceptable carrier.

28. A method for treating a cancer in a mammal in need thereof comprising administering to the mammal a therapeutically effective amount of a compound of claim 1, or a pharmaceutically acceptable salt, solvate, or hydrate thereof.

29. (canceled)

30. (canceled)

31. The compound of claim 2, or a pharmaceutically acceptable salt, solvate, or hydrate thereof, wherein R¹ is selected from the structures below:

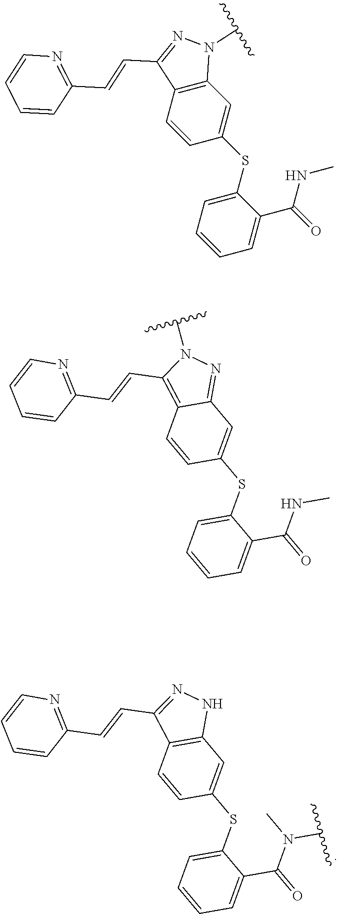 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1 ; *N(C)C(=O)c1ccccc1Sc1ccc2c(/C=C/c3ccccn3)n[nH]c2c1]

32. A pharmaceutical composition comprising a therapeutically effective amount of a compound claim 2, or a pharmaceutically acceptable salt, solvate, or hydrate thereof, and a pharmaceutically acceptable carrier.

33. A method for treating a cancer in a mammal in need thereof comprising administering to the mammal a therapeutically effective amount of a compound of claim 2, or a pharmaceutically acceptable salt, solvate, or hydrate thereof.

34. A method for treating a cancer in a mammal in need thereof comprising administering to the mammal a therapeutically effective amount of a pharmaceutical composition of claim 27, optionally wherein the pharmaceutical composition is administered to the mammal parenterally, transdermally, orally, intranasally, topically, rectally, or via an intra-tumoral administration in a pharmaceutical composition; optionally, wherein the cancer is renal cell carcinoma or metastatic renal cell carcinoma.

35. A method for treating a cancer in a mammal in need thereof comprising administering to the mammal a therapeutically effective amount of a pharmaceutical composition of claim 32, optionally wherein the pharmaceutical composition is administered to the mammal parenterally, transdermally, orally, intranasally, topically, rectally, or via an intra-tumoral administration in a pharmaceutical composition; optionally, wherein the cancer is renal cell carcinoma or metastatic renal cell carcinoma.
